SEQUENCE LISTING (22 sequences)

SEQ ID NO: 1 - DNA, 2271 bp, Homo sapiens
gaattaaagtgcagtaaaatgtccactaggaccccattgccaacggtgaatgaacgagacactgaaaaccacacgtcacatggagatgggcgtcaagaagttacctctcgtaccagccgctcaggagctcggtgtagaaactctatagcctcctgtgcagatgaacaacctcacatcggaaactacagactgttgaaaacaatcggcaaggggaattttgcaaaagtaaaattggcaagacatatccttacaggcagagaggttgcaataaaaataattgacaaaactcagttgaatccaacaagtctacaaaagctcttcagagaagtaagaataatgaagattttaaatcatcccaatatagtgaagttattcgaagtcattgaaaccgaaaaaacactctacctaatcatggaatatgcaagtggaggtgaagtatttgactatttggttgcacatggcaggatgaaggaaaaagaagcaagatctaaatttagacagattgtgtctgcagttcaatactgccatcagaaacggatcgtacatcgagacctcaaggctgaaaatctattgttagatgccgatatgaacattaaaatagcagatttcggttttagcaatgaatttactgttggcggtaaactcgacacgttttgtggcagtcctccatacgcagcacctgagctcttccagggcaagaaatatgacgggccagaagtggatgtgtggagtctgggggtcattttatacacactagtcagtggctcacttccctttgatgggcaaaacctaaaggaactgagagagagagtattaagagggaaatacagaattcccttctacatgtctacagactgtgaaaaccttctcaaacgtttcctggtgctaaatccaattaaacgcggcactctagagcaaatcatgaaggacaggtggatcaatgcagggcatgaagaagatgaactcaaaccatttgttgaaccagagctagacatctcagaccaaaaaagaatagatattatggtgggaatgggatattcacaagaagaaattcaagaatctcttagtaagatgaaatacgatgaaatcacagctacatatttgttattggggagaaaatcttcagagctggatgctagtgattccagttctagcagcaatctttcacttgctaaggttaggccgagcagtgatctcaacaacagtactggccagtctcctcaccacaaagtgcagagaagtgtttcttcaagccaaaagcaaagacgctacagtgaccatgctggaccagctattccttctgttgtggcgtatccgaaaaggagtcagaccagcactgcagatagtgacctcaaagaagatggaatttcctcccggaaatcaagtggcagtgctgttggaggaaagggaattgctccagccagtcccatgcttgggaatgcaagtaatcctaataaggcggatattcctgaacgcaagaaaagctccactgtccctagtagtaacacagcatctggtggaatgacacgacgaaatacttatgtttgcagtgagagaactacagctgatagacactcagtgattcagaatggcaaagaaaacagcactattcctgatcagagaactccagttgcttcaacacacagtatcagtagtgcagccaccccagatcgaatccgcttcccaagaggcactgccagtcgtagcactttccacggccagccccgggaacggcgaaccgcaacatataatggccctcctgcctctcccagcctgtcccatgaagccacaccattgtcccagactcgaagccgaggctccactaatctctttagtaaattaacttcaaaactcacaaggaggcttccaactgaatatgagaggaacgggagatatgagggctcaagtcgcaatgtatctgctgagcaaaaagatgaaaacaaagaagcaaagcctcgatccctacgcttcacctggagcatgaaaaccactagttcaatggatcccggggacatgatgcgggaaatccgcaaagtgttggacgccaataactgcgactatgagcagagggagcgcttcttgctcttctgcgtccacggagatgggcacgcggagaacctcgtgcagtgggaaatggaagtgtgcaagctgccaagactgtctctgaacggggtccggtttaagcggatatcggggacatccatagccttcaaaaatattgcttccaaaattgccaatgagctaaagctgtaacccagtgattatgatgta

SEQ ID NO: 2 - DNA, 2271 bp, Homo sapiens
cttaatttcacgtcattttacaggtgatcctggggtaacggttgccacttacttgctctgtgacttttggtgtgcagtgtacctctacccgcagttcttcaatggagagcatggtcggcgagtcctcgagccacatctttgagatatcggaggacacgtctacttgttggagtgtagcctttgatgtctgacaacttttgttagccgttccccttaaaacgttttcattttaaccgttctgtataggaatgtccgtctctccaacgttatttttattaactgttttgagtcaacttaggttgttcagatgttttcgagaagtctcttcattcttattacttctaaaatttagtagggttatatcacttcaataagcttcagtaactttggcttttttgtgagatggattagtaccttatacgttcacctccacttcataaactgataaaccaacgtgtaccgtcctacttcctttttcttcgttctagatttaaatctgtctaacacagacgtcaagttatgacggtagtctttgcctagcatgtagctctggagttccgacttttagataacaatctacggctatacttgtaattttatcgtctaaagccaaaatcgttacttaaatgacaaccgccatttgagctgtgcaaaacaccgtcaggaggtatgcgtcgtggactcgagaaggtcccgttctttatactgcccggtcttcacctacacacctcagacccccagtaaaatatgtgtgatcagtcaccgagtgaagggaaactacccgttttggatttccttgactctctctctcataattctccctttatgtcttaagggaagatgtacagatgtctgacacttttggaagagtttgcaaaggaccacgatttaggttaatttgcgccgtgagatctcgtttagtacttcctgtccacctagttacgtcccgtacttcttctacttgagtttggtaaacaacttggtctcgatctgtagagtctggttttttcttatctataataccacccttaccctataagtgttcttctttaagttcttagagaatcattctactttatgctactttagtgtcgatgtataaacaataacccctcttttagaagtctcgacctacgatcactaaggtcaagatcgtcgttagaaagtgaacgattccaatccggctcgtcactagagttgttgtcatgaccggtcagaggagtggtgtttcacgtctcttcacaaagaagttcggttttcgtttctgcgatgtcactggtacgacctggtcgataaggaagacaacaccgcataggcttttcctcagtctggtcgtgacgtctatcactggagtttcttctaccttaaaggagggcctttagttcaccgtcacgacaacctcctttcccttaacgaggtcggtcagggtacgaacccttacgttcattaggattattccgcctataaggacttgcgttcttttcgaggtgacagggatcatcattgtgtcgtagaccaccttactgtgctgctttatgaatacaaacgtcactctcttgatgtcgactatctgtgagtcactaagtcttaccgtttcttttgtcgtgataaggactagtctcttgaggtcaacgaagttgtgtgtcatagtcatcacgtcggtggggtctagcttaggcgaagggttctccgtgacggtcagcatcgtgaaaggtgccggtcggggcccttgccgcttggcgttgtatattaccgggaggacggagagggtcggacagggtacttcggtgtggtaacagggtctgagcttcggctccgaggtgattagagaaatcatttaattgaagttttgagtgttcctccgaaggttgacttatactctccttgccctctatactcccgagttcagcgttacatagacgactcgtttttctacttttgtttcttcgtttcggagctagggatgcgaagtggacctcgtacttttggtgatcaagttacctagggcccctgtactacgccctttaggcgtttcacaacctgcggttattgacgctgatactcgtctccctcgcgaagaacgagaagacgcaggtgcctctacccgtgcgcctcttggagcacgtcaccctttaccttcacacgttcgacggttctgacagagacttgccccaggccaaattcgcctatagcccctgtaggtatcggaagtttttataacgaaggttttaacggttactcgatttcgacattgggtcactaatactacat

SEQ ID NO: 3 - protein, 744 aa, Homo sapiens
MSTRTPLPTVNERDTENHTSHGDGRQEVTSRTSRSGARCRNSIASCADEQPHIGNYRLLKTIGKGNFAKVKLARHILTGREVAIKIIDKTQLNPTSLQKLFREVRIMKILNHPNIVKLFEVIETEKTLYLIMEYASGGEVFDYLVAHGRMKEKEARSKFRQIVSAVQYCHQKRIVHRDLKAENLLLDADMNIKIADFGFSNEFTVGGKLDTFCGSPPYAAPELFQGKKYDGPEVDVWSLGVILYTLVSGSLPFDGQNLKELRERVLRGKYRIPFYMSTDCENLLKRFLVLNPIKRGTLEQIMKDRWINAGHEEDELKPFVEPELDISDQKRIDIMVGMGYSQEEIQESLSKMKYDEITATYLLLGRKSSELDASDSSSSSNLSLAKVRPSSDLNNSTGQSPHHKVQRSVSSSQKQRRYSDHAGPAIPSVVAYPKRSQTSTADSDLKEDGISSRKSSGSAVGGKGIAPASPMLGNASNPNKADIPERKKSSTVPSSNTASGGMTRRNTYVCSERTTADRHSVIQNGKENSTIPDQRTPVASTHSISSAATPDRIRFPRGTASRSTFHGQPRERRTATYNGPPASPSLSHEATPLSQTRSRGSTNLFSKLTSKLTRRLPTEYERNGRYEGSSRNVSAEQKDENKEAKPRSLRFTWSMKTTSSMDPGDMMREIRKVLDANNCDYEQRERFLLFCVHGDGHAENLVQWEMEVCKLPRLSLNGVRFKRISGTSIAFKNIASKIANELKL

SEQ ID NO: 4 - DNA, 2112 bp, Homo sapiens
cccagcagtaagtccaacatgattcggggccgcaactcagccacctctgctgatgagcagccccacattggaaactaccggctcctcaagaccattggcaagggtaattttgccaaggtgaagttggcccgacacatcctgactgggaaagaggtagctgtgaagatcattgacaagactcaactgaactcctccagcctccagaaactattccgcgaagtaagaataatgaaggttttgaatcatcccaacatagttaaattatttgaagtgattgagactgagaaaacgctctaccttgtcatggagtacgctagtggcggagaggtatttgattacctagtggctcatggcaggatgaaagaaaaagaggctcgagccaaattccgccagatagtgtctgctgtgcagtactgtcaccagaagtttattgtccatagagacttaaaggcagaaaacctgctcttggatgctgatatgaacatcaagattgcagactttggcttcagcaatgaattcacctttgggaacaagctggacaccttctgtggcagtcccccttatgctgccccagaactcttccagggcaaaaaatatgatggacccgaggtggatgtgtggagcctaggagttatcctctatacactggtcagcggatccctgccttttgatggacagaacctcaaggagctgcgggaacgggtactgaggggaaaataccgtattccattctacatgtccacggactgtgaaaacctgcttaagaaatttctcattcttaatcccagcaagagaggcactttagagcaaatcatgaaagatcgatggatgaatgtgggtcacgaagatgatgaactaaagccttacgtggagccactccctgactacaaggacccccggcggacagagctgatggtgtccatgggttatacacgggaagagatccaggactcgctggtgggccagagatacaacgaggtgatggccacctatctgctcctgggctacaagagctccgagctggaaggcgacaccatcaccctgaaaccccggccttcagctgatctgaccaatagcagcgccccatccccatcccacaaggtacagcgcagcgtgtcggccaatcccaagcagcggcgcttcagcgaccaggctggtcctgccattcccacctctaattcttactctaagaagactcagagtaacaacgcagaaaataagcggcctgaggaggaccgggagtcagggcggaaagccagcagcacagccaaggtgcctgccagccccctgcccggtctggagaggaagaagaccaccccaaccccctccacgaacagcgtcctctccaccagcacaaatcgaagcaggaattccccacttttggagcgggccagcctcggccaggcctccatccagaatggcaaagacagcacagccccccagcgtgtccctgttgcctccccatccgcccacaacatcagcagcagtggtggagccccagaccgaactaacttcccccggggtgtgtccagccgaagcaccttccatgctgggcagctccgacaggtgcgggaccagcagaatttgccctacggtgtgaccccagcctctccctctggccacagccagggccggcggggggcctctgggagcatcttcagcaagttcacctccaagtttgtacgcaggaacctgaatgaacctgaaagcaaagaccgagtggagacgctcagacctcacgtggtgggcagtggcggcaacgacaaagaaaaggaagaatttcgggaggccaagccccgctccctccgcttcacgtggagtatgaagaccacgagctccatggagcccaacgagatgatgcgggagatccgcaaggtgctggacgcgaacagctgccagagcgagctgcatgagaagtacatgctgctgtgcatgcacggcacgccgggccacgaggacttcgtgcagtgggagatggaggtgtgcaaactgccgcggctctctctcaacggggttcgatttaagcggatatcgggcacctccatggccttcaaaaacattgcctccaaaatagccaacgagctgaagctttaacaggctgccaggagcggg

SEQ ID NO: 5 - DNA, 2112 bp, Homo sapiens
gggtcgtcattcaggttgtactaagccccggcgttgagtcggtggagacgactactcgtcggggtgtaacctttgatggccgaggagttctggtaaccgttcccattaaaacggttccacttcaaccgggctgtgtaggactgaccctttctccatcgacacttctagtaactgttctgagttgacttgaggaggtcggaggtctttgataaggcgcttcattcttattacttccaaaacttagtagggttgtatcaatttaataaacttcactaactctgactcttttgcgagatggaacagtacctcatgcgatcaccgcctctccataaactaatggatcaccgagtaccgtcctactttctttttctccgagctcggtttaaggcggtctatcacagacgacacgtcatgacagtggtcttcaaataacaggtatctctgaatttccgtcttttggacgagaacctacgactatacttgtagttctaacgtctgaaaccgaagtcgttacttaagtggaaacccttgttcgacctgtggaagacaccgtcagggggaatacgacggggtcttgagaaggtcccgttttttatactacctgggctccacctacacacctcggatcctcaataggagatatgtgaccagtcgcctagggacggaaaactacctgtcttggagttcctcgacgcccttgcccatgactccccttttatggcataaggtaagatgtacaggtgcctgacacttttggacgaattctttaaagagtaagaattagggtcgttctctccgtgaaatctcgtttagtactttctagctacctacttacacccagtgcttctactacttgatttcggaatgcacctcggtgagggactgatgttcctgggggccgcctgtctcgactaccacaggtacccaatatgtgcccttctctaggtcctgagcgaccacccggtctctatgttgctccactaccggtggatagacgaggacccgatgttctcgaggctcgaccttccgctgtggtagtgggactttggggccggaagtcgactagactggttatcgtcgcggggtaggggtagggtgttccatgtcgcgtcgcacagccggttagggttcgtcgccgcgaagtcgctggtccgaccaggacggtaagggtggagattaagaatgagattcttctgagtctcattgttgcgtcttttattcgccggactcctcctggccctcagtcccgcctttcggtcgtcgtgtcggttccacggacggtcgggggacgggccagacctctccttcttctggtggggttgggggaggtgcttgtcgcaggagaggtggtcgtgtttagcttcgtccttaaggggtgaaaacctcgcccggtcggagccggtccggaggtaggtcttaccgtttctgtcgtgtcggggggtcgcacagggacaacggaggggtaggcgggtgttgtagtcgtcgtcaccacctcggggtctggcttgattgaagggggccccacacaggtcggcttcgtggaaggtacgacccgtcgaggctgtccacgccctggtcgtcttaaacgggatgccacactggggtcggagagggagaccggtgtcggtcccggccgccccccggagaccctcgtagaagtcgttcaagtggaggttcaaacatgcgtccttggacttacttggactttcgtttctggctcacctctgcgagtctggagtgcaccacccgtcaccgccgttgctgtttcttttccttcttaaagccctccggttcggggcgagggaggcgaagtgcacctcatacttctggtgctcgaggtacctcgggttgctctactacgccctctaggcgttccacgacctgcgcttgtcgacggtctcgctcgacgtactcttcatgtacgacgacacgtacgtgccgtgcggcccggtgctcctgaagcacgtcaccctctacctccacacgtttgacggcgccgagagagagttgccccaagctaaattcgcctatagcccgtggaggtaccggaagtttttgtaacggaggttttatcggttgctcgacttcgaaattgtccgacggtcctcgccc

SEQ ID NO: 6 - protein, 691 aa, Homo sapiens
MIRGRNSATSADEQPHIGNYRLLKTIGKGNFAKVKLARHILTGKEVAVKIIDKTQLNSSSLQKLFREVRIMKVLNHPNIVKLFEVIETEKTLYLVMEYASGGEVFDYLVAHGRMKEKEARAKFRQIVSAVQYCHQKFIVHRDLKAENLLLDADMNIKIADFGFSNEFTFGNKLDTFCGSPPYAAPELFQGKKYDGPEVDVWSLGVILYTLVSGSLPFDGQNLKELRERVLRGKYRIPFYMSTDCENLLKKFLILNPSKRGTLEQIMKDRWMNVGHEDDELKPYVEPLPDYKDPRRTELMVSMGYTREEIQDSLVGQRYNEVMATYLLLGYKSSELEGDTITLKPRPSADLTNSSAPSPSHKVQRSVSANPKQRRFSDQAGPAIPTSNSYSKKTQSNNAENKRPEEDRESGRKASSTAKVPASPLPGLERKKTTPTPSTNSVLSTSTNRSRNSPLLERASLGQASIQNGKDSTAPQRVPVASPSAHNISSSGGAPDRTNFPRGVSSRSTFHAGQLRQVRDQQNLPYGVTPASPSGHSQGRRGASGSIFSKFTSKFVRRNLNEPESKDRVETLRPHVVGSGGNDKEKEEFREAKPRSLRFTWSMKTTSSMEPNEMMREIRKVLDANSCQSELHEKYMLLCMHGTPGHEDFVQWEMEVCKLPRLSLNGVRFKRISGTSMAFKNIASKIANELKL

SEQ ID NO: 7 - DNA, 2222 bp, Homo sapiens
tcccttcctccaagcttctcggttccctcccccgagataccggcgccatgtccagcgctcggacccccctacccacgctgaacgagagggacacggagcagcccaccttgggacaccttgactccaagcccagcagtaagtccaacatgattcggggccgcaactcagccacctctgctgatgagcagccccacattggaaactaccggctcctcaagaccattggcaagggtaattttgccaaggtgaagttggcccgacacatcctgactgggaaagaggtagctgtgaagatcattgacaagactcaactgaactcctccagcctccagaaactattccgcgaagtaagaataatgaaggttttgaatcatcccaacatagttaaattatttgaagtgattgagactgagaaaacgctctaccttgtcatggagtacgctagtggcggagaggtatttgattacctagtggctcatggcaggatgaaagaaaaagaggctcgagccaaattccgccagatagtgtctgctgtgcagtactgtcaccagaagtttattgtccatagagacttaaaggcagaaaacctgctcttggatgctgatatgaacatcaagattgcagactttggcttcagcaatgaattcacctttgggaacaagctggacaccttctgtggcagtcccccttatgctgccccagaactcttccagggcaaaaaatatgatggacccgaggtggatgtgtggagcctaggagttatcctctatacactggtcagcggatccctgccttttgatggacagaacctcaaggagctgcgggaacgggtactgaggggaaaataccgtattccattctacatgtccacggactgtgaaaacctgcttaagaaatttctcattcttaatcccagcaagagaggcactttagagcaaatcatgaaagatcgatggatgaatgtgggtcacgaagatgatgaactaaagccttacgtggagccactccctgactacaaggacccccggcggacagagctgatggtgtccatgggttatacacgggaagagatccaggactcgctggtgggccagagatacaacgaggtgatggccacctatctgctcctgggctacaagagctccgagctggaaggcgacaccatcaccctgaaaccccggccttcagctgatctgaccaatagcagcgccccatccccatcccacaaggtacagcgcagcgtgtcggccaatcccaagcagcggcgcttcagcgaccaggctggtcctgccattcccacctctaattcttactctaagaagactcagagtaacaacgcagaaaataagcggcctgaggaggaccgggagtcagggcggaaagccagcagcacagccaaggtgcctgccagccccctgcccggtctggagaggaagaagaccaccccaaccccctccacgaacagcgtcctctccaccagcacaaatcgaagcaggaattccccacttttggagcgggccagcctcggccaggcctccatccagaatggcaaagacagcacagccccccagcgtgtccctgttgcctccccatccgcccacaacatcagcagcagtggtggagccccagaccgaactaacttcccccggggtgtgtccagccgaagcaccttccatgctgggcagctccgacaggtgcgggaccagcagaatttgccctacggtgtgaccccagcctctccctctggccacagccagggccggcggggggcctctgggagcatcttcagcaagttcacctccaagtttgtacgcaggaacctgaatgaacctgaaagcaaagaccgagtggagacgctcagacctcacgtggtgggcagtggcggcaacgacaaagaaaaggaagaatttcgggaggccaagccccgctccctccgcttcacgtggagtatgaagaccacgagctccatggagcccaacgagatgatgcgggagatccgcaaggtgctggacgcgaacagctgccagagcgagctgcatgagaagtacatgctgctgtgcatgcacggcacgccgggccacgaggacttcgtgcagtgggagatggaggtgtgcaaactgccgcggctctctctcaacggggttcgatttaagcggatatcgggcacctccatggccttcaaaaacattgcctccaaaatagccaacgagctgaagctttaa

SEQ ID NO: 8 - DNA, 2222 bp, Homo sapiens
agggaaggaggttcgaagagccaagggagggggctctatggccgcggtacaggtcgcgagcctggggggatgggtgcgacttgctctccctgtgcctcgtcgggtggaaccctgtggaactgaggttcgggtcgtcattcaggttgtactaagccccggcgttgagtcggtggagacgactactcgtcggggtgtaacctttgatggccgaggagttctggtaaccgttcccattaaaacggttccacttcaaccgggctgtgtaggactgaccctttctccatcgacacttctagtaactgttctgagttgacttgaggaggtcggaggtctttgataaggcgcttcattcttattacttccaaaacttagtagggttgtatcaatttaataaacttcactaactctgactcttttgcgagatggaacagtacctcatgcgatcaccgcctctccataaactaatggatcaccgagtaccgtcctactttctttttctccgagctcggtttaaggcggtctatcacagacgacacgtcatgacagtggtcttcaaataacaggtatctctgaatttccgtcttttggacgagaacctacgactatacttgtagttctaacgtctgaaaccgaagtcgttacttaagtggaaacccttgttcgacctgtggaagacaccgtcagggggaatacgacggggtcttgagaaggtcccgttttttatactacctgggctccacctacacacctcggatcctcaataggagatatgtgaccagtcgcctagggacggaaaactacctgtcttggagttcctcgacgcccttgcccatgactccccttttatggcataaggtaagatgtactccacggactgtgaaaacctgcttaagaaatttctcattcttaatcccagcaagagaggcactttactcgtttagtactttctagctacctacttacacccagtgcttctactacttgatttcggaatgcacctcggtgagggactgatgttcctgggggccgcctgtctcgactaccacaggtacccaatatgtgcccttctctaggtcctgagcgaccacccggtctctatgttgctccactaccggtggatagacgaggacccgatgttctcgaggctcgaccttccgctgtggtagtgggactttggggccggaagtcgactagactggttatcgtcgcggggtaggggtagggtgttccatgtcgcgtcgcacagccggttagggttcgtcgccgcgaagtcgctggtccgaccaggacggtaagggtggagattaagaatgagattcttctgagtctcattgttgcgtcttttattcgccggactcctcctggccctcagtcccgcctttcggtcgtcgtgtcggttccacggacggtcgggggacgggccagacctctccttcttctggtggggttgggggaggtgcttgtcgcaggagaggtggtcgtgtttagcttcgtccttaaggggtgaaaacctcgcccggtcggagccggtccggaggtaggtcttaccgtttctgtcgtgtcggggggtcgcacagggacaacggaggggtaggcgggtgttgtagtcgtcgtcaccacctcggggtctggcttgattgaagggggccccacacaggtcggcttcgtggaaggtacgacccgtcgaggctgtccacgccctggtcgtcttaaacgggatgccacactggggtcggagagggagaccggtgtcggtcccggccgccccccggagaccctcgtagaagtcgttcaagtggaggttcaaacatgcgtccttggacttacttggactttcgtttctggctcacctctgcgagtctggagtgcaccacccgtcaccgccgttgctgtttcttttccttcttaaagccctccggttcggggcgagggaggcgaagtgcacctcatacttctggtgctcgaggtacctcgggttgctctactacgccctctaggcgttccacgacctgcgcttgtcgacggtctcgctcgacgtactcttcatgtacgacgacacgtacgtgccgtgcggcccggtgctcctgaagcacgtcaccctctacctccacacgtttgacggcgccgagagagagttgccccaagctaaattcgcctatagcccgtggaggtaccggaagtttttgtaacggaggttttatcggttgctcgacttcgaaatt

SEQ ID NO: 9 - protein, 724 aa, Homo sapiens
MSSARTPLPTLNERDTEQPTLGHLDSKPSSKSNMIRGRNSATSADEQPHIGNYRLLKTIGKGNFAKVKLARHILTGKEVAVKIIDKTQLNSSSLQKLFREVRIMKVLNHPNIVKLFEVIETEKTLYLVMEYASGGEVFDYLVAHGRMKEKEARAKFRQIVSAVQYCHQKFIVHRDLKAENLLLDADMNIKIADFGFSNEFTFGNKLDTFCGSPPYAAPELFQGKKYDGPEVDVWSLGVILYTLVSGSLPFDGQNLKELRERVLRGKYRIPFYMSTDCENLLKKFLILNPSKRGTLEQIMKDRWMNVGHEDDELKPYVEPLPDYKDPRRTELMVSMGYTREEIQDSLVGQRYNEVMATYLLLGYKSSELEGDTITLKPRPSADLTNSSAPSPSHKVQRSVSANPKQRRFSDQAGPAIPTSNSYSKKTQSNNAENKRPEEDRESGRKASSTAKVPASPLPGLERKKTTPTPSTNSVLSTSTNRSRNSPLLERASLGQASIQNGKDSTAPQRVPVASPSAHNISSSGGAPDRTNFPRGVSSRSTFHAGQLRQVRDQQNLPYGVTPASPSGHSQGRRGASGSIFSKFTSKFVRRNLNEPESKDRVETLRPHVVGSGGNDKEKEEFREAKPRSLRFTWSMKTTSSMEPNEMMREIRKVLDANSCQSELHEKYMLLCMHGTPGHEDFVQWEMEVCKLPRLSLNGVRFKRISGTSMAFKNIASKIANELKL

SEQ ID NO: 10 - DNA, 2706 bp, Homo sapiens
tgcccgcacaaaatgtcggcccggacgccattgccgacggtgaacgagcgggacacggaaaatcatacatctgtggatggatatactgaaccacacatccagcctaccaagtcgagtagcagacagaacatcccccggtgtagaaactccattacgtcagcaacagatgaacagcctcacattggaaattaccgtttacaaaaaacaatagggaagggaaattttgccaaagtcaaattggcaagacacgttctaactggtagagaggttgctgtgaaaataatagacaaaactcagctaaatcctaccagtctacaaaagttatttcgagaagtacggataatgaagatactgaatcatcctaatatagtaaaattgtttgaagttattgaaacagagaagactctctatttagtcatggaatacgcgagtgggggtgaagtatttgattacttagttgcccatggaagaatgaaagagaaagaggcccgtgcaaaatttaggcagattgtatctgctgtacagtattgtcatcaaaagtacattgttcaccgtgatcttaaggctgaaaaccttctccttgatggtgatatgaatattaaaattgctgactttggttttagtaatgaatttacagttgggaacaaattggacacattttgtggaagcccaccctatgctgctcccgagcttttccaaggaaagaagtatgatgggcctgaagtggatgtgtggagtctgggcgtcattctctatacattagtcagtggctccttgcctttcgatggccagaatttaaaggaactgcgagagcgagttttacgagggaagtaccgtattcccttctatatgtccacagactgtgaaaatcttctgaagaaattattagtcctgaatccaataaagagaggcagcttggaacaaataatgaaagatcgatggatgaatgttggtcatgaagaggaagaactaaagccatatactgagcctgatccggatttcaatgacacaaaaagaatagacattatggtcaccatgggctttgcacgagatgaaataaatgatgccttaataaatcagaagtatgatgaagttatggctacttatattcttctaggtagaaaaccacctgaatttgaaggtggtgaatcgttatccagtggaaacttgtgtcagaggtcccggcccagtagtgacttaaacaacagcactcttcagtcccctgctcacctgaaggtccagagaagtatctcagcaaatcagaagcagcggcgtttcagtgatcatgctggtccatccattcctcctgctgtatcatataccaaaagacctcaggctaacagtgtggaaagtgaacagaaagaggagtgggacaaagatgtggctcgaaaacttggcagcacaacagttggatcaaaaagcgagatgactgcaagccctcttgtagggccagagaggaaaaaatcttcaactattccaagtaacaatgtgtattctggaggtagcatggcaagaaggaatacatatgtctgtgaaaggaccacagatcgatacgtagcattgcagaatggaaaagacagcagccttacggagatgtctgtgagtagcatatcttctgcaggctcttctgtggcctctgctgtcccctcagcacgaccccgccaccagaagtccatgtccacttctggtcatcctattaaagtcacactgccaaccattaaagacggctctgaagcttaccggcctggtacaacccagagagtgcctgctgcttccccatctgctcacagtattagtactgcgactccagaccggacccgttttccccgagggagctcaagccgaagcactttccatggtgaacagctccgggagcgacgcagcgttgcttataatgggccacctgcttcaccatcccatgaaacgggtgcatttgcacatgccagaaggggaacgtcaactggtataataagcaaaatcacatccaaatttgttcgcagggatccaagtgaaggcgaagccagtggcagaaccgacacctcaagaagtacatcaggggaaccaaaagaaagagacaaggaagagggtaaagattctaagccgcgttctttgcggttcacatggagtatgaagaccactagttcaatggaccctaatgacatgatgagagaaatccgaaaagtgttagatgcaaataactgtgattatgagcaaaaagagagatttttgcttttctgtgtccatggagacgctagacaggatagcctcgtgcagtgggagatggaagtctgcaagttgccacgactgtcacttaatggggttcgcttcaagcgaatatctgggacatctattgcctttaagaacattgcatcaaaaatagcaaatgagcttaagctgtaaagaagtccaaatttacaggttcagggaagatacatacatatatgaggtacagtttttgaatgtactggtaatgcctaatgtggtctgcctgtgaatctccccatgtagaatttgcccttaatgcaataaggttatacatagttatgaactgtaaaattaaagtcagtatgaactataataaatatctgtagcttaaaaagtaggttcacatgtacaggtaagtatattgtgtatttctgttcattttctgttcatagagttgtataataaaacatgattgcttaaaaaaaaaaaaaaaaaaaaaaa

SEQ ID NO: 11 - DNA, 2706 bp, Homo sapiens
acgggcgtgttttacagccgggcctgcggtaacggctgccacttgctcgccctgtgccttttagtatgtagacacctacctatatgacttggtgtgtaggtcggatggttcagctcatcgtctgtcttgtagggggccacatctttgaggtaatgcagtcgttgtctacttgtcggagtgtaacctttaatggcaaatgttttttgttatcccttccctttaaaacggtttcagtttaaccgttctgtgcaagattgaccatctctccaacgacacttttattatctgttttgagtcgatttaggatggtcagatgttttcaataaagctcttcatgcctattacttctatgacttagtaggattatatcattttaacaaacttcaataactttgtctcttctgagagataaatcagtaccttatgcgctcacccccacttcataaactaatgaatcaacgggtaccttcttactttctctttctccgggcacgttttaaatccgtctaacatagacgacatgtcataacagtagttttcatgtaacaagtggcactagaattccgacttttggaagaggaactaccactatacttataattttaacgactgaaaccaaaatcattacttaaatgtcaacccttgtttaacctgtgtaaaacaccttcgggtgggatacgacgagggctcgaaaaggttcctttcttcatactacccggacttcacctacacacctcagacccgcagtaagagatatgtaatcagtcaccgaggaacggaaagctaccggtcttaaatttccttgacgctctcgctcaaaatgctcccttcatggcataagggaagatatacaggtgtctgacacttttagaagacttctttaataatcaggacttaggttatttctctccgtcgaaccttgtttattactttctagctacctacttacaaccagtacttctccttcttgatttcggtatatgactcggactaggcctaaagttactgtgtttttcttatctgtaataccagtggtacccgaaacgtgctctactttatttactacggaattatttagtcttcatactacttcaataccgatgaatataagaagatccatcttttggtggacttaaacttccaccacttagcaataggtcacctttgaacacagtctccagggccgggtcatcactgaatttgttgtcgtgagaagtcaggggacgagtggacttccaggtctcttcatagagtcgtttagtcttcgtcgccgcaaagtcactagtacgaccaggtaggtaaggaggacgacatagtatatggttttctggagtccgattgtcacacctttcacttgtctttctcctcaccctgtttctacaccgagcttttgaaccgtcgtgttgtcaacctagtttttcgctctactgacgttcgggagaacatcccggtctctccttttttagaagttgataaggttcattgttacacataagacctccatcgtaccgttcttccttatgtatacagacactttcctggtgtctagctatgcatcgtaacgtcttaccttttctgtcgtcggaatgcctctacagacactcatcgtatagaagacgtccgagaagacaccggagacgacaggggagtcgtgctggggcggtggtcttcaggtacaggtgaagaccagtaggataatttcagtgtgacggttggtaatttctgccgagacttcgaatggccggaccatgttgggtctctcacggacgacgaaggggtagacgagtgtcataatcatgacgctgaggtctggcctgggcaaaaggggctccctcgagttcggcttcgtgaaaggtaccacttgtcgaggccctcgctgcgtcgcaacgaatattacccggtggacgaagtggtagggtactttgcccacgtaaacgtgtacggtcttccccttgcagttgaccatattattcgttttagtgtaggtttaaacaagcgtccctaggttcacttccgcttcggtcaccgtcttggctgtggagttcttcatgtagtccccttggttttctttctctgttccttctcccatttctaagattcggcgcaagaaacgccaagtgtacctcatacttctggtgatcaagttacctgggattactgtactactctctttaggcttttcacaatctacgtttattgacactaatactcgtttttctctctaaaaacgaaaagacacaggtacctctgcgatctgtcctatcggagcacgtcaccctctaccttcagacgttcaacggtgctgacagtgaattaccccaagcgaagttcgcttatagaccctgtagataacggaaattcttgtaacgtagtttttatcgtttactcgaattcgacatttcttcaggtttaaatgtccaagtcccttctatgtatgtatatactccatgtcaaaaacttacatgaccattacggattacaccagacggacacttagaggggtacatcttaaacgggaattacgttattccaatatgtatcaatacttgacattttaatttcagtcatacttgatattatttatagacatcgaatttttcatccaagtgtacatgtccattcatataacacataaagacaagtaaaagacaagtatctcaacatattattttgtactaacgaattttttttttttttttttttttt

SEQ ID NO: 12 - protein, 795 aa, Homo sapiens
MSARTPLPTVNERDTENHTSVDGYTEPHIQPTKSSSRQNIPRCRNSITSATDEQPHIGNYRLQKTIGKGNFAKVKLARHVLTGREVAVKIIDKTQLNPTSLQKLFREVRIMKILNHPNIVKLFEVIETEKTLYLVMEYASGGEVFDYLVAHGRMKEKEARAKFRQIVSAVQYCHQKYIVHRDLKAENLLLDGDMNIKIADFGFSNEFTVGNKLDTFCGSPPYAAPELFQGKKYDGPEVDVWSLGVILYTLVSGSLPFDGQNLKELRERVLRGKYRIPFYMSTDCENLLKKLLVLNPIKRGSLEQIMKDRWMNVGHEEEELKPYTEPDPDFNDTKRIDIMVTMGFARDEINDALINQKYDEVMATYILLGRKPPEFEGGESLSSGNLCQRSRPSSDLNNSTLQSPAHLKVQRSISANQKQRRFSDHAGPSIPPAVSYTKRPQANSVESEQKEEWDKDVARKLGSTTVGSKSEMTASPLVGPERKKSSTIPSNNVYSGGSMARRNTYVCERTTDRYVALQNGKDSSLTEMSVSSISSAGSSVASAVPSARPRHQKSMSTSGHPIKVTLPTIKDGSEAYRPGTTQRVPAASPSAHSISTATPDRTRFPRGSSSRSTFHGEQLRERRSVAYNGPPASPSHETGAFAHARRGTSTGIISKITSKFVRRDPSEGEASGRTDTSRSTSGEPKERDKEEGKDSKPRSLRFTWSMKTTSSMDPNDMMREIRKVLDANNCDYEQKERFLLFCVHGDARQDSLVQWEMEVCKLPRLSLNGVRFKRISGTSIAFKNIASKIANELKL

SEQ ID NO: 13 - DNA, 25 bp, Artificial Sequence
  Antisense oligonucleotide
cgtatggaggactgccacaaaacgt

SEQ ID NO: 14 - DNA, 25 bp, Artificial Sequence
  Antisense oligonucleotide
tgcaaaacaccgtcaggaggtatgc

SEQ ID NO: 15 - DNA, 24 bp, Artificial Sequence
  Antisense oligonucleotide
tgaggtctgagcgtctccactcgg

SEQ ID NO: 16 - DNA, 24 bp, Artificial Sequence
  Antisense oligonucleotide
ggctcacctctgcgagtctggagt

SEQ ID NO: 17 - DNA, 25 bp, Artificial Sequence
  Antisense oligonucleotide
gagaatgacgcccagactccacaca

SEQ ID NO: 18 - DNA, 25 bp, Artificial Sequence
  Antisense oligonucleotide
acacacctcagacccgcagtaagag

SEQ ID NO: 19 - DNA, 3154 bp, Drosophilia sp.
gcagcgaccatcagctagcgactcctctctgagcgagagagctaatagcttttcagctttagcttttcttgggccaatcggaaattgtatttcattgatgtgaaggagtaccacggatgatagaaacccattgggcatttgactacttttaagcaccgaaacctgaaagactcccgaaaatactcgaatctcacgtgcagaatctctaagaatccctattggactgtttaaaaatatgtcgacagcaatgcgcaccacactgcagtcagttcctgaggccctgccagcggatagcgtgtccaatggcacagcatccaatgtagcagcaccggcggcgccagtatcgagcgcaacaaacgcggtgccaccactggccgccgtctccagcacaaccgccacctacgccaccaactcgatcagcacatcctcgcattcggtcaaggatcagcagcagcaacagcagcagcagcagcatgattcggccaatgcaaacattgtgtcactgccaccaacgacaacgccagtcgccaacactaacacaatgatgcccattgtaacgtcctcgaattcggccaccagcaatagcactgcggccacgcccacgccggcctcgggggcggcagcgacaggtggagtgggatcagtttcccagggtccagcgaccgtttcggcgtcagcggccaacaccaatcactcgcaccagcacagccaccaacaccaccaccatgtggccaacaacatgaccaccgacggtgcccgcttgtccagcaacaattcggcggtggtggcgagctcagcgattaaccaccaccatcaccacacccccggcagtggagtggcgcccaccgtcaacaagaacgtgcttagcacccactcggctcatccctccgcgatcaagcaacgaacctcgtccgccaagggttcgcctaacatgcaaatgcggagtagtgctcctatgcgatggcgtgctactgaggagcatattggcaaatacaaactcataaagacgatcggcaagggcaattttgccaaggtgaaactagcgaaacacctgcccactggcaaggaggtcgccatcaagataattgacaagacccaactcaatcctgggtcactacagaaactctttagagaggttagaataatgaagatgctggatcaccccaacatagttaaattgttccaagtaatcgaaacggagaagacgctctatctgatcatggagtacgcatctggcggagaagtcttcgactacctggttctccacggacgcatgaaggagaaggaggcgcgagttaagtttcgacaaatcgtctcagccgtgcaatattgtcatcaaaaaagaataattcacagggacttaaaagctgaaaaccttttgctggacagcgaactgaacatcaaaatcgctgactttggcttttcgaacgagttcacacccggctcaaagctggacacgttctgcggtagcccgccatatgcggcaccggagctgtttcagggcaaaaagtacgacggaccggaggtcgatgtttggtcgctgggcgtcatcctgtatacgttagtgagcggttccctgcccttcgacggctccaccttgagggagttgcgtgaacgcgtgctcagaggcaaatatagaattcccttctatatgtcgactgactgcgaaaacttgctccgcaaattcttagtactgaatcccgcaaagcgtgctagtctggaaacaatcatgggcgacaagtggatgaacatggggtttgaggaggacgaactcaagccctatattgagcccaaagccgatttagccgatcccaagcggatagaagctctagtcgcgatgggctacaatcgatcggagatcgaggcttcgctctcccaggtgcgctacgacgatgttttcgccacatatttgctgctgggtcgcaagagtacagacccggaaagtgacggatcgcggtctggctcctcgctctcactgcgcaacatctcgggtaatgatgcgggcgccaatgctggtagtgcgagtgttcagagtcccacgcacagaggagtccacaggagcatatcggcgtctagcacgaagccaagtcgccgagcctcgtctggtgcggaaactttgcgtgttggaccgacaaatgcggcagcaacagttgcggcggccacgggagccgttggtgcggttaatccaagcaataactacaatgctgcaggatcagcggcggatcgagcatcagttggcagcaactttaagcgacagaacacaatcgactcggctacgattaaggagaacacagcgcgactggccgctcaaaatcagagacccgcttcggccacacaaaagatgctcaccacggcagacaccacactgaacagtcccgccaagccgcgaacggcaacgaagtacgatccgacgaatggcaatcgcacggtcagcggcacaagtggcatcattccacgtcgctccaccacgctttatgaaaagacttcgtcgacggagaaaaccaacgttattcctgcagagacaaaaatggcatcggctgttaaatcaagcagacactttccaaggaatgttccatcacgttcaacctttcactctggtcaaaccagagcacgaaacaacacagcgctggaatactcgggcaccagcggtgcctccggcgactcctcccatccgggtcgcatgagcttcttctccaaactctcctcacgttttagcaaacggccaaaccagtaattaacaaaacaagcattaactacttcttgttaatagttctaaaactgaaactgaaacaaacgattcccctagagtaaacgcgcgtgacggagaggttcagatatgaacagacagacacagatatggtcgaatccaatcggatcgctcggatcggatcagatcgggaaacgatactgttcacgttgccgttgccgatccgaaatcgctttcgaattccatttcgagttcagatccgtttccggtttcgattcgaaccccttcaaatgaacaccgacaacgttgagttccattgcgttaattgaaatttcacaaatacgcctatgttttattacaattattaactaattatacatataaatttatataaattaaagatacatatacatatatttaaaagtaaagcaaccacaaacagaaatt

SEQ ID NO: 20 - DNA, 3154 bp, Drosophila sp.
cgtcgctggtagtcgatcgctgaggagagactcgctctctcgattatcgaaaagtcgaaatcgaaaagaacccggttagcctttaacataaagtaactacacttcctcatggtgcctactatctttgggtaacccgtaaactgatgaaaattcgtggctttggactttctgagggcttttatgagcttagagtgcacgtcttagagattcttagggataacctgacaaatttttatacagctgtcgttacgcgtggtgtgacgtcagtcaaggactccgggacggtcgcctatcgcacaggttaccgtgtcgtaggttacatcgtcgtggccgccgcggtcatagctcgcgttgtttgcgccacggtggtgaccggcggcagaggtcgtgttggcggtggatgcggtggttgagctagtcgtgtaggagcgtaagccagttcctagtcgtcgtcgttgtcgtcgtcgtcgtcgtactaagccggttacgtttgtaacacagtgacggtggttgctgttgcggtcagcggttgtgattgtgttactacgggtaacattgcaggagcttaagccggtggtcgttatcgtgacgccggtgcgggtgcggccggagcccccgccgtcgctgtccacctcaccctagtcaaagggtcccaggtcgctggcaaagccgcagtcgccggttgtggttagtgagcgtggtcgtgtcggtggttgtggtggtggtacaccggttgttgtactggtggctgccacgggcgaacaggtcgttgttaagccgccaccaccgctcgagtcgctaattggtggtggtagtggtgtgggggccgtcacctcaccgcgggtggcagttgttcttgcacgaatcgtgggtgagccgagtagggaggcgctagttcgttgcttggagcaggcggttcccaagcggattgtacgtttacgcctcatcacgaggatacgctaccgcacgatgactcctcgtataaccgtttatgtttgagtatttctgctagccgttcccgttaaaacggttccactttgatcgctttgtggacgggtgaccgttcctccagcggtagttctattaactgttctgggttgagttaggacccagtgatgtctttgagaaatctctccaatcttattacttctacgacctagtggggttgtatcaatttaacaaggttcattagctttgcctcttctgcgagatagactagtacctcatgcgtagaccgcctcttcagaagctgatggaccaagaggtgcctgcgtacttcctcttcctccgcgctcaattcaaagctgtttagcagagtcggcacgttataacagtagttttttcttattaagtgtccctgaattttcgacttttggaaaacgacctgtcgcttgacttgtagttttagcgactgaaaccgaaaagcttgctcaagtgtgggccgagtttcgacctgtgcaagacgccatcgggcggtatacgccgtggcctcgacaaagtcccgtttttcatgctgcctggcctccagctacaaaccagcgacccgcagtaggacatatgcaatcactcgccaagggacgggaagctgccgaggtggaactccctcaacgcacttgcgcacgagtctccgtttatatcttaagggaagatatacagctgactgacgcttttgaacgaggcgtttaagaatcatgacttagggcgtttcgcacgatcagacctttgttagtacccgctgttcacctacttgtaccccaaactcctcctgcttgagttcgggatataactcgggtttcggctaaatcggctagggttcgcctatcttcgagatcagcgctacccgatgttagctagcctctagctccgaagcgagagggtccacgcgatgctgctacaaaagcggtgtataaacgacgacccagcgttctcatgtctgggcctttcactgcctagcgccagaccgaggagcgagagtgacgcgttgtagagcccattactacgcccgcggttacgaccatcacgctcacaagtctcagggtgcgtgtctcctcaggtgtcctcgtatagccgcagatcgtgcttcggttcagcggctcggagcagaccacgcctttgaaacgcacaacctggctgtttacgccgtcgttgtcaacgccgccggtgccctcggcaaccacgccaattaggttcgttattgatgttacgacgtcctagtcgccgcctagctcgtagtcaaccgtcgttgaaattcgctgtcttgtgttagctgagccgatgctaattcctcttgtgtcgcgctgaccggcgagttttagtctctgggcgaagccggtgtgttttctacgagtggtgccgtctgtggtgtgacttgtcagggcggttcggcgcttgccgttgcttcatgctaggctgcttaccgttagcgtgccagtcgccgtgttcaccgtagtaaggtgcagcgaggtggtgcgaaatacttttctgaagcagctgcctcttttggttgcaataaggacgtctctgtttttaccgtagccgacaatttagttcgtctgtgaaaggttccttacaaggtagtgcaagttggaaagtgagaccagtttggtctcgtgctttgttgtgtcgcgaccttatgagcccgtggtcgccacggaggccgctgaggagggtaggcccagcgtactcgaagaagaggtttgagaggagtgcaaaatcgtttgccggtttggtcattaattgttttgttcgtaattgatgaagaacaattatcaagattttgactttgactttgtttgctaaggggatctcatttgcgcgcactgcctctccaagtctatacttgtctgtctgtgtctataccagcttaggttagcctagcgagcctagcctagtctagccctttgctatgacaagtgcaacggcaacggctaggctttagcgaaagcttaaggtaaagctcaagtctaggcaaaggccaaagctaagcttggggaagtttacttgtggctgttgcaactcaaggtaacgcaattaactttaaagtgtttatgcggatacaaaataatgttaataattgattaatatgtatatttaaatatatttaatttctatgtatatgtatataaattttcatttcgttggtgtttgtctttaa

SEQ ID NO: 21 - protein, 832 aa, Drosophila sp.
MSTAMRTTLQSVPEALPADSVSNGTASNVAAPAAPVSSATNAVPPLAAVSSTTATYATNSISTSSHSVKDQQQQQQQQQHDSANANIVSLPPTTTPVANTNTMMPIVTSSNSATSNSTAATPTPASGAAATGGVGSVSQGPATVSASAANTNHSHQHSHQHHHHVANNMTTDGARLSSNNSAVVASSAINHHHHHTPGSGVAPTVNKNVLSTHSAHPSAIKQRTSSAKGSPNMQMRSSAPMRWRATEEHIGKYKLIKTIGKGNFAKVKLAKHLPTGKEVAIKIIDKTQLNPGSLQKLFREVRIMKMLDHPNIVKLFQVIETEKTLYLIMEYASGGEVFDYLVLHGRMKEKEARVKFRQIVSAVQYCHQKRIIHRDLKAENLLLDSELNIKIADFGFSNEFTPGSKLDTFCGSPPYAAPELFQGKKYDGPEVDVWSLGVILYTLVSGSLPFDGSTLRELRERVLRGKYRIPFYMSTDCENLLRKFLVLNPAKRASLETIMGDKWMNMGFEEDELKPYIEPKADLADPKRIEALVAMGYNRSEIEASLSQVRYDDVFATYLLLGRKSTDPESDGSRSGSSLSLRNISGNDAGANAGSASVQSPTHRGVHRSISASSTKPSRRASSGAETLRVGPTNAAATVAAATGAVGAVNPSNNYNAAGSAADRASVGSNFKRQNTIDSATIKENTARLAAQNQRPASATQKMLTTADTTLNSPAKPRTATKYDPTNGNRTVSGTSGIIPRRSTTLYEKTSSTEKTNVIPAETKMASAVKSSRHFPRNVPSRSTFHSGQTRARNNTALEYSGTSGASGDSSHPGRMSFFSKLSSRFSKRPNQ

SEQ ID NO: 22 - protein, 36 aa, Homo sapiens
QRLQVRKKPQRRKKRAPSMSRTSSYSSITDSTMSLN

(19) United States
(12) Patent Application Publication
(10) Pub. No.: US 2002/0115167 A1
(43) Pub. Date: Aug. 22, 2002

(54) Isolation of drosophila and human polynucleotides encoding PAR-1 kinase, polypeptides encoded by the polynucleotides and methods utilizing the polynucleotides and polypeptides

(76) Inventors: Tian-Qiang Sun, San Francisco, CA (US); Jia-Jia Feng, San Francisco, CA (US); Christoph Reinhard, Alameda, CA (US); Wendy J. Fantl, San Francisco, CA (US); Lewis T. Williams, Mill Valley, CA (US)

Correspondence Address: Chiron Corporation, Intellectual Property R338, P.O. Box 8097, Emeryville, CA, 94662-8097, US

(21) Appl. No.: 09/919,585

(22) Filed: Jul. 30, 2001

Related U.S. Application Data
(60) Provisional application No. 60/221,860, filed on Jul. 28, 2000.

(51) Int. Cl. C12N 9/00; C07H 21/04; C12P 21/02; C12N 5/06

(52) U.S. Cl. 435/183; 435/69.1; 435/325; 435/320.1; 536/23.2

(57) ABSTRACT

Isolated nucleic acid molecules comprising polynucleotide having sequences that encode human and Drosophila PAR-1 kinases. Also provided are proteins and polypeptides encoded by the nucleic acid molecules, methods of modulating PAR-1 expression and function, and methods of modulating the Wnt signaling pathway.

### CROSS REFERENCE TO RELATION APPLICATION

This application claims the benefit of U.S. Provisional Patent Application No. 60/221,860 filed Jul. 28, 2000, where this provisional application is incorporated herein by reference in its entirety.

### BACKGROUND OF THE INVENTION

1. Technical Field

The invention relates to genes encoding proteins involved in the Wnt signaling pathway, to fragments of the proteins, and to methods of using the genes and gene products. More specifically, this invention relates to the discovery of a new effector, a Dishevelled associated kinase referred to as PAR-1, in Drosophila, and to the discovery and cloning of three structural and functional human homologues of PAR-1, referred to as PAR-1 A, PAR-B (α and β), and PAR-1C.

2. Description of Related Art

The Wnt signaling pathway regulates β-catenin-dependent developmental processes through the Dishevelled (Dsh) protein. Dsh was originally identified in Drosophila. Dsh is well conserved in relation to its vertebrate homologs. All Dsh studied to date have three highly conserved domains. An amino-terminal Dishevelled and Axin (DIX) domain, an internal PSD-95/Dlg/ZO-1 (PDZ) domain that has been shown to be a protein-protein interactive domain and a carboxy-terminal disheveled-egl 10-pleckstrin (DEP) domain that has been implicated in G protein signaling. Dsh is also required in the planar polarity pathway in Drosophila, where it activates c-Jun N-Terminal Kinase (JNK). Several lines of evidence indicate that Dsh is differentially recruited into these two different pathways. The third known function of Dsh is that it interacts with Notch, and possibly blocks Notch signaling.

The Wnt pathway plays critical roles in many development processes, such as determination of cell fate, cell polarity and cell proliferation (K. M. Cadigan, R. Nusse, Genes Dev. 11, 3286 (1997)). Aberrant regulation of the Wnt pathway results in oncogenic events in mammals (K. W. Kinzler, B. Vogelstein, Cell 87, 159 (1996); M. Peifer, P. Polakis, Science 287, 1606 (2000)). Wnt interacts with receptors of the Frizzled family to enhance the ability of Dishevelled (Dsh) protein to antagonize the activity of GSK3β. The net effect of this pathway is to stabilize cytosolic β-catenin. β-catenin then translocates to the nucleus and combines with the LEF1/TCF transcription factor to regulate responsive genes such as c-myc and cyclin D1 (K. M. Cadigan, R. Nusse, Genes Dev. 11, 3286 (1997); J. D. Brown, R. T. Moon, Curr. Opin. Cell Biol. 10, 182 (1998); T. C. He et al., Science 281, 1509 (1998); 0. Tetsu, F. McCormick, Nature 398, 422 (1999); M. Shtutman et al., Proc. Natl. Acad. Sci. USA. 96, 5522 (1999)). Although Dsh plays an important role in Wnt signaling, little is known about its mechanism of action (J. Klingensmith, R. Nusse, N. Perrimon, Genes Dev. 8, 118 (1994); H. Theisen et al., Development 120, 347 (1994); S. Y. Sokol, J. Klingensmith, N. Perrimon, K. Itoh, Development 121, 3487 (1995); J. Klingensmith et al., Mech. Dev. 58, 15 (1996)). Accordingly, the identification and isolation of the kinase that phosphorylates Dsh will increase our understanding of the mechanism controlling this signaling pathway and may prove to be an important effector of Dsh function.

### SUMMARY OF THE INVENTION

This invention relates to the discovery of a new effector, a Dishevelled associated kinase referred to as PAR-1, in Drosophila, and to the discovery and cloning of three structural and functional human homologues of PAR-1, referred to as PAR-1A, PAR-B (α and β), and PAR-1C, whose mRNA levels increase in response to Wnt. According to the invention, PAR-1 activates the Wnt pathway and is required for Wnt signaling in mammalian cells.

The kinase activity of the PAR-1 is also stimulated during Wnt signaling. PAR-1 activates the Wnt pathway through its interaction with Dsh in mammalian cells. Suppression of endogenous PAR-1 function inhibits Wnt signaling in mammalian cells and in Xenopus. Importantly, suppression of endogenous PAR-1 significantly reduces the number of colonies of human colon cancer cells. The data indicate a key role of PAR-1 as a positive regulator of the Wnt pathway and in the maintenance of a cancer phenotype.

Accordingly, the invention relates to novel human kinases that associate with the Dishevelled protein, and are referred to as PAR-1.

The invention further relates to four human forms of PAR-1, referred to as PAR-1A, PAR-1Bα, PAR-1Bβ, and PAR-1 C.

The invention still further relates to a Drosophila homolog of PAR-1.

The invention further relates to polynucleotides encoding PAR-1.

The invention also relates to variants and homologs of the polynucleotides encoding PAR-1.

The invention still further relates to proteins sharing the biological function of PAR-1, but having at least one amino acid substitution, addition, or deletion relative to corresponding native PAR-1.

The invention also relates to fragments of PAR-1, wherein the fragments retain at least one biological activity of the native protein.

The invention further relates to antibodies capable of specifically binding to at least one of the proteins PAR-1.

The invention still further relates to a complex comprising a Dishevelled protein or a fragment thereof, and at least one of the proteins PAR-1, or a fragment thereof capable of binding to the Dishevelled protein or fragment of the Dishevelled protein.

The invention also relates to a method of modulating the Wnt pathway using PAR-1.

The invention still further relates to a method of modulating Wnt signaling in a mammalian cell by expressing a variant of PAR-1, in the mammalian cell.

The invention also relates to agonists and antagonists of these PAR-1 proteins, knock-outs of the genes, gene therapy, antisense and ribozymes that target PAR-1 mRNA, and blocking antibodies.

(q) The present invention provides, in one embodiment, an isolated nucleic acid molecule comprising a polynucleotide having a sequence selected from the group consisting of: (a) a sequence encoding amino acids from about 1 to about 744 of SEQ ID NO:3; (b) a sequence encoding amino acids from about 2 to about 744 of SEQ ID NO:3; (c) a sequence encoding amino acids from about 1 to about 691 of SEQ ID NO:6; (d) a sequence encoding amino acids from about 2 to about 691 of SEQ ID NO:6; (e) a sequence encoding amino acids from about 1 to about 724 of SEQ ID NO:9; (f) a sequence encoding amino acids from about 2 to about 724 of SEQ ID NO:9; (g) a sequence encoding amino acids from about 1 to about 795 of SEQ ID NO:12; (h) a sequence encoding amino acids from about 2 to about 795 of SEQ ID NO:12; (i) complements of the sequences of (a)-(h); (j) a sequence having 50-2232 contiguous nucleotides from the coding region of SEQ ID NO:1; (k) a sequence having 50-2073 contiguous nucleotides from the coding region of SEQ ID NO:4; (l) a sequence having 50-2172 contiguous nucleotides from the coding region of SEQ ID NO:7; (m) a sequence having 50-2385 contiguous nucleotides from the coding region of SEQ ID NO:10; (n) sequences having at least 90% identity to the sequences of (a)-(m); (o) sequences having 100-1500 contiguous nucleotides from the coding region of SEQ ID NO:1, SEQ ID NO:4, SEQ ID NO:7 or SEQ ID NO:10; (p) sequences having 500-1000 contiguous nucleotides from the coding region of SEQ ID NO:1, SEQ ID NO:4, SEQ ID NO:7 or SEQ ID NO:10; (q) sequences of (a)-(h), except for at least one amino acid substitution in the encoded amino acid sequence; and (r) sequences of (a)-(h), expect for a conversion of a conserved lysine to an alanine at an ATP binding site of the encoded amino acid sequence.

The invention provides, in another embodiment, an isolated nucleic acid molecule comprising a polynucleotide encoding a polypeptide wherein, except for at least one amino acid substitution, said polypeptide has an amino acid sequence selected from the group consisting of: (a) amino acids from about 1 to about 744 of SEQ ID NO:3; (b) amino acids from about 2 to about 744 of SEQ ID NO:3; (c) amino acids from about 1 to about 691 of SEQ ID NO:6; (d) amino acids from about 2 to about 691 of SEQ ID NO:6;(e) amino acids from about 1 to about 724 of SEQ ID NO:9; (f) amino acids from about 2 to about 724 of SEQ ID NO:9; (g) amino acids from about 1 to about 795 of SEQ ID NO:12; and (h) amino acids from about 2 to about 795 of SEQ ID NO:12. An example of such an amino acid substitution would be to make a conservative amino acid subsitution, whereby the polypeptide retains to same function as the non-substituted polypeptide.

The invention also provides, in another emdodiment, an isolated nucleic acid molecule comprising a polynucleotide encoding a polypeptide wherein, expect for a conversion of a conserved lysine to an alanine at the ATP binding site of said polypeptide, said polypeptide has an amino acid sequence selected from the group consisting of: (a) amino acids from about 1 to about 744 of SEQ ID NO:3; (b) amino acids from about 2 to about 744 of SEQ ID NO:3; (c) amino acids from about 1 to about 691 of SEQ ID NO:6; (d) amino acids from about 2 to about 691 of SEQ ID NO:6;(e) amino acids from about 1 to about 724 of SEQ ID NO:9; (f) amino acids from about 2 to about 724 of SEQ ID NO:9; (g) amino acids from about 1 to about 795 of SEQ ID NO:12; and (h) amino acids from about 2 to about 795 of SEQ ID NO:12.

In another embodiment, the invention provides a method of making a vector comprising by inserting a nucleic acid molecule as described above into a vector in an operable linkage to a promoter, a vector produced by this method, a method of making a host cell comprising introducing the vector into a cell, and a host cell produced by this method.

In still another embodiment, the invention provides a method of making a polypeptide, comprising culturing the host cell under conditions such that the polypeptide is expressed and recovering said polypeptide.

In further embodiments, the invention provides an isolated polypeptide comprising amino acids at least 95% identical to amino acids selected from the group consisting of (a) amino acids from about 1 to about 744 of SEQ ID NO:3; (b) amino acids from about 2 to about 744 of SEQ ID NO:3; (c) amino acids from about 2 to about 691 of SEQ ID NO:6; (d) amino acids from about 2 to about 691 of SEQ ID NO:6; (e) amino acids from about 1 to about 724 of SEQ ID NO:9; (f) amino acids from about 2 to about 724 of SEQ ID NO:9; (g) amino acids from about 1 to about 795 of SEQ ID NO:12; and (h) amino acids from about 2 to about 795 of SEQ ID NO:12.

In another embodiment, the invention provides an isolated polypeptide wherein, expect for at least one conservative amino acid substitution, said polypeptide has an amino acid sequence selected from the group consisting of (a) amino acids from about 2 to about 744 of SEQ ID NO:3; (b) amino acids from about 2 to about 744 of SEQ ID NO:3; (c) amino acids from about 2 to about 691 of SEQ ID NO:6; (d) amino acids from about 2 to about 691 of SEQ ID NO:6;(e) amino acids from about 1 to about 724 of SEQ ID NO:9; (f) amino acids from about 2 to about 724 of SEQ ID NO:9; (g) amino acids from about 1 to about 795 of SEQ ID NO:12; and (h) amino acids from about 2 to about 795 of SEQ ID NO:12.

In a still further embodiment, the invention provides an isolated polypeptide comprising amino acids selected from the group consisting of (a) amino acids from about 1 to about 744 of SEQ ID NO:3; (b) amino acids from about 2 to about 744 of SEQ ID NO:3; (c) amino acids from about 2 to about 691 of SEQ ID NO:6; (d) amino acids from about 2 to about 691 of SEQ ID NO:6;(e) amino acids from about 1 to about 724 of SEQ ID NO:9; (f) amino acids from about 2 to about 724 of SEQ ID NO:9; (g) amino acids from about 2 to about 795 of SEQ ID NO:12; and (h) amino acids from about 2 to about 795 of SEQ ID NO:12.

In another embodiment, the invention provides an isolated polypeptide wherein, expect for a conversion of a conserved lysine to an alanine at the ATP binding site of said polypeptide, said polypeptide has an amino acid sequence selected from the group consisting of (a) amino acids from about 2 to about 744 of SEQ ID NO:3; (b) amino acids from about 2 to about 744 of SEQ ID NO:3; (c) amino acids from about 2 to about 691 of SEQ ID NO:6; (d) amino acids from about 2 to about 691 of SEQ ID NO:6;(e) amino acids from about 1 to about 724 of SEQ ID NO:9; (f) amino acids from about 2 to about 724 of SEQ ID NO:9; (g) amino acids from about 2 to about 795 of SEQ ID NO:12; and (h) amino acids from about 2 to about 795 of SEQ ID NO:12.

The invention also provides, in another embodiment, an epitope-bearing portion of a polypeptide selected from the group consisting of SEQ ID NO:3, SEQ ID NO:6, SEQ ID NO:9, and SEQ ID NO:12. The epitope-bearing portion comprises preferably about 5 to about 50, and more preferably about 10 to about 20, contiguous amino acids.

In another embodiment, the invention provides an isolated antibody that binds to a polypeptide as described above.

In a further embodiment, the invention provides a complex that comprises a polypeptide as described above and a Dishevelled protein.

In a still further embodiment, the invention provides a complex that comprises a fragment of a polypeptide as described above and a Dishevelled protein.

In a further embodiment, the invention provides a method of identifying an inhibitor or enhancer of PAR-1 phosphorylation activity. This method comprises contacting a cell transfected with at least an expression vector encoding Wnt with a candidate inhibitor or enhancer; and detecting an increase or decrease in Dsh/Dvl phosphorylation, wherein a decrease in Dsh/Dvl phosporylation indicates the presence of an inhibitor and an increase in Dsh/Dvl phosphorylation indicates the presence of an enhancer.

In a further embodiment, the invention provides a method of treating a mammal with a disease or disorder associated with a PAR-1 polypeptide, comprising administering to the mammal a composition including a therapeutically effective amount of a polypeptide having an amino sequence at least 95% identity to the amino acid sequence provided in SEQ ID NO:22.

In a still further embodiment, the invention provides a method of treating a mammal with a disease or disorder associated with a PAR-1 polypeptide, comprising administering to the mammal a composition including a therapeutically effective amount of a polynucleotide having a sequence capable of binding a mammalian PAR-1 polynucleotide or complement thereof. Preferably, the polynucleotide is an antisense oligonucleotide or a ribozyme construct. The antisense oligonucleotide can be selected, but not limited to, the group consisting of SEQ ID NO:13, SEQ ID NO:15 and SEQ ID NO:17.

The present also provides, in another embodiment an isolated PAR-1 modulator selected from the group consisting of an antisense oligonucleotide, a ribozyme, a protein, a polypeptide, and a small molecule. An example of a PAR-1 modulator is an antisense molecule or the complement thereof that comprises at least 15 consecutive nucleic acids of the sequence of SEQ ID NO:3, SEQ ID NO:6, SEQ ID NO:9 or SEQ ID NO:12. The antisense molecule or the complement thereof can also be a sequence that hybridizes under high stringency conditions to the at least 15 consecutive nucleic acids of the sequence of SEQ ID NO:3, SEQ ID NO:6, SEQ ID NO:9 or SEQ ID NO:12. The antisense oligonucleotide can also be selected, but not limited to, the group consisting of SEQ ID NO:13, SEQ ID NO:15 and SEQ ID NO:17. Another example of a PAR-1 modulator is an antibody or an antibody fragment. Preferably, the antibody or antibody fragment is a humanized monoclonal. A further example of the PAR-1 modulator is a polypeptide having an amino sequence with at least 95% identity to the amino acid sequence provided in SEQ ID NO:22.

In another embodiment, the invention provides a composition, comprising a therapeutically effective amount of a PAR-1 modulator as described above in a pharmaceutically acceptable carrier. The composition can comprise two or more PAR-1 modulators.

In another embodiment, the invention provides a method of decreasing the expression of PAR-1 in a mammalian cell, comprising administering to the cell, a PAR-1modulator as described above. The PAR-1 modulator can be administered ex vivo to the mammalian cell.

In a still further embodiment, the invention provides a method of treating neoplastic disease. This method comprises administering to a mammalian cell a PAR-1modulator as described above such that said neoplastic disease is reduced in severity.

### DETAILED DESCRIPTION OF THE INVENTION

The term “biologically equivalent” is intended to mean that the compositions of the present invention are capable of demonstrating some or all of the same biological properties in a similar fashion, not necessarily to the same degree as the PAR-1 isolated as described herein or recombinantly produced human PAR-1 of the invention.

Sequence identity or percent identity is intended to mean the percentage of same residues between two sequences, when the two sequences are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NDRF, Washington, Vol. 5, suppl. 3, p. 345, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human PAR-1 when determining percent conservation with non-human PAR-1, and referenced to PAR-1 when determining percent conservation with non-PAR-1 Dishevelled-associated proteins. Conservative amino acid changes satisfying this requirement are: R-K; E-D, Y-F, L-M; V-I, Q-H.

Polypeptide Fragments

The invention provides polypeptide fragments of PAR-1. Polypeptide fragments of the invention can comprise at least 8, 9, 10, 12, 15, 18, 19, 20, 25, 50, 75, 100, 125, 130, 140, 150, 160, 170, 180, 190, 200, 250, 300, 350, 400, 450, 500, 550, 600, 650, 675, 700, 750 or 795 contiguous amino acids selected from SEQ ID NO:3, 6, 9, 12 or 21. Also included are all intermediate length fragments in this range, such as 101, 102, 103, etc.; 170, 171, 172, etc.; and 710, 711, 712, etc., which are exemplary only and not limiting. These polypeptide fragments are useful in vaccines, to raise antibodies against or as building blocks of the protein.

Exemplary polypeptides include the following 9-mer polypeptide of the 744 amino acid residues of SEQ ID NO:3: 1-9, 2-10, 3-11, 4-12, 5-13, 6-14, 7-15, 8-16, 9-17, 10-18, 11-19, 12-20, 13-21, 14-22, 15-23, 16-24, 17-25, 18-26, 19-27, 20-28, 21-29, 22-30, 23-31, 24-32, 25-33, 26-34, 27-35, 28-36, 29-37, 30-38, 31-39, 32-40, 33-41, 34-42, 35-43, 36-44, 37-45, 38-46, 39-47, 40-48, 41-49, 42-50, 43-51, 44-52, 45-53, 46-54, 47-55, 48-56, 49-57, 50-58, 51-59, 52-60, 53-61, 54-62, 55-63, 56-64, 57-65, 58-66, 59-67, 60-68, 61-69, 62-70, 63-71, 64-72, 65-73, 66-74, 67-75, 68-76, 69-77, 70-78, 71-79, 72-80, 73-81, 74-82, 75-83, 76-84, 77-85, 78-86, 79-87, 80-88, 81-89, 82-90, 83-91, 84-92, 85-93, 86-94, 87-95, 88-96, 89-97, 90-98, 91-99, 92-100, 93-101, 94-102, 95-103, 96-104, 97-105, 98-106, 99-107, 100-108, 101-109, 102-110, 103-111, 104-112, 105-113, 106-114, 107-115, 108-116, 109-117, 110-118, 111-119, 112-120, 113-121, 114-122, 115-123, 116-124, 117-125, 118-126, 119-127, 120-128, 121-129, 122-130, 123-131, 124-132, 125-133, 126-134, 127-135, 128-136, 129-137, 130-138, 131-139, 132-140, 133-141, 134-142, 135-143, 136-144, 137-145, 138-146, 139-147, 140-148, 141-149, 142-150, 143-151, 144-152, 145-153, 146-154, 147-155, 148-156, 149-157, 150-158, 151-159, 152-160, 153-161, 154-162, 155-163, 156-164, 157-165, 158-166, 159-167, 160-168, 161-169, 162-170, 163-171, 164-172, 165-173, 166-174, 167-175, 168-176, 169-177, 170-178, 171-179, 172-180, 173-181, 174-182, 175-183, 176-184, 177-185, 178-186, 179-187, 180-188, 181-189, 182-190, 183-191, 184-192, 185-193, 186-194, 187-195, 188-196, 189-197, 190-198, 191-199, 192-200, 193-201, 194-202, 195-203, 196-204, 197-205, 198-206, 199-207, 200-208, 201-209, 202-210, 203-211, 204-212, 205-213, 206-214, 207-215, 208-216, 209-217, 210-218, 211-219, 212-220, 213-221, 214-222, 215-223, 216-224, 217-225, 218-226, 219-227, 220-228, 221-229, 222-230, 223-231, 224-232, 225-233, 226-234, 227-235, 228-236, 229-237, 230-238, 231-239, 232-240, 233-241, 234-242, 235-243, 236-244, 237-245, 238-246, 239-247, 240-248, 241-249, 242-250, 243-251, 244-252, 245-253, 246-254, 247-255, 248-256, 249-257, 250-258, 251-259, 252-260, 253-261, 254-262, 255-263, 256-264, 257-265, 258-266, 259-267, 260-268, 261-269, 262-270, 263-271, 264-272, 265-273, 266-274, 267-275, 268-276, 269-277, 270-278, 271-279, 272-280, 273-281, 274-282, 275-283, 276-284, 277-285, 278-286, 279-287, 280-288, 281-289, 282-290, 283-291, 284-292, 285-293, 286-294, 287-295, 288-296, 289-297, 290-298, 291-299, 292-300, 293-301, 294-302, 295-303, 296-304, 297-305, 298-306, 299-307, 300-308, 301-309, 302-310, 303-311, 304-312, 305-313, 306-314, 307-315, 308-316, 309-317, 310-318, 311-319, 312-320, 313-321, 314-322, 315-323, 316-324, 317-325, 318-326, 319-327, 320-328, 321-329, 322-330, 323-331, 324-332, 325-333, 326-334, 327-335, 328-336, 329-337, 330-338, 331-339, 332-340, 333-341, 334-342, 335-343, 336-344, 337-345, 338-346, 339-347, 340-348, 341-349, 342-350, 343-351, 344-352, 345-353, 346-354, 347-355, 348-356, 349-357, 350-358, 351-359, 352-360, 353-361, 354-362, 355-363, 356-364, 357-365, 358-366, 359-367, 360-368, 361-369, 362-370, 363-371, 364-372, 365-373, 366-374, 367-375, 368-376, 369-377, 370-378, 371-379, 372-380, 373-381, 374-382, 375-383, 376-384, 377-385, 378-386, 379-387, 380-388, 381-389, 382-390, 383-391, 384-392, 385-393, 386-394, 387-395, 388-396, 389-397, 390-398, 391-399, 392-400, 393-401, 394-402, 395-403, 396-404, 397-405, 398-406, 399-407, 400-408, 401-409, 402-410, 403-411, 404-412, 405-413, 406-414, 407-415, 408-416, 409-417, 410-418, 411-419, 412-420, 413-421, 414-422, 415-423, 416-424, 417-425, 418-426, 419-427, 420-428, 421-429, 422-430, 423-431, 424-432, 425-433, 426-434, 427-435, 428-436, 429-437, 430-438, 431-439, 432-440, 433-441, 434-442, 435-443, 436-444, 437-445, 438-446, 439-447, 440-448, 441-449, 442-450, 443-451, 444-452, 445-453, 446-454, 447-455, 448-456, 449-457, 450-458, 451-459, 452-460, 453-461, 454-462, 455-463, 456-464, 457-465, 458-466, 459-467, 460-468, 461-469, 462-470, 463-471, 464-472, 465-473, 466-474, 467-475, 468-476, 469-477, 470-478, 471-479, 472-480, 473-481, 474-482, 475-483, 476-484, 477-485, 478-486, 479-487, 480-488, 481-489, 482-490, 483-491, 484-492, 485-493, 486-494, 487-495, 488-496, 489-497, 490-498, 491-499, 492-500, 493-501, 494-502, 495-503, 496-504, 497-505, 498-506, 499-507, 500-508, 501-509, 502-510, 503-511, 504-512, 505-513, 506-514, 507-515, 508-516, 509-517, 510-518, 511-519, 512-520, 513-521, 514-522, 515-523, 516-524, 517-525, 518-526, 519-527, 520-528, 521-529, 522-530, 523-531, 524-532, 525-533, 526-534, 527-535, 528-536, 529-537, 530-538, 531-539, 532-540, 533-541, 534-542, 535-543, 536-544, 537-545, 538-546, 539-547, 540-548, 541-549, 542-550, 543-551, 544-552, 545-553, 546-554, 547-555, 548-556, 549-557, 550-558, 551-559, 552-560, 553-561, 554-562, 555-563, 556-564, 557-565, 558-566, 559-567, 560-568, 561-569, 562-570, 563-571, 564-572, 565-573, 566-574, 567-575, 568-576, 569-577, 570-578, 571-579, 572-580, 573-581, 574-582, 575-583, 576-584, 577-585, 578-586, 579-587, 580-588, 581-589, 582-590, 583-591, 584-592, 585-593, 586-594, 587-595, 588-596, 589-597, 590-598, 591-599, 592-600, 593-601, 594-602, 595-603, 596-604, 597-605, 598-606, 599-607, 600-608, 601-609, 602-610, 603-611, 604-612, 605-613, 606-614, 607-615, 608-616, 609-617, 610-618, 611-619, 612-620, 613-621, 614-622, 615-623, 616-624, 617-625, 618-626, 619-627, 620-628, 621-629, 622-630, 623-631, 624-632, 625-633, 626-634, 627-635, 628-636, 629-637, 630-638, 631-639, 632-640, 633-641, 634-642, 635-643, 636-644, 637-645, 638-646, 639-647, 640-648, 641-649, 642-650, 643-651, 644-652, 645-653, 646-654, 647-655, 648-656, 649-657, 650-658, 651-659, 652-660, 653-661, 654-662, 655-663, 656-664, 657-665, 658-666, 659-667, 660-668, 661-669, 662-670, 663-671, 664-672, 665-673, 666-674, 667-675, 668-676, 669-677, 670-678, 671-679, 672-680, 673-681, 674-682, 675-683, 676-684, 677-685, 678-686, 679-687, 680-688, 681-689, 682-690, 683-691, 684-692, 685-693, 686-694, 687-695, 688-696, 689-697, 690-698, 691-699, 692-700, 693-701, 694-702, 695-703, 696-704, 697-705, 698-706, 699-707, 700-708, 701-709, 702-710, 703-711, 704-712, 705-713, 706-714, 707-715, 708-716, 709-717, 710-718, 711-719, 712-720, 713-721, 714-722, 715-723, 716-724, 717-725, 718-726, 719-727, 720-728, 721-729, 722-730, 723-731, 724-732, 725-733, 726-734, 727-735, 728-736, 729-737, 730-738, 731-739, 732-740, 733-741, 734-742, 735-743 and 736-744.

Exemplary polypeptides include the following 12-mer polypeptide of the 744 amino acid residues of SEQ ID NO:3: 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, 22-33, 23-34, 24-35, 25-36, 26-37, 27-38, 28-39, 29-40, 30-41, 31-42, 32-43, 33-44, 34-45, 35-46, 36-47, 37-48, 38-49, 39-50, 40-51, 41-52, 42-53, 43-54, 44-55, 45-56, 46-57, 47-58, 48-59, 49-60, 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723, 713-724, 714-725, 715-726, 716-727, 717-728, 718-729, 719-730, 720-731, 721-732, 722-733, 723-734, 724-735, 725-736, 726-737, 727-738, 728-739, 729-740, 730-741, 731-742, 732-743 and 733-744.

Exemplary polypeptides include the following 15-mer polypeptide of the 744 amino acid residues of SEQ ID NO:3: 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 46-60, 47-61, 48-62, 49-63, 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723, 710-724, 711-725, 712-726, 713-727, 714-728, 715-729, 716-730, 717-731, 718-732, 719-733, 720-734, 721-735, 722-736, 723-737, 724-738, 725-739, 726-740, 727-741, 728-742, 729-743 and 730-744.

Exemplary polypeptides include the following 20-mer polypeptide of the 744 amino acid residues of SEQ ID NO:3: 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743 and 725-744.

Exemplary polypeptides include the following 25-mer polypeptide of the 744 amino acid residues of SEQ ID NO:3: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-24, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487. 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743 and 720-744.

Exemplary polypeptides include the following 9-mer polypeptide of the 691 amino acid residues of SEQ ID NO:6: 1-9, 2-10, 3-11, 4-12, 5-13, 6-14, 7-15, 8-16, 9-17, 10-18, 11-19, 12-20, 13-21, 14-22, 15-23, 16-24, 17-25, 18-26, 19-27, 20-28, 21-29, 22-30, 23-31, 24-32, 25-33, 26-34, 27-35, 28-36, 29-37, 30-38, 31-39, 32-40, 33-41, 34-42, 35-43, 36-44, 37-45, 38-46, 39-47, 40-48, 41-49, 42-50, 43-51, 44-52, 45-53, 46-54, 47-55, 48-56, 49-57, 50-58, 51-59, 52-60, 53-61, 54-62, 55-63, 56-64, 57-65, 58-66, 59-67, 60-68, 61-69, 62-70, 63-71, 64-72, 65-73, 66-74, 67-75, 68-76, 69-77, 70-78, 71-79, 72-80, 73-81, 74-82, 75-83, 76-84, 77-85, 78-86, 79-87, 80-88, 81-89, 82-90, 83-91, 84-92, 85-93, 86-94, 87-95, 88-96, 89-97, 90-98, 91-99, 92-100, 93-101, 94-102, 95-103, 96-104, 97-105, 98-106, 99-107, 100-108, 101-109, 102-110, 103-111, 104-112, 105-113, 106-114, 107-115, 108-116, 109-117, 110-118, 111-119, 112-120, 113-121, 114-122, 115-123, 116-124, 117-125, 118-126, 119-127, 120-128, 121-129, 122-130, 123-131, 124-132, 125-133, 126-134, 127-135, 128-136, 129-137, 130-138, 131-139, 132-140, 133-141, 134-142, 135-143, 136-144, 137-145, 138-146, 139-147, 140-148, 141-149, 142-150, 143-151, 144-152, 145-153, 146-154, 147-155, 148-156, 149-157, 150-158, 151-159, 152-160, 153-161, 154-162, 155-163, 156-164, 157-165, 158-166, 159-167, 160-168, 161-169, 162-170, 163-171, 164-172, 165-173, 166-174, 167-175, 168-176, 169-177, 170-178, 171-179, 172-180, 173-181, 174-182, 175-183, 176-184, 177-185, 178-186, 179-187, 180-188, 181-189, 182-190, 183-191, 184-192, 185-193, 186-194, 187-195, 188-196, 189-197, 190-198, 191-199, 192-200, 193-201, 194-202, 195-203, 196-204, 197-205, 198-206, 199-207, 200-208, 201-209, 202-210, 203-211, 204-212, 205-213, 206-214, 207-215, 208-216, 209-217, 210-218, 211-219, 212-220, 213-221, 214-222, 215-223, 216-224, 217-225, 218-226, 219-227, 220-228, 221-229, 222-230, 223-231, 224-232, 225-233, 226-234, 227-235, 228-236, 229-237, 230-238, 231-239, 232-240, 233-241, 234-242, 235-243, 236-244, 237-245, 238-246, 239-247, 240-248, 241-249, 242-250, 243-251, 244-252, 245-253, 246-254, 247-255, 248-256, 249-257, 250-258, 251-259, 252-260, 253-261, 254-262, 255-263, 256-264, 257-265, 258-266, 259-267, 260-268, 261-269, 262-270, 263-271, 264-272, 265-273, 266-274, 267-275, 268-276, 269-277, 270-278, 271-279, 272-280, 273-281, 274-282, 275-283, 276-284, 277-285, 278-286, 279-287, 280-288, 281-289, 282-290, 283-291, 284-292, 285-293, 286-294, 287-295, 288-296, 289-297, 290-298, 291-299, 292-300, 293-301, 294-302, 295-303, 296-304, 297-305, 298-306, 299-307, 300-308, 301-309, 302-310, 303-311, 304-312, 305-313, 306-314, 307-315, 308-316, 309-317, 310-318, 311-319, 312-320, 313-321, 314-322, 315-323, 316-324, 317-325, 318-326, 319-327, 320-328, 321-329, 322-330, 323-331, 324-332, 325-333, 326-334, 327-335, 328-336, 329-337, 330-338, 331-339, 332-340, 333-341, 334-342, 335-343, 336-344, 337-345, 338-346, 339-347, 340-348, 341-349, 342-350, 343-351, 344-352, 345-353, 346-354, 347-355, 348-356, 349-357, 350-358, 351-359, 352-360, 353-361, 354-362, 355-363, 356-364, 357-365, 358-366, 359-367, 360-368, 361-369, 362-370, 363-371, 364-372, 365-373, 366-374, 367-375, 368-376, 369-377, 370-378, 371-379, 372-380, 373-381, 374-382, 375-383, 376-384, 377-385, 378-386, 379-387, 380-388, 381-389, 382-390, 383-391, 384-392, 385-393, 386-394, 387-395, 388-396, 389-397, 390-398, 391-399, 392-400, 393-401, 394-402, 395-403, 396-404, 397-405, 398-406, 399-407, 400-408, 401-409, 402-410, 403-411, 404-412, 405-413, 406-414, 407-415, 408-416, 409-417, 410-418, 411-419, 412-420, 413-421, 414-422, 415-423, 416-424, 417-425, 418-426, 419-427, 420-428, 421-429, 422-430, 423-431, 424-432, 425-433, 426-434, 427-435, 428-436, 429-437, 430-438, 431-439, 432-440, 433-441, 434-442, 435-443, 436-444, 437-445, 438-446, 439-447, 440-448, 441-449, 442-450, 443-451, 444-452, 445-453, 446-454, 447-455, 448-456, 449-457, 450-458, 451-459, 452-460, 453-461, 454-462, 455-463, 456-464, 457-465, 458-466, 459-467, 460-468, 461-469, 462-470, 463-471, 464-472, 465-473, 466-474, 467-475, 468-476, 469-477, 470-478, 471-479, 472-480, 473-481, 474-482, 475-483, 476-484, 477-485, 478-486, 479-487, 480-488, 481-489, 482-490, 483-491, 484-492, 485-493, 486-494, 487-495, 488-496, 489-497, 490-498, 491-499, 492-500, 493-501, 494-502, 495-503, 496-504, 497-505, 498-506, 499-507, 500-508, 501-509, 502-510, 503-511, 504-512, 505-513, 506-514, 507-515, 508-516, 509-517, 510-518, 511-519, 512-520, 513-521, 514-522, 515-523, 516-524, 517-525, 518-526, 519-527, 520-528, 521-529, 522-530, 523-531, 524-532, 525-533, 526-534, 527-535, 528-536, 529-537, 530-538, 531-539, 532-540, 533-541, 534-542, 535-543, 536-544, 537-545, 538-546, 539-547, 540-548, 541-549, 542-550, 543-551, 544-552, 545-553, 546-554, 547-555, 548-556, 549-557, 550-558, 551-559, 552-560, 553-561, 554-562, 555-563, 556-564, 557-565, 558-566, 559-567, 560-568, 561-569, 562-570, 563-571, 564-572, 565-573, 566-574, 567-575, 568-576, 569-577, 570-578, 571-579, 572-580, 573-581, 574-582, 575-583, 576-584, 577-585, 578-586, 579-587, 580-588, 581-589, 582-590, 583-591, 584-592, 585-593, 586-594, 587-595, 588-596, 589-597, 590-598, 591-599, 592-600, 593-601, 594-602, 595-603, 596-604, 597-605, 598-606, 599-607, 600-608, 601-609, 602-610, 603-611, 604-612, 605-613, 606-614, 607-615, 608-616, 609-617, 610-618, 611-619, 612-620, 613-621, 614-622, 615-623, 616-624, 617-625, 618-626, 619-627, 620-628, 621-629, 622-630, 623-631, 624-632, 625-633, 626-634, 627-635, 628-636, 629-637, 630-638, 631-639, 632-640, 633-641, 634-642, 635-643, 636-644, 637-645, 638-646, 639-647, 640-648, 641-649, 642-650, 643-651, 644-652, 645-653, 646-654, 647-655, 648-656, 649-657, 650-658, 651-659, 652-660, 653-661, 654-662, 655-663, 656-664, 657-665, 658-666, 659-667, 660-668, 661-669, 662-670, 663-671, 664-672, 665-673, 666-674, 667-675, 668-676, 669-677, 670-678, 671-679, 672-680, 673-681, 674-682, 675-683, 676-684, 677-685, 678-686, 679-687, 680-688, 681-689, 682-690 and 683-691.

Exemplary polypeptides include the following 12-mer polypeptide of the 691 amino acid residues of SEQ ID NO:6: 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, 22-33, 23-34, 24-35, 25-36, 26-37, 27-38, 28-39, 29-40, 30-41, 31-42, 32-43, 33-44, 34-45, 35-46, 36-47, 37-48, 38-49, 39-50, 40-51, 41-52, 42-53, 43-54, 44-55, 45-56, 46-57, 47-58, 48-59, 49-60, 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690 and 680-691.

Exemplary polypeptides include the following 15-mer polypeptide of the 691 amino acid residues of SEQ ID NO:6: 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 46-60, 47-61, 48-62, 49-63, 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690 and 677-691.

Exemplary polypeptides include the following 20-mer polypeptide of the 691 amino acid residues of SEQ ID NO:6: 1-20, 2-21, 3-24-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690 and 672-691.

Exemplary polypeptides include the following 25-mer polypeptide of the 691 amino acid residues of SEQ ID NO:6: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690 and 667-691.

Exemplary polypeptides include the following 9-mer polypeptide of the 724 amino acid residues of SEQ ID NO:9: 1-9, 2-10, 3-11, 4-12, 5-13, 6-14, 7-15, 8-16, 9-17, 10-18, 11-19, 12-20, 13-21, 14-22, 15-23, 16-24, 17-25, 18-26, 19-27, 20-28, 21-29, 22-30, 23-31, 24-32, 25-33, 26-34, 27-35, 28-36, 29-37, 30-38, 31-39, 32-40, 33-41, 34-42, 35-43, 36-44, 37-45, 38-46, 39-47, 40-48, 41-49, 42-50, 43-51, 44-52, 45-53, 46-54, 47-55, 48-56, 49-57, 50-58, 51-59, 52-60, 53-61, 54-62, 55-63, 56-64, 57-65, 58-66, 59-67, 60-68, 61-69, 62-70, 63-71, 64-72, 65-73, 66-74, 67-75, 68-76, 69-77, 70-78, 71-79, 72-80, 73-81, 74-82, 75-83, 76-84, 77-85, 78-86, 79-87, 80-88, 81-89, 82-90, 83-91, 84-92, 85-93, 86-94, 87-95, 88-96, 89-97, 90-98, 91-99, 92-100, 93-101, 94-102, 95-103, 96-104, 97-105, 98-106, 99-107, 100-108, 101-109, 102-110, 103-111, 104-112, 105-113, 106-114, 107-115, 108-116, 109-117, 110-118, 111-119, 112-120, 113-121, 114-122, 115-123, 116-124, 117-125, 118-126, 119-127, 120-128, 121-129, 122-130, 123-131, 124-132, 125-133, 126-134, 127-135, 128-136, 129-137, 130-138, 131-139, 132-140, 133-141, 134-142, 135-143, 136-144, 137-145, 138-146, 139-147, 140-148, 141-149, 142-150, 143-151, 144-152, 145-153, 146-154, 147-155, 148-156, 149-157, 150-158, 151-159, 152-160, 153-161, 154-162, 155-163, 156-164, 157-165, 158-166, 159-167, 160-168, 161-169, 162-170, 163-171, 164-172, 165-173, 166-174, 167-175, 168-176, 169-177, 170-178, 171-179, 172-180, 173-181, 174-182, 175-183, 176-184, 177-185, 178-186, 179-187, 180-188, 181-189, 182-190, 183-191, 184-192, 185-193, 186-194, 187-195, 188-196, 189-197, 190-198, 191-199, 192-200, 193-201, 194-202, 195-203, 196-204, 197-205, 198-206, 199-207, 200-208, 201-209, 202-210, 203-211, 204-212, 205-213, 206-214, 207-215, 208-216, 209-217, 210-218, 211-219, 212-220, 213-221, 214-222, 215-223, 216-224, 217-225, 218-226, 219-227, 220-228, 221-229, 222-230, 223-231, 224-232, 225-233, 226-234, 227-235, 228-236, 229-237, 230-238, 231-239, 232-240, 233-241, 234-242, 235-243, 236-244, 237-245, 238-246, 239-247, 240-248, 241-249, 242-250, 243-251, 244-252, 245-253, 246-254, 247-255, 248-256, 249-257, 250-258, 251-259, 252-260, 253-261, 254-262, 255-263, 256-264, 257-265, 258-266, 259-267, 260-268, 261-269, 262-270, 263-271, 264-272, 265-273, 266-274, 267-275, 268-276, 269-277, 270-278, 271-279, 272-280, 273-281, 274-282, 275-283, 276-284, 277-285, 278-286, 279-287, 280-288, 281-289, 282-290, 283-291, 284-292, 285-293, 286-294, 287-295, 288-296, 289-297, 290-298, 291-299, 292-300, 293-301, 294-302, 295-303, 296-304, 297-305, 298-306, 299-307, 300-308, 301-309, 302-310, 303-311, 304-312, 305-313, 306-314, 307-315, 308-316, 309-317, 310-318, 311-319, 312-320, 313-321, 314-322, 315-323, 316-324, 317-325, 318-326, 319-327, 320-328, 321-329, 322-330, 323-331, 324-332, 325-333, 326-334, 327-335, 328-336, 329-337, 330-338, 331-339, 332-340, 333-341, 334-342, 335-343, 336-344, 337-345, 338-346, 339-347, 340-348, 341-349, 342-350, 343-351, 344-352, 345-353, 346-354, 347-355, 348-356, 349-357, 350-358, 351-359, 352-360, 353-361, 354-362, 355-363, 356-364, 357-365, 358-366, 359-367, 360-368, 361-369, 362-370, 363-371, 364-372, 365-373, 366-374, 367-375, 368-376, 369-377, 370-378, 371-379, 372-380, 373-381, 374-382, 375-383, 376-384, 377-385, 378-386, 379-387, 380-388, 381-389, 382-390, 383-391, 384-392, 385-393, 386-394, 387-395, 388-396, 389-397, 390-398, 391-399, 392-400, 393-401, 394-402, 395-403, 396-404, 397-405, 398-406, 399-407, 400-408, 401-409, 402-410, 403-411, 404-412, 405-413, 406-414, 407-415, 408-416, 409-417, 410-418, 411-419, 412-420, 413-421, 414-422, 415-423, 416-424, 417-425, 418-426, 419-427, 420-428, 421-429, 422-430, 423-431, 424-432, 425-433, 426-434, 427-435, 428-436, 429-437, 430-438, 431-439, 432-440, 433-441, 434-442, 435-443, 436-444, 437-445, 438-446, 439-447, 440-448, 441-449, 442-450, 443-451, 444-452, 445-453, 446-454, 447-455, 448-456, 449-457, 450-458, 451-459, 452-460, 453-461, 454-462, 455-463, 456-464, 457-465, 458-466, 459-467, 460-468, 461-469, 462-470, 463-471, 464-472, 465-473, 466-474, 467-475, 468-476, 469-477, 470-478, 471-479, 472-480, 473-481, 474-482, 475-483, 476-484, 477-485, 478-486, 479-487, 480-488, 481-489, 482-490, 483-491, 484-492, 485-493, 486-494, 487-495, 488-496, 489-497, 490-498, 491-499, 492-500, 493-501, 494-502, 495-503, 496-504, 497-505, 498-506, 499-507, 500-508, 501-509, 502-510, 503-511, 504-512, 505-513, 506-514, 507-515, 508-516, 509-517, 510-518, 511-519, 512-520, 513-521, 514-522, 515-523, 516-524, 517-525, 518-526, 519-527, 520-528, 521-529, 522-530, 523-531, 524-532, 525-533, 526-534, 527-535, 528-536, 529-537, 530-538, 531-539, 532-540, 533-541, 534-542, 535-543, 536-544, 537-545, 538-546, 539-547, 540-548, 541-549, 542-550, 543-551, 544-552, 545-553, 546-554, 547-555, 548-556, 549-557, 550-558, 551-559, 552-560, 553-561, 554-562, 555-563, 556-564, 557-565, 558-566, 559-567, 560-568, 561-569, 562-570, 563-571, 564-572, 565-573, 566-574, 567-575, 568-576, 569-577, 570-578, 571-579, 572-580, 573-581, 574-582, 575-583, 576-584, 577-585, 578-586, 579-587, 580-588, 581-589, 582-590, 583-591, 584-592, 585-593, 586-594, 587-595, 588-596, 589-597, 590-598, 591-599, 592-600, 593-601, 594-602, 595-603, 596-604, 597-605, 598-606, 599-607, 600-608, 601-609, 602-610, 603-611, 604-612, 605-613, 606-614, 607-615, 608-616, 609-617, 610-618, 611-619, 612-620, 613-621, 614-622, 615-623, 616-624, 617-625, 618-626, 619-627, 620-628, 621-629, 622-630, 623-631, 624-632, 625-633, 626-634, 627-635, 628-636, 629-637, 630-638, 631-639, 632-640, 633-641, 634-642, 635-643, 636-644, 637-645, 638-646, 639-647, 640-648, 641-649, 642-650, 643-651, 644-652, 645-653, 646-654, 647-655, 648-656, 649-657, 650-658, 651-659, 652-660, 653-661, 654-662, 655-663, 656-664, 657-665, 658-666, 659-667, 660-668, 661-669, 662-670, 663-671, 664-672, 665-673, 666-674, 667-675, 668-676, 669-677, 670-678, 671-679, 672-680, 673-681, 674-682, 675-683, 676-684, 677-685, 678-686, 679-687, 680-688, 681-689, 682-690, 683-691, 684-692, 685-693, 686-694, 687-695, 688-696, 689-697, 690-698, 691-699, 692-700, 693-701, 694-702, 695-703, 696-704, 697-705, 698-706, 699-707, 700-708, 701-709, 702-710, 703-711, 704-712, 705-713, 706-714, 707-715, 708-716, 709-717, 710-718, 711-719, 712-720, 713-721, 714-722, 715-723 and 716-724.

Exemplary polypeptides include the following 12-mer polypeptide of the 724 amino acid residues of SEQ ID NO:9: 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, 22-33, 23-34, 24-35, 25-36, 26-37, 27-38, 28-39, 29-40, 30-41, 31-42, 32-43, 33-44, 34-45, 35-46, 36-47, 37-48, 38-49, 39-50, 40-51, 41-52, 42-53, 43-54, 44-55, 45-56, 46-57, 47-58, 48-59, 49-60, 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723 and 713-724.

Exemplary polypeptides include the following 15-mer polypeptide of the 724 amino acid residues of SEQ ID NO:9: 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 46-60, 47-61, 48-62, 49-63, 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723 and 710-724.

Exemplary polypeptides include the following 20-mer polypeptide of the 724 amino acid residues of SEQ ID NO:9: 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723 and 705-724.

Exemplary polypeptides include the following 25-mer polypeptide of the 724 amino acid residues of SEQ ID NO:9: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723 and 700-724.

Exemplary polypeptides include the following 9-mer polypeptide of the 795 amino acid residues of SEQ ID NO:12: 1-9, 2-10, 3-11, 4-12, 5-13, 6-14, 7-15, 8-16, 9-17, 10-18, 11-19, 12-20, 13-21, 14-22, 15-23, 16-24, 17-25, 18-26, 19-27, 20-28, 21-29, 22-30, 23-31, 24-32, 25-33, 26-34, 27-35, 28-36, 29-37, 30-38, 31-39, 32-40, 33-41, 34-42, 35-43, 36-44, 37-45, 38-46, 39-47, 40-48, 41-49, 42-50, 43-51, 44-52, 45-53, 46-54, 47-55, 48-56, 49-57, 50-58, 51-59, 52-60, 53-61, 54-62, 55-63, 56-64, 57-65, 58-66, 59-67, 60-68, 61-69, 62-70, 63-71, 64-72, 65-73, 66-74, 67-75, 68-76, 69-77, 70-78, 71-79, 72-80, 73-81, 74-82, 75-83, 76-84, 77-85, 78-86, 79-87, 80-88, 81-89, 82-90, 83-91, 84-92, 85-93, 86-94, 87-95, 88-96, 89-97, 90-98, 91-99, 92-100, 93-101, 94-102, 95-103, 96-104, 97-105, 98-106, 99-107, 100-108, 101-109, 102-110, 103-111, 104-112, 105-113, 106-114, 107-115, 108-116, 109-117, 110-118, 111-119, 112-120, 113-121, 114-122, 115-123, 116-124, 117-125, 118-126, 119-127, 120-128, 121-129, 122-130, 123-131, 124-132, 125-133, 126-134, 127-135, 128-136, 129-137, 130-138, 131-139, 132-140, 133-141, 134-142, 135-143, 136-144, 137-145, 138-146, 139-147, 140-148, 141-149, 142-150, 143-151, 144-152, 145-153, 146-154, 147-155, 148-156, 149-157, 150-158, 151-159, 152-160, 153-161, 154-162, 155-163, 156-164, 157-165, 158-166, 159-167, 160-168, 161-169, 162-170, 163-171, 164-172, 165-173, 166-174, 167-175, 168-176, 169-177, 170-178, 171-179, 172-180, 173-181, 174-182, 175-183, 176-184, 177-185, 178-186, 179-187, 180-188, 181-189, 182-190, 183-191, 184-192, 185-193, 186-194, 187-195, 188-196, 189-197, 190-198, 191-199, 192-200, 193-201, 194-202, 195-203, 196-204, 197-205, 198-206, 199-207, 200-208, 201-209, 202-210, 203-211, 204-212, 205-213, 206-214, 207-215, 208-216, 209-217, 210-218, 211-219, 212-220, 213-221, 214-222, 215-223, 216-224, 217-225, 218-226, 219-227, 220-228, 221-229, 222-230, 223-231, 224-232, 225-233, 226-234, 227-235, 228-236, 229-237, 230-238, 231-239, 232-240, 233-241, 234-242, 235-243, 236-244, 237-245, 238-246, 239-247, 240-248, 241-249, 242-250, 243-251, 244-252, 245-253, 246-254, 247-255, 248-256, 249-257, 250-258, 251-259, 252-260, 253-261, 254-262, 255-263, 256-264, 257-265, 258-266, 259-267, 260-268, 261-269, 262-270, 263-271, 264-272, 265-273, 266-274, 267-275, 268-276, 269-277, 270-278, 271-279, 272-280, 273-281, 274-282, 275-283, 276-284, 277-285, 278-286, 279-287, 280-288, 281-289, 282-290, 283-291, 284-292, 285-293, 286-294, 287-295, 288-296, 289-297, 290-298, 291-299, 292-300, 293-301, 294-302, 295-303, 296-304, 297-305, 298-306, 299-307, 300-308, 301-309, 302-310, 303-311, 304-312, 305-313, 306-314, 307-315, 308-316, 309-317, 310-318, 311-319, 312-320, 313-321, 314-322, 315-323, 316-324, 317-325, 318-326, 319-327, 320-328, 321-329, 322-330, 323-331, 324-332, 325-333, 326-334, 327-335, 328-336, 329-337, 330-338, 331-339, 332-340, 333-341, 334-342, 335-343, 336-344, 337-345, 338-346, 339-347, 340-348, 341-349, 342-350, 343-351, 344-352, 345-353, 346-354, 347-355, 348-356, 349-357, 350-358, 351-359, 352-360, 353-361, 354-362, 355-363, 356-364, 357-365, 358-366, 359-367, 360-368, 361-369, 362-370, 363-371, 364-372, 365-373, 366-374, 367-375, 368-376, 369-377, 370-378, 371-379, 372-380, 373-381, 374-382, 375-383, 376-384, 377-385, 378-386, 379-387, 380-388, 381-389, 382-390, 383-391, 384-392, 385-393, 386-394, 387-395, 388-396, 389-397, 390-398, 391-399, 392-400, 393-401, 394-402, 395-403, 396-404, 397-405, 398-406, 399-407, 400-408, 401-409, 402-410, 403-411, 404-412, 405-413, 406-414, 407-415, 408-416, 409-417, 410-418, 411-419, 412-420, 413-421, 414-422, 415-423, 416-424, 417-425, 418-426, 419-427, 420-428, 421-429, 422-430, 423-431, 424-432, 425-433, 426-434, 427-435, 428-436, 429-437, 430-438, 431-439, 432-440, 433-441, 434-442, 435-443, 436-444, 437-445, 438-446, 439-447, 440-448, 441-449, 442-450, 443-451, 444-452, 445-453, 446-454, 447-455, 448-456, 449-457, 450-458, 451-459, 452-460, 453-461, 454-462, 455-463, 456-464, 457-465, 458-466, 459-467, 460-468, 461-469, 462-470, 463-471, 464-472, 465-473, 466-474, 467-475, 468-476, 469-477, 470-478, 471-479, 472-480, 473-481, 474-482, 475-483, 476-484, 477-485, 478-486, 479-487, 480-488, 481-489, 482-490, 483-491, 484-492, 485-493, 486-494, 487-495, 488-496, 489-497, 490-498, 491-499, 492-500, 493-501, 494-502, 495-503, 496-504, 497-505, 498-506, 499-507, 500-508, 501-509, 502-510, 503-511, 504-512, 505-513, 506-514, 507-515, 508-516, 509-517, 510-518, 511-519, 512-520, 513-521, 514-522, 515-523, 516-524, 517-525, 518-526, 519-527, 520-528, 521-529, 522-530, 523-531, 524-532, 525-533, 526-534, 527-535, 528-536, 529-537, 530-538, 531-539, 532-540, 533-541, 534-542, 535-543, 536-544, 537-545, 538-546, 539-547, 540-548, 541-549, 542-550, 543-551, 544-552, 545-553, 546-554, 547-555, 548-556, 549-557, 550-558, 551-559, 552-560, 553-561, 554-562, 555-563, 556-564, 557-565, 558-566, 559-567, 560-568, 561-569, 562-570, 563-571, 564-572, 565-573, 566-574, 567-575, 568-576, 569-577, 570-578, 571-579, 572-580, 573-581, 574-582, 575-583, 576-584, 577-585, 578-586, 579-587, 580-588, 581-589, 582-590, 583-591, 584-592, 585-593, 586-594, 587-595, 588-596, 589-597, 590-598, 591-599, 592-600, 593-601, 594-602, 595-603, 596-604, 597-605, 598-606, 599-607, 600-608, 601-609, 602-610, 603-611, 604-612, 605-613, 606-614, 607-615, 608-616, 609-617, 610-618, 611-619, 612-620, 613-621, 614-622, 615-623, 616-624, 617-625, 618-626, 619-627, 620-628, 621-629, 622-630, 623-631, 624-632, 625-633, 626-634, 627-635, 628-636, 629-637, 630-638, 631-639, 632-640, 633-641, 634-642, 635-643, 636-644, 637-645, 638-646, 639-647, 640-648, 641-649, 642-650, 643-651, 644-652, 645-653, 646-654, 647-655, 648-656, 649-657, 650-658, 651-659, 652-660, 653-661, 654-662, 655-663, 656-664, 657-665, 658-666, 659-667, 660-668, 661-669, 662-670, 663-671, 664-672, 665-673, 666-674, 667-675, 668-676, 669-677, 670-678, 671-679, 672-680, 673-681, 674-682, 675-683, 676-684, 677-685, 678-686, 679-687, 680-688, 681-689, 682-690, 683-691, 684-692, 685-693, 686-694, 687-695, 688-696, 689-697, 690-698, 691-699, 692-700, 693-701, 694-702, 695-703, 696-704, 697-705, 698-706, 699-707, 700-708, 701-709, 702-710, 703-711, 704-712, 705-713, 706-714, 707-715, 708-716, 709-717, 710-718, 711-719, 712-720, 713-721, 714-722, 715-723, 716-724, 717-725, 718-726, 719-727, 720-728, 721-729, 722-730, 723-731, 724-732, 725-733, 726-734, 727-735, 728-736, 729-737, 730-738, 731-739, 732-740, 733-741, 734-742, 735-743, 736-744, 737-745, 738-746, 739-747, 740-748, 741-749, 742-750, 743-751, 744-752, 745-753, 746-754, 747-755, 748-756, 749-757, 750-758, 751-759, 752-760, 753-761, 754-762, 755-763, 756-764, 757-765, 758-766, 759-767, 760-768, 761-769, 762-770, 763-771, 764-772, 765-773, 766-774, 767-775, 768-776, 769-777, 770-778, 771-779, 772-780, 773-781, 774-782, 775-783, 776-784, 777-785, 778-786, 779-787, 780-788, 781-789, 782-790, 783-791, 784-792, 785-793, 786-794 and 787-795.

Exemplary polypeptides include the following 12-mer polypeptide of the 795 amino acid residues of SEQ ID NO:12: 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, 22-33, 23-34, 24-35, 25-36, 26-37, 27-38, 28-39, 29-40, 30-41, 31-42, 32-43, 33-44, 34-45, 35-46, 36-47, 37-48, 38-49, 39-50, 40-51, 41-52, 42-53, 43-54, 44-55, 45-56, 46-57, 47-58, 48-59, 49-60, 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723, 713-724, 714-725, 715-726, 716-727, 717-728, 718-729, 719-730, 720-731, 721-732, 722-733, 723-734, 724-735, 725-736, 726-737, 727-738, 728-739, 729-740, 730-741, 731-742, 732-743, 733-744, 734-745, 735-746, 736-747, 737-748, 738-749, 739-750, 740-751, 741-752, 742-753, 743-754, 744-755, 745-756, 746-757, 747-758, 748-759, 749-760, 750-761, 751-762, 752-763, 753-764, 754-765, 755-766, 756-767, 757-768, 758-769, 759-770, 760-771, 761-772, 762-773, 763-774, 764-775, 765-776, 766-777, 767-778, 768-779, 769-780, 770-781, 771-782, 772-783, 773-784, 774-785, 775-786, 776-787, 777-788, 778-789, 779-790, 780-791, 781-792, 782-793, 783-794 and 784-795.

Exemplary polypeptides include the following 5-mer polypeptide of the 795 amino acid residues of SEQ ID NO:12: 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 46-60, 47-61, 48-62, 49-63, 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597 , 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723, 710-724, 711-725, 712-726, 713-727, 714-728, 715-729, 716-730, 717-731, 718-732, 719-733, 720-734, 721-735, 722-736, 723-737, 724-738, 725-739, 726-740, 727-741, 728-742, 729-743, 730-744, 731-745, 732-746, 733-747, 734-748, 735-749, 736-750, 737-751, 738-752, 739-753, 740-754, 741-755, 742-756, 743-757, 744-758, 745-759, 746-760, 747-761, 748-762, 749-763, 750-764, 751-765, 752-766, 753-767, 754-768, 755-769, 756-770, 757-771, 758-772, 759-773, 760-774, 761-775, 762-776, 763-777, 764-778, 765-779, 766-780, 767-781, 768-782, 769-783, 770-784, 771-785, 772-786, 773-787, 774-788, 775-789, 776-790, 777-791, 778-792, 779-793, 780-794 and 781-795.

Exemplary polypeptides include the following 20-mer polypeptide of the 795 amino acid residues of SEQ ID NO:12: 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794 and 776-795.

Exemplary polypeptides include the following 25-mer polypeptide of the 795 amino acid residues of SEQ ID NO:12: 1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794 and 771-795.

Biologically Active Variants

Variants of the protein and polypeptides disclosed herein can also occur. Variants can be naturally or non-naturally occurring. Naturally occurring variants are found in humans or other species and comprise amino acid sequences which are substantially identical to the amino acid sequence shown in SEQ ID NO:3, 6, 9, 12 or 21. Species homologs of the protein can be obtained using subgenomic polynucleotides of the invention, as described below, to make suitable probes or primers to screening cDNA expression libraries from other species, such as mice, monkeys, yeast, or bacteria, identifying cDNAs which encode homologs of the protein, and expressing the cDNAs as is known in the art.

Non-naturally occurring variants that retain substantially the same biological activities as naturally occurring protein variants, specifically the four transmembrane configuration and the interaction with other cell surface proteins, are also included here. Preferably, naturally or non-naturally occurring variants have amino acid sequences which are at least 85%, 90%, or 95% identical to the amino acid sequence shown in SEQ ID NO:3, 6, 9, 12 or 21. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981)2:482-489.

Guidance in determining which amino acid residues can be substituted, inserted, or deleted without abolishing biological or immunological activity can be found using computer programs well known in the art, such as DNASTAR software. Preferably, amino acid changes in secreted protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Variants of the PAR-1 protein disclosed herein include glycosylated forms, aggregative conjugates with other molecules, and covalent conjugates with unrelated chemical moieties. Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al. U.S. Pat. No. 4,959,314.

Preferably, amino acid changes in the PAR-1 protein or polypeptide variants are conservative amino acid changes, ie., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of PAR-1 protein or polypeptide variants are of the same type as a protein comprising the amino acid sequence encoded by the nucleotide sequence shown in SEQ ID NO:1, 2, 4, 5, 7, 8, 10, 11, 19 or 20, although the properties and functions of variants can differ in degree.

PAR-1 protein variants include glycosylated forms, aggregative conjugates with other molecules, and covalent conjugates with unrelated chemical moieties. PAR-1 protein variants also include allelic variants, species variants, and muteins. Truncations or deletions of regions which do not affect the differential expression of the PAR-1 protein gene are also variants. Covalent variants can be prepared by linking functionalities to groups which are found in the amino acid chain or at the N- or C-terminal residue, as is known in the art.

It will be recognized in the art that some amino acid sequence of the PAR-1 protein of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the PAR-1 polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J.U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Fusion Proteins

Fusion proteins comprising proteins or polypeptide fragments of PAR-1 can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of PAR-1 or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can be utilize the amino acid sequence shown in SEQ ID NO:3, 6, 9, 12 or 21 or can be prepared from biologically active variants of SEQ ID NO:3, 6, 9, 12 or 21, such as those described above. The first protein segment can consist of a full-length PAR-1.

Other first protein segments can consist of at least 8, 10, 12, 15, 18, 19, 20, 25, 50, 75, 100, 125, 130, 140, 150, 160, 170, 180, 190, 200, 250, 300, 350, 400, 450, 500, 550, 600, 650, or 675 contiguous amino acids selected from SEQ ID NO:3, 6, 9, 12 or 21 or at least amino acids 1-675 of SEQ ID NO:3, 6, 9, 12 or 21.

The second protein segment can be a full-length protein or a polypeptide fragment. Proteins commonly used in fusion protein construction include βgalactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence of SEQ ID NO:1, 2, 4, 5, 7, 8, 10, 11, 19 or 20 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Isolation and Production of PAR-1

PAR-1 is expressed in a variety of human cells and can be extracted from these cells or from other human cells, such as recombinant cells comprising SEQ ID NO:1, 2, 4, 5, 7, 8, 10 or 11, using standard biochemical methods. These methods include, but are not limited to, size exclusion chromatography, ammonium sulfate fractionation, ion exchange chromatography, affinity chromatography, crystallization, electrofocusing, and preparative gel electrophoresis. The isolated and purified protein or polypeptide is separated from other compounds which normally associate with the protein or polypeptide in a cell, such as other proteins, carbohydrates, lipids, or subcellular organelles. A preparation of isolated and purified protein or polypeptide is at least 80% pure; preferably, the preparations are 90%, 95%, or 99% pure. Purity of the preparations can be assessed by any means known in the art. For example, the purity of a preparation can be assessed by examining electrophoretograms of protein or polypeptide preparations at several pH values and at several polyacrylamide concentrations, as is known in the art.

Proteins, fusion proteins, or polypeptides of the invention can be produced by recombinant DNA methods. For production of recombinant proteins, fusion proteins, or polypeptides, a coding sequence of the nucleotide sequence shown in SEQ ID NO:1, 2, 4, 5, 7, 8, 10, 11, 19 or 20 can be expressed in prokaryotic or eukaryotic host cells using expression systems known in the art. These expression systems include bacterial, yeast, insect, and mammalian cells.

The resulting expressed PAR-1 protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

PAR-1 protein or polypeptide of the invention can also be expressed in cultured host cells in a form that will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule. Variants can be similarly produced.

Polynucleotide Sequences

A gene which encode the PAR-1 protein of the invention has the coding sequence shown in SEQ ID NO:1 and 2 (hPAR-1A), 4 and 5 (hPAR-1Bα), 7 and 8 (hPR-1Bβ), 10 and 11 (hPAR-1C) and 13 and 14 (dPAR-1). Polynucleotide molecules of the invention contain less than a whole chromosome and can be single- or double-stranded. Preferably, the polynucleotide molecules are intron-free. Polynucleotide molecules of the invention can comprise at least 11, 12, 13, 15, 18, 21, 30, 33, 42, 54, 60, 66, 72, 84, 90, 100, 120, 140, 160, 180, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150 or 2200 or more contiguous nucleotides selected from the nucleotides of SEQ ID NO:1, 2, 4, 5, 7, 8, 10, 11, 19 or 20, or the complements thereof. The complement of the nucleotide sequence shown in SEQ ID NO:1, 2, 4, 5, 7, 8, 10, 11, 19 or 20 is a contiguous nucleotide sequence which forms Watson-Crick base pairs with a contiguous nucleotide sequence as shown in SEQ ID NO:1, 2, 4, 5, 7, 8, 10, 11, 19 or 20.

Exemplary polynucleotide molecules include the following 12-mer fragments of the polynucleotide sequence from the sequence of SEQ ID NO:1: 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723, 713-724, 714-725, 715-726, 716-727, 717-728, 718-729, 719-730, 720-731, 721-732, 722-733, 723-734, 724-735, 725-736, 726-737, 727-738, 728-739, 729-740, 730-741, 731-742, 732-743, 733-744, 734-745, 735-746, 736-747, 737-748, 738-749, 739-750, 740-751, 741-752, 742-753, 743-754, 744-755, 745-756, 746-757, 747-758, 748-759, 749-760, 750-761, 751-762, 752-763, 753-764, 754-765, 755-766, 756-767, 757-768, 758-769, 759-770, 760-771, 761-772, 762-773, 763-774, 764-775, 765-776, 766-777, 767-778, 768-779, 769-780, 770-781, 771-782, 772-783, 773-784, 774-785, 775-786, 776-787, 777-788, 778-789, 779-790, 780-791, 781-792, 782-793, 783-794, 784-795, 785-796, 786-797, 787-798, 788-799, 789-800, 790-801, 791-802, 792-803, 793-804, 794-805, 795-806, 796-807, 797-808, 798-809, 799-810, 800-811, 801-812, 802-813, 803-814, 804-815, 805-816, 806-817, 807-818, 808-819, 809-820, 810-821, 811-822, 812-823, 813-824, 814-825, 815-826, 816-827, 817-828, 818-829, 819-830, 820-831, 821-832, 822-833, 823-834, 824-835, 825-836, 826-837, 827-838, 828-839, 829-840, 830-841, 831-842, 832-843, 833-844, 834-845, 835-846, 836-847, 837-848, 838-849, 839-850, 840-851, 841-852, 842-853, 843-854, 844-855, 845-856, 846-857, 847-858, 848-859, 849-860, 850-861, 851-862, 852-863, 853-864, 854-865, 855-866, 856-867, 857-868, 858-869, 859-870, 860-871, 861-872, 862-873, 863-874, 864-875, 865-876, 866-877, 867-878, 868-879, 869-880, 870-881, 871-882, 872-883, 873-884, 874-885, 875-886, 876-887, 877-888, 878-889, 879-890, 880-891, 881-892, 882-893, 883-894, 884-895, 885-896, 886-897, 887-898, 888-899, 889-900, 890-901, 891-902, 892-903, 893-904, 894-905, 895-906, 896-907, 897-908, 898-909, 899-910, 900-911, 901-912, 902-913, 903-914, 904-915, 905-916, 906-917, 907-918, 908-919, 909-920, 910-921, 911-922, 912-923, 913-924, 914-925, 915-926, 916-927, 917-928, 918-929, 919-930, 920-931, 921-932, 922-933, 923-934, 924-935, 925-936, 926-937, 927-938, 928-939, 929-940, 930-941, 931-942, 932-943, 933-944, 934-945, 935-946, 936-947, 937-948, 938-949, 939-950, 940-951, 941-952, 942-953, 943-954, 944-955, 945-956, 946-957, 947-958, 948-959, 949-960, 950-961, 951-962, 952-963, 953-964, 954-965, 955-966, 956-967, 957-968, 958-969, 959-970, 960-971, 961-972, 962-973, 963-974, 964-975, 965-976, 966-977, 967-978, 968-979, 969-980, 970-981, 971-982, 972-983, 973-984, 974-985, 975-986, 976-987, 977-988, 978-989, 979-990, 980-991, 981-992, 982-993, 983-994, 984-995, 985-996, 986-997, 987-998, 988-999, 989-1000, 990-1001, 991-1002, 992-1003, 993-1004, 994-1005, 995-1006, 996-1007, 997-1008, 998-1009, 999-1010, 1000-1011, 1001-1012, 1002-1013, 1003-1014, 1004-1015, 1005-1016, 1006-1017, 1007-1018, 1008-1019, 1009-1020, 1010-1021, 1011-1022, 1012-1023, 1013-1024, 1014-1025, 1015-1026, 1016-1027, 1017-1028, 1018-1029, 1019-1030, 1020-1031, 1021-1032, 1022-1033, 1023-1034, 1024-1035, 1025-1036, 1026-1037, 1027-1038, 1028-1039, 1029-1040, 1030-1041, 1031-1042, 1032-1043, 1033-1044, 1034-1045, 1035-1046, 1036-1047, 1037-1048, 1038-1049, 1039-1050, 1040-1051, 1041-1052, 1042-1053, 1043-1054, 1044-1055, 1045-1056, 1046-1057, 1047-1058, 1048-1059, 1049-1060, 1050-1061, 1051-1062, 1052-1063, 1053-1064, 1054-1065, 1055-1066, 1056-1067, 1057-1068, 1058-1069, 1059-1070, 1060-1071, 1061-1072, 1062-1073, 1063-1074, 1064-1075, 1065-1076, 1066-1077, 1067-1078, 1068-1079, 1069-1080, 1070-1081, 1071-1082, 1072-1083, 1073-1084, 1074-1085, 1075-1086, 1076-1087, 1077-1088, 1078-1089, 1079-1090, 1080-1091, 1081-1092, 1082-1093, 1083-1094, 1084-1095, 1085-1096, 1086-1097, 1087-1098, 1088-1099, 1089-1100, 1090-1101, 1091-1102, 1092-1103, 1093-1104, 1094-1105, 1095-1106, 1096-1107, 1097-1108, 1098-1109, 1099-1110, 1100-1111, 1101-1112, 1102-1113, 1103-1114, 1104-1115, 1105-1116, 1106-1117, 1107-1118, 1108-1119, 1109-1120, 1110-1121, 1111-1122, 1112-1123, 1113-1124, 1114-1125, 1115-1126, 1116-1127, 1117-1128, 1118-1129, 1119-1130, 1120-1131, 1121-1132, 1122-1133, 1123-1134, 1124-1135, 1125-1136, 1126-1137, 1127-1138, 1128-1139, 1129-1140, 1130-1141, 1131-1142, 1132-1143, 1133-1144, 1134-1145, 1135-1146, 1136-1147, 1137-1148, 1138-1149, 1139-1150, 1140-1151, 1141-1152, 1142-1153, 1143-1154, 1144-1155, 1145-1156, 1146-1157, 1147-1158, 1148-1159, 1149-1160, 1150-1161, 1151-1162, 1152-1163, 1153-1164, 1154-1165, 1155-1166, 1156-1167, 1157-1168, 1158-1169, 1159-1170, 1160-1171, 1161-1172, 1162-1173, 1163-1174, 1164-1175, 1165-1176, 1166-1177, 1167-1178, 1168-1179, 1169-1180, 1170-1181, 1171-1182, 1172-1183, 1173-1184, 1174-1185, 1175-1186, 1176-1187, 1177-1188, 1178-1189, 1179-1190, 1180-1191, 1181-1192, 1182-1193, 1183-1194, 1184-1195, 1185-1196, 1186-1197, 1187-1198, 1188-1199, 1189-1200, 1190-1201, 1191-1202, 1192-1203, 1193-1204, 1194-1205, 1195-1206, 1196-1207, 1197-1208, 1198-1209, 1199-1210, 1200-1211, 1201-1212, 1202-1213, 1203-1214, 1204-1215, 1205-1216, 1206-1217, 1207-1218, 1208-1219, 1209-1220, 1210-1221, 1211-1222, 1212-1223, 1213-1224, 1214-1225, 1215-1226, 1216-1227, 1217-1228, 1218-1229, 1219-1230, 1220-1231, 1221-1232, 1222-1233, 1223-1234, 1224-1235, 1225-1236, 1226-1237, 1227-1238, 1228-1239, 1229-1240, 1230-1241, 1231-1242, 1232-1243, 1233-1244, 1234-1245, 1235-1246, 1236-1247, 1237-1248, 1238-1249, 1239-1250, 1240-1251, 1241-1252, 1242-1253, 1243-1254, 1244-1255, 1245-1256, 1246-1257, 1247-1258, 1248-1259, 1249-1260, 1250-1261, 1251-1262, 1252-1263, 1253-1264, 1254-1265, 1255-1266, 1256-1267, 1257-1268, 1258-1269, 1259-1270, 1260-1271, 1261-1272, 1262-1273, 1263-1274, 1264-1275, 1265-1276, 1266-1277, 1267-1278, 1268-1279, 1269-1280, 1270-1281, 1271-1282, 1272-1283, 1273-1284, 1274-1285, 1275-1286, 1276-1287, 1277-1288, 1278-1289, 1279-1290, 1280-1291, 1281-1292, 1282-1293, 1283-1294, 1284-1295, 1285-1296, 1286-1297, 1287-1298, 1288-1299, 1289-1300, 1290-1301, 1291-1302, 1292-1303, 1293-1304, 1294-1305, 1295-1306, 1296-1307, 1297-1308, 1298-1309, 1299-1310, 1300-1311, 1301-1312, 1302-1313, 1303-1314, 1304-1315, 1305-1316, 1306-1317, 1307-1318, 1308-1319, 1309-1320, 1310-1321, 1311-1322, 1312-1323, 1313-1324, 1314-1325, 1315-1326, 1316-1327, 1317-1328, 1318-1329, 1319-1330, 1320-1331, 1321-1332, 1322-1333, 1323-1334, 1324-1335, 1325-1336, 1326-1337, 1327-1338, 1328-1339, 1329-1340, 1330-1341, 1331-1342, 1332-1343, 1333-1344, 1334-1345, 1335-1346, 1336-1347, 1337-1348, 1338-1349, 1339-1350, 1340-1351, 1341-1352, 1342-1353, 1343-1354, 1344-1355, 1345-1356, 1346-1357, 1347-1358, 1348-1359, 1349-1360, 1350-1361, 1351-1362, 1352-1363, 1353-1364, 1354-1365, 1355-1366, 1356-1367, 1357-1368, 1358-1369, 1359-1370, 1360-1371, 1361-1372, 1362-1373, 1363-1374, 1364-1375, 1365-1376, 1366-1377, 1367-1378, 1368-1379, 1369-1380, 1370-1381, 1371-1382, 1372-1383, 1373-1384, 1374-1385, 1375-1386, 1376-1387, 1377-1388, 1378-1389, 1379-1390, 1380-1391, 1381-1392, 1382-1393, 1383-1394, 1384-1395, 1385-1396, 1386-1397, 1387-1398, 1388-1399, 1389-1400, 1390-1401, 1391-1402, 1392-1403, 1393-1404, 1394-1405, 1395-1406, 1396-1407, 1397-1408, 1398-1409, 1399-1410, 1400-1411, 1401-1412, 1402-1413, 1403-1414, 1404-1415, 1405-1416, 1406-1417, 1407-1418, 1408-1419, 1409-1420, 1410-1421, 1411-1422, 1412-1423, 1413-1424, 1414-1425, 1415-1426, 1416-1427, 1417-1428, 1418-1429, 1419-1430, 1420-1431, 1421-1432, 1422-1433, 1423-1434, 1424-1435, 1425-1436, 1426-1437, 1427-1438, 1428-1439, 1429-1440, 1430-1441, 1431-1442, 1432-1443, 1433-1444, 1434-1445, 1435-1446, 1436-1447, 1437-1448, 1438-1449, 1439-1450, 1440-1451, 1441-1452, 1442-1453, 1443-1454, 1444-1455, 1445-1456, 1446-1457, 1447-1458, 1448-1459, 1449-1460, 1450-1461, 1451-1462, 1452-1463, 1453-1464, 1454-1465, 1455-1466, 1456-1467, 1457-1468, 1458-1469, 1459-1470, 1460-1471, 1461-1472, 1462-1473, 1463-1474, 1464-1475, 1465-1476, 1466-1477, 1467-1478, 1468-1479, 1469-1480, 1470-1481, 1471-1482, 1472-1483, 1473-1484, 1474-1485, 1475-1486, 1476-1487, 1477-1488, 1478-1489, 1479-1490, 1480-1491, 1481-1492, 1482-1493, 1483-1494, 1484-1495, 1485-1496, 1486-1497, 1487-1498, 1488-1499, 1489-1500, 1490-1501, 1491-1502, 1492-1503, 1493-1504, 1494-1505, 1495-1506, 1496-1507, 1497-1508, 1498-1509, 1499-1510, 1500-1511, 1501-1512, 1502-1513, 1503-1514, 1504-1515, 1505-1516, 1506-1517, 1507-1518, 1508-1519, 1509-1520, 1510-1521, 1511-1522, 1512-1523, 1513-1524, 1514-1525, 1515-1526, 1516-1527, 1517-1528, 1518-1529, 1519-1530, 1520-1531, 1521-1532, 1522-1533, 1523-1534, 1524-1535, 1525-1536, 1526-1537, 1527-1538, 1528-1539, 1529-1540, 1530-1541, 1531-1542, 1532-1543, 1533-1544, 1534-1545, 1535-1546, 1536-1547, 1537-1548, 1538-1549, 1539-1550, 1540-1551, 1541-1552, 1542-1553, 1543-1554, 1544-1555, 1545-1556, 1546-1557, 1547-1558, 1548-1559, 1549-1560, 1550-1561, 1551-1562, 1552-1563, 1553-1564, 1554-1565, 1555-1566, 1556-1567, 1557-1568, 1558-1569, 1559-1570, 1560-1571, 1561-1572, 1562-1573, 1563-1574, 1564-1575, 1565-1576, 1566-1577, 1567-1578, 1568-1579, 1569-1580, 1570-1581, 1571-1582, 1572-1583, 1573-1584, 1574-1585, 1575-1586, 1576-1587, 1577-1588, 1578-1589, 1579-1590, 1580-1591, 1581-1592, 1582-1593, 1583-1594, 1584-1595, 1585-1596, 1586-1597, 1587-1598, 1588-1599, 1589-1600, 1590-1601, 1591-1602, 1592-1603, 1593-1604, 1594-1605, 1595-1606, 1596-1607, 1597-1608, 1598-1609, 1599-1610, 1600-1611, 1601-1612, 1602-1613, 1603-1614, 1604-1615, 1605-1616, 1606-1617, 1607-1618, 1608-1619, 1609-1620, 1610-1621, 1611-1622, 1612-1623, 1613-1624, 1614-1625, 1615-1626, 1616-1627, 1617-1628, 1618-1629, 1619-1630, 1620-1631, 1621-1632, 1622-1633, 1623-1634, 1624-1635, 1625-1636, 1626-1637, 1627-1638, 1628-1639, 1629-1640, 1630-1641, 1631-1642, 1632-1643, 1633-1644, 1634-1645, 1635-1646, 1636-1647, 1637-1648, 1638-1649, 1639-1650, 1640-1651, 1641-1652, 1642-1653, 1643-1654, 1644-1655, 1645-1656, 1646-1657, 1647-1658, 1648-1659, 1649-1660, 1650-1661, 1651-1662, 1652-1663, 1653-1664, 1654-1665, 1655-1666, 1656-1667, 1657-1668, 1658-1669, 1659-1670, 1660-1671, 1661-1672, 1662-1673, 1663-1674, 1664-1675, 1665-1676, 1666-1677, 1667-1678, 1668-1679, 1669-1680, 1670-1681, 1671-1682, 1672-1683, 1673-1684, 1674-1685, 1675-1686, 1676-1687, 1677-1688, 1678-1689, 1679-1690, 1680-1691, 1681-1692, 1682-1693, 1683-1694, 1684-1695, 1685-1696, 1686-1697, 1687-1698, 1688-1699, 1689-1700, 1690-1701, 1691-1702, 1692-1703, 1693-1704, 1694-1705, 1695-1706, 1696-1707, 1697-1708, 1698-1709, 1699-1710, 1700-1711, 1701-1712, 1702-1713, 1703-1714, 1704-1715, 1705-1716, 1706-1717, 1707-1718, 1708-1719, 1709-1720, 1710-1721, 1711-1722, 1712-1723, 1713-1724, 1714-1725, 1715-1726, 1716-1727, 1717-1728, 1718-1729, 1719-1730, 1720-1731, 1721-1732, 1722-1733, 1723-1734, 1724-1735, 1725-1736, 1726-1737, 1727-1738, 1728-1739, 1729-1740, 1730-1741, 1731-1742, 1732-1743, 1733-1744, 1734-1745, 1735-1746, 1736-1747, 1737-1748, 1738-1749, 1739-1750, 1740-1751, 1741-1752, 1742-1753, 1743-1754, 1744-1755, 1745-1756, 1746-1757, 1747-1758, 1748-1759, 1749-1760, 1750-1761, 1751-1762, 1752-1763, 1753-1764, 1754-1765, 1755-1766, 1756-1767, 1757-1768, 1758-1769, 1759-1770, 1760-1771, 1761-1772, 1762-1773, 1763-1774, 1764-1775, 1765-1776, 1766-1777, 1767-1778, 1768-1779, 1769-1780, 1770-1781, 1771-1782, 1772-1783, 1773-1784, 1774-1785, 1775-1786, 1776-1787, 1777-1788, 1778-1789, 1779-1790, 1780-1791, 1781-1792, 1782-1793, 1783-1794, 1784-1795, 1785-1796, 1786-1797, 1787-1798, 1788-1799, 1789-1800, 1790-1801, 1791-1802, 1792-1803, 1793-1804, 1794-1805, 1795-1806, 1796-1807, 1797-1808, 1798-1809, 1799-1810, 1800-1811, 1801-1812, 1802-1813, 1803-1814, 1804-1815, 1805-1816, 1806-1817, 1807-1818, 1808-1819, 1809-1820, 1810-1821, 1811-1822, 1812-1823, 1813-1824, 1814-1825, 1815-1826, 1816-1827, 1817-1828, 1818-1829, 1819-1830, 1820-1831, 1821-1832, 1822-1833, 1823-1834, 1824-1835, 1825-1836, 1826-1837, 1827-1838, 1828-1839, 1829-1840, 1830-1841, 1831-1842, 1832-1843, 1833-1844, 1834-1845, 1835-1846, 1836-1847, 1837-1848, 1838-1849, 1839-1850, 1840-1851, 1841-1852, 1842-1853, 1843-1854, 1844-1855, 1845-1856, 1846-1857, 1847-1858, 1848-1859, 1849-1860, 1850-1861, 1851-1862, 1852-1863, 1853-1864, 1854-1865, 1855-1866, 1856-1867, 1857-1868, 1858-1869, 1859-1870, 1860-1871, 1861-1872, 1862-1873, 1863-1874, 1864-1875, 1865-1876, 1866-1877, 1867-1878, 1868-1879, 1869-1880, 1870-1881, 1871-1882, 1872-1883, 1873-1884, 1874-1885, 1875-1886, 1876-1887, 1877-1888, 1878-1889, 1879-1890, 1880-1891, 1881-1892, 1882-1893, 1883-1894, 1884-1895, 1885-1896, 1886-1897, 1887-1898, 1888-1899, 1889-1900, 1890-1901, 1891-1902, 1892-1903, 1893-1904, 1894-1905, 1895-1906, 1896-1907, 1897-1908, 1898-1909, 1899-1910, 1900-1911, 1901-1912, 1902-1913, 1903-1914, 1904-1915, 1905-1916, 1906-1917, 1907-1918, 1908-1919, 1909-1920, 1910-1921, 1911-1922, 1912-1923, 1913-1924, 1914-1925, 1915-1926, 1916-1927, 1917-1928, 1918-1929, 1919-1930, 1920-1931, 1921-1932, 1922-1933, 1923-1934, 1924-1935, 1925-1936, 1926-1937, 1927-1938, 1928-1939, 1929-1940, 1930-1941, 1931-1942, 1932-1943, 1933-1944, 1934-1945, 1935-1946, 1936-1947, 1937-1948, 1938-1949, 1939-1950, 1940-1951, 1941-1952, 1942-1953, 1943-1954, 1944-1955, 1945-1956, 1946-1957, 1947-1958, 1948-1959, 1949-1960, 1950-1961, 1951-1962, 1952-1963, 1953-1964, 1954-1965, 1955-1966, 1956-1967, 1957-1968, 1958-1969, 1959-1970, 1960-1971, 1961-1972, 1962-1973, 1963-1974, 1964-1975, 1965-1976, 1966-1977, 1967-1978, 1968-1979, 1969-1980, 1970-1981, 1971-1982, 1972-1983, 1973-1984, 1974-1985, 1975-1986, 1976-1987, 1977-1988, 1978-1989, 1979-1990, 1980-1991, 1981-1992, 1982-1993, 1983-1994, 1984-1995, 1985-1996, 1986-1997, 1987-1998, 1988-1999, 1989-2000, 1990-2001, 1991-2002, 1992-2003, 1993-2004, 1994-2005, 1995-2006, 1996-2007, 1997-2008, 1998-2009, 1999-2010, 2000-2011, 2001-2012, 2002-2013, 2003-2014, 2004-2015, 2005-2016, 2006-2017, 2007-2018, 2008-2019, 2009-2020, 2010-2021, 2011-2022, 2012-2023, 2013-2024, 2014-2025, 2015-2026, 2016-2027, 2017-2028, 2018-2029, 2019-2030, 2020-2031, 2021-2032, 2022-2033, 2023-2034, 2024-2035, 2025-2036, 2026-2037, 2027-2038, 2028-2039, 2029-2040, 2030-2041, 2031-2042, 2032-2043, 2033-2044, 2034-2045, 2035-2046, 2036-2047, 2037-2048, 2038-2049, 2039-2050, 2040-2051, 2041-2052, 2042-2053, 2043-2054, 2044-2055, 2045-2056, 2046-2057, 2047-2058, 2048-2059, 2049-2060, 2050-2061, 2051-2062, 2052-2063, 2053-2064, 2054-2065, 2055-2066, 2056-2067, 2057-2068, 2058-2069, 2059-2070, 2060-2071, 2061-2072, 2062-2073, 2063-2074, 2064-2075, 2065-2076, 2066-2077, 2067-2078, 2068-2079, 2069-2080, 2070-2081, 2071-2082, 2072-2083, 2073-2084, 2074-2085, 2075-2086, 2076-2087, 2077-2088, 2078-2089, 2079-2090, 2080-2091, 2081-2092, 2082-2093, 2083-2094, 2084-2095, 2085-2096, 2086-2097, 2087-2098, 2088-2099, 2089-2100, 2090-2101, 2091-2102, 2092-2103, 2093-2104, 2094-2105, 2095-2106, 2096-2107, 2097-2108, 2098-2109, 2099-2110, 2100-2111, 2101-2112, 2102-2113, 2103-2114, 2104-2115, 2105-2116, 2106-2117, 2107-2118, 2108-2119, 2109-2120, 2110-2121, 2111-2122, 2112-2123, 2113-2124, 2114-2125, 2115-2126, 2116-2127, 2117-2128, 2118-2129, 2119-2130, 2120-2131, 2121-2132, 2122-2133, 2123-2134, 2124-2135, 2125-2136, 2126-2137, 2127-2138, 2128-2139, 2129-2140, 2130-2141, 2131-2142, 2132-2143, 2133-2144, 2134-2145, 2135-2146, 2136-2147, 2137-2148, 2138-2149, 2139-2150, 2140-2151, 2141-2152, 2142-2153, 2143-2154, 2144-2155, 2145-2156, 2146-2157, 2147-2158, 2148-2159, 2149-2160, 2150-2161, 2151-2162, 2152-2163, 2153-2164, 2154-2165, 2155-2166, 2156-2167, 2157-2168, 2158-2169, 2159-2170, 2160-2171, 2161-2172, 2162-2173, 2163-2174, 2164-2175, 2165-2176, 2166-2177, 2167-2178, 2168-2179, 2169-2180, 2170-2181, 2171-2182, 2172-2183, 2173-2184, 2174-2185, 2175-2186, 2176-2187, 2177-2188, 2178-2189, 2179-2190, 2180-2191, 2181-2192, 2182-2193, 2183-2194, 2184-2195, 2185-2196, 2186-2197, 2187-2198, 2188-2199, 2189-2200, 2190-2201, 2191-2202, 2192-2203, 2193-2204, 2194-2205, 2195-2206, 2196-2207, 2197-2208, 2198-2209, 2199-2210, 2200-2211, 2201-2212, 2202-2213, 2203-2214, 2204-2215, 2205-2216, 2206-2217, 2207-2218, 2208-2219, 2209-2220, 2210-2221, 2211-2222, 2212-2223, 2213-2224, 2214-2225, 2215-2226, 2216-2227, 2217-2228, 2218-2229, 2219-2230, 2220-2231 and 2221-2232.

Exemplary polynucleotide molecules include the following 15-mer fragments of the polynucleotide sequence from the sequence of SEQ ID NO:1: 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723, 710-724, 711-725, 712-726, 713-727, 714-728, 715-729, 716-730, 717-731, 718-732, 719-733, 720-734, 721-735, 722-736, 723-737, 724-738, 725-739, 726-740, 727-741, 728-742, 729-743, 730-744, 731-745, 732-746, 733-747, 734-748, 735-749, 736-750, 737-751, 738-752, 739-753, 740-754, 741-755, 742-756, 743-757, 744-758, 745-759, 746-760, 747-761, 748-762, 749-763, 750-764, 751-765, 752-766, 753-767, 754-768, 755-769, 756-770, 757-771, 758-772, 759-773, 760-774, 761-775, 762-776, 763-777, 764-778, 765-779, 766-780, 767-781, 768-782, 769-783, 770-784, 771-785, 772-786, 773-787, 774-788, 775-789, 776-790, 777-791, 778-792, 779-793, 780-794, 781-795, 782-796, 783-797, 784-798, 785-799, 786-800, 787-801, 788-802, 789-803, 790-804, 791-805, 792-806, 793-807, 794-808, 795-809, 796-810, 797-811, 798-812, 799-813, 800-814, 801-815, 802-816, 803-817, 804-818, 805-819, 806-820, 807-821, 808-822, 809-823, 810-824, 811-825, 812-826, 813-827, 814-828, 815-829, 816-830, 817-831, 818-832, 819-833, 820-834, 821-835, 822-836, 823-837, 824-838, 825-839, 826-840, 827-841, 828-842, 829-843, 830-844, 831-845, 832-846, 833-847, 834-848, 835-849, 836-850, 837-851, 838-852, 839-853, 840-854, 841-855, 842-856, 843-857, 844-858, 845-859, 846-860, 847-861, 848-862, 849-863, 850-864, 851-865, 852-866, 853-867, 854-868, 855-869, 856-870, 857-871, 858-872, 859-873, 860-874, 861-875, 862-876, 863-877, 864-878, 865-879, 866-880, 867-881, 868-882, 869-883, 870-884, 871-885, 872-886, 873-887, 874-888, 875-889, 876-890, 877-891, 878-892, 879-893, 880-894, 881-895, 882-896, 883-897, 884-898, 885-899, 886-900, 887-901, 888-902, 889-903, 890-904, 891-905, 892-906, 893-907, 894-908, 895-909, 896-910, 897-911, 898-912, 899-913, 900-914, 901-915, 902-916, 903-917, 904-918, 905-919, 906-920, 907-921, 908-922, 909-923, 910-924, 911-925, 912-926, 913-927, 914-928, 915-929, 916-930, 917-931, 918-932, 919-933, 920-934, 921-935, 922-936, 923-937, 924-938, 925-939, 926-940, 927-941, 928-942, 929-943, 930-944, 931-945, 932-946, 933-947, 934-948, 935-949, 936-950, 937-951, 938-952, 939-953, 940-954, 941-955, 942-956, 943-957, 944-958, 945-959, 946-960, 947-961, 948-962, 949-963, 950-964, 951-965, 952-966, 953-967, 954-968, 955-969, 956-970, 957-971, 958-972, 959-973, 960-974, 961-975, 962-976, 963-977, 964-978, 965-979, 966-980, 967-981, 968-982, 969-983, 970-984, 971-985, 972-986, 973-987, 974-988, 975-989, 976-990, 977-991, 978-992, 979-993, 980-994, 981-995, 982-996, 983-997, 984-998, 985-999, 986-1000, 987-1001, 988-1002, 989-1003, 990-1004, 991-1005, 992-1006, 993-1007, 994-1008, 995-1009, 996-1010, 997-1011, 998-1012, 999-1013, 1000-1014, 1001-1015, 1002-1016, 1003-1017, 1004-1018, 1005-1019, 1006-1020, 1007-1021, 1008-1022, 1009-1023, 1010-1024, 1011-1025, 1012-1026, 1013-1027, 1014-1028, 1015-1029, 1016-1030, 1017-1031, 1018-1032, 1019-1033, 1020-1034, 1021-1035, 1022-1036, 1023-1037, 1024-1038, 1025-1039, 1026-1040, 1027-1041, 1028-1042, 1029-1043, 1030-1044, 1031-1045, 1032-1046, 1033-1047, 1034-1048, 1035-1049, 1036-1050, 1037-1051, 1038-1052, 1039-1053, 1040-1054, 1041-1055, 1042-1056, 1043-1057, 1044-1058, 1045-1059, 1046-1060, 1047-1061, 1048-1062, 1049-1063, 1050-1064, 1051-1065, 1052-1066, 1053-1067, 1054-1068, 1055-1069, 1056-1070, 1057-1071, 1058-1072, 1059-1073, 1060-1074, 1061-1075, 1062-1076, 1063-1077, 1064-1078, 1065-1079, 1066-1080, 1067-1081, 1068-1082, 1069-1083, 1070-1084, 1071-1085, 1072-1086, 1073-1087, 1074-1088, 1075-1089, 1076-1090, 1077-1091, 1078-1092, 1079-1093, 1080-1094, 1081-1095, 1082-1096, 1083-1097, 1084-1098, 1085-1099, 1086-1100, 1087-1101, 1088-1102, 1089-1103, 1090-1104, 1091-1105, 1092-1106, 1093-1107, 1094-1108, 1095-1109, 1096-1110, 1097-1111, 1098-1112, 1099-1113, 1100-1114, 1101-1115, 1102-1116, 1103-1117, 1104-1118, 1105-1119, 1106-1120, 1107-1121, 1108-1122, 1109-1123, 1110-1124, 1111-1125, 1112-1126, 1113-1127, 1114-1128, 1115-1129, 1116-1130, 1117-1131, 1118-1132, 1119-1133, 1120-1134, 1121-1135, 1122-1136, 1123-1137, 1124-1138, 1125-1139, 1126-1140, 1127-1141, 1128-1142, 1129-1143, 1130-1144, 1131-1145, 1132-1146, 1133-1147, 1134-1148, 1135-1149, 1136-1150, 1137-1151, 1138-1152, 1139-1153, 1140-1154, 1141-1155, 1142-1156, 1143-1157, 1144-1158, 1145-1159, 1146-1160, 1147-1161, 1148-1162, 1149-1163, 1150-1164, 1151-1165, 1152-1166, 1153-1167, 1154-1168, 1155-1169, 1156-1170, 1157-1171, 1158-1172, 1159-1173, 1160-1174, 1161-1175, 1162-1176, 1163-1177, 1164-1178, 1165-1179, 1166-1180, 1167-1181, 1168-1182, 1169-1183, 1170-1184, 1171-1185, 1172-1186, 1173-1187, 1174-1188, 1175-1189, 1176-1190, 1177-1191, 1178-1192, 1179-1193, 1180-1194, 1181-1195, 1182-1196, 1183-1197, 1184-1198, 1185-1199, 1186-1200, 1187-1201, 1188-1202, 1189-1203, 1190-1204, 1191-1205, 1192-1206, 1193-1207, 1194-1208, 1195-1209, 1196-1210, 1197-1211, 1198-1212, 1199-1213, 1200-1214, 1201-1215, 1202-1216, 1203-1217, 1204-1218, 1205-1219, 1206-1220, 1207-1221, 1208-1222, 1209-1223, 1210-1224, 1211-1225, 1212-1226, 1213-1227, 1214-1228, 1215-1229, 1216-1230, 1217-1231, 1218-1232, 1219-1233, 1220-1234, 1221-1235, 1222-1236, 1223-1237, 1224-1238, 1225-1239, 1226-1240, 1227-1241, 1228-1242, 1229-1243, 1230-1244, 1231-1245, 1232-1246, 1233-1247, 1234-1248, 1235-1249, 1236-1250, 1237-1251, 1238-1252, 1239-1253, 1240-1254, 1241-1255, 1242-1256, 1243-1257, 1244-1258, 1245-1259, 1246-1260, 1247-1261, 1248-1262, 1249-1263, 1250-1264, 1251-1265, 1252-1266, 1253-1267, 1254-1268, 1255-1269, 1256-1270, 1257-1271, 1258-1272, 1259-1273, 1260-1274, 1261-1275, 1262-1276, 1263-1277, 1264-1278, 1265-1279, 1266-1280, 1267-1281, 1268-1282, 1269-1283, 1270-1284, 1271-1285, 1272-1286, 1273-1287, 1274-1288, 1275-1289, 1276-1290, 1277-1291, 1278-1292, 1279-1293, 1280-1294, 1281-1295, 1282-1296, 1283-1297, 1284-1298, 1285-1299, 1286-1300, 1287-1301, 1288-1302, 1289-1303, 1290-1304, 1291-1305, 1292-1306, 1293-1307, 1294-1308, 1295-1309, 1296-1310, 1297-1311, 1298-1312, 1299-1313, 1300-1314, 1301-1315, 1302-1316, 1303-1317, 1304-1318, 1305-1319, 1306-1320, 1307-1321, 1308-1322, 1309-1323, 1310-1324, 1311-1325, 1312-1326, 1313-1327, 1314-1328, 1315-1329, 1316-1330, 1317-1331, 1318-1332, 1319-1333, 1320-1334, 1321-1335, 1322-1336, 1323-1337, 1324-1338, 1325-1339, 1326-1340, 1327-1341, 1328-1342, 1329-1343, 1330-1344, 1331-1345, 1332-1346, 1333-1347, 1334-1348, 1335-1349, 1336-1350, 1337-1351, 1338-1352, 1339-1353, 1340-1354, 1341-1355, 1342-1356, 1343-1357, 1344-1358, 1345-1359, 1346-1360, 1347-1361, 1348-1362, 1349-1363, 1350-1364, 1351-1365, 1352-1366, 1353-1367, 1354-1368, 1355-1369, 1356-1370, 1357-1371, 1358-1372, 1359-1373, 1360-1374, 1361-1375, 1362-1376, 1363-1377, 1364-1378, 1365-1379, 1366-1380, 1367-1381, 1368-1382, 1369-1383, 1370-1384, 1371-1385, 1372-1386, 1373-1387, 1374-1388, 1375-1389, 1376-1390, 1377-1391, 1378-1392, 1379-1393, 1380-1394, 1381-1395, 1382-1396, 1383-1397, 1384-1398, 1385-1399, 1386-1400, 1387-1401, 1388-1402, 1389-1403, 1390-1404, 1391-1405, 1392-1406, 1393-1407, 1394-1408, 1395-1409, 1396-1410, 1397-1411, 1398-1412, 1399-1413, 1400-1414, 1401-1415, 1402-1416, 1403-1417, 1404-1418, 1405-1419, 1406-1420, 1407-1421, 1408-1422, 1409-1423, 1410-1424, 1411-1425, 1412-1426, 1413-1427, 1414-1428, 1415-1429, 1416-1430, 1417-1431, 1418-1432, 1419-1433, 1420-1434, 1421-1435, 1422-1436, 1423-1437, 1424-1438, 1425-1439, 1426-1440, 1427-1441, 1428-1442, 1429-1443, 1430-1444, 1431-1445, 1432-1446, 1433-1447, 1434-1448, 1435-1449, 1436-1450, 1437-1451, 1438-1452, 1439-1453, 1440-1454, 1441-1455, 1442-1456, 1443-1457, 1444-1458, 1445-1459, 1446-1460, 1447-1461, 1448-1462, 1449-1463, 1450-1464, 1451-1465, 1452-1466, 1453-1467, 1454-1468, 1455-1469, 1456-1470, 1457-1471, 1458-1472, 1459-1473, 1460-1474, 1461-1475, 1462-1476, 1463-1477, 1464-1478, 1465-1479, 1466-1480, 1467-1481, 1468-1482, 1469-1483, 1470-1484, 1471-1485, 1472-1486, 1473-1487, 1474-1488, 1475-1489, 1476-1490, 1477-1491, 1478-1492, 1479-1493, 1480-1494, 1481-1495, 1482-1496, 1483-1497, 1484-1498, 1485-1499, 1486-1500, 1487-1501, 1488-1502, 1489-1503, 1490-1504, 1491-1505, 1492-1506, 1493-1507, 1494-1508, 1495-1509, 1496-1510, 1497-1511, 1498-1512, 1499-1513, 1500-1514, 1501-1515, 1502-1516, 1503-1517, 1504-1518, 1505-1519, 1506-1520, 1507-1521, 1508-1522, 1509-1523, 1510-1524, 1511-1525, 1512-1526, 1513-1527, 1514-1528, 1515-1529, 1516-1530, 1517-1531, 1518-1532, 1519-1533, 1520-1534, 1521-1535, 1522-1536, 1523-1537, 1524-1538, 1525-1539, 1526-1540, 1527-1541, 1528-1542, 1529-1543, 1530-1544, 1531-1545, 1532-1546, 1533-1547, 1534-1548, 1535-1549, 1536-1550, 1537-1551, 1538-1552, 1539-1553, 1540-1554, 1541-1555, 1542-1556, 1543-1557, 1544-1558, 1545-1559, 1546-1560, 1547-1561, 1548-1562, 1549-1563, 1550-1564, 1551-1565, 1552-1566, 1553-1567, 1554-1568, 1555-1569, 1556-1570, 1557-1571, 1558-1572, 1559-1573, 1560-1574, 1561-1575, 1562-1576, 1563-1577, 1564-1578, 1565-1579, 1566-1580, 1567-1581, 1568-1582, 1569-1583, 1570-1584, 1571-1585, 1572-1586, 1573-1587, 1574-1588, 1575-1589, 1576-1590, 1577-1591, 1578-1592, 1579-1593, 1580-1594, 1581-1595, 1582-1596, 1583-1597, 1584-1598, 1585-1599, 1586-1600, 1587-1601, 1588-1602, 1589-1603, 1590-1604, 1591-1605, 1592-1606, 1593-1607, 1594-1608, 1595-1609, 1596-1610, 1597-1611, 1598-1612, 1599-1613, 1600-1614, 1601-1615, 1602-1616, 1603-1617, 1604-1618, 1605-1619, 1606-1620, 1607-1621, 1608-1622, 1609-1623, 1610-1624, 1611-1625, 1612-1626, 1613-1627, 1614-1628, 1615-1629, 1616-1630, 1617-1631, 1618-1632, 1619-1633, 1620-1634, 1621-1635, 1622-1636, 1623-1637, 1624-1638, 1625-1639, 1626-1640, 1627-1641, 1628-1642, 1629-1643, 1630-1644, 1631-1645, 1632-1646, 1633-1647, 1634-1648, 1635-1649, 1636-1650, 1637-1651, 1638-1652, 1639-1653, 1640-1654, 1641-1655, 1642-1656, 1643-1657, 1644-1658, 1645-1659, 1646-1660, 1647-1661, 1648-1662, 1649-1663, 1650-1664, 1651-1665, 1652-1666, 1653-1667, 1654-1668, 1655-1669, 1656-1670, 1657-1671, 1658-1672, 1659-1673, 1660-1674, 1661-1675, 1662-1676, 1663-1677, 1664-1678, 1665-1679, 1666-1680, 1667-1681, 1668-1682, 1669-1683, 1670-1684, 1671-1685, 1672-1686, 1673-1687, 1674-1688, 1675-1689, 1676-1690, 1677-1691, 1678-1692, 1679-1693, 1680-1694, 1681-1695, 1682-1696, 1683-1697, 1684-1698, 1685-1699, 1686-1700, 1687-1701, 1688-1702, 1689-1703, 1690-1704, 1691-1705, 1692-1706, 1693-1707, 1694-1708, 1695-1709, 1696-1710, 1697-1711, 1698-1712, 1699-1713, 1700-1714, 1701-1715, 1702-1716, 1703-1717, 1704-1718, 1705-1719, 1706-1720, 1707-1721, 1708-1722, 1709-1723, 1710-1724, 1711-1725, 1712-1726, 1713-1727, 1714-1728, 1715-1729, 1716-1730, 1717-1731, 1718-1732, 1719-1733, 1720-1734, 1721-1735, 1722-1736, 1723-1737, 1724-1738, 1725-1739, 1726-1740, 1727-1741, 1728-1742, 1729-1743, 1730-1744, 1731-1745, 1732-1746, 1733-1747, 1734-1748, 1735-1749, 1736-1750, 1737-1751, 1738-1752, 1739-1753, 1740-1754, 1741-1755, 1742-1756, 1743-1757, 1744-1758, 1745-1759, 1746-1760, 1747-1761, 1748-1762, 1749-1763, 1750-1764, 1751-1765, 1752-1766, 1753-1767, 1754-1768, 1755-1769, 1756-1770, 1757-1771, 1758-1772, 1759-1773, 1760-1774, 1761-1775, 1762-1776, 1763-1777, 1764-1778, 1765-1779, 1766-1780, 1767-1781, 1768-1782, 1769-1783, 1770-1784, 1771-1785, 1772-1786, 1773-1787, 1774-1788, 1775-1789, 1776-1790, 1777-1791, 1778-1792, 1779-1793, 1780-1794, 1781-1795, 1782-1796, 1783-1797, 1784-1798, 1785-1799, 1786-1800, 1787-1801, 1788-1802, 1789-1803, 1790-1804, 1791-1805, 1792-1806, 1793-1807, 1794-1808, 1795-1809, 1796-1810, 1797-1811, 1798-1812, 1799-1813, 1800-1814, 1801-1815, 1802-1816, 1803-1817, 1804-1818, 1805-1819, 1806-1820, 1807-1821, 1808-1822, 1809-1823, 1810-1824, 1811-1825, 1812-1826, 1813-1827, 1814-1828, 1815-1829, 1816-1830, 1817-1831, 1818-1832, 1819-1833, 1820-1834, 1821-1835, 1822-1836, 1823-1837, 1824-1838, 1825-1839, 1826-1840, 1827-1841, 1828-1842, 1829-1843, 1830-1844, 1831-1845, 1832-1846, 1833-1847, 1834-1848, 1835-1849, 1836-1850, 1837-1851, 1838-1852, 1839-1853, 1840-1854, 1841-1855, 1842-1856, 1843-1857, 1844-1858, 1845-1859, 1846-1860, 1847-1861, 1848-1862, 1849-1863, 1850-1864, 1851-1865, 1852-1866, 1853-1867, 1854-1868, 1855-1869, 1856-1870, 1857-1871, 1858-1872, 1859-1873, 1860-1874, 1861-1875, 1862-1876, 1863-1877, 1864-1878, 1865-1879, 1866-1880, 1867-1881, 1868-1882, 1869-1883, 1870-1884, 1871-1885, 1872-1886, 1873-1887, 1874-1888, 1875-1889, 1876-1890, 1877-1891, 1878-1892, 1879-1893, 1880-1894, 1881-1895, 1882-1896, 1883-1897, 1884-1898, 1885-1899, 1886-1900, 1887-1901, 1888-1902, 1889-1903, 1890-1904, 1891-1905, 1892-1906, 1893-1907, 1894-1908, 1895-1909, 1896-1910, 1897-1911, 1898-1912, 1899-1913, 1900-1914, 1901-1915, 1902-1916, 1903-1917, 1904-1918, 1905-1919, 1906-1920, 1907-1921, 1908-1922, 1909-1923, 1910-1924, 1911-1925, 1912-1926, 1913-1927, 1914-1928, 1915-1929, 1916-1930, 1917-1931, 1918-1932, 1919-1933, 1920-1934, 1921-1935, 1922-1936, 1923-1937, 1924-1938, 1925-1939, 1926-1940, 1927-1941, 1928-1942, 1929-1943, 1930-1944, 1931-1945, 1932-1946, 1933-1947, 1934-1948, 1935-1949, 1936-1950, 1937-1951, 1938-1952, 1939-1953, 1940-1954, 1941-1955, 1942-1956, 1943-1957, 1944-1958, 1945-1959, 1946-1960, 1947-1961, 1948-1962, 1949-1963, 1950-1964, 1951-1965, 1952-1966, 1953-1967, 1954-1968, 1955-1969, 1956-1970, 1957-1971, 1958-1972, 1959-1973, 1960-1974, 1961-1975, 1962-1976, 1963-1977, 1964-1978, 1965-1979, 1966-1980, 1967-1981, 1968-1982, 1969-1983, 1970-1984, 1971-1985, 1972-1986, 1973-1987, 1974-1988, 1975-1989, 1976-1990, 1977-1991, 1978-1992, 1979-1993, 1980-1994, 1981-1995, 1982-1996, 1983-1997, 1984-1998, 1985-1999, 1986-2000, 1987-2001, 1988-2002, 1989-2003, 1990-2004, 1991-2005, 1992-2006, 1993-2007, 1994-2008, 1995-2009, 1996-2010, 1997-2011, 1998-2012, 1999-2013, 2000-2014, 2001-2015, 2002-2016, 2003-2017, 2004-2018, 2005-2019, 2006-2020, 2007-2021, 2008-2022, 2009-2023, 2010-2024, 2011-2025, 2012-2026, 2013-2027, 2014-2028, 2015-2029, 2016-2030, 2017-2031, 2018-2032, 2019-2033, 2020-2034, 2021-2035, 2022-2036, 2023-2037, 2024-2038, 2025-2039, 2026-2040, 2027-2041, 2028-2042, 2029-2043, 2030-2044, 2031-2045, 2032-2046, 2033-2047, 2034-2048, 2035-2049, 2036-2050, 2037-2051, 2038-2052, 2039-2053, 2040-2054, 2041-2055, 2042-2056, 2043-2057, 2044-2058, 2045-2059, 2046-2060, 2047-2061, 2048-2062, 2049-2063, 2050-2064, 2051-2065, 2052-2066, 2053-2067, 2054-2068, 2055-2069, 2056-2070, 2057-2071, 2058-2072, 2059-2073, 2060-2074, 2061-2075, 2062-2076, 2063-2077, 2064-2078, 2065-2079, 2066-2080, 2067-2081, 2068-2082, 2069-2083, 2070-2084, 2071-2085, 2072-2086, 2073-2087, 2074-2088, 2075-2089, 2076-2090, 2077-2091, 2078-2092, 2079-2093, 2080-2094, 2081-2095, 2082-2096, 2083-2097, 2084-2098, 2085-2099, 2086-2100, 2087-2101, 2088-2102, 2089-2103, 2090-2104, 2091-2105, 2092-2106, 2093-2107, 2094-2108, 2095-2109, 2096-2110, 2097-2111, 2098-2112, 2099-2113, 2100-2114, 2101-2115, 2102-2116, 2103-2117, 2104-2118, 2105-2119, 2106-2120, 2107-2121, 2108-2122, 2109-2123, 2110-2124, 2111-2125, 2112-2126, 2113-2127, 2114-2128, 2115-2129, 2116-2130, 2117-2131, 2118-2132, 2119-2133, 2120-2134, 2121-2135, 2122-2136, 2123-2137, 2124-2138, 2125-2139, 2126-2140, 2127-2141, 2128-2142, 2129-2143, 2130-2144, 2131-2145, 2132-2146, 2133-2147, 2134-2148, 2135-2149, 2136-2150, 2137-2151, 2138-2152, 2139-2153, 2140-2154, 2141-2155, 2142-2156, 2143-2157, 2144-2158, 2145-2159, 2146-2160, 2147-2161, 2148-2162, 2149-2163, 2150-2164, 2151-2165, 2152-2166, 2153-2167, 2154-2168, 2155-2169, 2156-2170, 2157-2171, 2158-2172, 2159-2173, 2160-2174, 2161-2175, 2162-2176, 2163-2177, 2164-2178, 2165-2179, 2166-2180, 2167-2181, 2168-2182, 2169-2183, 2170-2184, 2171-2185, 2172-2186, 2173-2187, 2174-2188, 2175-2189, 2176-2190, 2177-2191, 2178-2192, 2179-2193, 2180-2194, 2181-2195, 2182-2196, 2183-2197, 2184-2198, 2185-2199, 2186-2200, 2187-2201, 2188-2202, 2189-2203, 2190-2204, 2191-2205, 2192-2206, 2193-2207, 2194-2208, 2195-2209, 2196-2210, 2197-2211, 2198-2212, 2199-2213, 2200-2214, 2201-2215, 2202-2216, 2203-2217, 2204-2218, 2205-2219, 2206-2220, 2207-2221, 2208-2222, 2209-2223, 2210-2224, 2211-2225, 2212-2226, 2213-2227, 2214-2228, 2215-2229, 2216-2230, 2217-2231 and 2218-2232.

Exemplary polynucleotide molecules include the following 20-mer fragments of the polynucleotide sequence from the sequence of SEQ ID NO:1: 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-1420, 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, 1581-1600, 1582-1601, 1583-1602, 1584-1603, 1585-1604, 1586-1605, 1587-1606, 1588-1607, 1589-1608, 1590-1609, 1591-1610, 1592-1611, 1593-1612, 1594-1613, 1595-1614, 1596-1615, 1597-1616, 1598-1617, 1599-1618, 1600-1619, 1601-1620, 1602-1621, 1603-1622, 1604-1623, 1605-1624, 1606-1625, 1607-1626, 1608-1627, 1609-1628, 1610-1629, 1611-1630, 1612-1631, 1613-1632, 1614-1633, 1615-1634, 1616-1635, 1617-1636, 1618-1637, 1619-1638, 1620-1639, 1621-1640, 1622-1641, 1623-1642, 1624-1643, 1625-1644, 1626-1645, 1627-1646, 1628-1647, 1629-1648, 1630-1649, 1631-1650, 1632-1651, 1633-1652, 1634-1653, 1635-1654, 1636-1655, 1637-1656, 1638-1657, 1639-1658, 1640-1659, 1641-1660, 1642-1661, 1643-1662, 1644-1663, 1645-1664, 1646-1665, 1647-1666, 1648-1667, 1649-1668, 1650-1669, 1651-1670, 1652-1671, 1653-1672, 1654-1673, 1655-1674, 1656-1675, 1657-1676, 1658-1677, 1659-1678, 1660-1679, 1661-1680, 1662-1681, 1663-1682, 1664-1683, 1665-1684, 1666-1685, 1667-1686, 1668-1687, 1669-1688, 1670-1689, 1671-1690, 1672-1691, 1673-1692, 1674-1693, 1675-1694, 1676-1695, 1677-1696, 1678-1697, 1679-1698, 1680-1699, 1681-1700, 1682-1701, 1683-1702, 1684-1703, 1685-1704, 1686-1705, 1687-1706, 1688-1707, 1689-1708, 1690-1709, 1691-1710, 1692-1711, 1693-1712, 1694-1713, 1695-1714, 1696-1715, 1697-1716, 1698-1717, 1699-1718, 1700-1719, 1701-1720, 1702-1721, 1703-1722, 1704-1723, 1705-1724, 1706-1725, 1707-1726, 1708-1727, 1709-1728, 1710-1729, 1711-1730, 1712-1731, 1713-1732, 1714-1733, 1715-1734, 1716-1735, 1717-1736, 1718-1737, 1719-1738, 1720-1739, 1721-1740, 1722-1741, 1723-1742, 1724-1743, 1725-1744, 1726-1745, 1727-1746, 1728-1747, 1729-1748, 1730-1749, 1731-1750, 1732-1751, 1733-1752, 1734-1753, 1735-1754, 1736-1755, 1737-1756, 1738-1757, 1739-1758, 1740-1759, 1741-1760, 1742-1761, 1743-1762, 1744-1763, 1745-1764, 1746-1765, 1747-1766, 1748-1767, 1749-1768, 1750-1769, 1751-1770, 1752-1771, 1753-1772, 1754-1773, 1755-1774, 1756-1775, 1757-1776, 1758-1777, 1759-1778, 1760-1779, 1761-1780, 1762-1781, 1763-1782, 1764-1783, 1765-1784, 1766-1785, 1767-1786, 1768-1787, 1769-1788, 1770-1789, 1771-1790, 1772-1791, 1773-1792, 1774-1793, 1775-1794, 1776-1795, 1777-1796, 1778-1797, 1779-1798, 1780-1799, 1781-1800, 1782-1801, 1783-1802, 1784-1803, 1785-1804, 1786-1805, 1787-1806, 1788-1807, 1789-1808, 1790-1809, 1791-1810, 1792-1811, 1793-1812, 1794-1813, 1795-1814, 1796-1815, 1797-1816, 1798-1817, 1799-1818, 1800-1819, 1801-1820, 1802-1821, 1803-1822, 1804-1823, 1805-1824, 1806-1825, 1807-1826, 1808-1827, 1809-1828, 1810-1829, 1811-1830, 1812-1831, 1813-1832, 1814-1833, 1815-1834, 1816-1835, 1817-1836, 1818-1837, 1819-1838, 1820-1839, 1821-1840, 1822-1841, 1823-1842, 1824-1843, 1825-1844, 1826-1845, 1827-1846, 1828-1847, 1829-1848, 1830-1849, 1831-1850, 1832-1851, 1833-1852, 1834-1853, 1835-1854, 1836-1855, 1837-1856, 1838-1857, 1839-1858, 1840-1859, 1841-1860, 1842-1861, 1843-1862, 1844-1863, 1845-1864, 1846-1865, 1847-1866, 1848-1867, 1849-1868, 1850-1869, 1851-1870, 1852-1871, 1853-1872, 1854-1873, 1855-1874, 1856-1875, 1857-1876, 1858-1877, 1859-1878, 1860-1879, 1861-1880, 1862-1881, 1863-1882, 1864-1883, 1865-1884, 1866-1885, 1867-1886, 1868-1887, 1869-1888, 1870-1889, 1871-1890, 1872-1891, 1873-1892, 1874-1893, 1875-1894, 1876-1895, 1877-1896, 1878-1897, 1879-1898, 1880-1899, 1881-1900, 1882-1901, 1883-1902, 1884-1903, 1885-1904, 1886-1905, 1887-1906, 1888-1907, 1889-1908, 1890-1909, 1891-1910, 1892-1911, 1893-1912, 1894-1913, 1895-1914, 1896-1915, 1897-1916, 1898-1917, 1899-1918, 1900-1919, 1901-1920, 1902-1921, 1903-1922, 1904-1923, 1905-1924, 1906-1925, 1907-1926, 1908-1927, 1909-1928, 1910-1929, 1911-1930, 1912-1931, 1913-1932, 1914-1933, 1915-1934, 1916-1935, 1917-1936, 1918-1937, 1919-1938, 1920-1939, 1921-1940, 1922-1941, 1923-1942, 1924-1943, 1925-1944, 1926-1945, 1927-1946, 1928-1947, 1929-1948, 1930-1949, 1931-1950, 1932-1951, 1933-1952, 1934-1953, 1935-1954, 1936-1955, 1937-1956, 1938-1957, 1939-1958, 1940-1959, 1941-1960, 1942-1961, 1943-1962, 1944-1963, 1945-1964, 1946-1965, 1947-1966, 1948-1967, 1949-1968, 1950-1969, 1951-1970, 1952-1971, 1953-1972, 1954-1973, 1955-1974, 1956-1975, 1957-1976, 1958-1977, 1959-1978, 1960-1979, 1961-1980, 1962-1981, 1963-1982, 1964-1983, 1965-1984, 1966-1985, 1967-1986, 1968-1987, 1969-1988, 1970-1989, 1971-1990, 1972-1991, 1973-1992, 1974-1993, 1975-1994, 1976-1995, 1977-1996, 1978-1997, 1979-1998, 1980-1999, 1981-2000, 1982-2001, 1983-2002, 1984-2003, 1985-2004, 1986-2005, 1987-2006, 1988-2007, 1989-2008, 1990-2009, 1991-2010, 1992-2011, 1993-2012, 1994-2013, 1995-2014, 1996-2015, 1997-2016, 1998-2017, 1999-2018, 2000-2019, 2001-2020, 2002-2021, 2003-2022, 2004-2023, 2005-2024, 2006-2025, 2007-2026, 2008-2027, 2009-2028, 2010-2029, 2011-2030, 2012-2031, 2013-2032, 2014-2033, 2015-2034, 2016-2035, 2017-2036, 2018-2037, 2019-2038, 2020-2039, 2021-2040, 2022-2041, 2023-2042, 2024-2043, 2025-2044, 2026-2045, 2027-2046, 2028-2047, 2029-2048, 2030-2049, 2031-2050, 2032-2051, 2033-2052, 2034-2053, 2035-2054, 2036-2055, 2037-2056, 2038-2057, 2039-2058, 2040-2059, 2041-2060, 2042-2061, 2043-2062, 2044-2063, 2045-2064, 2046-2065, 2047-2066, 2048-2067, 2049-2068, 2050-2069, 2051-2070, 2052-2071, 2053-2072, 2054-2073, 2055-2074, 2056-2075, 2057-2076, 2058-2077, 2059-2078, 2060-2079, 2061-2080, 2062-2081, 2063-2082, 2064-2083, 2065-2084, 2066-2085, 2067-2086, 2068-2087, 2069-2088, 2070-2089, 2071-2090, 2072-2091, 2073-2092, 2074-2093, 2075-2094, 2076-2095, 2077-2096, 2078-2097, 2079-2098, 2080-2099, 2081-2100, 2082-2101, 2083-2102, 2084-2103, 2085-2104, 2086-2105, 2087-2106, 2088-2107, 2089-2108, 2090-2109, 2091-2110, 2092-2111, 2093-2112, 2094-2113, 2095-2114, 2096-2115, 2097-2116, 2098-2117, 2099-2118, 2100-2119, 2101-2120, 2102-2121, 2103-2122, 2104-2123, 2105-2124, 2106-2125, 2107-2126, 2108-2127, 2109-2128, 2110-2129, 2111-2130, 2112-2131, 2113-2132, 2114-2133, 2115-2134, 2116-2135, 2117-2136, 2118-2137, 2119-2138, 2120-2139, 2121-2140, 2122-2141, 2123-2142, 2124-2143, 2125-2144, 2126-2145, 2127-2146, 2128-2147, 2129-2148, 2130-2149, 2131-2150, 2132-2151, 2133-2152, 2134-2153, 2135-2154, 2136-2155, 2137-2156, 2138-2157, 2139-2158, 2140-2159, 2141-2160, 2142-2161, 2143-2162, 2144-2163, 2145-2164, 2146-2165, 2147-2166, 2148-2167, 2149-2168, 2150-2169, 2151-2170, 2152-2171, 2153-2172, 2154-2173, 2155-2174, 2156-2175, 2157-2176, 2158-2177, 2159-2178, 2160-2179, 2161-2180, 2162-2181, 2163-2182, 2164-2183, 2165-2184, 2166-2185, 2167-2186, 2168-2187, 2169-2188, 2170-2189, 2171-2190, 2172-2191, 2173-2192, 2174-2193, 2175-2194, 2176-2195, 2177-2196, 2178-2197, 2179-2198, 2180-2199, 2181-2200, 2182-2201, 2183-2202, 2184-2203, 2185-2204, 2186-2205, 2187-2206, 2188-2207, 2189-2208, 2190-2209, 2191-2210, 2192-2211, 2193-2212, 2194-2213, 2195-2214, 2196-2215, 2197-2216, 2198-2217, 2199-2218, 2200-2219, 2201-2220, 2202-2221, 2203-2222, 2204-2223, 2205-2224, 2206-2225, 2207-2226, 2208-2227, 2209-2228, 2210-2229, 2211-2230, 2212-2231 and 2213-2232.

Exemplary polynucleotide molecules include the following 25-mer fragments of the polynucleotide sequence from the sequence of SEQ ID NO:1: 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815, 792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1472, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-1501, 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568, 1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600, 1577-1601, 1578-1602, 1579-1603, 1580-1604, 1581-1605, 1582-1606, 1583-1607, 1584-1608, 1585-1609, 1586-1610, 1587-1611, 1588-1612, 1589-1613, 1590-1614, 1591-1615, 1592-1616, 1593-1617, 1594-1618, 1595-1619, 1596-1620, 1597-1621, 1598-1622, 1599-1623, 1600-1624, 1601-1625, 1602-1626, 1603-1627, 1604-1628, 1605-1629, 1606-1630, 1607-1631, 1608-1632, 1609-1633, 1610-1634, 1611-1635, 1612-1636, 1613-1637, 1614-1638, 1615-1639, 1616-1640, 1617-1641, 1618-1642, 1619-1643, 1620-1644, 1621-1645, 1622-1646, 1623-1647, 1624-1648, 1625-1649, 1626-1650, 1627-1651, 1628-1652, 1629-1653, 1630-1654, 1631-1655, 1632-1656, 1633-1657, 1634-1658, 1635-1659, 1636-1660, 1637-1661, 1638-1662, 1639-1663, 1640-1664, 1641-1665, 1642-1666, 1643-1667, 1644-1668, 1645-1669, 1646-1670, 1647-1671, 1648-1672, 1649-1673, 1650-1674, 1651-1675, 1652-1676, 1653-1677, 1654-1678, 1655-1679, 1656-1680, 1657-1681, 1658-1682, 1659-1683, 1660-1684, 1661-1685, 1662-1686, 1663-1687, 1664-1688, 1665-1689, 1666-1690, 1667-1691, 1668-1692, 1669-1693, 1670-1694, 1671-1695, 1672-1696, 1673-1697, 1674-1698, 1675-1699, 1676-1700, 1677-1701, 1678-1702, 1679-1703, 1680-1704, 1681-1705, 1682-1706, 1683-1707, 1684-1708, 1685-1709, 1686-1710, 1687-1711, 1688-1712, 1689-1713, 1690-1714, 1691-1715, 1692-1716, 1693-1717, 1694-1718, 1695-1719, 1696-1720, 1697-1721, 1698-1722, 1699-1723, 1700-1724, 1701-1725, 1702-1726, 1703-1727, 1704-1728, 1705-1729, 1706-1730, 1707-1731, 1708-1732, 1709-1733, 1710-1734, 1711-1735, 1712-1736, 1713-1737, 1714-1738, 1715-1739, 1716-1740, 1717-1741, 1718-1742, 1719-1743, 1720-1744, 1721-1745, 1722-1746, 1723-1747, 1724-1748, 1725-1749, 1726-1750, 1727-1751, 1728-1752, 1729-1753, 1730-1754, 1731-1755, 1732-1756, 1733-1757, 1734-1758, 1735-1759, 1736-1760, 1737-1761, 1738-1762, 1739-1763, 1740-1764, 1741-1765, 1742-1766, 1743-1767, 1744-1768, 1745-1769, 1746-1770, 1747-1771, 1748-1772, 1749-1773, 1750-1774, 1751-1775, 1752-1776, 1753-1777, 1754-1778, 1755-1779, 1756-1780, 1757-1781, 1758-1782, 1759-1783, 1760-1784, 1761-1785, 1762-1786, 1763-1787, 1764-1788, 1765-1789, 1766-1790, 1767-1791, 1768-1792, 1769-1793, 1770-1794, 1771-1795, 1772-1796, 1773-1797, 1774-1798, 1775-1799, 1776-1800, 1777-1801, 1778-1802, 1779-1803, 1780-1804, 1781-1805, 1782-1806, 1783-1807, 1784-1808, 1785-1809, 1786-1810, 1787-1811, 1788-1812, 1789-1813, 1790-1814, 1791-1815, 1792-1816, 1793-1817, 1794-1818, 1795-1819, 1796-1820, 1797-1821, 1798-1822, 1799-1823, 1800-1824, 1801-1825, 1802-1826, 1803-1827, 1804-1828, 1805-1829, 1806-1830, 1807-1831, 1808-1832, 1809-1833, 1810-1834, 1811-1835, 1812-1836, 1813-1837, 1814-1838, 1815-1839, 1816-1840, 1817-1841, 1818-1842, 1819-1843, 1820-1844, 1821-1845, 1822-1846, 1823-1847, 1824-1848, 1825-1849, 1826-1850, 1827-1851, 1828-1852, 1829-1853, 1830-1854, 1831-1855, 1832-1856, 1833-1857, 1834-1858, 1835-1859, 1836-1860, 1837-1861, 1838-1862, 1839-1863, 1840-1864, 1841-1865, 1842-1866, 1843-1867, 1844-1868, 1845-1869, 1846-1870, 1847-1871, 1848-1872, 1849-1873, 1850-1874, 1851-1875, 1852-1876, 1853-1877, 1854-1878, 1855-1879, 1856-1880, 1857-1881, 1858-1882, 1859-1883, 1860-1884, 1861-1885, 1862-1886, 1863-1887, 1864-1888, 1865-1889, 1866-1890, 1867-1891, 1868-1892, 1869-1893, 1870-1894, 1871-1895, 1872-1896, 1873-1897, 1874-1898, 1875-1899, 1876-1900, 1877-1901, 1878-1902, 1879-1903, 1880-1904, 1881-1905, 1882-1906, 1883-1907, 1884-1908, 1885-1909, 1886-1910, 1887-1911, 1888-1912, 1889-1913, 1890-1914, 1891-1915, 1892-1916, 1893-1917, 1894-1918, 1895-1919, 1896-1920, 1897-1921, 1898-1922, 1899-1923, 1900-1924, 1901-1925, 1902-1926, 1903-1927, 1904-1928, 1905-1929, 1906-1930, 1907-1931, 1908-1932, 1909-1933, 1910-1934, 1911-1935, 1912-1936, 1913-1937, 1914-1938, 1915-1939, 1916-1940, 1917-1941, 1918-1942, 1919-1943, 1920-1944, 1921-1945, 1922-1946, 1923-1947, 1924-1948, 1925-1949, 1926-1950, 1927-1951, 1928-1952, 1929-1953, 1930-1954, 1931-1955, 1932-1956, 1933-1957, 1934-1958, 1935-1959, 1936-1960, 1937-1961, 1938-1962, 1939-1963, 1940-1964, 1941-1965, 1942-1966, 1943-1967, 1944-1968, 1945-1969, 1946-1970, 1947-1971, 1948-1972, 1949-1973, 1950-1974, 1951-1975, 1952-1976, 1953-1977, 1954-1978, 1955-1979, 1956-1980, 1957-1981, 1958-1982, 1959-1983, 1960-1984, 1961-1985, 1962-1986, 1963-1987, 1964-1988, 1965-1989, 1966-1990, 1967-1991, 1968-1992, 1969-1993, 1970-1994, 1971-1995, 1972-1996, 1973-1997, 1974-1998, 1975-1999, 1976-2000, 1977-2001, 1978-2002, 1979-2003, 1980-2004, 1981-2005, 1982-2006, 1983-2007, 1984-2008, 1985-2009, 1986-2010, 1987-2011, 1988-2012, 1989-2013, 1990-2014, 1991-2015, 1992-2016, 1993-2017, 1994-2018, 1995-2019, 1996-2020, 1997-2021, 1998-2022, 1999-2023, 2000-2024, 2001-2025, 2002-2026, 2003-2027, 2004-2028, 2005-2029, 2006-2030, 2007-2031, 2008-2032, 2009-2033, 2010-2034, 2011-2035, 2012-2036, 2013-2037, 2014-2038, 2015-2039, 2016-2040, 2017-2041, 2018-2042, 2019-2043, 2020-2044, 2021-2045, 2022-2046, 2023-2047, 2024-2048, 2025-2049, 2026-2050, 2027-2051, 2028-2052, 2029-2053, 2030-2054, 2031-2055, 2032-2056. 2033-2057, 2034-2058, 2035-2059, 2036-2060, 2037-2061, 2038-2062, 2039-2063, 2040-2064, 2041-2065, 2042-2066, 2043-2067, 2044-2068, 2045-2069, 2046-2070, 2047-2071, 2048-2072, 2049-2073, 2050-2074, 2051-2075, 2052-2076, 2053-2077, 2054-2078, 2055-2079, 2056-2080, 2057-2081, 2058-2082, 2059-2083, 2060-2084, 2061-2085, 2062-2086, 2063-2087, 2064-2088, 2065-2089, 2066-2090, 2067-2091, 2068-2092, 2069-2093, 2070-2094, 2071-2095, 2072-2096, 2073-2097, 2074-2098, 2075-2099, 2076-2100, 2077-2101, 2078-2102, 2079-2103, 2080-2104, 2081-2105, 2082-2106, 2083-2107, 2084-2108, 2085-2109, 2086-2110, 2087-2111, 2088-2112, 2089-2113, 2090-2114, 2091-2115, 2092-2116, 2093-2117, 2094-2118, 2095-2119, 2096-2120, 2097-2121, 2098-2122, 2099-2123, 2100-2124, 2101-2125, 2102-2126, 2103-2127, 2104-2128, 2105-2129, 2106-2130, 2107-2131, 2108-2132, 2109-2133, 2110-2134, 2111-2135, 2112-2136, 2113-2137, 2114-2138, 2115-2139, 2116-2140, 2117-2141, 2118-2142, 2119-2143, 2120-2144, 2121-2145, 2122-2146, 2123-2147, 2124-2148, 2125-2149, 2126-2150, 2127-2151, 2128-2152, 2129-2153, 2130-2154, 2131-2155, 2132-2156, 2133-2157, 2134-2158, 2135-2159, 2136-2160, 2137-2161, 2138-2162, 2139-2163, 2140-2164, 2141-2165, 2142-2166, 2143-2167, 2144-2168, 2145-2169, 2146-2170, 2147-2171, 2148-2172, 2149-2173, 2150-2174, 2151-2175, 2152-2176, 2153-2177, 2154-2178, 2155-2179, 2156-2180, 2157-2181, 2158-2182, 2159-2183, 2160-2184, 2161-2185, 2162-2186, 2163-2187, 2164-2188, 2165-2189, 2166-2190, 2167-2191, 2168-2192, 2169-2193, 2170-2194, 2171-2195, 2172-2196, 2173-2197, 2174-2198, 2175-2199, 2176-2200, 2177-2201, 2178-2202, 2179-2203, 2180-2204, 2181-2205, 2182-2206, 2183-2207, 2184-2208, 2185-2209, 2186-2210, 2187-2211, 2188-2212, 2189-2213, 2190-2214, 2191-2215, 2192-2216, 2193-2217, 2194-2218, 2195-2219, 2196-2220, 2197-2221, 2198-2222, 2199-2223, 2200-2224, 2201-2225, 2202-2226, 2203-2227, 2204-2228, 2205-2229, 2206-2230, 2207-2231 and 2208-2232.

Exemplary polynucleotide molecules include the following 12-mer fragments of the polynucleotide sequence from the sequence of SEQ ID NO:4: 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723, 713-724, 714-725, 715-726, 716-727, 717-728, 718-729, 719-730, 720-731, 721-732, 722-733, 723-734, 724-735, 725-736, 726-737, 727-738, 728-739, 729-740, 730-741, 731-742, 732-743, 733-744, 734-745, 735-746, 736-747, 737-748, 738-749, 739-750, 740-751, 741-752, 742-753, 743-754, 744-755, 745-756, 746-757, 747-758, 748-759, 749-760, 750-761, 751-762, 752-763, 753-764, 754-765, 755-766, 756-767, 757-768, 758-769, 759-770, 760-771, 761-772, 762-773, 763-774, 764-775, 765-776, 766-777, 767-778, 768-779, 769-780, 770-781, 771-782, 772-783, 773-784, 774-785, 775-786, 776-787, 777-788, 778-789, 779-790, 780-791, 781-792, 782-793, 783-794, 784-795, 785-796, 786-797, 787-798, 788-799, 789-800, 790-801, 791-802, 792-803, 793-804, 794-805, 795-806, 796-807, 797-808, 798-809, 799-810, 800-811, 801-812, 802-813, 803-814, 804-815, 805-816, 806-817, 807-818, 808-819, 809-820, 810-821, 811-822, 812-823, 813-824, 814-825, 815-826, 816-827, 817-828, 818-829, 819-830, 820-831, 821-832, 822-833, 823-834, 824-835, 825-836, 826-837, 827-838, 828-839, 829-840, 830-841, 831-842, 832-843, 833-844, 834-845, 835-846, 836-847, 837-848, 838-849, 839-850, 840-851, 841-852, 842-853, 843-854, 844-855, 845-856, 846-857, 847-858, 848-859, 849-860, 850-861, 851-862, 852-863, 853-864, 854-865, 855-866, 856-867, 857-868, 858-869, 859-870, 860-871, 861-872, 862-873, 863-874, 864-875, 865-876, 866-877, 867-878, 868-879, 869-880, 870-881, 871-882, 872-883, 873-884, 874-885, 875-886, 876-887, 877-888, 878-889, 879-890, 880-891, 881-892, 882-893, 883-894, 884-895, 885-896, 886-897, 887-898, 888-899, 889-900, 890-901, 891-902, 892-903, 893-904, 894-905, 895-906, 896-907, 897-908, 898-909, 899-910, 900-911, 901-912, 902-913, 903-914, 904-915, 905-916, 906-917, 907-918, 908-919, 909-920, 910-921, 911-922, 912-923, 913-924, 914-925, 915-926, 916-927, 917-928, 918-929, 919-930, 920-931, 921-932, 922-933, 923-934, 924-935, 925-936, 926-937, 927-938, 928-939, 929-940, 930-941, 931-942, 932-943, 933-944, 934-945, 935-946, 936-947, 937-948, 938-949, 939-950, 940-951, 941-952, 942-953, 943-954, 944-955, 945-956, 946-957, 947-958, 948-959, 949-960, 950-961, 951-962, 952-963, 953-964, 954-965, 955-966, 956-967, 957-968, 958-969, 959-970, 960-971, 961-972, 962-973, 963-974, 964-975, 965-976, 966-977, 967-978, 968-979, 969-980, 970-981, 971-982, 972-983, 973-984, 974-985, 975-986, 976-987, 977-988, 978-989, 979-990, 980-991, 981-992, 982-993, 983-994, 984-995, 985-996, 986-997, 987-998, 988-999, 989-1000, 990-1001, 991-1002, 992-1003, 993-1004, 994-1005, 995-1006, 996-1007, 997-1008, 998-1009, 999-1010, 1000-1011, 1001-1012, 1002-1013, 1003-1014, 1004-1015, 1005-1016, 1006-1017, 1007-1018, 1008-1019, 1009-1020, 1010-1021, 1011-1022, 1012-1023, 1013-1024, 1014-1025, 1015-1026, 1016-1027, 1017-1028, 1018-1029, 1019-1030, 1020-1031, 1021-1032, 1022-1033, 1023-1034, 1024-1035, 1025-1036, 1026-1037, 1027-1038, 1028-1039, 1029-1040, 1030-1041, 1031-1042, 1032-1043, 1033-1044, 1034-1045, 1035-1046, 1036-1047, 1037-1048, 1038-1049, 1039-1050, 1040-1051, 1041-1052, 1042-1053, 1043-1054, 1044-1055, 1045-1056, 1046-1057, 1047-1058, 1048-1059, 1049-1060, 1050-1061, 1051-1062, 1052-1063, 1053-1064, 1054-1065, 1055-1066, 1056-1067, 1057-1068, 1058-1069, 1059-1070, 1060-1071, 1061-1072, 1062-1073, 1063-1074, 1064-1075, 1065-1076, 1066-1077, 1067-1078, 1068-1079, 1069-1080, 1070-1081, 1071-1082, 1072-1083, 1073-1084, 1074-1085, 1075-1086, 1076-1087, 1077-1088, 1078-1089, 1079-1090, 1080-1091, 1081-1092, 1082-1093, 1083-1094, 1084-1095, 1085-1096, 1086-1097, 1087-1098, 1088-1099, 1089-1100, 1090-1101, 1091-1102, 1092-1103, 1093-1104, 1094-1105, 1095-1106, 1096-1107, 1097-1108, 1098-1109, 1099-1110, 1100-1111, 1101-1112, 1102-1113, 1103-1114, 1104-1115, 1105-1116, 1106-1117, 1107-1118, 1108-1119, 1109-1120, 1110-1121, 1111-1122, 1112-1123, 1113-1124, 1114-1125, 1115-1126, 1116-1127, 1117-1128, 1118-1129, 1119-1130, 1120-1131, 1121-1132, 1122-1133, 1123-1134, 1124-1135, 1125-1136, 1126-1137, 1127-1138, 1128-1139, 1129-1140, 1130-1141, 1131-1142, 1132-1143, 1133-1144, 1134-1145, 1135-1146, 1136-1147, 1137-1148, 1138-1149, 1139-1150, 1140-1151, 1141-1152, 1142-1153, 1143-1154, 1144-1155, 1145-1156, 1146-1157, 1147-1158, 1148-1159, 1149-1160, 1150-1161, 1151-1162, 1152-1163, 1153-1164, 1154-1165, 1155-1166, 1156-1167, 1157-1168, 1158-1169, 1159-1170, 1160-1171, 1161-1172, 1162-1173, 1163-1174, 1164-1175, 1165-1176, 1166-1177, 1167-1178, 1168-1179, 1169-1180, 1170-1181, 1171-1182, 1172-1183, 1173-1184, 1174-1185, 1175-1186, 1176-1187, 1177-1188, 1178-1189, 1179-1190, 1180-1191, 1181-1192, 1182-1193, 1183-1194, 1184-1195, 1185-1196, 1186-1197, 1187-1198, 1188-1199, 1189-1200, 1190-1201, 1191-1202, 1192-1203, 1193-1204, 1194-1205, 1195-1206, 1196-1207, 1197-1208, 1198-1209, 1199-1210, 1200-1211, 1201-1212, 1202-1213, 1203-1214, 1204-1215, 1205-1216, 1206-1217, 1207-1218, 1208-1219, 1209-1220, 1210-1221, 1211-1222, 1212-1223, 1213-1224, 1214-1225, 1215-1226, 1216-1227, 1217-1228, 1218-1229, 1219-1230, 1220-1231, 1221-1232, 1222-1233, 1223-1234, 1224-1235, 1225-1236, 1226-1237, 1227-1238, 1228-1239, 1229-1240, 1230-1241, 1231-1242, 1232-1243, 1233-1244, 1234-1245, 1235-1246, 1236-1247, 1237-1248, 1238-1249, 1239-1250, 1240-1251, 1241-1252, 1242-1253, 1243-1254, 1244-1255, 1245-1256, 1246-1257, 1247-1258, 1248-1259, 1249-1260, 1250-1261, 1251-1262, 1252-1263, 1253-1264, 1254-1265, 1255-1266, 1256-1267, 1257-1268, 1258-1269, 1259-1270, 1260-1271, 1261-1272, 1262-1273, 1263-1274, 1264-1275, 1265-1276, 1266-1277, 1267-1278, 1268-1279, 1269-1280, 1270-1281, 1271-1282, 1272-1283, 1273-1284, 1274-1285, 1275-1286, 1276-1287, 1277-1288, 1278-1289, 1279-1290, 1280-1291, 1281-1292, 1282-1293, 1283-1294, 1284-1295, 1285-1296, 1286-1297, 1287-1298, 1288-1299, 1289-1300, 1290-1301, 1291-1302, 1292-1303, 1293-1304, 1294-1305, 1295-1306, 1296-1307, 1297-1308, 1298-1309, 1299-1310, 1300-1311, 1301-1312, 1302-1313, 1303-1314, 1304-1315, 1305-1316, 1306-1317, 1307-1318, 1308-1319, 1309-1320, 1310-1321, 1311-1322, 1312-1323, 1313-1324, 1314-1325, 1315-1326, 1316-1327, 1317-1328, 1318-1329, 1319-1330, 1320-1331, 1321-1332, 1322-1333, 1323-1334, 1324-1335, 1325-1336, 1326-1337, 1327-1338, 1328-1339, 1329-1340, 1330-1341, 1331-1342, 1332-1343, 1333-1344, 1334-1345, 1335-1346, 1336-1347, 1337-1348, 1338-1349, 1339-1350, 1340-1351, 1341-1352, 1342-1353, 1343-1354, 1344-1355, 1345-1356, 1346-1357, 1347-1358, 1348-1359, 1349-1360, 1350-1361, 1351-1362, 1352-1363, 1353-1364, 1354-1365, 1355-1366, 1356-1367, 1357-1368, 1358-1369, 1359-1370, 1360-1371, 1361-1372, 1362-1373, 1363-1374, 1364-1375, 1365-1376, 1366-1377, 1367-1378, 1368-1379, 1369-1380, 1370-1381, 1371-1382, 1372-1383, 1373-1384, 1374-1385, 1375-1386, 1376-1387, 1377-1388, 1378-1389, 1379-1390, 1380-1391, 1381-1392, 1382-1393, 1383-1394, 1384-1395, 1385-1396, 1386-1397, 1387-1398, 1388-1399, 1389-1400, 1390-1401, 1391-1402, 1392-1403, 1393-1404, 1394-1405, 1395-1406, 1396-1407, 1397-1408, 1398-1409, 1399-1410, 1400-1411, 1401-1412, 1402-1413, 1403-1414, 1404-1415, 1405-1416, 1406-1417, 1407-1418, 1408-1419, 1409-1420, 1410-1421, 1411-1422, 1412-1423, 1413-1424, 1414-1425, 1415-1426, 1416-1427, 1417-1428, 1418-1429, 1419-1430, 1420-1431, 1421-1432, 1422-1433, 1423-1434, 1424-1435, 1425-1436, 1426-1437, 1427-1438, 1428-1439, 1429-1440, 1430-1441, 1431-1442, 1432-1443, 1433-1444, 1434-1445, 1435-1446, 1436-1447, 1437-1448, 1438-1449, 1439-1450, 1440-1451, 1441-1452, 1442-1453, 1443-1454, 1444-1455, 1445-1456, 1446-1457, 1447-1458, 1448-1459, 1449-1460, 1450-1461, 1451-1462, 1452-1463, 1453-1464, 1454-1465, 1455-1466, 1456-1467, 1457-1468, 1458-1469, 1459-1470, 1460-1471, 1461-1472, 1462-1473, 1463-1474, 1464-1475, 1465-1476, 1466-1477, 1467-1478, 1468-1479, 1469-1480, 1470-1481, 1471-1482, 1472-1483, 1473-1484, 1474-1485, 1475-1486, 1476-1487, 1477-1488, 1478-1489, 1479-1490, 1480-1491, 1481-1492, 1482-1493, 1483-1494, 1484-1495, 1485-1496, 1486-1497, 1487-1498, 1488-1499, 1489-1500, 1490-1501, 1491-1502, 1492-1503, 1493-1504, 1494-1505, 1495-1506, 1496-1507, 1497-1508, 1498-1509, 1499-1510, 1500-1511, 1501-1512, 1502-1513, 1503-1514, 1504-1515, 1505-1516, 1506-1517, 1507-1518, 1508-1519, 1509-1520, 1510-1521, 1511-1522, 1512-1523, 1513-1524, 1514-1525, 1515-1526, 1516-1527, 1517-1528, 1518-1529, 1519-1530, 1520-1531, 1521-1532, 1522-1533, 1523-1534, 1524-1535, 1525-1536, 1526-1537, 1527-1538, 1528-1539, 1529-1540, 1530-1541, 1531-1542, 1532-1543, 1533-1544, 1534-1545, 1535-1546, 1536-1547, 1537-1548, 1538-1549, 1539-1550, 1540-1551, 1541-1552, 1542-1553, 1543-1554, 1544-1555, 1545-1556, 1546-1557, 1547-1558, 1548-1559, 1549-1560, 1550-1561, 1551-1562, 1552-1563, 1553-1564, 1554-1565, 1555-1566, 1556-1567, 1557-1568, 1558-1569, 1559-1570, 1560-1571, 1561-1572, 1562-1573, 1563-1574, 1564-1575, 1565-1576, 1566-1577, 1567-1578, 1568-1579, 1569-1580, 1570-1581, 1571-1582, 1572-1583, 1573-1584, 1574-1585, 1575-1586, 1576-1587, 1577-1588, 1578-1589, 1579-1590, 1580-1591, 1581-1592, 1582-1593, 1583-1594, 1584-1595, 1585-1596, 1586-1597, 1587-1598, 1588-1599, 1589-1600, 1590-1601, 1591-1602, 1592-1603, 1593-1604, 1594-1605, 1595-1606, 1596-1607, 1597-1608, 1598-1609, 1599-1610, 1600-1611, 1601-1612, 1602-1613, 1603-1614, 1604-1615, 1605-1616, 1606-1617, 1607-1618, 1608-1619, 1609-1620, 1610-1621, 1611-1622, 1612-1623, 1613-1624, 1614-1625, 1615-1626, 1616-1627, 1617-1628, 1618-1629, 1619-1630, 1620-1631, 1621-1632, 1622-1633, 1623-1634, 1624-1635, 1625-1636, 1626-1637, 1627-1638, 1628-1639, 1629-1640, 1630-1641, 1631-1642, 1632-1643, 1633-1644, 1634-1645, 1635-1646, 1636-1647, 1637-1648, 1638-1649, 1639-1650, 1640-1651, 1641-1652, 1642-1653, 1643-1654, 1644-1655, 1645-1656, 1646-1657, 1647-1658, 1648-1659, 1649-1660, 1650-1661, 1651-1662, 1652-1663, 1653-1664, 1654-1665, 1655-1666, 1656-1667, 1657-1668, 1658-1669, 1659-1670, 1660-1671, 1661-1672, 1662-1673, 1663-1674, 1664-1675, 1665-1676, 1666-1677, 1667-1678, 1668-1679, 1669-1680, 1670-1681, 1671-1682, 1672-1683, 1673-1684, 1674-1685, 1675-1686, 1676-1687, 1677-1688, 1678-1689, 1679-1690, 1680-1691, 1681-1692, 1682-1693, 1683-1694, 1684-1695, 1685-1696, 1686-1697, 1687-1698, 1688-1699, 1689-1700, 1690-1701, 1691-1702, 1692-1703, 1693-1704, 1694-1705, 1695-1706, 1696-1707, 1697-1708, 1698-1709, 1699-1710, 1700-1711, 1701-1712, 1702-1713, 1703-1714, 1704-1715, 1705-1716, 1706-1717, 1707-1718, 1708-1719, 1709-1720, 1710-1721, 1711-1722, 1712-1723, 1713-1724, 1714-1725, 1715-1726, 1716-1727, 1717-1728, 1718-1729, 1719-1730, 1720-1731, 1721-1732, 1722-1733, 1723-1734, 1724-1735, 1725-1736, 1726-1737, 1727-1738, 1728-1739, 1729-1740, 1730-1741, 1731-1742, 1732-1743, 1733-1744, 1734-1745, 1735-1746, 1736-1747, 1737-1748, 1738-1749, 1739-1750, 1740-1751, 1741-1752, 1742-1753, 1743-1754, 1744-1755, 1745-1756, 1746-1757, 1747-1758, 1748-1759, 1749-1760, 1750-1761, 1751-1762, 1752-1763, 1753-1764, 1754-1765, 1755-1766, 1756-1767, 1757-1768, 1758-1769, 1759-1770, 1760-1771, 1761-1772, 1762-1773, 1763-1774, 1764-1775, 1765-1776, 1766-1777, 1767-1778, 1768-1779, 1769-1780, 1770-1781, 1771-1782, 1772-1783, 1773-1784, 1774-1785, 1775-1786, 1776-1787, 1777-1788, 1778-1789, 1779-1790, 1780-1791, 1781-1792, 1782-1793, 1783-1794, 1784-1795, 1785-1796, 1786-1797, 1787-1798, 1788-1799, 1789-1800, 1790-1801, 1791-1802, 1792-1803, 1793-1804, 1794-1805, 1795-1806, 1796-1807, 1797-1808, 1798-1809, 1799-1810, 1800-1811, 1801-1812, 1802-1813, 1803-1814, 1804-1815, 1805-1816, 1806-1817, 1807-1818, 1808-1819, 1809-1820, 1810-1821, 1811-1822, 1812-1823, 1813-1824, 1814-1825, 1815-1826, 1816-1827, 1817-1828, 1818-1829, 1819-1830, 1820-1831, 1821-1832, 1822-1833, 1823-1834, 1824-1835, 1825-1836, 1826-1837, 1827-1838, 1828-1839, 1829-1840, 1830-1841, 1831-1842, 1832-1843, 1833-1844, 1834-1845, 1835-1846, 1836-1847, 1837-1848, 1838-1849, 1839-1850, 1840-1851, 1841-1852, 1842-1853, 1843-1854, 1844-1855, 1845-1856, 1846-1857, 1847-1858, 1848-1859, 1849-1860, 1850-1861, 1851-1862, 1852-1863, 1853-1864, 1854-1865, 1855-1866, 1856-1867, 1857-1868, 1858-1869, 1859-1870, 1860-1871, 1861-1872, 1862-1873, 1863-1874, 1864-1875, 1865-1876, 1866-1877, 1867-1878, 1868-1879, 1869-1880, 1870-1881, 1871-1882, 1872-1883, 1873-1884, 1874-1885, 1875-1886, 1876-1887, 1877-1888, 1878-1889, 1879-1890, 1880-1891, 1881-1892, 1882-1893, 1883-1894, 1884-1895, 1885-1896, 1886-1897, 1887-1898, 1888-1899, 1889-1900, 1890-1901, 1891-1902, 1892-1903, 1893-1904, 1894-1905, 1895-1906, 1896-1907, 1897-1908, 1898-1909, 1899-1910, 1900-1911, 1901-1912, 1902-1913, 1903-1914, 1904-1915, 1905-1916, 1906-1917, 1907-1918, 1908-1919, 1909-1920, 1910-1921, 1911-1922, 1912-1923, 1913-1924, 1914-1925, 1915-1926, 1916-1927, 1917-1928, 1918-1929, 1919-1930, 1920-1931, 1921-1932, 1922-1933, 1923-1934, 1924-1935, 1925-1936, 1926-1937, 1927-1938, 1928-1939, 1929-1940, 1930-1941, 1931-1942, 1932-1943, 1933-1944, 1934-1945, 1935-1946, 1936-1947, 1937-1948, 1938-1949, 1939-1950, 1940-1951, 1941-1952, 1942-1953, 1943-1954, 1944-1955, 1945-1956, 1946-1957, 1947-1958, 1948-1959, 1949-1960, 1950-1961, 1951-1962, 1952-1963, 1953-1964, 1954-1965, 1955-1966, 1956-1967, 1957-1968, 1958-1969, 1959-1970, 1960-1971, 1961-1972, 1962-1973, 1963-1974, 1964-1975, 1965-1976, 1966-1977, 1967-1978, 1968-1979, 1969-1980, 1970-1981, 1971-1982, 1972-1983, 1973-1984, 1974-1985, 1975-1986, 1976-1987, 1977-1988, 1978-1989, 1979-1990, 1980-1991, 1981-1992, 1982-1993, 1983-1994, 1984-1995, 1985-1996, 1986-1997, 1987-1998, 1988-1999, 1989-2000, 1990-2001, 1991-2002, 1992-2003, 1993-2004, 1994-2005, 1995-2006, 1996-2007, 1997-2008, 1998-2009, 1999-2010, 2000-2011, 2001-2012, 2002-2013, 2003-2014, 2004-2015, 2005-2016, 2006-2017, 2007-2018, 2008-2019, 2009-2020, 2010-2021, 2011-2022, 2012-2023, 2013-2024, 2014-2025, 2015-2026, 2016-2027, 2017-2028, 2018-2029, 2019-2030, 2020-2031, 2021-2032, 2022-2033, 2023-2034, 2024-2035, 2025-2036, 2026-2037, 2027-2038, 2028-2039, 2029-2040, 2030-2041, 2031-2042, 2032-2043, 2033-2044, 2034-2045, 2035-2046, 2036-2047, 2037-2048, 2038-2049, 2039-2050, 2040-2051, 2041-2052, 2042-2053, 2043-2054, 2044-2055, 2045-2056, 2046-2057, 2047-2058, 2048-2059, 2049-2060, 2050-2061, 2051-2062, 2052-2063, 2053-2064, 2054-2065, 2055-2066, 2056-2067, 2057-2068, 2058-2069, 2059-2070, 2060-2071, 2061-2072 and 2062-2073.

Exemplary polynucleotide molecules include the following 15-mer fragments of the polynucleotide sequence from the sequence of SEQ ID NO:4: 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723, 710-724, 711-725, 712-726, 713-727, 714-728, 715-729, 716-730, 717-731, 718-732, 719-733, 720-734, 721-735, 722-736, 723-737, 724-738, 725-739, 726-740, 727-741, 728-742, 729-743, 730-744, 731-745, 732-746, 733-747, 734-748, 735-749, 736-750, 737-751, 738-752, 739-753, 740-754, 741-755, 742-756, 743-757, 744-758, 745-759, 746-760, 747-761, 748-762, 749-763, 750-764, 751-765, 752-766, 753-767, 754-768, 755-769, 756-770, 757-771, 758-772, 759-773, 760-774, 761-775, 762-776, 763-777, 764-778, 765-779, 766-780, 767-781, 768-782, 769-783, 770-784, 771-785, 772-786, 773-787, 774-788, 775-789, 776-790, 777-791, 778-792, 779-793, 780-794, 781-795, 782-796, 783-797, 784-798, 785-799, 786-800, 787-801, 788-802, 789-803, 790-804, 791-805, 792-806, 793-807, 794-808, 795-809, 796-810, 797-811, 798-812, 799-813, 800-814, 801-815, 802-816, 803-817, 804-818, 805-819, 806-820, 807-821, 808-822, 809-823, 810-824, 811-825, 812-826, 813-827, 814-828, 815-829, 816-830, 817-831, 818-832, 819-833, 820-834, 821-835, 822-836, 823-837, 824-838, 825-839, 826-840, 827-841, 828-842, 829-843, 830-844, 831-845, 832-846, 833-847, 834-848, 835-849, 836-850, 837-851, 838-852, 839-853, 840-854, 841-855, 842-856, 843-857, 844-858, 845-859, 846-860, 847-861, 848-862, 849-863, 850-864, 851-865, 852-866, 853-867, 854-868, 855-869, 856-870, 857-871, 858-872, 859-873, 860-874, 861-875, 862-876, 863-877, 864-878, 865-879, 866-880, 867-881, 868-882, 869-883, 870-884, 871-885, 872-886, 873-887, 874-888, 875-889, 876-890, 877-891, 878-892, 879-893, 880-894, 881-895, 882-896, 883-897, 884-898, 885-899, 886-900, 887-901, 888-902, 889-903, 890-904, 891-905, 892-906, 893-907, 894-908, 895-909, 896-910, 897-911, 898-912, 899-913, 900-914, 901-915, 902-916, 903-917, 904-918, 905-919, 906-920, 907-921, 908-922, 909-923, 910-924, 911-925, 912-926, 913-927, 914-928, 915-929, 916-930, 917-931, 918-932, 919-933, 920-934, 921-935, 922-936, 923-937, 924-938, 925-939, 926-940, 927-941, 928-942, 929-943, 930-944, 931-945, 932-946, 933-947, 934-948, 935-949, 936-950, 937-951, 938-952, 939-953, 940-954, 941-955, 942-956, 943-957, 944-958, 945-959, 946-960, 947-961, 948-962, 949-963, 950-964, 951-965, 952-966, 953-967, 954-968, 955-969, 956-970, 957-971, 958-972, 959-973, 960-974, 961-975, 962-976, 963-977, 964-978, 965-979, 966-980, 967-981, 968-982, 969-983, 970-984, 971-985, 972-986, 973-987, 974-988, 975-989, 976-990, 977-991, 978-992, 979-993, 980-994, 981-995, 982-996, 983-997, 984-998, 985-999, 986-1000, 987-1001, 988-1002, 989-1003, 990-1004, 991-1005, 992-1006, 993-1007, 994-1008, 995-1009, 996-1010, 997-1011, 998-1012, 999-1013, 1000-1014, 1001-1015, 1002-1016, 1003-1017, 1004-1018, 1005-1019, 1006-1020, 1007-1021, 1008-1022, 1009-1023, 1010-1024, 1011-1025, 1012-1026, 1013-1027, 1014-1028, 1015-1029, 1016-1030, 1017-1031, 1018-1032, 1019-1033, 1020-1034, 1021-1035, 1022-1036, 1023-1037, 1024-1038, 1025-1039, 1026-1040, 1027-1041, 1028-1042, 1029-1043, 1030-1044, 1031-1045, 1032-1046, 1033-1047, 1034-1048, 1035-1049, 1036-1050, 1037-1051, 1038-1052, 1039-1053, 1040-1054, 1041-1055, 1042-1056, 1043-1057, 1044-1058, 1045-1059, 1046-1060, 1047-1061, 1048-1062, 1049-1063, 1050-1064, 1051-1065, 1052-1066, 1053-1067, 1054-1068, 1055-1069, 1056-1070, 1057-1071, 1058-1072, 1059-1073, 1060-1074, 1061-1075, 1062-1076, 1063-1077, 1064-1078, 1065-1079, 1066-1080, 1067-1081, 1068-1082, 1069-1083, 1070-1084, 1071-1085, 1072-1086, 1073-1087, 1074-1088, 1075-1089, 1076-1090, 1077-1091, 1078-1092, 1079-1093, 1080-1094, 1081-1095, 1082-1096, 1083-1097, 1084-1098, 1085-1099, 1086-1100, 1087-1101, 1088-1102, 1089-1103, 1090-1104, 1091-1105, 1092-1106, 1093-1107, 1094-1108, 1095-1109, 1096-1110, 1097-1111, 1098-1112, 1099-1113, 1100-1114, 1101-1115, 1102-1116, 1103-1117, 1104-1118, 1105-1119, 1106-1120, 1107-1121, 1108-1122, 1109-1123, 1110-1124, 1111-1125, 1112-1126, 1113-1127, 1114-1128, 1115-1129, 1116-1130, 1117-1131, 1118-1132, 1119-1133, 1120-1134, 1121-1135, 1122-1136, 1123-1137, 1124-1138, 1125-1139, 1126-1140, 1127-1141, 1128-1142, 1129-1143, 1130-1144, 1131-1145, 1132-1146, 1133-1147, 1134-1148, 1135-1149, 1136-1150, 1137-1151, 1138-1152, 1139-1153, 1140-1154, 1141-1155, 1142-1156, 1143-1157, 1144-1158, 1145-1159, 1146-1160, 1147-1161, 1148-1162, 1149-1163, 1150-1164, 1151-1165, 1152-1166, 1153-1167, 1154-1168, 1155-1169, 1156-1170, 1157-1171, 1158-1172, 1159-1173, 1160-1174, 1161-1175, 1162-1176, 1163-1177, 1164-1178, 1165-1179, 1166-1180, 1167-1181, 1168-1182, 1169-1183, 1170-1184, 1171-1185, 1172-1186, 1173-1187, 1174-1188, 1175-1189, 1176-1190, 1177-1191, 1178-1192, 1179-1193, 1180-1194, 1181-1195, 1182-1196, 1183-1197, 1184-1198, 1185-1199, 1186-1200, 1187-1201, 1188-1202, 1189-1203, 1190-1204, 1191-1205, 1192-1206, 1193-1207, 1194-1208, 1195-1209, 1196-1210, 1197-1211, 1198-1212, 1199-1213, 1200-1214, 1201-1215, 1202-1216, 1203-1217, 1204-1218, 1205-1219, 1206-1220, 1207-1221, 1208-1222, 1209-1223, 1210-1224, 1211-1225, 1212-1226, 1213-1227, 1214-1228, 1215-1229, 1216-1230, 1217-1231, 1218-1232, 1219-1233, 1220-1234, 1221-1235, 1222-1236, 1223-1237, 1224-1238, 1225-1239, 1226-1240, 1227-1241, 1228-1242, 1229-1243, 1230-1244, 1231-1245, 1232-1246, 1233-1247, 1234-1248, 1235-1249, 1236-1250, 1237-1251, 1238-1252, 1239-1253, 1240-1254, 1241-1255, 1242-1256, 1243-1257, 1244-1258, 1245-1259, 1246-1260, 1247-1261, 1248-1262, 1249-1263, 1250-1264, 1251-1265, 1252-1266, 1253-1267, 1254-1268, 1255-1269, 1256-1270, 1257-1271, 1258-1272, 1259-1273, 1260-1274, 1261-1275, 1262-1276, 1263-1277, 1264-1278, 1265-1279, 1266-1280, 1267-1281, 1268-1282, 1269-1283, 1270-1284, 1271-1285, 1272-1286, 1273-1287, 1274-1288, 1275-1289, 1276-1290, 1277-1291, 1278-1292, 1279-1293, 1280-1294, 1281-1295, 1282-1296, 1283-1297, 1284-1298, 1285-1299, 1286-1300, 1287-1301, 1288-1302, 1289-1303, 1290-1304, 1291-1305, 1292-1306, 1293-1307, 1294-1308, 1295-1309, 1296-1310, 1297-1311, 1298-1312, 1299-1313, 1300-1314, 1301-1315, 1302-1316, 1303-1317, 1304-1318, 1305-1319, 1306-1320, 1307-1321, 1308-1322, 1309-1323, 1310-1324, 1311-1325, 1312-1326, 1313-1327, 1314-1328, 1315-1329, 1316-1330, 1317-1331, 1318-1332, 1319-1333, 1320-1334, 1321-1335, 1322-1336, 1323-1337, 1324-1338, 1325-1339, 1326-1340, 1327-1341, 1328-1342, 1329-1343, 1330-1344, 1331-1345, 1332-1346, 1333-1347, 1334-1348, 1335-1349, 1336-1350, 1337-1351, 1338-1352, 1339-1353, 1340-1354, 1341-1355, 1342-1356, 1343-1357, 1344-1358, 1345-1359, 1346-1360, 1347-1361, 1348-1362, 1349-1363, 1350-1364, 1351-1365, 1352-1366, 1353-1367, 1354-1368, 1355-1369, 1356-1370, 1357-1371, 1358-1372, 1359-1373, 1360-1374, 1361-1375, 1362-1376, 1363-1377, 1364-1378, 1365-1379, 1366-1380, 1367-1381, 1368-1382, 1369-1383, 1370-1384, 1371-1385, 1372-1386, 1373-1387, 1374-1388, 1375-1389, 1376-1390, 1377-1391, 1378-1392, 1379-1393, 1380-1394, 1381-1395, 1382-1396, 1383-1397, 1384-1398, 1385-1399, 1386-1400, 1387-1401, 1388-1402, 1389-1403, 1390-1404, 1391-1405, 1392-1406, 1393-1407, 1394-1408, 1395-1409, 1396-1410, 1397-1411, 1398-1412, 1399-1413, 1400-1414, 1401-1415, 1402-1416, 1403-1417, 1404-1418, 1405-1419, 1406-1420, 1407-1421, 1408-1422, 1409-1423, 1410-1424, 1411-1425, 1412-1426, 1413-1427, 1414-1428, 1415-1429, 1416-1430, 1417-1431, 1418-1432, 1419-1433, 1420-1434, 1421-1435, 1422-1436, 1423-1437, 1424-1438, 1425-1439, 1426-1440, 1427-1441, 1428-1442, 1429-1443, 1430-1444, 1431-1445, 1432-1446, 1433-1447, 1434-1448, 1435-1449, 1436-1450, 1437-1451, 1438-1452, 1439-1453, 1440-1454, 1441-1455, 1442-1456, 1443-1457, 1444-1458, 1445-1459, 1446-1460, 1447-1461, 1448-1462, 1449-1463, 1450-1464, 1451-1465, 1452-1466, 1453-1467, 1454-1468, 1455-1469, 1456-1470, 1457-1471, 1458-1472, 1459-1473, 1460-1474, 1461-1475, 1462-1476, 1463-1477, 1464-1478, 1465-1479, 1466-1480, 1467-1481, 1468-1482, 1469-1483, 1470-1484, 1471-1485, 1472-1486, 1473-1487, 1474-1488, 1475-1489, 1476-1490, 1477-1491, 1478-1492, 1479-1493, 1480-1494, 1481-1495, 1482-1496, 1483-1497, 1484-1498, 1485-1499, 1486-1500, 1487-1501, 1488-1502, 1489-1503, 1490-1504, 1491-1505, 1492-1506, 1493-1507, 1494-1508, 1495-1509, 1496-1510, 1497-1511, 1498-1512, 1499-1513, 1500-1514, 1501-1515, 1502-1516, 1503-1517, 1504-1518, 1505-1519, 1506-1520, 1507-1521, 1508-1522, 1509-1523, 1510-1524, 1511-1525, 1512-1526, 1513-1527, 1514-1528, 1515-1529, 1516-1530, 1517-1531, 1518-1532, 1519-1533, 1520-1534, 1521-1535, 1522-1536, 1523-1537, 1524-1538, 1525-1539, 1526-1540, 1527-1541, 1528-1542, 1529-1543, 1530-1544, 1531-1545, 1532-1546, 1533-1547, 1534-1548, 1535-1549, 1536-1550, 1537-1551, 1538-1552, 1539-1553, 1540-1554, 1541-1555, 1542-1556, 1543-1557, 1544-1558, 1545-1559, 1546-1560, 1547-1561, 1548-1562, 1549-1563, 1550-1564, 1551-1565, 1552-1566, 1553-1567, 1554-1568, 1555-1569, 1556-1570, 1557-1571, 1558-1572, 1559-1573, 1560-1574, 1561-1575, 1562-1576, 1563-1577, 1564-1578, 1565-1579, 1566-1580, 1567-1581, 1568-1582, 1569-1583, 1570-1584, 1571-1585, 1572-1586, 1573-1587, 1574-1588, 1575-1589, 1576-1590, 1577-1591, 1578-1592, 1579-1593, 1580-1594, 1581-1595, 1582-1596, 1583-1597, 1584-1598, 1585-1599, 1586-1600, 1587-1601, 1588-1602, 1589-1603, 1590-1604, 1591-1605, 1592-1606, 1593-1607, 1594-1608, 1595-1609, 1596-1610, 1597-1611, 1598-1612, 1599-1613, 1600-1614, 1601-1615, 1602-1616, 1603-1617, 1604-1618, 1605-1619, 1606-1620, 1607-1621, 1608-1622, 1609-1623, 1610-1624, 1611-1625, 1612-1626, 1613-1627, 1614-1628, 1615-1629, 1616-1630, 1617-1631, 1618-1632, 1619-1633, 1620-1634, 1621-1635, 1622-1636, 1623-1637, 1624-1638, 1625-1639, 1626-1640, 1627-1641, 1628-1642, 1629-1643, 1630-1644, 1631-1645, 1632-1646, 1633-1647, 1634-1648, 1635-1649, 1636-1650, 1637-1651, 1638-1652, 1639-1653, 1640-1654, 1641-1655, 1642-1656, 1643-1657, 1644-1658, 1645-1659, 1646-1660, 1647-1661, 1648-1662, 1649-1663, 1650-1664, 1651-1665, 1652-1666, 1653-1667, 1654-1668, 1655-1669, 1656-1670, 1657-1671, 1658-1672, 1659-1673, 1660-1674, 1661-1675, 1662-1676, 1663-1677, 1664-1678, 1665-1679, 1666-1680, 1667-1681, 1668-1682, 1669-1683, 1670-1684, 1671-1685, 1672-1686, 1673-1687, 1674-1688, 1675-1689, 1676-1690, 1677-1691, 1678-1692, 1679-1693, 1680-1694, 1681-1695, 1682-1696, 1683-1697, 1684-1698, 1685-1699, 1686-1700, 1687-1701, 1688-1702, 1689-1703, 1690-1704, 1691-1705, 1692-1706, 1693-1707, 1694-1708, 1695-1709, 1696-1710, 1697-1711, 1698-1712, 1699-1713, 1700-1714, 1701-1715, 1702-1716, 1703-1717, 1704-1718, 1705-1719, 1706-1720, 1707-1721, 1708-1722, 1709-1723, 1710-1724, 1711-1725, 1712-1726, 1713-1727, 1714-1728, 1715-1729, 1716-1730, 1717-1731, 1718-1732, 1719-1733, 1720-1734, 1721-1735, 1722-1736, 1723-1737, 1724-1738, 1725-1739, 1726-1740, 1727-1741, 1728-1742, 1729-1743, 1730-1744, 1731-1745, 1732-1746, 1733-1747, 1734-1748, 1735-1749, 1736-1750, 1737-1751, 1738-1752, 1739-1753, 1740-1754, 1741-1755, 1742-1756, 1743-1757, 1744-1758, 1745-1759, 1746-1760, 1747-1761, 1748-1762, 1749-1763, 1750-1764, 1751-1765, 1752-1766, 1753-1767, 1754-1768, 1755-1769, 1756-1770, 1757-1771, 1758-1772, 1759-1773, 1760-1774, 1761-1775, 1762-1776, 1763-1777, 1764-1778, 1765-1779, 1766-1780, 1767-1781, 1768-1782, 1769-1783, 1770-1784, 1771-1785, 1772-1786, 1773-1787, 1774-1788, 1775-1789, 1776-1790, 1777-1791, 1778-1792, 1779-1793, 1780-1794, 1781-1795, 1782-1796, 1783-1797, 1784-1798, 1785-1799, 1786-1800, 1787-1801, 1788-1802, 1789-1803, 1790-1804, 1791-1805, 1792-1806, 1793-1807, 1794-1808, 1795-1809, 1796-1810, 1797-1811, 1798-1812, 1799-1813, 1800-1814, 1801-1815, 1802-1816, 1803-1817, 1804-1818, 1805-1819, 1806-1820, 1807-1821, 1808-1822, 1809-1823, 1810-1824, 1811-1825, 1812-1826, 1813-1827, 1814-1828, 1815-1829, 1816-1830, 1817-1831, 1818-1832, 1819-1833, 1820-1834, 1821-1835, 1822-1836, 1823-1837, 1824-1838, 1825-1839, 1826-1840, 1827-1841, 1828-1842, 1829-1843, 1830-1844, 1831-1845, 1832-1846, 1833-1847, 1834-1848, 1835-1849, 1836-1850, 1837-1851, 1838-1852, 1839-1853, 1840-1854, 1841-1855, 1842-1856, 1843-1857, 1844-1858, 1845-1859, 1846-1860, 1847-1861, 1848-1862, 1849-1863, 1850-1864, 1851-1865, 1852-1866, 1853-1867, 1854-1868, 1855-1869, 1856-1870, 1857-1871, 1858-1872, 1859-1873, 1860-1874, 1861-1875, 1862-1876, 1863-1877, 1864-1878, 1865-1879, 1866-1880, 1867-1881, 1868-1882, 1869-1883, 1870-1884, 1871-1885, 1872-1886, 1873-1887, 1874-1888, 1875-1889, 1876-1890, 1877-1891, 1878-1892, 1879-1893, 1880-1894, 1881-1895, 1882-1896, 1883-1897, 1884-1898, 1885-1899, 1886-1900, 1887-1901, 1888-1902, 1889-1903, 1890-1904, 1891-1905, 1892-1906, 1893-1907, 1894-1908, 1895-1909, 1896-1910, 1897-1911, 1898-1912, 1899-1913, 1900-1914, 1901-1915, 1902-1916, 1903-1917, 1904-1918, 1905-1919, 1906-1920, 1907-1921, 1908-1922, 1909-1923, 1910-1924, 1911-1925, 1912-1926, 1913-1927, 1914-1928, 1915-1929, 1916-1930, 1917-1931, 1918-1932, 1919-1933, 1920-1934, 1921-1935, 1922-1936, 1923-1937, 1924-1938, 1925-1939, 1926-1940, 1927-1941, 1928-1942, 1929-1943, 1930-1944, 1931-1945, 1932-1946, 1933-1947, 1934-1948, 1935-1949, 1936-1950, 1937-1951, 1938-1952, 1939-1953, 1940-1954, 1941-1955, 1942-1956, 1943-1957, 1944-1958, 1945-1959, 1946-1960, 1947-1961, 1948-1962, 1949-1963, 1950-1964, 1951-1965, 1952-1966, 1953-1967, 1954-1968, 1955-1969, 1956-1970, 1957-1971, 1958-1972, 1959-1973, 1960-1974, 1961-1975, 1962-1976, 1963-1977, 1964-1978, 1965-1979, 1966-1980, 1967-1981, 1968-1982, 1969-1983, 1970-1984, 1971-1985, 1972-1986, 1973-1987, 1974-1988, 1975-1989, 1976-1990, 1977-1991, 1978-1992, 1979-1993, 1980-1994, 1981-1995, 1982-1996, 1983-1997, 1984-1998, 1985-1999, 1986-2000, 1987-2001, 1988-2002, 1989-2003, 1990-2004, 1991-2005, 1992-2006, 1993-2007, 1994-2008, 1995-2009, 1996-2010, 1997-2011, 1998-2012, 1999-2013, 2000-2014, 2001-2015, 2002-2016, 2003-2017, 2004-2018, 2005-2019, 2006-2020, 2007-2021, 2008-2022, 2009-2023, 2010-2024, 2011-2025, 2012-2026, 2013-2027, 2014-2028, 2015-2029, 2016-2030, 2017-2031, 2018-2032, 2019-2033, 2020-2034, 2021-2035, 2022-2036, 2023-2037, 2024-2038, 2025-2039, 2026-2040, 2027-2041, 2028-2042, 2029-2043, 2030-2044, 2031-2045, 2032-2046, 2033-2047, 2034-2048, 2035-2049, 2036-2050, 2037-2051, 2038-2052, 2039-2053, 2040-2054, 2041-2055, 2042-2056, 2043-2057, 2044-2058, 2045-2059, 2046-2060, 2047-2061, 2048-2062, 2049-2063, 2050-2064, 2051-2065, 2052-2066, 2053-2067, 2054-2068, 2055-2069, 2056-2070, 2057-2071, 2058-2072 and 2059-2073.

Exemplary polynucleotide molecules include the following 20-mer fragments of the polynucleotide sequence from the sequence of SEQ ID NO:4: 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-1420, 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, 1581-1600, 1582-1601, 1583-1602, 1584-1603, 1585-1604, 1586-1605, 1587-1606, 1588-1607, 1589-1608, 1590-1609, 1591-1610, 1592-1611, 1593-1612, 1594-1613, 1595-1614, 1596-1615, 1597-1616, 1598-1617, 1599-1618, 1600-1619, 1601-1620, 1602-1621, 1603-1622, 1604-1623, 1605-1624, 1606-1625, 1607-1626, 1608-1627, 1609-1628, 1610-1629, 1611-1630, 1612-1631, 1613-1632, 1614-1633, 1615-1634, 1616-1635, 1617-1636, 1618-1637, 1619-1638, 1620-1639, 1621-1640, 1622-1641, 1623-1642, 1624-1643, 1625-1644, 1626-1645, 1627-1646, 1628-1647, 1629-1648, 1630-1649, 1631-1650, 1632-1651, 1633-1652, 1634-1653, 1635-1654, 1636-1655, 1637-1656, 1638-1657, 1639-1658, 1640-1659, 1641-1660, 1642-1661, 1643-1662, 1644-1663, 1645-1664, 1646-1665, 1647-1666, 1648-1667, 1649-1668, 1650-1669, 1651-1670, 1652-1671, 1653-1672, 1654-1673, 1655-1674, 1656-1675, 1657-1676, 1658-1677, 1659-1678, 1660-1679, 1661-1680, 1662-1681, 1663-1682, 1664-1683, 1665-1684, 1666-1685, 1667-1686, 1668-1687, 1669-1688, 1670-1689, 1671-1690, 1672-1691, 1673-1692, 1674-1693, 1675-1694, 1676-1695, 1677-1696, 1678-1697, 1679-1698, 1680-1699, 1681-1700, 1682-1701, 1683-1702, 1684-1703, 1685-1704, 1686-1705, 1687-1706, 1688-1707, 1689-1708, 1690-1709, 1691-1710, 1692-1711, 1693-1712, 1694-1713, 1695-1714, 1696-1715, 1697-1716, 1698-1717, 1699-1718, 1700-1719, 1701-1720, 1702-1721, 1703-1722, 1704-1723, 1705-1724, 1706-1725, 1707-1726, 1708-1727, 1709-1728, 1710-1729, 1711-1730, 1712-1731, 1713-1732, 1714-1733, 1715-1734, 1716-1735, 1717-1736, 1718-1737, 1719-1738, 1720-1739, 1721-1740, 1722-1741, 1723-1742, 1724-1743, 1725-1744, 1726-1745, 1727-1746, 1728-1747, 1729-1748, 1730-1749, 1731-1750, 1732-1751, 1733-1752, 1734-1753, 1735-1754, 1736-1755, 1737-1756, 1738-1757, 1739-1758, 1740-1759, 1741-1760, 1742-1761, 1743-1762, 1744-1763, 1745-1764, 1746-1765, 1747-1766, 1748-1767, 1749-1768, 1750-1769, 1751-1770, 1752-1771, 1753-1772, 1754-1773, 1755-1774, 1756-1775, 1757-1776, 1758-1777, 1759-1778, 1760-1779, 1761-1780, 1762-1781, 1763-1782, 1764-1783, 1765-1784, 1766-1785, 1767-1786, 1768-1787, 1769-1788, 1770-1789, 1771-1790, 1772-1791, 1773-1792, 1774-1793, 1775-1794, 1776-1795, 1777-1796, 1778-1797, 1779-1798, 1780-1799, 1781-1800, 1782-1801, 1783-1802, 1784-1803, 1785-1804, 1786-1805, 1787-1806, 1788-1807, 1789-1808, 1790-1809, 1791-1810, 1792-1811, 1793-1812, 1794-1813, 1795-1814, 1796-1815, 1797-1816, 1798-1817, 1799-1818, 1800-1819, 1801-1820, 1802-1821, 1803-1822, 1804-1823, 1805-1824, 1806-1825, 1807-1826, 1808-1827, 1809-1828, 1810-1829, 1811-1830, 1812-1831, 1813-1832, 1814-1833, 1815-1834, 1816-1835, 1817-1836, 1818-1837, 1819-1838, 1820-1839, 1821-1840, 1822-1841, 1823-1842, 1824-1843, 1825-1844, 1826-1845, 1827-1846, 1828-1847, 1829-1848, 1830-1849, 1831-1850, 1832-1851, 1833-1852, 1834-1853, 1835-1854, 1836-1855, 1837-1856, 1838-1857, 1839-1858, 1840-1859, 1841-1860, 1842-1861, 1843-1862, 1844-1863, 1845-1864, 1846-1865, 1847-1866, 1848-1867, 1849-1868, 1850-1869, 1851-1870, 1852-1871, 1853-1872, 1854-1873, 1855-1874, 1856-1875, 1857-1876, 1858-1877, 1859-1878, 1860-1879, 1861-1880, 1862-1881, 1863-1882, 1864-1883, 1865-1884, 1866-1885, 1867-1886, 1868-1887, 1869-1888, 1870-1889, 1871-1890, 1872-1891, 1873-1892, 1874-1893, 1875-1894, 1876-1895, 1877-1896, 1878-1897, 1879-1898, 1880-1899, 1881-1900, 1882-1901, 1883-1902, 1884-1903, 1885-1904, 1886-1905, 1887-1906, 1888-1907, 1889-1908, 1890-1909, 1891-1910, 1892-1911, 1893-1912, 1894-1913, 1895-1914, 1896-1915, 1897-1916, 1898-1917, 1899-1918, 1900-1919, 1901-1920, 1902-1921, 1903-1922, 1904-1923, 1905-1924, 1906-1925, 1907-1926, 1908-1927, 1909-1928, 1910-1929, 1911-1930, 1912-1931, 1913-1932, 1914-1933, 1915-1934, 1916-1935, 1917-1936, 1918-1937, 1919-1938, 1920-1939, 1921-1940, 1922-1941, 1923-1942, 1924-1943, 1925-1944, 1926-1945, 1927-1946, 1928-1947, 1929-1948, 1930-1949, 1931-1950, 1932-1951, 1933-1952, 1934-1953, 1935-1954, 1936-1955, 1937-1956, 1938-1957, 1939-1958, 1940-1959, 1941-1960, 1942-1961, 1943-1962, 1944-1963, 1945-1964, 1946-1965, 1947-1966, 1948-1967, 1949-1968, 1950-1969, 1951-1970, 1952-1971, 1953-1972, 1954-1973, 1955-1974, 1956-1975, 1957-1976, 1958-1977, 1959-1978, 1960-1979, 1961-1980, 1962-1981, 1963-1982, 1964-1983, 1965-1984, 1966-1985, 1967-1986, 1968-1987, 1969-1988, 1970-1989, 1971-1990, 1972-1991, 1973-1992, 1974-1993, 1975-1994, 1976-1995, 1977-1996, 1978-1997, 1979-1998, 1980-1999, 1981-2000, 1982-2001, 1983-2002, 1984-2003, 1985-2004, 1986-2005, 1987-2006, 1988-2007, 1989-2008, 1990-2009, 1991-2010, 1992-2011, 1993-2012, 1994-2013, 1995-2014, 1996-2015, 1997-2016, 1998-2017, 1999-2018, 2000-2019, 2001-2020, 2002-2021, 2003-2022, 2004-2023, 2005-2024, 2006-2025, 2007-2026, 2008-2027, 2009-2028, 2010-2029, 2011-2030, 2012-2031, 2013-2032, 2014-2033, 2015-2034, 2016-2035, 2017-2036, 2018-2037, 2019-2038, 2020-2039, 2021-2040, 2022-2041, 2023-2042, 2024-2043, 2025-2044, 2026-2045, 2027-2046, 2028-2047, 2029-2048, 2030-2049, 2031-2050, 2032-2051, 2033-2052, 2034-2053, 2035-2054, 2036-2055, 2037-2056, 2038-2057, 2039-2058, 2040-2059, 2041-2060, 2042-2061, 2043-2062, 2044-2063, 2045-2064, 2046-2065, 2047-2066, 2048-2067, 2049-2068, 2050-2069, 2051-2070, 2052-2071, 2053-2072 and 2054-2073.

Exemplary polynucleotide molecules include the following 25-mer fragments of the polynucleotide sequence from the sequence of SEQ ID NO:4: 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815, 792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1472, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-1501, 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568, 1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600, 1577-1601, 1578-1602, 1579-1603, 1580-1604, 1581-1605, 1582-1606, 1583-1607, 1584-1608, 1585-1609, 1586-1610, 1587-1611, 1588-1612, 1589-1613, 1590-1614, 1591-1615, 1592-1616, 1593-1617, 1594-1618, 1595-1619, 1596-1620, 1597-1621, 1598-1622, 1599-1623, 1600-1624, 1601-1625, 1602-1626, 1603-1627, 1604-1628, 1605-1629, 1606-1630, 1607-1631, 1608-1632, 1609-1633, 1610-1634, 1611-1635, 1612-1636, 1613-1637, 1614-1638, 1615-1639, 1616-1640, 1617-1641, 1618-1642, 1619-1643, 1620-1644, 1621-1645, 1622-1646, 1623-1647, 1624-1648, 1625-1649, 1626-1650, 1627-1651, 1628-1652, 1629-1653, 1630-1654, 1631-1655, 1632-1656, 1633-1657, 1634-1658, 1635-1659, 1636-1660, 1637-1661, 1638-1662, 1639-1663, 1640-1664, 1641-1665, 1642-1666, 1643-1667, 1644-1668, 1645-1669, 1646-1670, 1647-1671, 1648-1672, 1649-1673, 1650-1674, 1651-1675, 1652-1676, 1653-1677, 1654-1678, 1655-1679, 1656-1680, 1657-1681, 1658-1682, 1659-1683, 1660-1684, 1661-1685, 1662-1686, 1663-1687, 1664-1688, 1665-1689, 1666-1690, 1667-1691, 1668-1692, 1669-1693, 1670-1694, 1671-1695, 1672-1696, 1673-1697, 1674-1698, 1675-1699, 1676-1700, 1677-1701, 1678-1702, 1679-1703, 1680-1704, 1681-1705, 1682-1706, 1683-1707, 1684-1708, 1685-1709, 1686-1710, 1687-1711, 1688-1712, 1689-1713, 1690-1714, 1691-1715, 1692-1716, 1693-1717, 1694-1718, 1695-1719, 1696-1720, 1697-1721, 1698-1722, 1699-1723, 1700-1724, 1701-1725, 1702-1726, 1703-1727, 1704-1728, 1705-1729, 1706-1730, 1707-1731, 1708-1732, 1709-1733, 1710-1734, 1711-1735, 1712-1736, 1713-1737, 1714-1738, 1715-1739, 1716-1740, 1717-1741, 1718-1742, 1719-1743, 1720-1744, 1721-1745, 1722-1746, 1723-1747, 1724-1748, 1725-1749, 1726-1750, 1727-1751, 1728-1752, 1729-1753, 1730-1754, 1731-1755, 1732-1756, 1733-1757, 1734-1758, 1735-1759, 1736-1760, 1737-1761, 1738-1762, 1739-1763, 1740-1764, 1741-1765, 1742-1766, 1743-1767, 1744-1768, 1745-1769, 1746-1770, 1747-1771, 1748-1772, 1749-1773, 1750-1774, 1751-1775, 1752-1776, 1753-1777, 1754-1778, 1755-1779, 1756-1780, 1757-1781, 1758-1782, 1759-1783, 1760-1784, 1761-1785, 1762-1786, 1763-1787, 1764-1788, 1765-1789, 1766-1790, 1767-1791, 1768-1792, 1769-1793, 1770-1794, 1771-1795, 1772-1796, 1773-1797, 1774-1798, 1775-1799, 1776-1800, 1777-1801, 1778-1802, 1779-1803, 1780-1804, 1781-1805, 1782-1806, 1783-1807, 1784-1808, 1785-1809, 1786-1810, 1787-1811, 1788-1812, 1789-1813, 1790-1814, 1791-1815, 1792-1816, 1793-1817, 1794-1818, 1795-1819, 1796-1820, 1797-1821, 1798-1822, 1799-1823, 1800-1824, 1801-1825, 1802-1826, 1803-1827, 1804-1828, 1805-1829, 1806-1830, 1807-1831, 1808-1832, 1809-1833, 1810-1834, 1811-1835, 1812-1836, 1813-1837, 1814-1838, 1815-1839, 1816-1840, 1817-1841, 1818-1842, 1819-1843, 1820-1844, 1821-1845, 1822-1846, 1823-1847, 1824-1848, 1825-1849, 1826-1850, 1827-1851, 1828-1852, 1829-1853, 1830-1854, 1831-1855, 1832-1856, 1833-1857, 1834-1858, 1835-1859, 1836-1860, 1837-1861, 1838-1862, 1839-1863, 1840-1864, 1841-1865, 1842-1866, 1843-1867, 1844-1868, 1845-1869, 1846-1870, 1847-1871, 1848-1872, 1849-1873, 1850-1874, 1851-1875, 1852-1876, 1853-1877, 1854-1878, 1855-1879, 1856-1880, 1857-1881, 1858-1882, 1859-1883, 1860-1884, 1861-1885, 1862-1886, 1863-1887, 1864-1888, 1865-1889, 1866-1890, 1867-1891, 1868-1892, 1869-1893, 1870-1894, 1871-1895, 1872-1896, 1873-1897, 1874-1898, 1875-1899, 1876-1900, 1877-1901, 1878-1902, 1879-1903, 1880-1904, 1881-1905, 1882-1906, 1883-1907, 1884-1908, 1885-1909, 1886-1910, 1887-1911, 1888-1912, 1889-1913, 1890-1914, 1891-1915, 1892-1916, 1893-1917, 1894-1918, 1895-1919, 1896-1920, 1897-1921, 1898-1922, 1899-1923, 1900-1924, 1901-1925, 1902-1926, 1903-1927, 1904-1928, 1905-1929, 1906-1930, 1907-1931, 1908-1932, 1909-1933, 1910-1934, 1911-1935, 1912-1936, 1913-1937, 1914-1938, 1915-1939, 1916-1940, 1917-1941, 1918-1942, 1919-1943, 1920-1944, 1921-1945, 1922-1946, 1923-1947, 1924-1948, 1925-1949, 1926-1950, 1927-1951, 1928-1952, 1929-1953, 1930-1954, 1931-1955, 1932-1956, 1933-1957, 1934-1958, 1935-1959, 1936-1960, 1937-1961, 1938-1962, 1939-1963, 1940-1964, 1941-1965, 1942-1966, 1943-1967, 1944-1968, 1945-1969, 1946-1970, 1947-1971, 1948-1972, 1949-1973, 1950-1974, 1951-1975, 1952-1976, 1953-1977, 1954-1978, 1955-1979, 1956-1980, 1957-1981, 1958-1982, 1959-1983, 1960-1984, 1961-1985, 1962-1986, 1963-1987, 1964-1988, 1965-1989, 1966-1990, 1967-1991, 1968-1992, 1969-1993, 1970-1994, 1971-1995, 1972-1996, 1973-1997, 1974-1998, 1975-1999, 1976-2000, 1977-2001, 1978-2002, 1979-2003, 1980-2004, 1981-2005, 1982-2006, 1983-2007, 1984-2008, 1985-2009, 1986-2010, 1987-2011, 1988-2012, 1989-2013, 1990-2014, 1991-2015, 1992-2016, 1993-2017, 1994-2018, 1995-2019, 1996-2020, 1997-2021, 1998-2022, 1999-2023, 2000-2024, 2001-2025, 2002-2026, 2003-2027, 2004-2028, 2005-2029, 2006-2030, 2007-2031, 2008-2032, 2009-2033, 2010-2034, 2011-2035, 2012-2036, 2013-2037, 2014-2038, 2015-2039, 2016-2040, 2017-2041, 2018-2042, 2019-2043, 2020-2044, 2021-2045, 2022-2046, 2023-2047, 2024-2048, 2025-2049, 2026-2050, 2027-2051, 2028-2052, 2029-2053, 2030-2054, 2031-2055, 2032-2056, 2033-2057, 2034-2058, 2035-2059, 2036-2060, 2037-2061, 2038-2062, 2039-2063, 2040-2064, 2041-2065, 2042-2066, 2043-2067, 2044-2068, 2045-2069, 2046-2070, 2047-2071, 2048-2072 and 2049-2073.

Exemplary polynucleotide molecules include the following 12-mer fragments of the polynucleotide sequence from the sequence of SEQ ID NO:7: 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723, 713-724, 714-725, 715-726, 716-727, 717-728, 718-729, 719-730, 720-731, 721-732, 722-733, 723-734, 724-735, 725-736, 726-737, 727-738, 728-739, 729-740, 730-741, 731-742, 732-743, 733-744, 734-745, 735-746, 736-747, 737-748, 738-749, 739-750, 740-751, 741-752, 742-753, 743-754, 744-755, 745-756, 746-757, 747-758, 748-759, 749-760, 750-761, 751-762, 752-763, 753-764, 754-765, 755-766, 756-767, 757-768, 758-769, 759-770, 760-771, 761-772, 762-773, 763-774, 764-775, 765-776, 766-777, 767-778, 768-779, 769-780, 770-781, 771-782, 772-783, 773-784, 774-785, 775-786, 776-787, 777-788, 778-789, 779-790, 780-791, 781-792, 782-793, 783-794, 784-795, 785-796, 786-797, 787-798, 788-799, 789-800, 790-801, 791-802, 792-803, 793-804, 794-805, 795-806, 796-807, 797-808, 798-809, 799-810, 800-811, 801-812, 802-813, 803-814, 804-815, 805-816, 806-817, 807-818, 808-819, 809-820, 810-821, 811-822, 812-823, 813-824, 814-825, 815-826, 816-827, 817-828, 818-829, 819-830, 820-831, 821-832, 822-833, 823-834, 824-835, 825-836, 826-837, 827-838, 828-839, 829-840, 830-841, 831-842, 832-843, 833-844, 834-845, 835-846, 836-847, 837-848, 838-849, 839-850, 840-851, 841-852, 842-853, 843-854, 844-855, 845-856, 846-857, 847-858, 848-859, 849-860, 850-861, 851-862, 852-863, 853-864, 854-865, 855-866, 856-867, 857-868, 858-869, 859-870, 860-871, 861-872, 862-873, 863-874, 864-875, 865-876, 866-877, 867-878, 868-879, 869-880, 870-881, 871-882, 872-883, 873-884, 874-885, 875-886, 876-887, 877-888, 878-889, 879-890, 880-891, 881-892, 882-893, 883-894, 884-895, 885-896, 886-897, 887-898, 888-899, 889-900, 890-901, 891-902, 892-903, 893-904, 894-905, 895-906, 896-907, 897-908, 898-909, 899-910, 900-911, 901-912, 902-913, 903-914, 904-915, 905-916, 906-917, 907-918, 908-919, 909-920, 910-921, 911-922, 912-923, 913-924, 914-925, 915-926, 916-927, 917-928, 918-929, 919-930, 920-931, 921-932, 922-933, 923-934, 924-935, 925-936, 926-937, 927-938, 928-939, 929-940, 930-941, 931-942, 932-943, 933-944, 934-945, 935-946, 936-947, 937-948, 938-949, 939-950, 940-951, 941-952, 942-953, 943-954, 944-955, 945-956, 946-957, 947-958, 948-959, 949-960, 950-961, 951-962, 952-963, 953-964, 954-965, 955-966, 956-967, 957-968, 958-969, 959-970, 960-971, 961-972, 962-973, 963-974, 964-975, 965-976, 966-977, 967-978, 968-979, 969-980, 970-981, 971-982, 972-983, 973-984, 974-985, 975-986, 976-987, 977-988, 978-989, 979-990, 980-991, 981-992, 982-993, 983-994, 984-995, 985-996, 986-997, 987-998, 988-999, 989-1000, 990-1001, 991-1002, 992-1003, 993-1004, 994-1005, 995-1006, 996-1007, 997-1008, 998-1009, 999-1010, 1000-1011, 1001-1012, 1002-1013, 1003-1014, 1004-1015, 1005-1016, 1006-1017, 1007-1018, 1008-1019, 1009-1020, 1010-1021, 1011-1022, 1012-1023, 1013-1024, 1014-1025, 1015-1026, 1016-1027, 1017-1028, 1018-1029, 1019-1030, 1020-1031, 1021-1032, 1022-1033, 1023-1034, 1024-1035, 1025-1036, 1026-1037, 1027-1038, 1028-1039, 1029-1040, 1030-1041, 1031-1042, 1032-1043, 1033-1044, 1034-1045, 1035-1046, 1036-1047, 1037-1048, 1038-1049, 1039-1050, 1040-1051, 1041-1052, 1042-1053, 1043-1054, 1044-1055, 1045-1056, 1046-1057, 1047-1058, 1048-1059, 1049-1060, 1050-1061, 1051-1062, 1052-1063, 1053-1064, 1054-1065, 1055-1066, 1056-1067, 1057-1068, 1058-1069, 1059-1070, 1060-1071, 1061-1072, 1062-1073, 1063-1074, 1064-1075, 1065-1076, 1066-1077, 1067-1078, 1068-1079, 1069-1080, 1070-1081, 1071-1082, 1072-1083, 1073-1084, 1074-1085, 1075-1086, 1076-1087, 1077-1088, 1078-1089, 1079-1090, 1080-1091, 1081-1092, 1082-1093, 1083-1094, 1084-1095, 1085-1096, 1086-1097, 1087-1098, 1088-1099, 1089-1100, 1090-1101, 1091-1102, 1092-1103, 1093-1104, 1094-1105, 1095-1106, 1096-1107, 1097-1108, 1098-1109, 1099-1110, 1100-1111, 1101-1112, 1102-1113, 1103-1114, 1104-1115, 1105-1116, 1106-1117, 1107-1118, 1108-1119, 1109-1120, 1110-1121, 1111-1122, 1112-1123, 1113-1124, 1114-1125, 1115-1126, 1116-1127, 1117-1128, 1118-1129, 1119-1130, 1120-1131, 1121-1132, 1122-1133, 1123-1134, 1124-1135, 1125-1136, 1126-1137, 1127-1138, 1128-1139, 1129-1140, 1130-1141, 1131-1142, 1132-1143, 1133-1144, 1134-1145, 1135-1146, 1136-1147, 1137-1148, 1138-1149, 1139-1150, 1140-1151, 1141-1152, 1142-1153, 1143-1154, 1144-1155, 1145-1156, 1146-1157, 1147-1158, 1148-1159, 1149-1160, 1150-1161, 1151-1162, 1152-1163, 1153-1164, 1154-1165, 1155-1166, 1156-1167, 1157-1168, 1158-1169, 1159-1170, 1160-1171, 1161-1172, 1162-1173, 1163-1174, 1164-1175, 1165-1176, 1166-1177, 1167-1178, 1168-1179, 1169-1180, 1170-1181, 1171-1182, 1172-1183, 1173-1184, 1174-1185, 1175-1186, 1176-1187, 1177-1188, 1178-1189, 1179-1190, 1180-1191, 1181-1192, 1182-1193, 1183-1194, 1184-1195, 1185-1196, 1186-1197, 1187-1198, 1188-1199, 1189-1200, 1190-1201, 1191-1202, 1192-1203, 1193-1204, 1194-1205, 1195-1206, 1196-1207, 1197-1208, 1198-1209, 1199-1210, 1200-1211, 1201-1212, 1202-1213, 1203-1214, 1204-1215, 1205-1216, 1206-1217, 1207-1218, 1208-1219, 1209-1220, 1210-1221, 1211-1222, 1212-1223, 1213-1224, 1214-1225, 1215-1226, 1216-1227, 1217-1228, 1218-1229, 1219-1230, 1220-1231, 1221-1232, 1222-1233, 1223-1234, 1224-1235, 1225-1236, 1226-1237, 1227-1238, 1228-1239, 1229-1240, 1230-1241, 1231-1242, 1232-1243, 1233-1244, 1234-1245, 1235-1246, 1236-1247, 1237-1248, 1238-1249, 1239-1250, 1240-1251, 1241-1252, 1242-1253, 1243-1254, 1244-1255, 1245-1256, 1246-1257, 1247-1258, 1248-1259, 1249-1260, 1250-1261, 1251-1262, 1252-1263, 1253-1264, 1254-1265, 1255-1266, 1256-1267, 1257-1268, 1258-1269, 1259-1270, 1260-1271, 1261-1272, 1262-1273, 1263-1274, 1264-1275, 1265-1276, 1266-1277, 1267-1278, 1268-1279, 1269-1280, 1270-1281, 1271-1282, 1272-1283, 1273-1284, 1274-1285, 1275-1286, 1276-1287, 1277-1288, 1278-1289, 1279-1290, 1280-1291, 1281-1292, 1282-1293, 1283-1294, 1284-1295, 1285-1296, 1286-1297, 1287-1298, 1288-1299, 1289-1300, 1290-1301, 1291-1302, 1292-1303, 1293-1304, 1294-1305, 1295-1306, 1296-1307, 1297-1308, 1298-1309, 1299-1310, 1300-1311, 1301-1312, 1302-1313, 1303-1314, 1304-1315, 1305-1316, 1306-1317, 1307-1318, 1308-1319, 1309-1320, 1310-1321, 1311-1322, 1312-1323, 1313-1324, 1314-1325, 1315-1326, 1316-1327, 1317-1328, 1318-1329, 1319-1330, 1320-1331, 1321-1332, 1322-1333, 1323-1334, 1324-1335, 1325-1336, 1326-1337, 1327-1338, 1328-1339, 1329-1340, 1330-1341, 1331-1342, 1332-1343, 1333-1344, 1334-1345, 1335-1346, 1336-1347, 1337-1348, 1338-1349, 1339-1350, 1340-1351, 1341-1352, 1342-1353, 1343-1354, 1344-1355, 1345-1356, 1346-1357, 1347-1358, 1348-1359, 1349-1360, 1350-1361, 1351-1362, 1352-1363, 1353-1364, 1354-1365, 1355-1366, 1356-1367, 1357-1368, 1358-1369, 1359-1370, 1360-1371, 1361-1372, 1362-1373, 1363-1374, 1364-1375, 1365-1376, 1366-1377, 1367-1378, 1368-1379, 1369-1380, 1370-1381, 1371-1382, 1372-1383, 1373-1384, 1374-1385, 1375-1386, 1376-1387, 1377-1388, 1378-1389, 1379-1390, 1380-1391, 1381-1392, 1382-1393, 1383-1394, 1384-1395, 1385-1396, 1386-1397, 1387-1398, 1388-1399, 1389-1400, 1390-1401, 1391-1402, 1392-1403, 1393-1404, 1394-1405, 1395-1406, 1396-1407, 1397-1408, 1398-1409, 1399-1410, 1400-1411, 1401-1412, 1402-1413, 1403-1414, 1404-1415, 1405-1416, 1406-1417, 1407-1418, 1408-1419, 1409-1420, 1410-1421, 1411-1422, 1412-1423, 1413-1424, 1414-1425, 1415-1426, 1416-1427, 1417-1428, 1418-1429, 1419-1430, 1420-1431, 1421-1432, 1422-1433, 1423-1434, 1424-1435, 1425-1436, 1426-1437, 1427-1438, 1428-1439, 1429-1440, 1430-1441, 1431-1442, 1432-1443, 1433-1444, 1434-1445, 1435-1446, 1436-1447, 1437-1448, 1438-1449, 1439-1450, 1440-1451, 1441-1452, 1442-1453, 1443-1454, 1444-1455, 1445-1456, 1446-1457, 1447-1458, 1448-1459, 1449-1460, 1450-1461, 1451-1462, 1452-1463, 1453-1464, 1454-1465, 1455-1466, 1456-1467, 1457-1468, 1458-1469, 1459-1470, 1460-1471, 1461-1472, 1462-1473, 1463-1474, 1464-1475, 1465-1476, 1466-1477, 1467-1478, 1468-1479, 1469-1480, 1470-1481, 1471-1482, 1472-1483, 1473-1484, 1474-1485, 1475-1486, 1476-1487, 1477-1488, 1478-1489, 1479-1490, 1480-1491, 1481-1492, 1482-1493, 1483-1494, 1484-1495, 1485-1496, 1486-1497, 1487-1498, 1488-1499, 1489-1500, 1490-1501, 1491-1502, 1492-1503, 1493-1504, 1494-1505, 1495-1506, 1496-1507, 1497-1508, 1498-1509, 1499-1510, 1500-1511, 1501-1512, 1502-1513, 1503-1514, 1504-1515, 1505-1516, 1506-1517, 1507-1518, 1508-1519, 1509-1520, 1510-1521, 1511-1522, 1512-1523, 1513-1524, 1514-1525, 1515-1526, 1516-1527, 1517-1528, 1518-1529, 1519-1530, 1520-1531, 1521-1532, 1522-1533, 1523-1534, 1524-1535, 1525-1536, 1526-1537, 1527-1538, 1528-1539, 1529-1540, 1530-1541, 1531-1542, 1532-1543, 1533-1544, 1534-1545, 1535-1546, 1536-1547, 1537-1548, 1538-1549, 1539-1550, 1540-1551, 1541-1552, 1542-1553, 1543-1554, 1544-1555, 1545-1556, 1546-1557, 1547-1558, 1548-1559, 1549-1560, 1550-1561, 1551-1562, 1552-1563, 1553-1564, 1554-1565, 1555-1566, 1556-1567, 1557-1568, 1558-1569, 1559-1570, 1560-1571, 1561-1572, 1562-1573, 1563-1574, 1564-1575, 1565-1576, 1566-1577, 1567-1578, 1568-1579, 1569-1580, 1570-1581, 1571-1582, 1572-1583, 1573-1584, 1574-1585, 1575-1586, 1576-1587, 1577-1588, 1578-1589, 1579-1590, 1580-1591, 1581-1592, 1582-1593, 1583-1594, 1584-1595, 1585-1596, 1586-1597, 1587-1598, 1588-1599, 1589-1600, 1590-1601, 1591-1602, 1592-1603, 1593-1604, 1594-1605, 1595-1606, 1596-1607, 1597-1608, 1598-1609, 1599-1610, 1600-1611, 1601-1612, 1602-1613, 1603-1614, 1604-1615, 1605-1616, 1606-1617, 1607-1618, 1608-1619, 1609-1620, 1610-1621, 1611-1622, 1612-1623, 1613-1624, 1614-1625, 1615-1626, 1616-1627, 1617-1628, 1618-1629, 1619-1630, 1620-1631, 1621-1632, 1622-1633, 1623-1634, 1624-1635, 1625-1636, 1626-1637, 1627-1638, 1628-1639, 1629-1640, 1630-1641, 1631-1642, 1632-1643, 1633-1644, 1634-1645, 1635-1646, 1636-1647, 1637-1648, 1638-1649, 1639-1650, 1640-1651, 1641-1652, 1642-1653, 1643-1654, 1644-1655, 1645-1656, 1646-1657, 1647-1658, 1648-1659, 1649-1660, 1650-1661, 1651-1662, 1652-1663, 1653-1664, 1654-1665, 1655-1666, 1656-1667, 1657-1668, 1658-1669, 1659-1670, 1660-1671, 1661-1672, 1662-1673, 1663-1674, 1664-1675, 1665-1676, 1666-1677, 1667-1678, 1668-1679, 1669-1680, 1670-1681, 1671-1682, 1672-1683, 1673-1684, 1674-1685, 1675-1686, 1676-1687, 1677-1688, 1678-1689, 1679-1690, 1680-1691, 1681-1692, 1682-1693, 1683-1694, 1684-1695, 1685-1696, 1686-1697, 1687-1698, 1688-1699, 1689-1700, 1690-1701, 1691-1702, 1692-1703, 1693-1704, 1694-1705, 1695-1706, 1696-1707, 1697-1708, 1698-1709, 1699-1710, 1700-1711, 1701-1712, 1702-1713, 1703-1714, 1704-1715, 1705-1716, 1706-1717, 1707-1718, 1708-1719, 1709-1720, 1710-1721, 1711-1722, 1712-1723, 1713-1724, 1714-1725, 1715-1726, 1716-1727, 1717-1728, 1718-1729, 1719-1730, 1720-1731, 1721-1732, 1722-1733, 1723-1734, 1724-1735, 1725-1736, 1726-1737, 1727-1738, 1728-1739, 1729-1740, 1730-1741, 1731-1742, 1732-1743, 1733-1744, 1734-1745, 1735-1746, 1736-1747, 1737-1748, 1738-1749, 1739-1750, 1740-1751, 1741-1752, 1742-1753, 1743-1754, 1744-1755, 1745-1756, 1746-1757, 1747-1758, 1748-1759, 1749-1760, 1750-1761, 1751-1762, 1752-1763, 1753-1764, 1754-1765, 1755-1766, 1756-1767, 1757-1768, 1758-1769, 1759-1770, 1760-1771, 1761-1772, 1762-1773, 1763-1774, 1764-1775, 1765-1776, 1766-1777, 1767-1778, 1768-1779, 1769-1780, 1770-1781, 1771-1782, 1772-1783, 1773-1784, 1774-1785, 1775-1786, 1776-1787, 1777-1788, 1778-1789, 1779-1790, 1780-1791, 1781-1792, 1782-1793, 1783-1794, 1784-1795, 1785-1796, 1786-1797, 1787-1798, 1788-1799, 1789-1800, 1790-1801, 1791-1802, 1792-1803, 1793-1804, 1794-1805, 1795-1806, 1796-1807, 1797-1808, 1798-1809, 1799-1810, 1800-1811, 1801-1812, 1802-1813, 1803-1814, 1804-1815, 1805-1816, 1806-1817, 1807-1818, 1808-1819, 1809-1820, 1810-1821, 1811-1822, 1812-1823, 1813-1824, 1814-1825, 1815-1826, 1816-1827, 1817-1828, 1818-1829, 1819-1830, 1820-1831, 1821-1832, 1822-1833, 1823-1834, 1824-1835, 1825-1836, 1826-1837, 1827-1838, 1828-1839, 1829-1840, 1830-1841, 1831-1842, 1832-1843, 1833-1844, 1834-1845, 1835-1846, 1836-1847, 1837-1848, 1838-1849, 1839-1850, 1840-1851, 1841-1852, 1842-1853, 1843-1854, 1844-1855, 1845-1856, 1846-1857, 1847-1858, 1848-1859, 1849-1860, 1850-1861, 1851-1862, 1852-1863, 1853-1864, 1854-1865, 1855-1866, 1856-1867, 1857-1868, 1858-1869, 1859-1870, 1860-1871, 1861-1872, 1862-1873, 1863-1874, 1864-1875, 1865-1876, 1866-1877, 1867-1878, 1868-1879, 1869-1880, 1870-1881, 1871-1882, 1872-1883, 1873-1884, 1874-1885, 1875-1886, 1876-1887, 1877-1888, 1878-1889, 1879-1890, 1880-1891, 1881-1892, 1882-1893, 1883-1894, 1884-1895, 1885-1896, 1886-1897, 1887-1898, 1888-1899, 1889-1900, 1890-1901, 1891-1902, 1892-1903, 1893-1904, 1894-1905, 1895-1906, 1896-1907, 1897-1908, 1898-1909, 1899-1910, 1900-1911, 1901-1912, 1902-1913, 1903-1914, 1904-1915, 1905-1916, 1906-1917, 1907-1918, 1908-1919, 1909-1920, 1910-1921, 1911-1922, 1912-1923, 1913-1924, 1914-1925, 1915-1926, 1916-1927, 1917-1928, 1918-1929, 1919-1930, 1920-1931, 1921-1932, 1922-1933, 1923-1934, 1924-1935, 1925-1936, 1926-1937, 1927-1938, 1928-1939, 1929-1940, 1930-1941, 1931-1942, 1932-1943, 1933-1944, 1934-1945, 1935-1946, 1936-1947, 1937-1948, 1938-1949, 1939-1950, 1940-1951, 1941-1952, 1942-1953, 1943-1954, 1944-1955, 1945-1956, 1946-1957, 1947-1958, 1948-1959, 1949-1960, 1950-1961, 1951-1962, 1952-1963, 1953-1964, 1954-1965, 1955-1966, 1956-1967, 1957-1968, 1958-1969, 1959-1970, 1960-1971, 1961-1972, 1962-1973, 1963-1974, 1964-1975, 1965-1976, 1966-1977, 1967-1978, 1968-1979, 1969-1980, 1970-1981, 1971-1982, 1972-1983, 1973-1984, 1974-1985, 1975-1986, 1976-1987, 1977-1988, 1978-1989, 1979-1990, 1980-1991, 1981-1992, 1982-1993, 1983-1994, 1984-1995, 1985-1996, 1986-1997, 1987-1998, 1988-1999, 1989-2000, 1990-2001, 1991-2002, 1992-2003, 1993-2004, 1994-2005, 1995-2006, 1996-2007, 1997-2008, 1998-2009, 1999-2010, 2000-2011, 2001-2012, 2002-2013, 2003-2014, 2004-2015, 2005-2016, 2006-2017, 2007-2018, 2008-2019, 2009-2020, 2010-2021, 2011-2022, 2012-2023, 2013-2024, 2014-2025, 2015-2026, 2016-2027, 2017-2028, 2018-2029, 2019-2030, 2020-2031, 2021-2032, 2022-2033, 2023-2034, 2024-2035, 2025-2036, 2026-2037, 2027-2038, 2028-2039, 2029-2040, 2030-2041, 2031-2042, 2032-2043, 2033-2044, 2034-2045, 2035-2046, 2036-2047, 2037-2048, 2038-2049, 2039-2050, 2040-2051, 2041-2052, 2042-2053, 2043-2054, 2044-2055, 2045-2056, 2046-2057, 2047-2058, 2048-2059, 2049-2060, 2050-2061, 2051-2062, 2052-2063, 2053-2064, 2054-2065, 2055-2066, 2056-2067, 2057-2068, 2058-2069, 2059-2070, 2060-2071, 2061-2072, 2062-2073, 2063-2074, 2064-2075, 2065-2076, 2066-2077, 2067-2078, 2068-2079, 2069-2080, 2070-2081, 2071-2082, 2072-2083, 2073-2084, 2074-2085, 2075-2086, 2076-2087, 2077-2088, 2078-2089, 2079-2090, 2080-2091, 2081-2092, 2082-2093, 2083-2094, 2084-2095, 2085-2096, 2086-2097, 2087-2098, 2088-2099, 2089-2100, 2090-2101, 2091-2102, 2092-2103, 2093-2104, 2094-2105, 2095-2106, 2096-2107, 2097-2108, 2098-2109, 2099-2110, 2100-2111, 2101-2112, 2102-2113, 2103-2114, 2104-2115, 2105-2116, 2106-2117, 2107-2118, 2108-2119, 2109-2120, 2110-2121, 2111-2122, 2112-2123, 2113-2124, 2114-2125, 2115-2126, 2116-2127, 2117-2128, 2118-2129, 2119-2130, 2120-2131, 2121-2132, 2122-2133, 2123-2134, 2124-2135, 2125-2136, 2126-2137, 2127-2138, 2128-2139, 2129-2140, 2130-2141, 2131-2142, 2132-2143, 2133-2144, 2134-2145, 2135-2146, 2136-2147, 2137-2148, 2138-2149, 2139-2150, 2140-2151, 2141-2152, 2142-2153, 2143-2154, 2144-2155, 2145-2156, 2146-2157, 2147-2158, 2148-2159, 2149-2160, 2150-2161, 2151-2162, 2152-2163, 2153-2164, 2154-2165, 2155-2166, 2156-2167, 2157-2168, 2158-2169, 2159-2170, 2160-2171 and 2161-2172.

Exemplary polynucleotide molecules include the following 15-mer fragments of the polynucleotide sequence from the sequence of SEQ ID NO:7: 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723, 710-724, 711-725, 712-726, 713-727, 714-728, 715-729, 716-730, 717-731, 718-732, 719-733, 720-734, 721-735, 722-736, 723-737, 724-738, 725-739, 726-740, 727-741, 728-742, 729-743, 730-744, 731-745, 732-746, 733-747, 734-748, 735-749, 736-750, 737-751, 738-752, 739-753, 740-754, 741-755, 742-756, 743-757, 744-758, 745-759, 746-760, 747-761, 748-762, 749-763, 750-764, 751-765, 752-766, 753-767, 754-768, 755-769, 756-770, 757-771, 758-772, 759-773, 760-774, 761-775, 762-776, 763-777, 764-778, 765-779, 766-780, 767-781, 768-782, 769-783, 770-784, 771-785, 772-786, 773-787, 774-788, 775-789, 776-790, 777-791, 778-792, 779-793, 780-794, 781-795, 782-796, 783-797, 784-798, 785-799, 786-800, 787-801, 788-802, 789-803, 790-804, 791-805, 792-806, 793-807, 794-808, 795-809, 796-810, 797-811, 798-812, 799-813, 800-814, 801-815, 802-816, 803-817, 804-818, 805-819, 806-820, 807-821, 808-822, 809-823, 810-824, 811-825, 812-826, 813-827, 814-828, 815-829, 816-830, 817-831, 818-832, 819-833, 820-834, 821-835, 822-836, 823-837, 824-838, 825-839, 826-840, 827-841, 828-842, 829-843, 830-844, 831-845, 832-846, 833-847, 834-848, 835-849, 836-850, 837-851, 838-852, 839-853, 840-854, 841-855, 842-856, 843-857, 844-858, 845-859, 846-860, 847-861, 848-862, 849-863, 850-864, 851-865, 852-866, 853-867, 854-868, 855-869, 856-870, 857-871, 858-872, 859-873, 860-874, 861-875, 862-876, 863-877, 864-878, 865-879, 866-880, 867-881, 868-882, 869-883, 870-884, 871-885, 872-886, 873-887, 874-888, 875-889, 876-890, 877-891, 878-892, 879-893, 880-894, 881-895, 882-896, 883-897, 884-898, 885-899, 886-900, 887-901, 888-902, 889-903, 890-904, 891-905, 892-906, 893-907, 894-908, 895-909, 896-910, 897-911, 898-912, 899-913, 900-914, 901-915, 902-916, 903-917, 904-918, 905-919, 906-920, 907-921, 908-922, 909-923, 910-924, 911-925, 912-926, 913-927, 914-928, 915-929, 916-930, 917-931, 918-932, 919-933, 920-934, 921-935, 922-936, 923-937, 924-938, 925-939, 926-940, 927-941, 928-942, 929-943, 930-944, 931-945, 932-946, 933-947, 934-948, 935-949, 936-950, 937-951, 938-952, 939-953, 940-954, 941-955, 942-956, 943-957, 944-958, 945-959, 946-960, 947-961, 948-962, 949-963, 950-964, 951-965, 952-966, 953-967, 954-968, 955-969, 956-970, 957-971, 958-972, 959-973, 960-974, 961-975, 962-976, 963-977, 964-978, 965-979, 966-980, 967-981, 968-982, 969-983, 970-984, 971-985, 972-986, 973-987, 974-988, 975-989, 976-990, 977-991, 978-992, 979-993, 980-994, 981-995, 982-996, 983-997, 984-998, 985-999, 986-1000, 987-1001, 988-1002, 989-1003, 990-1004, 991-1005, 992-1006, 993-1007, 994-1008, 995-1009, 996-1010, 997-1011, 998-1012, 999-1013, 1000-1014, 1001-1015, 1002-1016, 1003-1017, 1004-1018, 1005-1019, 1006-1020, 1007-1021, 1008-1022, 1009-1023, 1010-1024, 1011-1025, 1012-1026, 1013-1027, 1014-1028, 1015-1029, 1016-1030, 1017-1031, 1018-1032, 1019-1033, 1020-1034, 1021-1035, 1022-1036, 1023-1037, 1024-1038, 1025-1039, 1026-1040, 1027-1041, 1028-1042, 1029-1043, 1030-1044, 1031-1045, 1032-1046, 1033-1047, 1034-1048, 1035-1049, 1036-1050, 1037-1051, 1038-1052, 1039-1053, 1040-1054, 1041-1055, 1042-1056, 1043-1057, 1044-1058, 1045-1059, 1046-1060, 1047-1061, 1048-1062, 1049-1063, 1050-1064, 1051-1065, 1052-1066, 1053-1067, 1054-1068, 1055-1069, 1056-1070, 1057-1071, 1058-1072, 1059-1073, 1060-1074, 1061-1075, 1062-1076, 1063-1077, 1064-1078, 1065-1079, 1066-1080, 1067-1081, 1068-1082, 1069-1083, 1070-1084, 1071-1085, 1072-1086, 1073-1087, 1074-1088, 1075-1089, 1076-1090, 1077-1091, 1078-1092, 1079-1093, 1080-1094, 1081-1095, 1082-1096, 1083-1097, 1084-1098, 1085-1099, 1086-1100, 1087-1101, 1088-1102, 1089-1103, 1090-1104, 1091-1105, 1092-1106, 1093-1107, 1094-1108, 1095-1109, 1096-1110, 1097-1111, 1098-1112, 1099-1113, 1100-1114, 1101-1115, 1102-1116, 1103-1117, 1104-1118, 1105-1119, 1106-1120, 1107-1121, 1108-1122, 1109-1123, 1110-1124, 1111-1125, 1112-1126, 1113-1127, 1114-1128, 1115-1129, 1116-1130, 1117-1131, 1118-1132, 1119-1133, 1120-1134, 1121-1135, 1122-1136, 1123-1137, 1124-1138, 1125-1139, 1126-1140, 1127-1141, 1128-1142, 1129-1143, 1130-1144, 1131-1145, 1132-1146, 1133-1147, 1134-1148, 1135-1149, 1136-1150, 1137-1151, 1138-1152, 1139-1153, 1140-1154, 1141-1155, 1142-1156, 1143-1157, 1144-1158, 1145-1159, 1146-1160, 1147-1161, 1148-1162, 1149-1163, 1150-1164, 1151-1165, 1152-1166, 1153-1167, 1154-1168, 1155-1169, 1156-1170, 1157-1171, 1158-1172, 1159-1173, 1160-1174, 1161-1175, 1162-1176, 1163-1177, 1164-1178, 1165-1179, 1166-1180, 1167-1181, 1168-1182, 1169-1183, 1170-1184, 1171-1185, 1172-1186, 1173-1187, 1174-1188, 1175-1189, 1176-1190, 1177-1191, 1178-1192, 1179-1193, 1180-1194, 1181-1195, 1182-1196, 1183-1197, 1184-1198, 1185-1199, 1186-1200, 1187-1201, 1188-1202, 1189-1203, 1190-1204, 1191-1205, 1192-1206, 1193-1207, 1194-1208, 1195-1209, 1196-1210, 1197-1211, 1198-1212, 1199-1213, 1200-1214, 1201-1215, 1202-1216, 1203-1217, 1204-1218, 1205-1219, 1206-1220, 1207-1221, 1208-1222, 1209-1223, 1210-1224, 1211-1225, 1212-1226, 1213-1227, 1214-1228, 1215-1229, 1216-1230, 1217-1231, 1218-1232, 1219-1233, 1220-1234, 1221-1235, 1222-1236, 1223-1237, 1224-1238, 1225-1239, 1226-1240, 1227-1241, 1228-1242, 1229-1243, 1230-1244, 1231-1245, 1232-1246, 1233-1247, 1234-1248, 1235-1249, 1236-1250, 1237-1251, 1238-1252, 1239-1253, 1240-1254, 1241-1255, 1242-1256, 1243-1257, 1244-1258, 1245-1259, 1246-1260, 1247-1261, 1248-1262, 1249-1263, 1250-1264, 1251-1265, 1252-1266, 1253-1267, 1254-1268, 1255-1269, 1256-1270, 1257-1271, 1258-1272, 1259-1273, 1260-1274, 1261-1275, 1262-1276, 1263-1277, 1264-1278, 1265-1279, 1266-1280, 1267-1281, 1268-1282, 1269-1283, 1270-1284, 1271-1285, 1272-1286, 1273-1287, 1274-1288, 1275-1289, 1276-1290, 1277-1291, 1278-1292, 1279-1293, 1280-1294, 1281-1295, 1282-1296, 1283-1297, 1284-1298, 1285-1299, 1286-1300, 1287-1301, 1288-1302, 1289-1303, 1290-1304, 1291-1305, 1292-1306, 1293-1307, 1294-1308, 1295-1309, 1296-1310, 1297-1311, 1298-1312, 1299-1313, 1300-1314, 1301-1315, 1302-1316, 1303-1317, 1304-1318, 1305-1319, 1306-1320, 1307-1321, 1308-1322, 1309-1323, 1310-1324, 1311-1325, 1312-1326, 1313-1327, 1314-1328, 1315-1329, 1316-1330, 1317-1331, 1318-1332, 1319-1333, 1320-1334, 1321-1335, 1322-1336, 1323-1337, 1324-1338, 1325-1339, 1326-1340, 1327-1341, 1328-1342, 1329-1343, 1330-1344, 1331-1345, 1332-1346, 1333-1347, 1334-1348, 1335-1349, 1336-1350, 1337-1351, 1338-1352, 1339-1353, 1340-1354, 1341-1355, 1342-1356, 1343-1357, 1344-1358, 1345-1359, 1346-1360, 1347-1361, 1348-1362, 1349-1363, 1350-1364, 1351-1365, 1352-1366, 1353-1367, 1354-1368, 1355-1369, 1356-1370, 1357-1371, 1358-1372, 1359-1373, 1360-1374, 1361-1375, 1362-1376, 1363-1377, 1364-1378, 1365-1379, 1366-1380, 1367-1381, 1368-1382, 1369-1383, 1370-1384, 1371-1385, 1372-1386, 1373-1387, 1374-1388, 1375-1389, 1376-1390, 1377-1391, 1378-1392, 1379-1393, 1380-1394, 1381-1395, 1382-1396, 1383-1397, 1384-1398, 1385-1399, 1386-1400, 1387-1401, 1388-1402, 1389-1403, 1390-1404, 1391-1405, 1392-1406, 1393-1407, 1394-1408, 1395-1409, 1396-1410, 1397-1411, 1398-1412, 1399-1413, 1400-1414, 1401-1415, 1402-1416, 1403-1417, 1404-1418, 1405-1419, 1406-1420, 1407-1421, 1408-1422, 1409-1423, 1410-1424, 1411-1425, 1412-1426, 1413-1427, 1414-1428, 1415-1429, 1416-1430, 1417-1431, 1418-1432, 1419-1433, 1420-1434, 1421-1435, 1422-1436, 1423-1437, 1424-1438, 1425-1439, 1426-1440, 1427-1441, 1428-1442, 1429-1443, 1430-1444, 1431-1445, 1432-1446, 1433-1447, 1434-1448, 1435-1449, 1436-1450, 1437-1451, 1438-1452, 1439-1453, 1440-1454, 1441-1455, 1442-1456, 1443-1457, 1444-1458, 1445-1459, 1446-1460, 1447-1461, 1448-1462, 1449-1463, 1450-1464, 1451-1465, 1452-1466, 1453-1467, 1454-1468, 1455-1469, 1456-1470, 1457-1471, 1458-1472, 1459-1473, 1460-1474, 1461-1475, 1462-1476, 1463-1477, 1464-1478, 1465-1479, 1466-1480, 1467-1481, 1468-1482, 1469-1483, 1470-1484, 1471-1485, 1472-1486, 1473-1487, 1474-1488, 1475-1489, 1476-1490, 1477-1491, 1478-1492, 1479-1493, 1480-1494, 1481-1495, 1482-1496, 1483-1497, 1484-1498, 1485-1499, 1486-1500, 1487-1501, 1488-1502, 1489-1503, 1490-1504, 1491-1505, 1492-1506, 1493-1507, 1494-1508, 1495-1509, 1496-1510, 1497-1511, 1498-1512, 1499-1513, 1500-1514, 1501-1515, 1502-1516, 1503-1517, 1504-1518, 1505-1519, 1506-1520, 1507-1521, 1508-1522, 1509-1523, 1510-1524, 1511-1525, 1512-1526, 1513-1527, 1514-1528, 1515-1529, 1516-1530, 1517-1531, 1518-1532, 1519-1533, 1520-1534, 1521-1535, 1522-1536, 1523-1537, 1524-1538, 1525-1539, 1526-1540, 1527-1541, 1528-1542, 1529-1543, 1530-1544, 1531-1545, 1532-1546, 1533-1547, 1534-1548, 1535-1549, 1536-1550, 1537-1551, 1538-1552, 1539-1553, 1540-1554, 1541-1555, 1542-1556, 1543-1557, 1544-1558, 1545-1559, 1546-1560, 1547-1561, 1548-1562, 1549-1563, 1550-1564, 1551-1565, 1552-1566, 1553-1567, 1554-1568, 1555-1569, 1556-1570, 1557-1571, 1558-1572, 1559-1573, 1560-1574, 1561-1575, 1562-1576, 1563-1577, 1564-1578, 1565-1579, 1566-1580, 1567-1581, 1568-1582, 1569-1583, 1570-1584, 1571-1585, 1572-1586, 1573-1587, 1574-1588, 1575-1589, 1576-1590, 1577-1591, 1578-1592, 1579-1593, 1580-1594, 1581-1595, 1582-1596, 1583-1597, 1584-1598, 1585-1599, 1586-1600, 1587-1601, 1588-1602, 1589-1603, 1590-1604, 1591-1605, 1592-1606, 1593-1607, 1594-1608, 1595-1609, 1596-1610, 1597-1611, 1598-1612, 1599-1613, 1600-1614, 1601-1615, 1602-1616, 1603-1617, 1604-1618, 1605-1619, 1606-1620, 1607-1621, 1608-1622, 1609-1623, 1610-1624, 1611-1625, 1612-1626, 1613-1627, 1614-1628, 1615-1629, 1616-1630, 1617-1631, 1618-1632, 1619-1633, 1620-1634, 1621-1635, 1622-1636, 1623-1637, 1624-1638, 1625-1639, 1626-1640, 1627-1641, 1628-1642, 1629-1643, 1630-1644, 1631-1645, 1632-1646, 1633-1647, 1634-1648, 1635-1649, 1636-1650, 1637-1651, 1638-1652, 1639-1653, 1640-1654, 1641-1655, 1642-1656, 1643-1657, 1644-1658, 1645-1659, 1646-1660, 1647-1661, 1648-1662, 1649-1663, 1650-1664, 1651-1665, 1652-1666, 1653-1667, 1654-1668, 1655-1669, 1656-1670, 1657-1671, 1658-1672, 1659-1673, 1660-1674, 1661-1675, 1662-1676, 1663-1677, 1664-1678, 1665-1679, 1666-1680, 1667-1681, 1668-1682, 1669-1683, 1670-1684, 1671-1685, 1672-1686, 1673-1687, 1674-1688, 1675-1689, 1676-1690, 1677-1691, 1678-1692, 1679-1693, 1680-1694, 1681-1695, 1682-1696, 1683-1697, 1684-1698, 1685-1699, 1686-1700, 1687-1701, 1688-1702, 1689-1703, 1690-1704, 1691-1705, 1692-1706, 1693-1707, 1694-1708, 1695-1709, 1696-1710, 1697-1711, 1698-1712, 1699-1713, 1700-1714, 1701-1715, 1702-1716, 1703-1717, 1704-1718, 1705-1719, 1706-1720, 1707-1721, 1708-1722, 1709-1723, 1710-1724, 1711-1725, 1712-1726, 1713-1727, 1714-1728, 1715-1729, 1716-1730, 1717-1731, 1718-1732, 1719-1733, 1720-1734, 1721-1735, 1722-1736, 1723-1737, 1724-1738, 1725-1739, 1726-1740, 1727-1741, 1728-1742, 1729-1743, 1730-1744, 1731-1745, 1732-1746, 1733-1747, 1734-1748, 1735-1749, 1736-1750, 1737-1751, 1738-1752, 1739-1753, 1740-1754, 1741-1755, 1742-1756, 1743-1757, 1744-1758, 1745-1759, 1746-1760, 1747-1761, 1748-1762, 1749-1763, 1750-1764, 1751-1765, 1752-1766, 1753-1767, 1754-1768, 1755-1769, 1756-1770, 1757-1771, 1758-1772, 1759-1773, 1760-1774, 1761-1775, 1762-1776, 1763-1777, 1764-1778, 1765-1779, 1766-1780, 1767-1781, 1768-1782, 1769-1783, 1770-1784, 1771-1785, 1772-1786, 1773-1787, 1774-1788, 1775-1789, 1776-1790, 1777-1791, 1778-1792, 1779-1793, 1780-1794, 1781-1795, 1782-1796, 1783-1797, 1784-1798, 1785-1799, 1786-1800, 1787-1801, 1788-1802, 1789-1803, 1790-1804, 1791-1805, 1792-1806, 1793-1807, 1794-1808, 1795-1809, 1796-1810, 1797-1811, 1798-1812, 1799-1813, 1800-1814, 1801-1815, 1802-1816, 1803-1817, 1804-1818, 1805-1819, 1806-1820, 1807-1821, 1808-1822, 1809-1823, 1810-1824, 1811-1825, 1812-1826, 1813-1827, 1814-1828, 1815-1829, 1816-1830, 1817-1831, 1818-1832, 1819-1833, 1820-1834, 1821-1835, 1822-1836, 1823-1837, 1824-1838, 1825-1839, 1826-1840, 1827-1841, 1828-1842, 1829-1843, 1830-1844, 1831-1845, 1832-1846, 1833-1847, 1834-1848, 1835-1849, 1836-1850, 1837-1851, 1838-1852, 1839-1853, 1840-1854, 1841-1855, 1842-1856, 1843-1857, 1844-1858, 1845-1859, 1846-1860, 1847-1861, 1848-1862, 1849-1863, 1850-1864, 1851-1865, 1852-1866, 1853-1867, 1854-1868, 1855-1869, 1856-1870, 1857-1871, 1858-1872, 1859-1873, 1860-1874, 1861-1875, 1862-1876, 1863-1877, 1864-1878, 1865-1879, 1866-1880, 1867-1881, 1868-1882, 1869-1883, 1870-1884, 1871-1885, 1872-1886, 1873-1887, 1874-1888, 1875-1889, 1876-1890, 1877-1891, 1878-1892, 1879-1893, 1880-1894, 1881-1895, 1882-1896, 1883-1897, 1884-1898, 1885-1899, 1886-1900, 1887-1901, 1888-1902, 1889-1903, 1890-1904, 1891-1905, 1892-1906, 1893-1907, 1894-1908, 1895-1909, 1896-1910, 1897-1911, 1898-1912, 1899-1913, 1900-1914, 1901-1915, 1902-1916, 1903-1917, 1904-1918, 1905-1919, 1906-1920, 1907-1921, 1908-1922, 1909-1923, 1910-1924, 1911-1925, 1912-1926, 1913-1927, 1914-1928, 1915-1929, 1916-1930, 1917-1931, 1918-1932, 1919-1933, 1920-1934, 1921-1935, 1922-1936, 1923-1937, 1924-1938, 1925-1939, 1926-1940, 1927-1941, 1928-1942, 1929-1943, 1930-1944, 1931-1945, 1932-1946. 1933-1947, 1934-1948, 1935-1949, 1936-1950, 1937-1951, 1938-1952, 1939-1953, 1940-1954, 1941-1955, 1942-1956, 1943-1957, 1944-1958, 1945-1959, 1946-1960, 1947-1961, 1948-1962, 1949-1963, 1950-1964, 1951-1965, 1952-1966, 1953-1967, 1954-1968, 1955-1969, 1956-1970, 1957-1971, 1958-1972, 1959-1973, 1960-1974, 1961-1975, 1962-1976, 1963-1977, 1964-1978, 1965-1979, 1966-1980, 1967-1981, 1968-1982, 1969-1983, 1970-1984, 1971-1985, 1972-1986, 1973-1987, 1974-1988, 1975-1989, 1976-1990, 1977-1991, 1978-1992, 1979-1993, 1980-1994, 1981-1995, 1982-1996, 1983-1997, 1984-1998, 1985-1999, 1986-2000, 1987-2001, 1988-2002, 1989-2003, 1990-2004, 1991-2005, 1992-2006, 1993-2007, 1994-2008, 1995-2009, 1996-2010, 1997-2011, 1998-2012, 1999-2013, 2000-2014, 2001-2015, 2002-2016, 2003-2017, 2004-2018, 2005-2019, 2006-2020, 2007-2021, 2008-2022, 2009-2023, 2010-2024, 2011-2025, 2012-2026, 2013-2027, 2014-2028, 2015-2029, 2016-2030, 2017-2031, 2018-2032, 2019-2033, 2020-2034, 2021-2035, 2022-2036, 2023-2037, 2024-2038, 2025-2039, 2026-2040, 2027-2041, 2028-2042, 2029-2043, 2030-2044, 2031-2045, 2032-2046, 2033-2047, 2034-2048, 2035-2049, 2036-2050, 2037-2051, 2038-2052, 2039-2053, 2040-2054, 2041-2055, 2042-2056, 2043-2057, 2044-2058, 2045-2059, 2046-2060, 2047-2061, 2048-2062, 2049-2063, 2050-2064, 2051-2065, 2052-2066, 2053-2067, 2054-2068, 2055-2069, 2056-2070, 2057-2071, 2058-2072, 2059-2073, 2060-2074, 2061-2075, 2062-2076, 2063-2077, 2064-2078, 2065-2079, 2066-2080, 2067-2081, 2068-2082, 2069-2083, 2070-2084, 2071-2085, 2072-2086, 2073-2087, 2074-2088, 2075-2089, 2076-2090, 2077-2091, 2078-2092, 2079-2093, 2080-2094, 2081-2095, 2082-2096, 2083-2097, 2084-2098, 2085-2099, 2086-2100, 2087-2101, 2088-2102, 2089-2103, 2090-2104, 2091-2105, 2092-2106, 2093-2107, 2094-2108, 2095-2109, 2096-2110, 2097-2111, 2098-2112, 2099-2113, 2100-2114, 2101-2115, 2102-2116, 2103-2117, 2104-2118, 2105-2119, 2106-2120, 2107-2121, 2108-2122, 2109-2123, 2110-2124, 2111-2125, 2112-2126, 2113-2127, 2114-2128, 2115-2129, 2116-2130, 2117-2131, 2118-2132, 2119-2133, 2120-2134, 2121-2135, 2122-2136, 2123-2137, 2124-2138, 2125-2139, 2126-2140, 2127-2141, 2128-2142, 2129-2143, 2130-2144, 2131-2145, 2132-2146, 2133-2147, 2134-2148, 2135-2149, 2136-2150, 2137-2151, 2138-2152, 2139-2153, 2140-2154, 2141-2155, 2142-2156, 2143-2157, 2144-2158, 2145-2159, 2146-2160, 2147-2161, 2148-2162, 2149-2163, 2150-2164, 2151-2165, 2152-2166, 2153-2167, 2154-2168, 2155-2169, 2156-2170, 2157-2171 and 2158-2172.

Exemplary polynucleotide molecules include the following 20-mer fragments of the polynucleotide sequence from the sequence of SEQ ID NO:7: 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182. 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-1420, 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, 1581-1600, 1582-1601, 1583-1602, 1584-1603, 1585-1604, 1586-1605, 1587-1606, 1588-1607, 1589-1608, 1590-1609, 1591-1610, 1592-1611, 1593-1612, 1594-1613, 1595-1614, 1596-1615, 1597-1616, 1598-1617, 1599-1618, 1600-1619, 1601-1620, 1602-1621, 1603-1622, 1604-1623, 1605-1624, 1606-1625, 1607-1626, 1608-1627, 1609-1628, 1610-1629, 1611-1630, 1612-1631, 1613-1632, 1614-1633, 1615-1634, 1616-1635, 1617-1636, 1618-1637, 1619-1638, 1620-1639, 1621-1640, 1622-1641, 1623-1642, 1624-1643, 1625-1644, 1626-1645, 1627-1646, 1628-1647, 1629-1648, 1630-1649, 1631-1650, 1632-1651, 1633-1652, 1634-1653, 1635-1654, 1636-1655, 1637-1656, 1638-1657, 1639-1658, 1640-1659, 1641-1660, 1642-1661, 1643-1662, 1644-1663, 1645-1664, 1646-1665, 1647-1666, 1648-1667, 1649-1668, 1650-1669, 1651-1670, 1652-1671, 1653-1672, 1654-1673, 1655-1674, 1656-1675, 1657-1676, 1658-1677, 1659-1678, 1660-1679, 1661-1680, 1662-1681, 1663-1682, 1664-1683, 1665-1684, 1666-1685, 1667-1686, 1668-1687, 1669-1688, 1670-1689, 1671-1690, 1672-1691, 1673-1692, 1674-1693, 1675-1694, 1676-1695, 1677-1696, 1678-1697, 1679-1698, 1680-1699, 1681-1700, 1682-1701, 1683-1702, 1684-1703, 1685-1704, 1686-1705, 1687-1706, 1688-1707, 1689-1708, 1690-1709, 1691-1710, 1692-1711, 1693-1712, 1694-1713, 1695-1714, 1696-1715, 1697-1716, 1698-1717, 1699-1718, 1700-1719, 1701-1720, 1702-1721, 1703-1722, 1704-1723, 1705-1724, 1706-1725, 1707-1726, 1708-1727, 1709-1728, 1710-1729, 1711-1730, 1712-1731, 1713-1732, 1714-1733, 1715-1734, 1716-1735, 1717-1736, 1718-1737, 1719-1738, 1720-1739, 1721-1740, 1722-1741, 1723-1742, 1724-1743, 1725-1744, 1726-1745, 1727-1746, 1728-1747, 1729-1748, 1730-1749, 1731-1750, 1732-1751, 1733-1752, 1734-1753, 1735-1754, 1736-1755, 1737-1756, 1738-1757, 1739-1758, 1740-1759, 1741-1760, 1742-1761, 1743-1762, 1744-1763, 1745-1764, 1746-1765, 1747-1766, 1748-1767, 1749-1768, 1750-1769, 1751-1770, 1752-1771, 1753-1772, 1754-1773, 1755-1774, 1756-1775, 1757-1776, 1758-1777, 1759-1778, 1760-1779, 1761-1780, 1762-1781, 1763-1782, 1764-1783, 1765-1784, 1766-1785, 1767-1786, 1768-1787, 1769-1788, 1770-1789, 1771-1790, 1772-1791, 1773-1792, 1774-1793, 1775-1794, 1776-1795, 1777-1796, 1778-1797, 1779-1798, 1780-1799, 1781-1800, 1782-1801, 1783-1802, 1784-1803, 1785-1804, 1786-1805, 1787-1806, 1788-1807, 1789-1808, 1790-1809, 1791-1810, 1792-1811, 1793-1812, 1794-1813, 1795-1814, 1796-1815, 1797-1816, 1798-1817, 1799-1818, 1800-1819, 1801-1820, 1802-1821, 1803-1822, 1804-1823, 1805-1824, 1806-1825, 1807-1826, 1808-1827, 1809-1828, 1810-1829, 1811-1830, 1812-1831, 1813-1832, 1814-1833, 1815-1834, 1816-1835, 1817-1836, 1818-1837, 1819-1838, 1820-1839, 1821-1840, 1822-1841, 1823-1842, 1824-1843, 1825-1844, 1826-1845, 1827-1846, 1828-1847, 1829-1848, 1830-1849, 1831-1850, 1832-1851, 1833-1852, 1834-1853, 1835-1854, 1836-1855, 1837-1856, 1838-1857, 1839-1858, 1840-1859, 1841-1860, 1842-1861, 1843-1862, 1844-1863, 1845-1864, 1846-1865, 1847-1866, 1848-1867, 1849-1868, 1850-1869, 1851-1870, 1852-1871, 1853-1872, 1854-1873, 1855-1874, 1856-1875, 1857-1876, 1858-1877, 1859-1878, 1860-1879, 1861-1880, 1862-1881, 1863-1882, 1864-1883, 1865-1884, 1866-1885, 1867-1886, 1868-1887, 1869-1888, 1870-1889, 1871-1890, 1872-1891, 1873-1892, 1874-1893, 1875-1894, 1876-1895, 1877-1896, 1878-1897, 1879-1898, 1880-1899, 1881-1900, 1882-1901, 1883-1902, 1884-1903, 1885-1904, 1886-1905, 1887-1906, 1888-1907, 1889-1908, 1890-1909, 1891-1910, 1892-1911, 1893-1912, 1894-1913, 1895-1914, 1896-1915, 1897-1916, 1898-1917, 1899-1918, 1900-1919, 1901-1920, 1902-1921, 1903-1922, 1904-1923, 1905-1924, 1906-1925, 1907-1926, 1908-1927, 1909-1928, 1910-1929, 1911-1930, 1912-1931, 1913-1932, 1914-1933, 1915-1934, 1916-1935, 1917-1936, 1918-1937, 1919-1938, 1920-1939, 1921-1940, 1922-1941, 1923-1942, 1924-1943, 1925-1944, 1926-1945, 1927-1946, 1928-1947, 1929-1948, 1930-1949, 1931-1950, 1932-1951, 1933-1952, 1934-1953, 1935-1954, 1936-1955, 1937-1956, 1938-1957, 1939-1958, 1940-1959, 1941-1960, 1942-1961, 1943-1962, 1944-1963, 1945-1964, 1946-1965, 1947-1966, 1948-1967, 1949-1968, 1950-1969, 1951-1970, 1952-1971, 1953-1972, 1954-1973, 1955-1974, 1956-1975, 1957-1976, 1958-1977, 1959-1978, 1960-1979, 1961-1980, 1962-1981, 1963-1982, 1964-1983, 1965-1984, 1966-1985, 1967-1986, 1968-1987, 1969-1988, 1970-1989, 1971-1990, 1972-1991, 1973-1992, 1974-1993, 1975-1994, 1976-1995, 1977-1996, 1978-1997, 1979-1998, 1980-1999, 1981-2000, 1982-2001, 1983-2002, 1984-2003, 1985-2004, 1986-2005, 1987-2006, 1988-2007, 1989-2008, 1990-2009, 1991-2010, 1992-2011, 1993-2012, 1994-2013, 1995-2014, 1996-2015, 1997-2016, 1998-2017, 1999-2018, 2000-2019, 2001-2020, 2002-2021, 2003-2022, 2004-2023, 2005-2024, 2006-2025, 2007-2026, 2008-2027, 2009-2028, 2010-2029, 2011-2030, 2012-2031, 2013-2032, 2014-2033, 2015-2034, 2016-2035, 2017-2036, 2018-2037, 2019-2038, 2020-2039, 2021-2040, 2022-2041, 2023-2042, 2024-2043, 2025-2044, 2026-2045, 2027-2046, 2028-2047, 2029-2048, 2030-2049, 2031-2050, 2032-2051, 2033-2052, 2034-2053, 2035-2054, 2036-2055, 2037-2056, 2038-2057, 2039-2058, 2040-2059, 2041-2060, 2042-2061, 2043-2062, 2044-2063, 2045-2064, 2046-2065, 2047-2066, 2048-2067, 2049-2068, 2050-2069, 2051-2070, 2052-2071, 2053-2072, 2054-2073, 2055-2074, 2056-2075, 2057-2076, 2058-2077, 2059-2078, 2060-2079, 2061-2080, 2062-2081, 2063-2082, 2064-2083, 2065-2084, 2066-2085, 2067-2086, 2068-2087, 2069-2088, 2070-2089, 2071-2090, 2072-2091, 2073-2092, 2074-2093, 2075-2094, 2076-2095, 2077-2096, 2078-2097, 2079-2098, 2080-2099, 2081-2100, 2082-2101, 2083-2102, 2084-2103, 2085-2104, 2086-2105, 2087-2106, 2088-2107, 2089-2108, 2090-2109, 2091-2110, 2092-2111, 2093-2112, 2094-2113, 2095-2114, 2096-2115, 2097-2116, 2098-2117, 2099-2118, 2100-2119, 2101-2120, 2102-2121, 2103-2122, 2104-2123, 2105-2124, 2106-2125, 2107-2126, 2108-2127, 2109-2128, 2110-2129, 2111-2130, 2112-2131, 2113-2132, 2114-2133, 2115-2134, 2116-2135, 2117-2136, 2118-2137, 2119-2138, 2120-2139, 2121-2140, 2122-2141, 2123-2142, 2124-2143, 2125-2144, 2126-2145, 2127-2146, 2128-2147, 2129-2148, 2130-2149, 2131-2150, 2132-2151, 2133-2152, 2134-2153, 2135-2154, 2136-2155, 2137-2156, 2138-2157, 2139-2158, 2140-2159, 2141-2160, 2142-2161, 2143-2162, 2144-2163, 2145-2164, 2146-2165, 2147-2166, 2148-2167, 2149-2168, 2150-2169, 2151-2170, 2152-2171 and 2153-2172.

Exemplary polynucleotide molecules include the following 25-mer fragments of the polynucleotide sequence from the sequence of SEQ ID NO:7: 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815, 792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1472, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-1501, 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568, 1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600, 1577-1601, 1578-1602, 1579-1603, 1580-1604, 1581-1605, 1582-1606, 1583-1607, 1584-1608, 1585-1609, 1586-1610, 1587-1611, 1588-1612, 1589-1613, 1590-1614, 1591-1615, 1592-1616, 1593-1617, 1594-1618, 1595-1619, 1596-1620, 1597-1621, 1598-1622, 1599-1623, 1600-1624, 1601-1625, 1602-1626, 1603-1627, 1604-1628, 1605-1629, 1606-1630, 1607-1631, 1608-1632, 1609-1633, 1610-1634, 1611-1635, 1612-1636, 1613-1637, 1614-1638, 1615-1639, 1616-1640, 1617-1641, 1618-1642, 1619-1643, 1620-1644, 1621-1645, 1622-1646, 1623-1647, 1624-1648, 1625-1649, 1626-1650, 1627-1651, 1628-1652, 1629-1653, 1630-1654, 1631-1655, 1632-1656, 1633-1657, 1634-1658, 1635-1659, 1636-1660, 1637-1661, 1638-1662, 1639-1663, 1640-1664, 1641-1665, 1642-1666, 1643-1667, 1644-1668, 1645-1669, 1646-1670, 1647-1671, 1648-1672, 1649-1673, 1650-1674, 1651-1675, 1652-1676, 1653-1677, 1654-1678, 1655-1679, 1656-1680, 1657-1681, 1658-1682, 1659-1683, 1660-1684, 1661-1685, 1662-1686, 1663-1687, 1664-1688, 1665-1689, 1666-1690, 1667-1691, 1668-1692, 1669-1693, 1670-1694, 1671-1695, 1672-1696, 1673-1697, 1674-1698, 1675-1699, 1676-1700, 1677-1701, 1678-1702, 1679-1703, 1680-1704, 1681-1705, 1682-1706, 1683-1707, 1684-1708, 1685-1709, 1686-1710, 1687-1711, 1688-1712, 1689-1713, 1690-1714, 1691-1715, 1692-1716, 1693-1717, 1694-1718, 1695-1719, 1696-1720, 1697-1721, 1698-1722, 1699-1723, 1700-1724, 1701-1725, 1702-1726, 1703-1727, 1704-1728, 1705-1729, 1706-1730, 1707-1731, 1708-1732, 1709-1733, 1710-1734, 1711-1735, 1712-1736, 1713-1737, 1714-1738, 1715-1739, 1716-1740, 1717-1741, 1718-1742, 1719-1743, 1720-1744, 1721-1745, 1722-1746, 1723-1747, 1724-1748, 1725-1749, 1726-1750, 1727-1751, 1728-1752, 1729-1753, 1730-1754, 1731-1755, 1732-1756, 1733-1757, 1734-1758, 1735-1759, 1736-1760, 1737-1761, 1738-1762, 1739-1763, 1740-1764, 1741-1765, 1742-1766, 1743-1767, 1744-1768, 1745-1769, 1746-1770, 1747-1771, 1748-1772, 1749-1773, 1750-1774, 1751-1775, 1752-1776, 1753-1777, 1754-1778, 1755-1779, 1756-1780, 1757-1781, 1758-1782, 1759-1783, 1760-1784, 1761-1785, 1762-1786, 1763-1787, 1764-1788, 1765-1789, 1766-1790, 1767-1791, 1768-1792, 1769-1793, 1770-1794, 1771-1795, 1772-1796, 1773-1797, 1774-1798, 1775-1799, 1776-1800, 1777-1801, 1778-1802, 1779-1803, 1780-1804, 1781-1805, 1782-1806, 1783-1807, 1784-1808, 1785-1809, 1786-1810, 1787-1811, 1788-1812, 1789-1813, 1790-1814, 1791-1815, 1792-1816, 1793-1817, 1794-1818, 1795-1819, 1796-1820, 1797-1821, 1798-1822, 1799-1823, 1800-1824, 1801-1825, 1802-1826, 1803-1827, 1804-1828, 1805-1829, 1806-1830, 1807-1831, 1808-1832, 1809-1833, 1810-1834, 1811-1835, 1812-1836, 1813-1837, 1814-1838, 1815-1839, 1816-1840, 1817-1841, 1818-1842, 1819-1843, 1820-1844, 1821-1845, 1822-1846, 1823-1847, 1824-1848, 1825-1849, 1826-1850, 1827-1851, 1828-1852, 1829-1853, 1830-1854, 1831-1855, 1832-1856, 1833-1857, 1834-1858, 1835-1859, 1836-1860, 1837-1861, 1838-1862, 1839-1863, 1840-1864, 1841-1865, 1842-1866, 1843-1867, 1844-1868, 1845-1869, 1846-1870, 1847-1871, 1848-1872, 1849-1873, 1850-1874, 1851-1875, 1852-1876, 1853-1877, 1854-1878, 1855-1879, 1856-1880, 1857-1881, 1858-1882, 1859-1883, 1860-1884, 1861-1885, 1862-1886, 1863-1887, 1864-1888, 1865-1889, 1866-1890, 1867-1891, 1868-1892, 1869-1893, 1870-1894, 1871-1895, 1872-1896, 1873-1897, 1874-1898, 1875-1899, 1876-1900, 1877-1901, 1878-1902, 1879-1903, 1880-1904, 1881-1905, 1882-1906, 1883-1907, 1884-1908, 1885-1909, 1886-1910, 1887-1911, 1888-1912, 1889-1913, 1890-1914, 1891-1915, 1892-1916, 1893-1917, 1894-1918, 1895-1919, 1896-1920, 1897-1921, 1898-1922, 1899-1923, 1900-1924, 1901-1925, 1902-1926, 1903-1927, 1904-1928, 1905-1929, 1906-1930, 1907-1931, 1908-1932, 1909-1933, 1910-1934, 1911-1935, 1912-1936, 1913-1937, 1914-1938, 1915-1939, 1916-1940, 1917-1941, 1918-1942, 1919-1943, 1920-1944, 1921-1945, 1922-1946, 1923-1947, 1924-1948, 1925-1949, 1926-1950, 1927-1951, 1928-1952, 1929-1953, 1930-1954, 1931-1955, 1932-1956, 1933-1957, 1934-1958, 1935-1959, 1936-1960, 1937-1961, 1938-1962, 1939-1963, 1940-1964, 1941-1965, 1942-1966, 1943-1967, 1944-1968, 1945-1969, 1946-1970, 1947-1971, 1948-1972, 1949-1973, 1950-1974, 1951-1975, 1952-1976, 1953-1977, 1954-1978, 1955-1979, 1956-1980, 1957-1981, 1958-1982, 1959-1983, 1960-1984, 1961-1985, 1962-1986, 1963-1987, 1964-1988, 1965-1989, 1966-1990, 1967-1991, 1968-1992, 1969-1993, 1970-1994, 1971-1995, 1972-1996, 1973-1997, 1974-1998, 1975-1999, 1976-2000, 1977-2001, 1978-2002, 1979-2003, 1980-2004, 1981-2005, 1982-2006, 1983-2007, 1984-2008, 1985-2009, 1986-2010, 1987-2011, 1988-2012, 1989-2013, 1990-2014, 1991-2015, 1992-2016, 1993-2017, 1994-2018, 1995-2019, 1996-2020, 1997-2021, 1998-2022, 1999-2023, 2000-2024, 2001-2025, 2002-2026, 2003-2027, 2004-2028, 2005-2029, 2006-2030, 2007-2031, 2008-2032, 2009-2033, 2010-2034, 2011-2035, 2012-2036, 2013-2037, 2014-2038, 2015-2039, 2016-2040, 2017-2041, 2018-2042, 2019-2043, 2020-2044, 2021-2045, 2022-2046, 2023-2047, 2024-2048, 2025-2049, 2026-2050, 2027-2051, 2028-2052, 2029-2053, 2030-2054, 2031-2055, 2032-2056, 2033-2057, 2034-2058, 2035-2059, 2036-2060, 2037-2061, 2038-2062, 2039-2063, 2040-2064, 2041-2065, 2042-2066, 2043-2067, 2044-2068, 2045-2069, 2046-2070, 2047-2071, 2048-2072, 2049-2073, 2050-2074, 2051-2075, 2052-2076, 2053-2077, 2054-2078, 2055-2079, 2056-2080, 2057-2081, 2058-2082, 2059-2083, 2060-2084, 2061-2085, 2062-2086, 2063-2087, 2064-2088, 2065-2089, 2066-2090, 2067-2091, 2068-2092, 2069-2093, 2070-2094, 2071-2095, 2072-2096, 2073-2097, 2074-2098, 2075-2099, 2076-2100, 2077-2101, 2078-2102, 2079-2103, 2080-2104, 2081-2105, 2082-2106, 2083-2107, 2084-2108, 2085-2109, 2086-2110, 2087-2111, 2088-2112, 2089-2113, 2090-2114, 2091-2115, 2092-2116, 2093-2117, 2094-2118, 2095-2119, 2096-2120, 2097-2121, 2098-2122, 2099-2123, 2100-2124, 2101-2125, 2102-2126, 2103-2127, 2104-2128, 2105-2129, 2106-2130, 2107-2131, 2108-2132, 2109-2133, 2110-2134, 2111-2135, 2112-2136, 2113-2137, 2114-2138, 2115-2139, 2116-2140, 2117-2141, 2118-2142, 2119-2143, 2120-2144, 2121-2145, 2122-2146, 2123-2147, 2124-2148, 2125-2149, 2126-2150, 2127-2151, 2128-2152, 2129-2153, 2130-2154, 2131-2155, 2132-2156, 2133-2157, 2134-2158, 2135-2159, 2136-2160, 2137-2161, 2138-2162, 2139-2163, 2140-2164, 2141-2165, 2142-2166, 2143-2167, 2144-2168, 2145-2169, 2146-2170, 2147-2171 and 2148-2172.

Exemplary polynucleotide molecules include the following 12-mer fragments of the polynucleotide sequence from the sequence of SEQ ID NO:10: 50-61, 51-62, 52-63, 53-64, 54-65, 55-66, 56-67, 57-68, 58-69, 59-70, 60-71, 61-72, 62-73, 63-74, 64-75, 65-76, 66-77, 67-78, 68-79, 69-80, 70-81, 71-82, 72-83, 73-84, 74-85, 75-86, 76-87, 77-88, 78-89, 79-90, 80-91, 81-92, 82-93, 83-94, 84-95, 85-96, 86-97, 87-98, 88-99, 89-100, 90-101, 91-102, 92-103, 93-104, 94-105, 95-106, 96-107, 97-108, 98-109, 99-110, 100-111, 101-112, 102-113, 103-114, 104-115, 105-116, 106-117, 107-118, 108-119, 109-120, 110-121, 111-122, 112-123, 113-124, 114-125, 115-126, 116-127, 117-128, 118-129, 119-130, 120-131, 121-132, 122-133, 123-134, 124-135, 125-136, 126-137, 127-138, 128-139, 129-140, 130-141, 131-142, 132-143, 133-144, 134-145, 135-146, 136-147, 137-148, 138-149, 139-150, 140-151, 141-152, 142-153, 143-154, 144-155, 145-156, 146-157, 147-158, 148-159, 149-160, 150-161, 151-162, 152-163, 153-164, 154-165, 155-166, 156-167, 157-168, 158-169, 159-170, 160-171, 161-172, 162-173, 163-174, 164-175, 165-176, 166-177, 167-178, 168-179, 169-180, 170-181, 171-182, 172-183, 173-184, 174-185, 175-186, 176-187, 177-188, 178-189, 179-190, 180-191, 181-192, 182-193, 183-194, 184-195, 185-196, 186-197, 187-198, 188-199, 189-200, 190-201, 191-202, 192-203, 193-204, 194-205, 195-206, 196-207, 197-208, 198-209, 199-210, 200-211, 201-212, 202-213, 203-214, 204-215, 205-216, 206-217, 207-218, 208-219, 209-220, 210-221, 211-222, 212-223, 213-224, 214-225, 215-226, 216-227, 217-228, 218-229, 219-230, 220-231, 221-232, 222-233, 223-234, 224-235, 225-236, 226-237, 227-238, 228-239, 229-240, 230-241, 231-242, 232-243, 233-244, 234-245, 235-246, 236-247, 237-248, 238-249, 239-250, 240-251, 241-252, 242-253, 243-254, 244-255, 245-256, 246-257, 247-258, 248-259, 249-260, 250-261, 251-262, 252-263, 253-264, 254-265, 255-266, 256-267, 257-268, 258-269, 259-270, 260-271, 261-272, 262-273, 263-274, 264-275, 265-276, 266-277, 267-278, 268-279, 269-280, 270-281, 271-282, 272-283, 273-284, 274-285, 275-286, 276-287, 277-288, 278-289, 279-290, 280-291, 281-292, 282-293, 283-294, 284-295, 285-296, 286-297, 287-298, 288-299, 289-300, 290-301, 291-302, 292-303, 293-304, 294-305, 295-306, 296-307, 297-308, 298-309, 299-310, 300-311, 301-312, 302-313, 303-314, 304-315, 305-316, 306-317, 307-318, 308-319, 309-320, 310-321, 311-322, 312-323, 313-324, 314-325, 315-326, 316-327, 317-328, 318-329, 319-330, 320-331, 321-332, 322-333, 323-334, 324-335, 325-336, 326-337, 327-338, 328-339, 329-340, 330-341, 331-342, 332-343, 333-344, 334-345, 335-346, 336-347, 337-348, 338-349, 339-350, 340-351, 341-352, 342-353, 343-354, 344-355, 345-356, 346-357, 347-358, 348-359, 349-360, 350-361, 351-362, 352-363, 353-364, 354-365, 355-366, 356-367, 357-368, 358-369, 359-370, 360-371, 361-372, 362-373, 363-374, 364-375, 365-376, 366-377, 367-378, 368-379, 369-380, 370-381, 371-382, 372-383, 373-384, 374-385, 375-386, 376-387, 377-388, 378-389, 379-390, 380-391, 381-392, 382-393, 383-394, 384-395, 385-396, 386-397, 387-398, 388-399, 389-400, 390-401, 391-402, 392-403, 393-404, 394-405, 395-406, 396-407, 397-408, 398-409, 399-410, 400-411, 401-412, 402-413, 403-414, 404-415, 405-416, 406-417, 407-418, 408-419, 409-420, 410-421, 411-422, 412-423, 413-424, 414-425, 415-426, 416-427, 417-428, 418-429, 419-430, 420-431, 421-432, 422-433, 423-434, 424-435, 425-436, 426-437, 427-438, 428-439, 429-440, 430-441, 431-442, 432-443, 433-444, 434-445, 435-446, 436-447, 437-448, 438-449, 439-450, 440-451, 441-452, 442-453, 443-454, 444-455, 445-456, 446-457, 447-458, 448-459, 449-460, 450-461, 451-462, 452-463, 453-464, 454-465, 455-466, 456-467, 457-468, 458-469, 459-470, 460-471, 461-472, 462-473, 463-474, 464-475, 465-476, 466-477, 467-478, 468-479, 469-480, 470-481, 471-482, 472-483, 473-484, 474-485, 475-486, 476-487, 477-488, 478-489, 479-490, 480-491, 481-492, 482-493, 483-494, 484-495, 485-496, 486-497, 487-498, 488-499, 489-500, 490-501, 491-502, 492-503, 493-504, 494-505, 495-506, 496-507, 497-508, 498-509, 499-510, 500-511, 501-512, 502-513, 503-514, 504-515, 505-516, 506-517, 507-518, 508-519, 509-520, 510-521, 511-522, 512-523, 513-524, 514-525, 515-526, 516-527, 517-528, 518-529, 519-530, 520-531, 521-532, 522-533, 523-534, 524-535, 525-536, 526-537, 527-538, 528-539, 529-540, 530-541, 531-542, 532-543, 533-544, 534-545, 535-546, 536-547, 537-548, 538-549, 539-550, 540-551, 541-552, 542-553, 543-554, 544-555, 545-556, 546-557, 547-558, 548-559, 549-560, 550-561, 551-562, 552-563, 553-564, 554-565, 555-566, 556-567, 557-568, 558-569, 559-570, 560-571, 561-572, 562-573, 563-574, 564-575, 565-576, 566-577, 567-578, 568-579, 569-580, 570-581, 571-582, 572-583, 573-584, 574-585, 575-586, 576-587, 577-588, 578-589, 579-590, 580-591, 581-592, 582-593, 583-594, 584-595, 585-596, 586-597, 587-598, 588-599, 589-600, 590-601, 591-602, 592-603, 593-604, 594-605, 595-606, 596-607, 597-608, 598-609, 599-610, 600-611, 601-612, 602-613, 603-614, 604-615, 605-616, 606-617, 607-618, 608-619, 609-620, 610-621, 611-622, 612-623, 613-624, 614-625, 615-626, 616-627, 617-628, 618-629, 619-630, 620-631, 621-632, 622-633, 623-634, 624-635, 625-636, 626-637, 627-638, 628-639, 629-640, 630-641, 631-642, 632-643, 633-644, 634-645, 635-646, 636-647, 637-648, 638-649, 639-650, 640-651, 641-652, 642-653, 643-654, 644-655, 645-656, 646-657, 647-658, 648-659, 649-660, 650-661, 651-662, 652-663, 653-664, 654-665, 655-666, 656-667, 657-668, 658-669, 659-670, 660-671, 661-672, 662-673, 663-674, 664-675, 665-676, 666-677, 667-678, 668-679, 669-680, 670-681, 671-682, 672-683, 673-684, 674-685, 675-686, 676-687, 677-688, 678-689, 679-690, 680-691, 681-692, 682-693, 683-694, 684-695, 685-696, 686-697, 687-698, 688-699, 689-700, 690-701, 691-702, 692-703, 693-704, 694-705, 695-706, 696-707, 697-708, 698-709, 699-710, 700-711, 701-712, 702-713, 703-714, 704-715, 705-716, 706-717, 707-718, 708-719, 709-720, 710-721, 711-722, 712-723, 713-724, 714-725, 715-726, 716-727, 717-728, 718-729, 719-730, 720-731, 721-732, 722-733, 723-734, 724-735, 725-736, 726-737, 727-738, 728-739, 729-740, 730-741, 731-742, 732-743, 733-744, 734-745, 735-746, 736-747, 737-748, 738-749, 739-750, 740-751, 741-752, 742-753, 743-754, 744-755, 745-756, 746-757, 747-758, 748-759, 749-760, 750-761, 751-762, 752-763, 753-764, 754-765, 755-766, 756-767, 757-768, 758-769, 759-770, 760-771, 761-772, 762-773, 763-774, 764-775, 765-776, 766-777, 767-778, 768-779, 769-780, 770-781, 771-782, 772-783, 773-784, 774-785, 775-786, 776-787, 777-788, 778-789, 779-790, 780-791, 781-792, 782-793, 783-794, 784-795, 785-796, 786-797, 787-798, 788-799, 789-800, 790-801, 791-802, 792-803, 793-804, 794-805, 795-806, 796-807, 797-808, 798-809, 799-810, 800-811, 801-812, 802-813, 803-814, 804-815, 805-816, 806-817, 807-818, 808-819, 809-820, 810-821, 811-822, 812-823, 813-824, 814-825, 815-826, 816-827, 817-828, 818-829, 819-830, 820-831, 821-832, 822-833, 823-834, 824-835, 825-836, 826-837, 827-838, 828-839, 829-840, 830-841, 831-842, 832-843, 833-844, 834-845, 835-846, 836-847, 837-848, 838-849, 839-850, 840-851, 841-852, 842-853, 843-854, 844-855, 845-856, 846-857, 847-858, 848-859, 849-860, 850-861, 851-862, 852-863, 853-864, 854-865, 855-866, 856-867, 857-868, 858-869, 859-870, 860-871, 861-872, 862-873, 863-874, 864-875, 865-876, 866-877, 867-878, 868-879, 869-880, 870-881, 871-882, 872-883, 873-884, 874-885, 875-886, 876-887, 877-888, 878-889, 879-890, 880-891, 881-892, 882-893, 883-894, 884-895, 885-896, 886-897, 887-898, 888-899, 889-900, 890-901, 891-902, 892-903, 893-904, 894-905, 895-906, 896-907, 897-908, 898-909, 899-910, 900-911, 901-912, 902-913, 903-914, 904-915, 905-916, 906-917, 907-918, 908-919, 909-920, 910-921, 911-922, 912-923, 913-924, 914-925, 915-926, 916-927, 917-928, 918-929, 919-930, 920-931, 921-932, 922-933, 923-934, 924-935, 925-936, 926-937, 927-938, 928-939, 929-940, 930-941, 931-942, 932-943, 933-944, 934-945, 935-946, 936-947, 937-948, 938-949, 939-950, 940-951, 941-952, 942-953, 943-954, 944-955, 945-956, 946-957, 947-958, 948-959, 949-960, 950-961, 951-962, 952-963, 953-964, 954-965, 955-966, 956-967, 957-968, 958-969, 959-970, 960-971, 961-972, 962-973, 963-974, 964-975, 965-976, 966-977, 967-978, 968-979, 969-980, 970-981, 971-982, 972-983, 973-984, 974-985, 975-986, 976-987, 977-988, 978-989, 979-990, 980-991, 981-992, 982-993, 983-994, 984-995, 985-996, 986-997, 987-998, 988-999, 989-1000, 990-1001, 991-1002, 992-1003, 993-1004, 994-1005, 995-1006, 996-1007, 997-1008, 998-1009, 999-1010, 1000-1011, 1001-1012, 1002-1013, 1003-1014, 1004-1015, 1005-1016, 1006-1017, 1007-1018, 1008-1019, 1009-1020, 1010-1021, 1011-1022, 1012-1023, 1013-1024, 1014-1025, 1015-1026, 1016-1027, 1017-1028, 1018-1029, 1019-1030, 1020-1031, 1021-1032, 1022-1033, 1023-1034, 1024-1035, 1025-1036, 1026-1037, 1027-1038, 1028-1039, 1029-1040, 1030-1041, 1031-1042, 1032-1043, 1033-1044, 1034-1045, 1035-1046, 1036-1047, 1037-1048, 1038-1049, 1039-1050, 1040-1051, 1041-1052, 1042-1053, 1043-1054, 1044-1055, 1045-1056, 1046-1057, 1047-1058, 1048-1059, 1049-1060, 1050-1061, 1051-1062, 1052-1063, 1053-1064, 1054-1065, 1055-1066, 1056-1067, 1057-1068, 1058-1069, 1059-1070, 1060-1071, 1061-1072, 1062-1073, 1063-1074, 1064-1075, 1065-1076, 1066-1077, 1067-1078, 1068-1079, 1069-1080, 1070-1081, 1071-1082, 1072-1083, 1073-1084, 1074-1085, 1075-1086, 1076-1087, 1077-1088, 1078-1089, 1079-1090, 1080-1091, 1081-1092, 1082-1093, 1083-1094, 1084-1095, 1085-1096, 1086-1097, 1087-1098, 1088-1099, 1089-1100, 1090-1101, 1091-1102, 1092-1103, 1093-1104, 1094-1105, 1095-1106, 1096-1107, 1097-1108, 1098-1109, 1099-1110, 1100-1111, 1101-1112, 1102-1113, 1103-1114, 1104-1115, 1105-1116, 1106-1117, 1107-1118, 1108-1119, 1109-1120, 1110-1121, 1111-1122, 1112-1123, 1113-1124, 1114-1125, 1115-1126, 1116-1127, 1117-1128, 1118-1129, 1119-1130, 1120-1131, 1121-1132, 1122-1133, 1123-1134, 1124-1135, 1125-1136, 1126-1137, 1127-1138, 1128-1139, 1129-1140, 1130-1141, 1131-1142, 1132-1143, 1133-1144, 1134-1145, 1135-1146, 1136-1147, 1137-1148, 1138-1149, 1139-1150, 1140-1151, 1141-1152, 1142-1153, 1143-1154, 1144-1155, 1145-1156, 1146-1157, 1147-1158, 1148-1159, 1149-1160, 1150-1161, 1151-1162, 1152-1163, 1153-1164, 1154-1165, 1155-1166, 1156-1167, 1157-1168, 1158-1169, 1159-1170, 1160-1171, 1161-1172, 1162-1173, 1163-1174, 1164-1175, 1165-1176, 1166-1177, 1167-1178, 1168-1179, 1169-1180, 1170-1181, 1171-1182, 1172-1183, 1173-1184, 1174-1185, 1175-1186, 1176-1187, 1177-1188, 1178-1189, 1179-1190, 1180-1191, 1181-1192, 1182-1193, 1183-1194, 1184-1195, 1185-1196, 1186-1197, 1187-1198, 1188-1199, 1189-1200, 1190-1201, 1191-1202, 1192-1203, 1193-1204, 1194-1205, 1195-1206, 1196-1207, 1197-1208, 1198-1209, 1199-1210, 1200-1211, 1201-1212, 1202-1213, 1203-1214, 1204-1215, 1205-1216, 1206-1217, 1207-1218, 1208-1219, 1209-1220, 1210-1221, 1211-1222, 1212-1223, 1213-1224, 1214-1225, 1215-1226, 1216-1227, 1217-1228, 1218-1229, 1219-1230, 1220-1231, 1221-1232, 1222-1233, 1223-1234, 1224-1235, 1225-1236, 1226-1237, 1227-1238, 1228-1239, 1229-1240, 1230-1241, 1231-1242, 1232-1243, 1233-1244, 1234-1245, 1235-1246, 1236-1247, 1237-1248, 1238-1249, 1239-1250, 1240-1251, 1241-1252, 1242-1253, 1243-1254, 1244-1255, 1245-1256, 1246-1257, 1247-1258, 1248-1259, 1249-1260, 1250-1261, 1251-1262, 1252-1263, 1253-1264, 1254-1265, 1255-1266, 1256-1267, 1257-1268, 1258-1269, 1259-1270, 1260-1271, 1261-1272, 1262-1273, 1263-1274, 1264-1275, 1265-1276, 1266-1277, 1267-1278, 1268-1279, 1269-1280, 1270-1281, 1271-1282, 1272-1283, 1273-1284, 1274-1285, 1275-1286, 1276-1287, 1277-1288, 1278-1289, 1279-1290, 1280-1291, 1281-1292, 1282-1293, 1283-1294, 1284-1295, 1285-1296, 1286-1297, 1287-1298, 1288-1299, 1289-1300, 1290-1301, 1291-1302, 1292-1303, 1293-1304, 1294-1305, 1295-1306, 1296-1307, 1297-1308, 1298-1309, 1299-1310, 1300-1311, 1301-1312, 1302-1313, 1303-1314, 1304-1315, 1305-1316, 1306-1317, 1307-1318, 1308-1319, 1309-1320, 1310-1321, 1311-1322, 1312-1323, 1313-1324, 1314-1325, 1315-1326, 1316-1327, 1317-1328, 1318-1329, 1319-1330, 1320-1331, 1321-1332, 1322-1333, 1323-1334, 1324-1335, 1325-1336, 1326-1337, 1327-1338, 1328-1339, 1329-1340, 1330-1341, 1331-1342, 1332-1343, 1333-1344, 1334-1345, 1335-1346, 1336-1347, 1337-1348, 1338-1349, 1339-1350, 1340-1351, 1341-1352, 1342-1353, 1343-1354, 1344-1355, 1345-1356, 1346-1357, 1347-1358, 1348-1359, 1349-1360, 1350-1361, 1351-1362, 1352-1363, 1353-1364, 1354-1365, 1355-1366, 1356-1367, 1357-1368, 1358-1369, 1359-1370, 1360-1371, 1361-1372, 1362-1373, 1363-1374, 1364-1375, 1365-1376, 1366-1377, 1367-1378, 1368-1379, 1369-1380, 1370-1381, 1371-1382, 1372-1383, 1373-1384, 1374-1385, 1375-1386, 1376-1387, 1377-1388, 1378-1389, 1379-1390, 1380-1391, 1381-1392, 1382-1393, 1383-1394, 1384-1395, 1385-1396, 1386-1397, 1387-1398, 1388-1399, 1389-1400, 1390-1401, 1391-1402, 1392-1403, 1393-1404, 1394-1405, 1395-1406, 1396-1407, 1397-1408, 1398-1409, 1399-1410, 1400-1411, 1401-1412, 1402-1413, 1403-1414, 1404-1415, 1405-1416, 1406-1417, 1407-1418, 1408-1419, 1409-1420, 1410-1421, 1411-1422, 1412-1423, 1413-1424, 1414-1425, 1415-1426, 1416-1427, 1417-1428, 1418-1429, 1419-1430, 1420-1431, 1421-1432, 1422-1433, 1423-1434, 1424-1435, 1425-1436, 1426-1437, 1427-1438, 1428-1439, 1429-1440, 1430-1441, 1431-1442, 1432-1443, 1433-1444, 1434-1445, 1435-1446, 1436-1447, 1437-1448, 1438-1449, 1439-1450, 1440-1451, 1441-1452, 1442-1453, 1443-1454, 1444-1455, 1445-1456, 1446-1457, 1447-1458, 1448-1459, 1449-1460, 1450-1461, 1451-1462, 1452-1463, 1453-1464, 1454-1465, 1455-1466, 1456-1467, 1457-1468, 1458-1469, 1459-1470, 1460-1471, 1461-1472, 1462-1473, 1463-1474, 1464-1475, 1465-1476, 1466-1477, 1467-1478, 1468-1479, 1469-1480, 1470-1481, 1471-1482, 1472-1483, 1473-1484, 1474-1485, 1475-1486, 1476-1487, 1477-1488, 1478-1489, 1479-1490, 1480-1491, 1481-1492, 1482-1493, 1483-1494, 1484-1495, 1485-1496, 1486-1497, 1487-1498, 1488-1499, 1489-1500, 1490-1501, 1491-1502, 1492-1503, 1493-1504, 1494-1505, 1495-1506, 1496-1507, 1497-1508, 1498-1509, 1499-1510, 1500-1511, 1501-1512, 1502-1513, 1503-1514, 1504-1515, 1505-1516, 1506-1517, 1507-1518, 1508-1519, 1509-1520, 1510-1521, 1511-1522, 1512-1523, 1513-1524, 1514-1525, 1515-1526, 1516-1527, 1517-1528, 1518-1529, 1519-1530, 1520-1531, 1521-1532, 1522-1533, 1523-1534, 1524-1535, 1525-1536, 1526-1537, 1527-1538, 1528-1539, 1529-1540, 1530-1541, 1531-1542, 1532-1543, 1533-1544, 1534-1545, 1535-1546, 1536-1547, 1537-1548, 1538-1549, 1539-1550, 1540-1551, 1541-1552, 1542-1553, 1543-1554, 1544-1555, 1545-1556, 1546-1557, 1547-1558, 1548-1559, 1549-1560, 1550-1561, 1551-1562, 1552-1563, 1553-1564, 1554-1565, 1555-1566, 1556-1567, 1557-1568, 1558-1569, 1559-1570, 1560-1571, 1561-1572, 1562-1573, 1563-1574, 1564-1575, 1565-1576, 1566-1577, 1567-1578, 1568-1579, 1569-1580, 1570-1581, 1571-1582, 1572-1583, 1573-1584, 1574-1585, 1575-1586, 1576-1587, 1577-1588, 1578-1589, 1579-1590, 1580-1591, 1581-1592, 1582-1593, 1583-1594, 1584-1595, 1585-1596, 1586-1597, 1587-1598, 1588-1599, 1589-1600, 1590-1601, 1591-1602, 1592-1603, 1593-1604, 1594-1605, 1595-1606, 1596-1607, 1597-1608, 1598-1609, 1599-1610, 1600-1611, 1601-1612, 1602-1613, 1603-1614, 1604-1615, 1605-1616, 1606-1617, 1607-1618, 1608-1619, 1609-1620, 1610-1621, 1611-1622, 1612-1623, 1613-1624, 1614-1625, 1615-1626, 1616-1627, 1617-1628, 1618-1629, 1619-1630, 1620-1631, 1621-1632, 1622-1633, 1623-1634, 1624-1635, 1625-1636, 1626-1637, 1627-1638, 1628-1639, 1629-1640, 1630-1641, 1631-1642, 1632-1643, 1633-1644, 1634-1645, 1635-1646, 1636-1647, 1637-1648, 1638-1649, 1639-1650, 1640-1651, 1641-1652, 1642-1653, 1643-1654, 1644-1655, 1645-1656, 1646-1657, 1647-1658, 1648-1659, 1649-1660, 1650-1661, 1651-1662, 1652-1663, 1653-1664, 1654-1665, 1655-1666, 1656-1667, 1657-1668, 1658-1669, 1659-1670, 1660-1671, 1661-1672, 1662-1673, 1663-1674, 1664-1675, 1665-1676, 1666-1677, 1667-1678, 1668-1679, 1669-1680, 1670-1681, 1671-1682, 1672-1683, 1673-1684, 1674-1685, 1675-1686, 1676-1687, 1677-1688, 1678-1689, 1679-1690, 1680-1691, 1681-1692, 1682-1693, 1683-1694, 1684-1695, 1685-1696, 1686-1697, 1687-1698, 1688-1699, 1689-1700, 1690-1701, 1691-1702, 1692-1703, 1693-1704, 1694-1705, 1695-1706, 1696-1707, 1697-1708, 1698-1709, 1699-1710, 1700-1711, 1701-1712, 1702-1713, 1703-1714, 1704-1715, 1705-1716, 1706-1717, 1707-1718, 1708-1719, 1709-1720, 1710-1721, 1711-1722, 1712-1723, 1713-1724, 1714-1725, 1715-1726, 1716-1727, 1717-1728, 1718-1729, 1719-1730, 1720-1731, 1721-1732, 1722-1733, 1723-1734, 1724-1735, 1725-1736, 1726-1737, 1727-1738, 1728-1739, 1729-1740, 1730-1741, 1731-1742, 1732-1743, 1733-1744, 1734-1745, 1735-1746, 1736-1747, 1737-1748, 1738-1749, 1739-1750, 1740-1751, 1741-1752, 1742-1753, 1743-1754, 1744-1755, 1745-1756, 1746-1757, 1747-1758, 1748-1759, 1749-1760, 1750-1761, 1751-1762, 1752-1763, 1753-1764, 1754-1765, 1755-1766, 1756-1767, 1757-1768, 1758-1769, 1759-1770, 1760-1771, 1761-1772, 1762-1773, 1763-1774, 1764-1775, 1765-1776, 1766-1777, 1767-1778, 1768-1779, 1769-1780, 1770-1781, 1771-1782, 1772-1783, 1773-1784, 1774-1785, 1775-1786, 1776-1787, 1777-1788, 1778-1789, 1779-1790, 1780-1791, 1781-1792, 1782-1793, 1783-1794, 1784-1795, 1785-1796, 1786-1797, 1787-1798, 1788-1799, 1789-1800, 1790-1801, 1791-1802, 1792-1803, 1793-1804, 1794-1805, 1795-1806, 1796-1807, 1797-1808, 1798-1809, 1799-1810, 1800-1811, 1801-1812, 1802-1813, 1803-1814, 1804-1815, 1805-1816, 1806-1817, 1807-1818, 1808-1819, 1809-1820, 1810-1821, 1811-1822, 1812-1823, 1813-1824, 1814-1825, 1815-1826, 1816-1827, 1817-1828, 1818-1829, 1819-1830, 1820-1831, 1821-1832, 1822-1833, 1823-1834, 1824-1835, 1825-1836, 1826-1837, 1827-1838, 1828-1839, 1829-1840, 1830-1841, 1831-1842, 1832-1843, 1833-1844, 1834-1845, 1835-1846, 1836-1847, 1837-1848, 1838-1849, 1839-1850, 1840-1851, 1841-1852, 1842-1853, 1843-1854, 1844-1855, 1845-1856, 1846-1857, 1847-1858, 1848-1859, 1849-1860, 1850-1861, 1851-1862, 1852-1863, 1853-1864, 1854-1865, 1855-1866, 1856-1867, 1857-1868, 1858-1869, 1859-1870, 1860-1871, 1861-1872, 1862-1873, 1863-1874, 1864-1875, 1865-1876, 1866-1877, 1867-1878, 1868-1879, 1869-1880, 1870-1881, 1871-1882, 1872-1883, 1873-1884, 1874-1885, 1875-1886, 1876-1887, 1877-1888, 1878-1889, 1879-1890, 1880-1891, 1881-1892, 1882-1893, 1883-1894, 1884-1895, 1885-1896, 1886-1897, 1887-1898, 1888-1899, 1889-1900, 1890-1901, 1891-1902, 1892-1903, 1893-1904, 1894-1905, 1895-1906, 1896-1907, 1897-1908, 1898-1909, 1899-1910, 1900-1911, 1901-1912, 1902-1913, 1903-1914, 1904-1915, 1905-1916, 1906-1917, 1907-1918, 1908-1919, 1909-1920, 1910-1921, 1911-1922, 1912-1923, 1913-1924, 1914-1925, 1915-1926, 1916-1927, 1917-1928, 1918-1929, 1919-1930, 1920-1931, 1921-1932, 1922-1933, 1923-1934, 1924-1935, 1925-1936, 1926-1937, 1927-1938, 1928-1939, 1929-1940, 1930-1941, 1931-1942, 1932-1943, 1933-1944, 1934-1945, 1935-1946, 1936-1947, 1937-1948, 1938-1949, 1939-1950, 1940-1951, 1941-1952, 1942-1953, 1943-1954, 1944-1955, 1945-1956, 1946-1957, 1947-1958, 1948-1959, 1949-1960, 1950-1961, 1951-1962, 1952-1963, 1953-1964, 1954-1965, 1955-1966, 1956-1967, 1957-1968, 1958-1969, 1959-1970, 1960-1971, 1961-1972, 1962-1973, 1963-1974, 1964-1975, 1965-1976, 1966-1977, 1967-1978, 1968-1979, 1969-1980, 1970-1981, 1971-1982, 1972-1983, 1973-1984, 1974-1985, 1975-1986, 1976-1987, 1977-1988, 1978-1989, 1979-1990, 1980-1991, 1981-1992, 1982-1993, 1983-1994, 1984-1995, 1985-1996, 1986-1997, 1987-1998, 1988-1999, 1989-2000, 1990-2001, 1991-2002, 1992-2003, 1993-2004, 1994-2005, 1995-2006, 1996-2007, 1997-2008, 1998-2009, 1999-2010, 2000-2011, 2001-2012, 2002-2013, 2003-2014, 2004-2015, 2005-2016, 2006-2017, 2007-2018, 2008-2019, 2009-2020, 2010-2021, 2011-2022, 2012-2023, 2013-2024, 2014-2025, 2015-2026, 2016-2027, 2017-2028, 2018-2029, 2019-2030, 2020-2031, 2021-2032, 2022-2033, 2023-2034, 2024-2035, 2025-2036, 2026-2037, 2027-2038, 2028-2039, 2029-2040, 2030-2041, 2031-2042, 2032-2043, 2033-2044, 2034-2045, 2035-2046, 2036-2047, 2037-2048, 2038-2049, 2039-2050, 2040-2051, 2041-2052, 2042-2053, 2043-2054, 2044-2055, 2045-2056, 2046-2057, 2047-2058, 2048-2059, 2049-2060, 2050-2061, 2051-2062, 2052-2063, 2053-2064, 2054-2065, 2055-2066, 2056-2067, 2057-2068, 2058-2069, 2059-2070, 2060-2071, 2061-2072, 2062-2073, 2063-2074, 2064-2075, 2065-2076, 2066-2077, 2067-2078, 2068-2079, 2069-2080, 2070-2081, 2071-2082, 2072-2083, 2073-2084, 2074-2085, 2075-2086, 2076-2087, 2077-2088, 2078-2089, 2079-2090, 2080-2091, 2081-2092, 2082-2093, 2083-2094, 2084-2095, 2085-2096, 2086-2097, 2087-2098, 2088-2099, 2089-2100, 2090-2101, 2091-2102, 2092-2103, 2093-2104, 2094-2105, 2095-2106, 2096-2107, 2097-2108, 2098-2109, 2099-2110, 2100-2111, 2101-2112, 2102-2113, 2103-2114, 2104-2115, 2105-2116, 2106-2117, 2107-2118, 2108-2119, 2109-2120, 2110-2121, 2111-2122, 2112-2123, 2113-2124, 2114-2125, 2115-2126, 2116-2127, 2117-2128, 2118-2129, 2119-2130, 2120-2131, 2121-2132, 2122-2133, 2123-2134, 2124-2135, 2125-2136, 2126-2137, 2127-2138, 2128-2139, 2129-2140, 2130-2141, 2131-2142, 2132-2143, 2133-2144, 2134-2145, 2135-2146, 2136-2147, 2137-2148, 2138-2149, 2139-2150, 2140-2151, 2141-2152, 2142-2153, 2143-2154, 2144-2155, 2145-2156, 2146-2157, 2147-2158, 2148-2159, 2149-2160, 2150-2161, 2151-2162, 2152-2163, 2153-2164, 2154-2165, 2155-2166, 2156-2167, 2157-2168, 2158-2169, 2159-2170, 2160-2171, 2161-2172, 2162-2173, 2163-2174, 2164-2175, 2165-2176, 2166-2177, 2167-2178, 2168-2179, 2169-2180, 2170-2181, 2171-2182, 2172-2183, 2173-2184, 2174-2185, 2175-2186, 2176-2187, 2177-2188, 2178-2189, 2179-2190, 2180-2191, 2181-2192, 2182-2193, 2183-2194, 2184-2195, 2185-2196, 2186-2197, 2187-2198, 2188-2199, 2189-2200, 2190-2201, 2191-2202, 2192-2203, 2193-2204, 2194-2205, 2195-2206, 2196-2207, 2197-2208, 2198-2209, 2199-2210, 2200-2211, 2201-2212, 2202-2213, 2203-2214, 2204-2215, 2205-2216, 2206-2217, 2207-2218, 2208-2219, 2209-2220, 2210-2221, 2211-2222, 2212-2223, 2213-2224, 2214-2225, 2215-2226, 2216-2227, 2217-2228, 2218-2229, 2219-2230, 2220-2231, 2221-2232, 2322-2333, 2323-2334, 2324-2335, 2325-2336, 2326-2337, 2327-2338, 2328-2339, 2329-2340, 2330-2341, 2331-2342, 2332-2343, 2333-2344, 2334-2345, 2335-2346, 2336-2347, 2337-2348, 2338-2349, 2339-2350, 2340-2351, 2341-2352, 2342-2353, 2343-2354, 2344-2355, 2345-2356, 2346-2357, 2347-2358, 2348-2359, 2349-2360, 2350-2361, 2351-2362, 2352-2363, 2353-2364, 2354-2365, 2355-2366, 2356-2367, 2357-2368, 2358-2369, 2359-2370, 2360-2371, 2361-2372, 2362-2373, 2363-2374, 2364-2375, 2365-2376, 2366-2377, 2367-2378, 2368-2379, 2369-2380, 2370-2381, 2371-2382, 2372-2383, 2373-2384 and 2374-2385.

Exemplary polynucleotide molecules include the following 15-mer fragments of the polynucleotide sequence from the sequence of SEQ ID NO:10: 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 95-109, 96-110, 97-111, 98-112, 99-113, 100-114, 101-115, 102-116, 103-117, 104-118, 105-119, 106-120, 107-121, 108-122, 109-123, 110-124, 111-125, 112-126, 113-127, 114-128, 115-129, 116-130, 117-131, 118-132, 119-133, 120-134, 121-135, 122-136, 123-137, 124-138, 125-139, 126-140, 127-141, 128-142, 129-143, 130-144, 131-145, 132-146, 133-147, 134-148, 135-149, 136-150, 137-151, 138-152, 139-153, 140-154, 141-155, 142-156, 143-157, 144-158, 145-159, 146-160, 147-161, 148-162, 149-163, 150-164, 151-165, 152-166, 153-167, 154-168, 155-169, 156-170, 157-171, 158-172, 159-173, 160-174, 161-175, 162-176, 163-177, 164-178, 165-179, 166-180, 167-181, 168-182, 169-183, 170-184, 171-185, 172-186, 173-187, 174-188, 175-189, 176-190, 177-191, 178-192, 179-193, 180-194, 181-195, 182-196, 183-197, 184-198, 185-199, 186-200, 187-201, 188-202, 189-203, 190-204, 191-205, 192-206, 193-207, 194-208, 195-209, 196-210, 197-211, 198-212, 199-213, 200-214, 201-215, 202-216, 203-217, 204-218, 205-219, 206-220, 207-221, 208-222, 209-223, 210-224, 211-225, 212-226, 213-227, 214-228, 215-229, 216-230, 217-231, 218-232, 219-233, 220-234, 221-235, 222-236, 223-237, 224-238, 225-239, 226-240, 227-241, 228-242, 229-243, 230-244, 231-245, 232-246, 233-247, 234-248, 235-249, 236-250, 237-251, 238-252, 239-253, 240-254, 241-255, 242-256, 243-257, 244-258, 245-259, 246-260, 247-261, 248-262, 249-263, 250-264, 251-265, 252-266, 253-267, 254-268, 255-269, 256-270, 257-271, 258-272, 259-273, 260-274, 261-275, 262-276, 263-277, 264-278, 265-279, 266-280, 267-281, 268-282, 269-283, 270-284, 271-285, 272-286, 273-287, 274-288, 275-289, 276-290, 277-291, 278-292, 279-293, 280-294, 281-295, 282-296, 283-297, 284-298, 285-299, 286-300, 287-301, 288-302, 289-303, 290-304, 291-305, 292-306, 293-307, 294-308, 295-309, 296-310, 297-311, 298-312, 299-313, 300-314, 301-315, 302-316, 303-317, 304-318, 305-319, 306-320, 307-321, 308-322, 309-323, 310-324, 311-325, 312-326, 313-327, 314-328, 315-329, 316-330, 317-331, 318-332, 319-333, 320-334, 321-335, 322-336, 323-337, 324-338, 325-339, 326-340, 327-341, 328-342, 329-343, 330-344, 331-345, 332-346, 333-347, 334-348, 335-349, 336-350, 337-351, 338-352, 339-353, 340-354, 341-355, 342-356, 343-357, 344-358, 345-359, 346-360, 347-361, 348-362, 349-363, 350-364, 351-365, 352-366, 353-367, 354-368, 355-369, 356-370, 357-371, 358-372, 359-373, 360-374, 361-375, 362-376, 363-377, 364-378, 365-379, 366-380, 367-381, 368-382, 369-383, 370-384, 371-385, 372-386, 373-387, 374-388, 375-389, 376-390, 377-391, 378-392, 379-393, 380-394, 381-395, 382-396, 383-397, 384-398, 385-399, 386-400, 387-401, 388-402, 389-403, 390-404, 391-405, 392-406, 393-407, 394-408, 395-409, 396-410, 397-411, 398-412, 399-413, 400-414, 401-415, 402-416, 403-417, 404-418, 405-419, 406-420, 407-421, 408-422, 409-423, 410-424, 411-425, 412-426, 413-427, 414-428, 415-429, 416-430, 417-431, 418-432, 419-433, 420-434, 421-435, 422-436, 423-437, 424-438, 425-439, 426-440, 427-441, 428-442, 429-443, 430-444, 431-445, 432-446, 433-447, 434-448, 435-449, 436-450, 437-451, 438-452, 439-453, 440-454, 441-455, 442-456, 443-457, 444-458, 445-459, 446-460, 447-461, 448-462, 449-463, 450-464, 451-465, 452-466, 453-467, 454-468, 455-469, 456-470, 457-471, 458-472, 459-473, 460-474, 461-475, 462-476, 463-477, 464-478, 465-479, 466-480, 467-481, 468-482, 469-483, 470-484, 471-485, 472-486, 473-487, 474-488, 475-489, 476-490, 477-491, 478-492, 479-493, 480-494, 481-495, 482-496, 483-497, 484-498, 485-499, 486-500, 487-501, 488-502, 489-503, 490-504, 491-505, 492-506, 493-507, 494-508, 495-509, 496-510, 497-511, 498-512, 499-513, 500-514, 501-515, 502-516, 503-517, 504-518, 505-519, 506-520, 507-521, 508-522, 509-523, 510-524, 511-525, 512-526, 513-527, 514-528, 515-529, 516-530, 517-531, 518-532, 519-533, 520-534, 521-535, 522-536, 523-537, 524-538, 525-539, 526-540, 527-541, 528-542, 529-543, 530-544, 531-545, 532-546, 533-547, 534-548, 535-549, 536-550, 537-551, 538-552, 539-553, 540-554, 541-555, 542-556, 543-557, 544-558, 545-559, 546-560, 547-561, 548-562, 549-563, 550-564, 551-565, 552-566, 553-567, 554-568, 555-569, 556-570, 557-571, 558-572, 559-573, 560-574, 561-575, 562-576, 563-577, 564-578, 565-579, 566-580, 567-581, 568-582, 569-583, 570-584, 571-585, 572-586, 573-587, 574-588, 575-589, 576-590, 577-591, 578-592, 579-593, 580-594, 581-595, 582-596, 583-597, 584-598, 585-599, 586-600, 587-601, 588-602, 589-603, 590-604, 591-605, 592-606, 593-607, 594-608, 595-609, 596-610, 597-611, 598-612, 599-613, 600-614, 601-615, 602-616, 603-617, 604-618, 605-619, 606-620, 607-621, 608-622, 609-623, 610-624, 611-625, 612-626, 613-627, 614-628, 615-629, 616-630, 617-631, 618-632, 619-633, 620-634, 621-635, 622-636, 623-637, 624-638, 625-639, 626-640, 627-641, 628-642, 629-643, 630-644, 631-645, 632-646, 633-647, 634-648, 635-649, 636-650, 637-651, 638-652, 639-653, 640-654, 641-655, 642-656, 643-657, 644-658, 645-659, 646-660, 647-661, 648-662, 649-663, 650-664, 651-665, 652-666, 653-667, 654-668, 655-669, 656-670, 657-671, 658-672, 659-673, 660-674, 661-675, 662-676, 663-677, 664-678, 665-679, 666-680, 667-681, 668-682, 669-683, 670-684, 671-685, 672-686, 673-687, 674-688, 675-689, 676-690, 677-691, 678-692, 679-693, 680-694, 681-695, 682-696, 683-697, 684-698, 685-699, 686-700, 687-701, 688-702, 689-703, 690-704, 691-705, 692-706, 693-707, 694-708, 695-709, 696-710, 697-711, 698-712, 699-713, 700-714, 701-715, 702-716, 703-717, 704-718, 705-719, 706-720, 707-721, 708-722, 709-723, 710-724, 711-725, 712-726, 713-727, 714-728, 715-729, 716-730, 717-731, 718-732, 719-733, 720-734, 721-735, 722-736, 723-737, 724-738, 725-739, 726-740, 727-741, 728-742, 729-743, 730-744, 731-745, 732-746, 733-747, 734-748, 735-749, 736-750, 737-751, 738-752, 739-753, 740-754, 741-755, 742-756, 743-757, 744-758, 745-759, 746-760, 747-761, 748-762, 749-763, 750-764, 751-765, 752-766, 753-767, 754-768, 755-769, 756-770, 757-771, 758-772, 759-773, 760-774, 761-775, 762-776, 763-777, 764-778, 765-779, 766-780, 767-781, 768-782, 769-783, 770-784, 771-785, 772-786, 773-787, 774-788, 775-789, 776-790, 777-791, 778-792, 779-793, 780-794, 781-795, 782-796, 783-797, 784-798, 785-799, 786-800, 787-801, 788-802, 789-803, 790-804, 791-805, 792-806, 793-807, 794-808, 795-809, 796-810, 797-811, 798-812, 799-813, 800-814, 801-815, 802-816, 803-817, 804-818, 805-819, 806-820, 807-821, 808-822, 809-823, 810-824, 811-825, 812-826, 813-827, 814-828, 815-829, 816-830, 817-831, 818-832, 819-833, 820-834, 821-835, 822-836, 823-837, 824-838, 825-839, 826-840, 827-841, 828-842, 829-843, 830-844, 831-845, 832-846, 833-847, 834-848, 835-849, 836-850, 837-851, 838-852, 839-853, 840-854, 841-855, 842-856, 843-857, 844-858, 845-859, 846-860, 847-861, 848-862, 849-863, 850-864, 851-865, 852-866, 853-867, 854-868, 855-869, 856-870, 857-871, 858-872, 859-873, 860-874, 861-875, 862-876, 863-877, 864-878, 865-879, 866-880, 867-881, 868-882, 869-883, 870-884, 871-885, 872-886, 873-887, 874-888, 875-889, 876-890, 877-891, 878-892, 879-893, 880-894, 881-895, 882-896, 883-897, 884-898, 885-899, 886-900, 887-901, 888-902, 889-903, 890-904, 891-905, 892-906, 893-907, 894-908, 895-909, 896-910, 897-911, 898-912, 899-913, 900-914, 901-915, 902-916, 903-917, 904-918, 905-919, 906-920, 907-921, 908-922, 909-923, 910-924, 911-925, 912-926, 913-927, 914-928, 915-929, 916-930, 917-931, 918-932, 919-933, 920-934, 921-935, 922-936, 923-937, 924-938, 925-939, 926-940, 927-941, 928-942, 929-943, 930-944, 931-945, 932-946, 933-947, 934-948, 935-949, 936-950, 937-951, 938-952, 939-953, 940-954, 941-955, 942-956, 943-957, 944-958, 945-959, 946-960, 947-961, 948-962, 949-963, 950-964, 951-965, 952-966, 953-967, 954-968, 955-969, 956-970, 957-971, 958-972, 959-973, 960-974, 961-975, 962-976, 963-977, 964-978, 965-979, 966-980, 967-981, 968-982, 969-983, 970-984, 971-985, 972-986, 973-987, 974-988, 975-989, 976-990, 977-991, 978-992, 979-993, 980-994, 981-995, 982-996, 983-997, 984-998, 985-999, 986-1000, 987-1001, 988-1002, 989-1003, 990-1004, 991-1005, 992-1006, 993-1007, 994-1008, 995-1009, 996-1010, 997-1011, 998-1012, 999-1013, 1000-1014, 1001-1015, 1002-1016, 1003-1017, 1004-1018, 1005-1019, 1006-1020, 1007-1021, 1008-1022, 1009-1023, 1010-1024, 1011-1025, 1012-1026, 1013-1027, 1014-1028, 1015-1029, 1016-1030, 1017-1031, 1018-1032, 1019-1033, 1020-1034, 1021-1035, 1022-1036, 1023-1037, 1024-1038, 1025-1039, 1026-1040, 1027-1041, 1028-1042, 1029-1043, 1030-1044, 1031-1045, 1032-1046, 1033-1047, 1034-1048, 1035-1049, 1036-1050, 1037-1051, 1038-1052, 1039-1053, 1040-1054, 1041-1055, 1042-1056, 1043-1057, 1044-1058, 1045-1059, 1046-1060, 1047-1061, 1048-1062, 1049-1063, 1050-1064, 1051-1065, 1052-1066, 1053-1067, 1054-1068, 1055-1069, 1056-1070, 1057-1071, 1058-1072, 1059-1073, 1060-1074, 1061-1075, 1062-1076, 1063-1077, 1064-1078, 1065-1079, 1066-1080, 1067-1081, 1068-1082, 1069-1083, 1070-1084, 1071-1085, 1072-1086, 1073-1087, 1074-1088, 1075-1089, 1076-1090, 1077-1091, 1078-1092, 1079-1093, 1080-1094, 1081-1095, 1082-1096, 1083-1097, 1084-1098, 1085-1099, 1086-1100, 1087-1101, 1088-1102, 1089-1103, 1090-1104, 1091-1105, 1092-1106, 1093-1107, 1094-1108, 1095-1109, 1096-1110, 1097-1111, 1098-1112, 1099-1113, 1100-1114, 1101-1115, 1102-1116, 1103-1117, 1104-1118, 1105-1119, 1106-1120, 1107-1121, 1108-1122, 1109-1123, 1110-1124, 1111-1125, 1112-1126, 1113-1127, 1114-1128, 1115-1129, 1116-1130, 1117-1131, 1118-1132, 1119-1133, 1120-1134, 1121-1135, 1122-1136, 1123-1137, 1124-1138, 1125-1139, 1126-1140, 1127-1141, 1128-1142, 1129-1143, 1130-1144, 1131-1145, 1132-1146, 1133-1147, 1134-1148, 1135-1149, 1136-1150, 1137-1151, 1138-1152, 1139-1153, 1140-1154, 1141-1155, 1142-1156, 1143-1157, 1144-1158, 1145-1159, 1146-1160, 1147-1161, 1148-1162, 1149-1163, 1150-1164, 1151-1165, 1152-1166, 1153-1167, 1154-1168, 1155-1169, 1156-1170, 1157-1171, 1158-1172, 1159-1173, 1160-1174, 1161-1175, 1162-1176, 1163-1177, 1164-1178, 1165-1179, 1166-1180, 1167-1181, 1168-1182, 1169-1183, 1170-1184, 1171-1185, 1172-1186, 1173-1187, 1174-1188, 1175-1189, 1176-1190, 1177-1191, 1178-1192, 1179-1193, 1180-1194, 1181-1195, 1182-1196, 1183-1197, 1184-1198, 1185-1199, 1186-1200, 1187-1201, 1188-1202, 1189-1203, 1190-1204, 1191-1205, 1192-1206, 1193-1207, 1194-1208, 1195-1209, 1196-1210, 1197-1211, 1198-1212, 1199-1213, 1200-1214, 1201-1215, 1202-1216, 1203-1217, 1204-1218, 1205-1219, 1206-1220, 1207-1221, 1208-1222, 1209-1223, 1210-1224, 1211-1225, 1212-1226, 1213-1227, 1214-1228, 1215-1229, 1216-1230, 1217-1231, 1218-1232, 1219-1233, 1220-1234, 1221-1235, 1222-1236, 1223-1237, 1224-1238, 1225-1239, 1226-1240, 1227-1241, 1228-1242, 1229-1243, 1230-1244, 1231-1245, 1232-1246, 1233-1247, 1234-1248, 1235-1249, 1236-1250, 1237-1251, 1238-1252, 1239-1253, 1240-1254, 1241-1255, 1242-1256, 1243-1257, 1244-1258, 1245-1259, 1246-1260, 1247-1261, 1248-1262, 1249-1263, 1250-1264, 1251-1265, 1252-1266, 1253-1267, 1254-1268, 1255-1269, 1256-1270, 1257-1271, 1258-1272, 1259-1273, 1260-1274, 1261-1275, 1262-1276, 1263-1277, 1264-1278, 1265-1279, 1266-1280, 1267-1281, 1268-1282, 1269-1283, 1270-1284, 1271-1285, 1272-1286, 1273-1287, 1274-1288, 1275-1289, 1276-1290, 1277-1291, 1278-1292, 1279-1293, 1280-1294, 1281-1295, 1282-1296, 1283-1297, 1284-1298, 1285-1299, 1286-1300, 1287-1301, 1288-1302, 1289-1303, 1290-1304, 1291-1305, 1292-1306, 1293-1307, 1294-1308, 1295-1309, 1296-1310, 1297-1311, 1298-1312, 1299-1313, 1300-1314, 1301-1315, 1302-1316, 1303-1317, 1304-1318, 1305-1319, 1306-1320, 1307-1321, 1308-1322, 1309-1323, 1310-1324, 1311-1325, 1312-1326, 1313-1327, 1314-1328, 1315-1329, 1316-1330, 1317-1331, 1318-1332, 1319-1333, 1320-1334, 1321-1335, 1322-1336, 1323-1337, 1324-1338, 1325-1339, 1326-1340, 1327-1341, 1328-1342, 1329-1343, 1330-1344, 1331-1345, 1332-1346, 1333-1347, 1334-1348, 1335-1349, 1336-1350, 1337-1351, 1338-1352, 1339-1353, 1340-1354, 1341-1355, 1342-1356, 1343-1357, 1344-1358, 1345-1359, 1346-1360, 1347-1361, 1348-1362, 1349-1363, 1350-1364, 1351-1365, 1352-1366, 1353-1367, 1354-1368, 1355-1369, 1356-1370, 1357-1371, 1358-1372, 1359-1373, 1360-1374, 1361-1375, 1362-1376, 1363-1377, 1364-1378, 1365-1379, 1366-1380, 1367-1381, 1368-1382, 1369-1383, 1370-1384, 1371-1385, 1372-1386, 1373-1387, 1374-1388, 1375-1389, 1376-1390, 1377-1391, 1378-1392, 1379-1393, 1380-1394, 1381-1395, 1382-1396, 1383-1397, 1384-1398, 1385-1399, 1386-1400, 1387-1401, 1388-1402, 1389-1403, 1390-1404, 1391-1405, 1392-1406, 1393-1407, 1394-1408, 1395-1409, 1396-1410, 1397-1411, 1398-1412, 1399-1413, 1400-1414, 1401-1415, 1402-1416, 1403-1417, 1404-1418, 1405-1419, 1406-1420, 1407-1421, 1408-1422, 1409-1423, 1410-1424, 1411-1425, 1412-1426, 1413-1427, 1414-1428, 1415-1429, 1416-1430, 1417-1431, 1418-1432, 1419-1433, 1420-1434, 1421-1435, 1422-1436, 1423-1437, 1424-1438, 1425-1439, 1426-1440, 1427-1441, 1428-1442, 1429-1443, 1430-1444, 1431-1445, 1432-1446, 1433-1447, 1434-1448, 1435-1449, 1436-1450, 1437-1451, 1438-1452, 1439-1453, 1440-1454, 1441-1455, 1442-1456, 1443-1457, 1444-1458, 1445-1459, 1446-1460, 1447-1461, 1448-1462, 1449-1463, 1450-1464, 1451-1465, 1452-1466, 1453-1467, 1454-1468, 1455-1469, 1456-1470, 1457-1471, 1458-1472, 1459-1473, 1460-1474, 1461-1475, 1462-1476, 1463-1477, 1464-1478, 1465-1479, 1466-1480, 1467-1481, 1468-1482, 1469-1483, 1470-1484, 1471-1485, 1472-1486, 1473-1487, 1474-1488, 1475-1489, 1476-1490, 1477-1491, 1478-1492, 1479-1493, 1480-1494, 1481-1495, 1482-1496, 1483-1497, 1484-1498, 1485-1499, 1486-1500, 1487-1501, 1488-1502, 1489-1503, 1490-1504, 1491-1505, 1492-1506, 1493-1507, 1494-1508, 1495-1509, 1496-1510, 1497-1511, 1498-1512, 1499-1513, 1500-1514, 1501-1515, 1502-1516, 1503-1517, 1504-1518, 1505-1519, 1506-1520, 1507-1521, 1508-1522, 1509-1523, 1510-1524, 1511-1525, 1512-1526, 1513-1527, 1514-1528, 1515-1529, 1516-1530, 1517-1531, 1518-1532, 1519-1533, 1520-1534, 1521-1535, 1522-1536, 1523-1537, 1524-1538, 1525-1539, 1526-1540, 1527-1541, 1528-1542, 1529-1543, 1530-1544, 1531-1545, 1532-1546, 1533-1547, 1534-1548, 1535-1549, 1536-1550, 1537-1551, 1538-1552, 1539-1553, 1540-1554, 1541-1555, 1542-1556, 1543-1557, 1544-1558, 1545-1559, 1546-1560, 1547-1561, 1548-1562, 1549-1563, 1550-1564, 1551-1565, 1552-1566, 1553-1567, 1554-1568, 1555-1569, 1556-1570, 1557-1571, 1558-1572, 1559-1573, 1560-1574, 1561-1575, 1562-1576, 1563-1577, 1564-1578, 1565-1579, 1566-1580, 1567-1581, 1568-1582, 1569-1583, 1570-1584, 1571-1585, 1572-1586, 1573-1587, 1574-1588, 1575-1589, 1576-1590, 1577-1591, 1578-1592, 1579-1593, 1580-1594, 1581-1595, 1582-1596, 1583-1597, 1584-1598, 1585-1599, 1586-1600, 1587-1601, 1588-1602, 1589-1603, 1590-1604, 1591-1605, 1592-1606, 1593-1607, 1594-1608, 1595-1609, 1596-1610, 1597-1611, 1598-1612, 1599-1613, 1600-1614, 1601-1615, 1602-1616, 1603-1617, 1604-1618, 1605-1619, 1606-1620, 1607-1621, 1608-1622, 1609-1623, 1610-1624, 1611-1625, 1612-1626, 1613-1627, 1614-1628, 1615-1629, 1616-1630, 1617-1631, 1618-1632, 1619-1633, 1620-1634, 1621-1635, 1622-1636, 1623-1637, 1624-1638, 1625-1639, 1626-1640, 1627-1641, 1628-1642, 1629-1643, 1630-1644, 1631-1645, 1632-1646, 1633-1647, 1634-1648, 1635-1649, 1636-1650, 1637-1651, 1638-1652, 1639-1653, 1640-1654, 1641-1655, 1642-1656, 1643-1657, 1644-1658, 1645-1659, 1646-1660, 1647-1661, 1648-1662, 1649-1663, 1650-1664, 1651-1665, 1652-1666, 1653-1667, 1654-1668, 1655-1669, 1656-1670, 1657-1671, 1658-1672, 1659-1673, 1660-1674, 1661-1675, 1662-1676, 1663-1677, 1664-1678, 1665-1679, 1666-1680, 1667-1681, 1668-1682, 1669-1683, 1670-1684, 1671-1685, 1672-1686, 1673-1687, 1674-1688, 1675-1689, 1676-1690, 1677-1691, 1678-1692, 1679-1693, 1680-1694, 1681-1695, 1682-1696, 1683-1697, 1684-1698, 1685-1699, 1686-1700, 1687-1701, 1688-1702, 1689-1703, 1690-1704, 1691-1705, 1692-1706, 1693-1707, 1694-1708, 1695-1709, 1696-1710, 1697-1711, 1698-1712, 1699-1713, 1700-1714, 1701-1715, 1702-1716, 1703-1717, 1704-1718, 1705-1719, 1706-1720, 1707-1721, 1708-1722, 1709-1723, 1710-1724, 1711-1725, 1712-1726, 1713-1727, 1714-1728, 1715-1729, 1716-1730, 1717-1731, 1718-1732, 1719-1733, 1720-1734, 1721-1735, 1722-1736, 1723-1737, 1724-1738, 1725-1739, 1726-1740, 1727-1741, 1728-1742, 1729-1743, 1730-1744, 1731-1745, 1732-1746, 1733-1747, 1734-1748, 1735-1749, 1736-1750, 1737-1751, 1738-1752, 1739-1753, 1740-1754, 1741-1755, 1742-1756, 1743-1757, 1744-1758, 1745-1759, 1746-1760, 1747-1761, 1748-1762, 1749-1763, 1750-1764, 1751-1765, 1752-1766, 1753-1767, 1754-1768, 1755-1769, 1756-1770, 1757-1771, 1758-1772, 1759-1773, 1760-1774, 1761-1775, 1762-1776, 1763-1777, 1764-1778, 1765-1779, 1766-1780, 1767-1781, 1768-1782, 1769-1783, 1770-1784, 1771-1785, 1772-1786, 1773-1787, 1774-1788, 1775-1789, 1776-1790, 1777-1791, 1778-1792, 1779-1793, 1780-1794, 1781-1795, 1782-1796, 1783-1797, 1784-1798, 1785-1799, 1786-1800, 1787-1801, 1788-1802, 1789-1803, 1790-1804, 1791-1805, 1792-1806, 1793-1807, 1794-1808, 1795-1809, 1796-1810, 1797-1811, 1798-1812, 1799-1813, 1800-1814, 1801-1815, 1802-1816, 1803-1817, 1804-1818, 1805-1819, 1806-1820, 1807-1821, 1808-1822, 1809-1823, 1810-1824, 1811-1825, 1812-1826, 1813-1827, 1814-1828, 1815-1829, 1816-1830, 1817-1831, 1818-1832, 1819-1833, 1820-1834, 1821-1835, 1822-1836, 1823-1837, 1824-1838, 1825-1839, 1826-1840, 1827-1841, 1828-1842, 1829-1843, 1830-1844, 1831-1845, 1832-1846, 1833-1847, 1834-1848, 1835-1849, 1836-1850, 1837-1851, 1838-1852, 1839-1853, 1840-1854, 1841-1855, 1842-1856, 1843-1857, 1844-1858, 1845-1859, 1846-1860, 1847-1861, 1848-1862, 1849-1863, 1850-1864, 1851-1865, 1852-1866, 1853-1867, 1854-1868, 1855-1869, 1856-1870, 1857-1871, 1858-1872, 1859-1873, 1860-1874, 1861-1875, 1862-1876, 1863-1877, 1864-1878, 1865-1879, 1866-1880, 1867-1881, 1868-1882, 1869-1883, 1870-1884, 1871-1885, 1872-1886, 1873-1887, 1874-1888, 1875-1889, 1876-1890, 1877-1891, 1878-1892, 1879-1893, 1880-1894, 1881-1895, 1882-1896, 1883-1897, 1884-1898, 1885-1899, 1886-1900, 1887-1901, 1888-1902, 1889-1903, 1890-1904, 1891-1905, 1892-1906, 1893-1907, 1894-1908, 1895-1909, 1896-1910, 1897-1911, 1898-1912, 1899-1913, 1900-1914, 1901-1915, 1902-1916, 1903-1917, 1904-1918, 1905-1919, 1906-1920, 1907-1921, 1908-1922, 1909-1923, 1910-1924, 1911-1925, 1912-1926, 1913-1927, 1914-1928, 1915-1929, 1916-1930, 1917-1931, 1918-1932, 1919-1933, 1920-1934, 1921-1935, 1922-1936, 1923-1937, 1924-1938, 1925-1939, 1926-1940, 1927-1941, 1928-1942, 1929-1943, 1930-1944, 1931-1945, 1932-1946, 1933-1947, 1934-1948, 1935-1949, 1936-1950, 1937-1951, 1938-1952, 1939-1953, 1940-1954, 1941-1955, 1942-1956, 1943-1957, 1944-1958, 1945-1959, 1946-1960, 1947-1961, 1948-1962, 1949-1963, 1950-1964, 1951-1965, 1952-1966, 1953-1967, 1954-1968, 1955-1969, 1956-1970, 1957-1971, 1958-1972, 1959-1973, 1960-1974, 1961-1975, 1962-1976, 1963-1977, 1964-1978, 1965-1979, 1966-1980, 1967-1981, 1968-1982, 1969-1983, 1970-1984, 1971-1985, 1972-1986, 1973-1987, 1974-1988, 1975-1989, 1976-1990, 1977-1991, 1978-1992, 1979-1993, 1980-1994, 1981-1995, 1982-1996, 1983-1997, 1984-1998, 1985-1999, 1986-2000, 1987-2001, 1988-2002, 1989-2003, 1990-2004, 1991-2005, 1992-2006, 1993-2007, 1994-2008, 1995-2009, 1996-2010, 1997-2011, 1998-2012, 1999-2013, 2000-2014, 2001-2015, 2002-2016, 2003-2017, 2004-2018, 2005-2019, 2006-2020, 2007-2021, 2008-2022, 2009-2023, 2010-2024, 2011-2025, 2012-2026, 2013-2027, 2014-2028, 2015-2029, 2016-2030, 2017-2031, 2018-2032, 2019-2033, 2020-2034, 2021-2035, 2022-2036, 2023-2037, 2024-2038, 2025-2039, 2026-2040, 2027-2041, 2028-2042, 2029-2043, 2030-2044, 2031-2045, 2032-2046, 2033-2047, 2034-2048, 2035-2049, 2036-2050, 2037-2051, 2038-2052, 2039-2053, 2040-2054, 2041-2055, 2042-2056, 2043-2057, 2044-2058, 2045-2059, 2046-2060, 2047-2061, 2048-2062, 2049-2063, 2050-2064, 2051-2065, 2052-2066, 2053-2067, 2054-2068, 2055-2069, 2056-2070, 2057-2071, 2058-2072, 2059-2073, 2060-2074, 2061-2075, 2062-2076, 2063-2077, 2064-2078, 2065-2079, 2066-2080, 2067-2081, 2068-2082, 2069-2083, 2070-2084, 2071-2085, 2072-2086, 2073-2087, 2074-2088, 2075-2089, 2076-2090, 2077-2091, 2078-2092, 2079-2093, 2080-2094, 2081-2095, 2082-2096, 2083-2097, 2084-2098, 2085-2099, 2086-2100, 2087-2101, 2088-2102, 2089-2103, 2090-2104, 2091-2105, 2092-2106, 2093-2107, 2094-2108, 2095-2109, 2096-2110, 2097-2111, 2098-2112, 2099-2113, 2100-2114, 2101-2115 ,2102-2116, 2103-2117, 2104-2118, 2105-2119, 2106-2120, 2107-2121, 2108-2122, 2109-2123, 2110-2124, 2111-2125, 2112-2126, 2113-2127, 2114-2128, 2115-2129, 2116-2130, 2117-2131, 2118-2132, 2119-2133, 2120-2134, 2121-2135, 2122-2136, 2123-2137, 2124-2138, 2125-2139, 2126-2140, 2127-2141, 2128-2142, 2129-2143, 2130-2144, 2131-2145, 2132-2146, 2133-2147, 2134-2148, 2135-2149, 2136-2150, 2137-2151, 2138-2152, 2139-2153, 2140-2154, 2141-2155, 2142-2156, 2143-2157, 2144-2158, 2145-2159, 2146-2160, 2147-2161, 2148-2162, 2149-2163, 2150-2164, 2151-2165, 2152-2166, 2153-2167, 2154-2168, 2155-2169, 2156-2170, 2157-2171, 2158-2172, 2159-2173, 2160-2174, 2161-2175, 2162-2176, 2163-2177, 2164-2178, 2165-2179, 2166-2180, 2167-2181, 2168-2182, 2169-2183, 2170-2184, 2171-2185, 2172-2186, 2173-2187, 2174-2188, 2175-2189, 2176-2190, 2177-2191, 2178-2192, 2179-2193, 2180-2194, 2181-2195, 2182-2196, 2183-2197, 2184-2198, 2185-2199, 2186-2200, 2187-2201, 2188-2202, 2189-2203, 2190-2204, 2191-2205, 2192-2206, 2193-2207, 2194-2208, 2195-2209, 2196-2210, 2197-2211, 2198-2212, 2199-2213, 2200-2214, 2201-2215, 2202-2216, 2203-2217, 2204-2218, 2205-2219, 2206-2220, 2207-2221, 2208-2222, 2209-2223, 2210-2224, 2211-2225, 2212-2226, 2213-2227, 2214-2228, 2215-2229, 2216-2230, 2217-2231, 2218-2232, 2219-2233, 2220-2234, 2221-2235, 2222-2236, 2223-2237, 2224-2238, 2225-2239, 2226-2240, 2227-2241, 2228-2242, 2229-2243, 2230-2244, 2231-2245, 2232-2246, 2233-2247, 2234-2248, 2235-2249, 2236-2250, 2237-2251, 2238-2252, 2239-2253, 2240-2254, 2241-2255, 2242-2256, 2243-2257, 2244-2258, 2245-2259, 2246-2260, 2247-2261, 2248-2262, 2249-2263, 2250-2264, 2251-2265, 2252-2266, 2253-2267, 2254-2268, 2255-2269, 2256-2270, 2257-2271, 2258-2272, 2259-2273, 2260-2274, 2261-2275, 2262-2276, 2263-2277, 2264-2278, 2265-2279, 2266-2280, 2267-2281, 2268-2282, 2269-2283, 2270-2284, 2271-2285, 2272-2286, 2273-2287, 2274-2288, 2275-2289, 2276-2290, 2277-2291, 2278-2292, 2279-2293, 2280-2294, 2281-2295, 2282-2296, 2283-2297, 2284-2298, 2285-2299, 2286-2300, 2287-2301, 2288-2302, 2289-2303, 2290-2304, 2291-2305, 2292-2306, 2293-2307, 2294-2308, 2295-2309, 2296-2310, 2297-2311, 2298-2312, 2299-2313, 2300-2314, 2301-2315, 2302-2316, 2303-2317, 2304-2318, 2305-2319, 2306-2320, 2307-2321, 2308-2322, 2309-2323, 2310-2324, 2311-2325, 2312-2326, 2313-2327, 2314-2328, 2315-2329, 2316-2330, 2317-2331, 2318-2332, 2319-2333, 2320-2334, 2321-2335, 2322-2336, 2323-2337, 2324-2338, 2325-2339, 2326-2340, 2327-2341, 2328-2342, 2329-2343, 2330-2344, 2331-2345, 2332-2346, 2333-2347, 2334-2348, 2335-2349, 2336-2350, 2337-2351, 2338-2352, 2339-2353, 2340-2354, 2341-2355, 2342-2356, 2343-2357, 2344-2358, 2345-2359, 2346-2360, 2347-2361, 2348-2362, 2349-2363, 2350-2364, 2351-2365, 2352-2366, 2353-2367, 2354-2368, 2355-2369, 2356-2370, 2357-2371, 2358-2372, 2359-2373, 2360-2374, 2361-2375, 2362-2376, 2363-2377, 2364-2378, 2365-2379, 2366-2380, 2367-2381, 2368-2382, 2369-2383, 2370-2384 and 2371-2385.

Exemplary polynucleotide molecules include the following 20-mer fragments of the polynucleotide sequence from the sequence of SEQ ID NO:10: 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 797-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040, 1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316, 1298-1317, 1299-1318, 1300-1319, 1301-1320, 1302-1321, 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-1420, 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, 1581-1600, 1582-1601, 1583-1602, 1584-1603, 1585-1604, 1586-1605, 1587-1606, 1588-1607, 1589-1608, 1590-1609, 1591-1610, 1592-1611, 1593-1612, 1594-1613, 1595-1614, 1596-1615, 1597-1616, 1598-1617, 1599-1618, 1600-1619, 1601-1620, 1602-1621, 1603-1622, 1604-1623, 1605-1624, 1606-1625, 1607-1626, 1608-1627, 1609-1628, 1610-1629, 1611-1630, 1612-1631, 1613-1632, 1614-1633, 1615-1634, 1616-1635, 1617-1636, 1618-1637, 1619-1638, 1620-1639, 1621-1640, 1622-1641, 1623-1642, 1624-1643, 1625-1644, 1626-1645, 1627-1646, 1628-1647, 1629-1648, 1630-1649, 1631-1650, 1632-1651, 1633-1652, 1634-1653, 1635-1654, 1636-1655, 1637-1656, 1638-1657, 1639-1658, 1640-1659, 1641-1660, 1642-1661, 1643-1662, 1644-1663, 1645-1664, 1646-1665, 1647-1666, 1648-1667, 1649-1668, 1650-1669, 1651-1670, 1652-1671, 1653-1672, 1654-1673, 1655-1674, 1656-1675, 1657-1676, 1658-1677, 1659-1678, 1660-1679, 1661-1680, 1662-1681, 1663-1682, 1664-1683, 1665-1684, 1666-1685, 1667-1686, 1668-1687, 1669-1688, 1670-1689, 1671-1690, 1672-1691, 1673-1692, 1674-1693, 1675-1694, 1676-1695, 1677-1696, 1678-1697, 1679-1698, 1680-1699, 1681-1700, 1682-1701, 1683-1702, 1684-1703, 1685-1704, 1686-1705, 1687-1706, 1688-1707, 1689-1708, 1690-1709, 1691-1710, 1692-1711, 1693-1712, 1694-1713, 1695-1714, 1696-1715, 1697-1716, 1698-1717, 1699-1718, 1700-1719, 1701-1720, 1702-1721, 1703-1722, 1704-1723, 1705-1724, 1706-1725, 1707-1726, 1708-1727, 1709-1728, 1710-1729, 1711-1730, 1712-1731, 1713-1732, 1714-1733, 1715-1734, 1716-1735, 1717-1736, 1718-1737, 1719-1738, 1720-1739, 1721-1740, 1722-1741, 1723-1742, 1724-1743, 1725-1744, 1726-1745, 1727-1746, 1728-1747, 1729-1748, 1730-1749, 1731-1750, 1732-1751, 1733-1752, 1734-1753, 1735-1754, 1736-1755, 1737-1756, 1738-1757, 1739-1758, 1740-1759, 1741-1760, 1742-1761, 1743-1762, 1744-1763, 1745-1764, 1746-1765, 1747-1766, 1748-1767, 1749-1768, 1750-1769, 1751-1770, 1752-1771, 1753-1772, 1754-1773, 1755-1774, 1756-1775, 1757-1776, 1758-1777, 1759-1778, 1760-1779, 1761-1780, 1762-1781, 1763-1782, 1764-1783, 1765-1784, 1766-1785, 1767-1786, 1768-1787, 1769-1788, 1770-1789, 1771-1790, 1772-1791, 1773-1792, 1774-1793, 1775-1794, 1776-1795, 1777-1796, 1778-1797, 1779-1798, 1780-1799, 1781-1800, 1782-1801, 1783-1802, 1784-1803, 1785-1804, 1786-1805, 1787-1806, 1788-1807, 1789-1808, 1790-1809, 1791-1810, 1792-1811, 1793-1812, 1794-1813, 1795-1814, 1796-1815, 1797-1816, 1798-1817, 1799-1818, 1800-1819, 1801-1820, 1802-1821, 1803-1822, 1804-1823, 1805-1824, 1806-1825, 1807-1826, 1808-1827, 1809-1828, 1810-1829, 1811-1830, 1812-1831, 1813-1832, 1814-1833, 1815-1834, 1816-1835, 1817-1836, 1818-1837, 1819-1838, 1820-1839, 1821-1840, 1822-1841, 1823-1842, 1824-1843, 1825-1844, 1826-1845, 1827-1846, 1828-1847, 1829-1848, 1830-1849, 1831-1850, 1832-1851, 1833-1852, 1834-1853, 1835-1854, 1836-1855, 1837-1856, 1838-1857, 1839-1858, 1840-1859, 1841-1860, 1842-1861, 1843-1862, 1844-1863, 1845-1864, 1846-1865, 1847-1866, 1848-1867, 1849-1868, 1850-1869, 1851-1870, 1852-1871, 1853-1872, 1854-1873, 1855-1874, 1856-1875, 1857-1876, 1858-1877, 1859-1878, 1860-1879, 1861-1880, 1862-1881, 1863-1882, 1864-1883, 1865-1884, 1866-1885, 1867-1886, 1868-1887, 1869-1888, 1870-1889, 1871-1890, 1872-1891, 1873-1892, 1874-1893, 1875-1894, 1876-1895, 1877-1896, 1878-1897, 1879-1898, 1880-1899, 1881-1900, 1882-1901, 1883-1902, 1884-1903, 1885-1904, 1886-1905, 1887-1906, 1888-1907, 1889-1908, 1890-1909, 1891-1910, 1892-1911, 1893-1912, 1894-1913, 1895-1914, 1896-1915, 1897-1916, 1898-1917, 1899-1918, 1900-1919, 1901-1920, 1902-1921, 1903-1922, 1904-1923, 1905-1924, 1906-1925, 1907-1926, 1908-1927, 1909-1928, 1910-1929, 1911-1930, 1912-1931, 1913-1932, 1914-1933, 1915-1934, 1916-1935, 1917-1936, 1918-1937, 1919-1938, 1920-1939, 1921-1940, 1922-1941, 1923-1942, 1924-1943, 1925-1944, 1926-1945, 1927-1946, 1928-1947, 1929-1948, 1930-1949, 1931-1950, 1932-1951, 1933-1952, 1934-1953, 1935-1954, 1936-1955, 1937-1956, 1938-1957, 1939-1958, 1940-1959, 1941-1960, 1942-1961, 1943-1962, 1944-1963, 1945-1964, 1946-1965, 1947-1966, 1948-1967, 1949-1968, 1950-1969, 1951-1970, 1952-1971, 1953-1972, 1954-1973, 1955-1974, 1956-1975, 1957-1976, 1958-1977, 1959-1978, 1960-1979, 1961-1980, 1962-1981, 1963-1982, 1964-1983, 1965-1984, 1966-1985, 1967-1986, 1968-1987, 1969-1988, 1970-1989, 1971-1990, 1972-1991, 1973-1992, 1974-1993, 1975-1994, 1976-1995, 1977-1996, 1978-1997, 1979-1998, 1980-1999, 1981-2000, 1982-2001, 1983-2002, 1984-2003, 1985-2004, 1986-2005, 1987-2006, 1988-2007, 1989-2008, 1990-2009, 1991-2010, 1992-2011, 1993-2012, 1994-2013, 1995-2014, 1996-2015, 1997-2016, 1998-2017, 1999-2018, 2000-2019, 2001-2020, 2002-2021, 2003-2022, 2004-2023, 2005-2024, 2006-2025, 2007-2026, 2008-2027, 2009-2028, 2010-2029, 2011-2030, 2012-2031, 2013-2032, 2014-2033, 2015-2034, 2016-2035, 2017-2036, 2018-2037, 2019-2038, 2020-2039, 2021-2040, 2022-2041, 2023-2042, 2024-2043, 2025-2044, 2026-2045, 2027-2046, 2028-2047, 2029-2048, 2030-2049, 2031-2050, 2032-2051, 2033-2052, 2034-2053, 2035-2054, 2036-2055, 2037-2056, 2038-2057, 2039-2058, 2040-2059, 2041-2060, 2042-2061, 2043-2062, 2044-2063, 2045-2064, 2046-2065, 2047-2066, 2048-2067, 2049-2068, 2050-2069, 2051-2070, 2052-2071, 2053-2072, 2054-2073, 2055-2074, 2056-2075, 2057-2076, 2058-2077, 2059-2078, 2060-2079, 2061-2080, 2062-2081, 2063-2082, 2064-2083, 2065-2084, 2066-2085, 2067-2086, 2068-2087, 2069-2088, 2070-2089, 2071-2090, 2072-2091, 2073-2092, 2074-2093, 2075-2094, 2076-2095, 2077-2096, 2078-2097, 2079-2098, 2080-2099, 2081-2100, 2082-2101, 2083-2102, 2084-2103, 2085-2104, 2086-2105, 2087-2106, 2088-2107, 2089-2108, 2090-2109, 2091-2110, 2092-2111, 2093-2112, 2094-2113, 2095-2114, 2096-2115, 2097-2116, 2098-2117, 2099-2118, 2100-2119, 2101-2120, 2102-2121, 2103-2122, 2104-2123, 2105-2124, 2106-2125, 2107-2126, 2108-2127, 2109-2128, 2110-2129, 2111-2130, 2112-2131, 2113-2132, 2114-2133, 2115-2134, 2116-2135, 2117-2136, 2118-2137, 2119-2138, 2120-2139, 2121-2140, 2122-2141, 2123-2142, 2124-2143, 2125-2144, 2126-2145, 2127-2146, 2128-2147, 2129-2148, 2130-2149, 2131-2150, 2132-2151, 2133-2152, 2134-2153, 2135-2154, 2136-2155, 2137-2156, 2138-2157, 2139-2158, 2140-2159, 2141-2160, 2142-2161, 2143-2162, 2144-2163, 2145-2164, 2146-2165, 2147-2166, 2148-2167, 2149-2168, 2150-2169, 2151-2170, 2152-2171, 2153-2172, 2154-2173, 2155-2174, 2156-2175, 2157-2176, 2158-2177, 2159-2178, 2160-2179, 2161-2180, 2162-2181, 2163-2182, 2164-2183, 2165-2184, 2166-2185, 2167-2186, 2168-2187, 2169-2188, 2170-2189, 2171-2190, 2172-2191, 2173-2192, 2174-2193, 2175-2194, 2176-2195, 2177-2196, 2178-2197, 2179-2198, 2180-2199, 2181-2200, 2182-2201, 2183-2202, 2184-2203, 2185-2204, 2186-2205, 2187-2206, 2188-2207, 2189-2208, 2190-2209, 2191-2210, 2192-2211, 2193-2212, 2194-2213, 2195-2214, 2196-2215, 2197-2216, 2198-2217, 2199-2218, 2200-2219, 2201-2220, 2202-2221, 2203-2222, 2204-2223, 2205-2224, 2206-2225, 2207-2226, 2208-2227, 2209-2228, 2210-2229, 2211-2230, 2212-2231, 2213-2232, 2214-2233, 2215-2234, 2216-2235, 2217-2236, 2218-2237, 2219-2238, 2220-2239, 2221-2240, 2222-2241, 2223-2242, 2224-2243, 2225-2244, 2226-2245, 2227-2246, 2228-2247, 2229-2248, 2230-2249, 2231-2250, 2232-2251, 2233-2252, 2234-2253, 2235-2254, 2236-2255, 2237-2256, 2238-2257, 2239-2258, 2240-2259, 2241-2260, 2242-2261, 2243-2262, 2244-2263, 2245-2264, 2246-2265, 2247-2266, 2248-2267, 2249-2268, 2250-2269, 2251-2270, 2252-2271, 2253-2272, 2254-2273, 2255-2274, 2256-2275, 2257-2276, 2258-2277, 2259-2278, 2260-2279, 2261-2280, 2262-2281, 2263-2282, 2264-2283, 2265-2284, 2266-2285, 2267-2286, 2268-2287, 2269-2288, 2270-2289, 2271-2290, 2272-2291, 2273-2292, 2274-2293, 2275-2294, 2276-2295, 2277-2296, 2278-2297, 2279-2298, 2280-2299, 2281-2300, 2282-2301, 2283-2302, 2284-2303, 2285-2304, 2286-2305, 2287-2306, 2288-2307, 2289-2308, 2290-2309, 2291-2310, 2292-2311, 2293-2312, 2294-2313, 2295-2314, 2296-2315, 2297-2316, 2298-2317, 2299-2318, 2300-2319, 2301-2320, 2302-2321, 2303-2322, 2304-2323, 2305-2324, 2306-2325, 2307-2326, 2308-2327, 2309-2328, 2310-2329, 2311-2330, 2312-2331, 2313-2332, 2314-2333, 2315-2334, 2316-2335, 2317-2336, 2318-2337, 2319-2338, 2320-2339, 2321-2340, 2322-2341, 2323-2342, 2324-2343, 2325-2344, 2326-2345, 2327-2346, 2328-2347, 2329-2348, 2330-2349, 2331-2350, 2332-2351, 2333-2352, 2334-2353, 2335-2354, 2336-2355, 2337-2356, 2338-2357, 2339-2358, 2340-2359, 2341-2360, 2342-2361, 2343-2362, 2344-2363, 2345-2364, 2346-2365, 2347-2366, 2348-2367, 2349-2368, 2350-2369, 2351-2370, 2352-2371, 2353-2372, 2354-2373, 2355-2374, 2356-2375, 2357-2376, 2358-2377, 2359-2378, 2360-2379, 2361-2380, 2362-2381, 2363-2382, 2364-2383, 2365-2384 and 2366-2385.

Exemplary polynucleotide molecules include the following 25-mer fragments of the polynucleotide sequence from the sequence of SEQ ID NO:10: 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 75-99, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 616-640, 617-641, 618-642, 619-643, 620-644, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 744-768, 745-769, 746-770, 747-771, 748-772, 749-773, 750-774, 751-775, 752-776, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815, 792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1262, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1472, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-1501, 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568, 1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600, 1577-1601, 1578-1602, 1579-1603, 1580-1604, 1581-1605, 1582-1606, 1583-1607, 1584-1608, 1585-1609, 1586-1610, 1587-1611, 1588-1612, 1589-1613, 1590-1614, 1591-1615, 1592-1616, 1593-1617, 1594-1618, 1595-1619, 1596-1620, 1597-1621, 1598-1622, 1599-1623, 1600-1624, 1601-1625, 1602-1626, 1603-1627, 1604-1628, 1605-1629, 1606-1630, 1607-1631, 1608-1632, 1609-1633, 1610-1634, 1611-1635, 1612-1636, 1613-1637, 1614-1638, 1615-1639, 1616-1640, 1617-1641, 1618-1642, 1619-1643, 1620-1644, 1621-1645, 1622-1646, 1623-1647, 1624-1648, 1625-1649, 1626-1650, 1627-1651, 1628-1652, 1629-1653, 1630-1654, 1631-1655, 1632-1656, 1633-1657, 1634-1658, 1635-1659, 1636-1660, 1637-1661, 1638-1662, 1639-1663, 1640-1664, 1641-1665, 1642-1666, 1643-1667, 1644-1668, 1645-1669, 1646-1670, 1647-1671, 1648-1672, 1649-1673, 1650-1674, 1651-1675, 1652-1676, 1653-1677, 1654-1678, 1655-1679, 1656-1680, 1657-1681, 1658-1682, 1659-1683, 1660-1684, 1661-1685, 1662-1686, 1663-1687, 1664-1688, 1665-1689, 1666-1690, 1667-1691, 1668-1692, 1669-1693, 1670-1694, 1671-1695, 1672-1696, 1673-1697, 1674-1698, 1675-1699, 1676-1700, 1677-1701, 1678-1702, 1679-1703, 1680-1704, 1681-1705, 1682-1706, 1683-1707, 1684-1708, 1685-1709, 1686-1710, 1687-1711, 1688-1712, 1689-1713, 1690-1714, 1691-1715, 1692-1716, 1693-1717, 1694-1718, 1695-1719, 1696-1720, 1697-1721, 1698-1722, 1699-1723, 1700-1724, 1701-1725, 1702-1726, 1703-1727, 1704-1728, 1705-1729, 1706-1730, 1707-1731, 1708-1732, 1709-1733, 1710-1734, 1711-1735, 1712-1736, 1713-1737, 1714-1738, 1715-1739, 1716-1740, 1717-1741, 1718-1742, 1719-1743, 1720-1744, 1721-1745, 1722-1746, 1723-1747, 1724-1748, 1725-1749, 1726-1750, 1727-1751, 1728-1752, 1729-1753, 1730-1754, 1731-1755, 1732-1756, 1733-1757, 1734-1758, 1735-1759, 1736-1760, 1737-1761, 1738-1762, 1739-1763, 1740-1764, 1741-1765, 1742-1766, 1743-1767, 1744-1768, 1745-1769, 1746-1770, 1747-1771, 1748-1772, 1749-1773, 1750-1774, 1751-1775, 1752-1776, 1753-1777, 1754-1778, 1755-1779, 1756-1780, 1757-1781, 1758-1782, 1759-1783, 1760-1784, 1761-1785, 1762-1786, 1763-1787, 1764-1788, 1765-1789, 1766-1790, 1767-1791, 1768-1792, 1769-1793, 1770-1794, 1771-1795, 1772-1796, 1773-1797, 1774-1798, 1775-1799, 1776-1800, 1777-1801, 1778-1802, 1779-1803, 1780-1804, 1781-1805, 1782-1806, 1783-1807, 1784-1808, 1785-1809, 1786-1810, 1787-1811, 1788-1812, 1789-1813, 1790-1814, 1791-1815, 1792-1816, 1793-1817, 1794-1818, 1795-1819, 1796-1820, 1797-1821, 1798-1822, 1799-1823, 1800-1824, 1801-1825, 1802-1826, 1803-1827, 1804-1828, 1805-1829, 1806-1830, 1807-1831, 1808-1832, 1809-1833, 1810-1834, 1811-1835, 1812-1836, 1813-1837, 1814-1838, 1815-1839, 1816-1840, 1817-1841, 1818-1842, 1819-1843, 1820-1844, 1821-1845, 1822-1846, 1823-1847, 1824-1848, 1825-1849, 1826-1850, 1827-1851, 1828-1852, 1829-1853, 1830-1854, 1831-1855, 1832-1856, 1833-1857, 1834-1858, 1835-1859, 1836-1860, 1837-1861, 1838-1862, 1839-1863, 1840-1864, 1841-1865, 1842-1866, 1843-1867, 1844-1868, 1845-1869, 1846-1870, 1847-1871, 1848-1872, 1849-1873, 1850-1874, 1851-1875, 1852-1876, 1853-1877, 1854-1878, 1855-1879, 1856-1880, 1857-1881, 1858-1882, 1859-1883, 1860-1884, 1861-1885, 1862-1886, 1863-1887, 1864-1888, 1865-1889, 1866-1890, 1867-1891, 1868-1892, 1869-1893, 1870-1894, 1871-1895, 1872-1896, 1873-1897, 1874-1898, 1875-1899, 1876-1900, 1877-1901, 1878-1902, 1879-1903, 1880-1904, 1881-1905, 1882-1906, 1883-1907, 1884-1908, 1885-1909, 1886-1910, 1887-1911, 1888-1912, 1889-1913, 1890-1914, 1891-1915, 1892-1916, 1893-1917, 1894-1918, 1895-1919, 1896-1920, 1897-1921, 1898-1922, 1899-1923, 1900-1924, 1901-1925, 1902-1926, 1903-1927, 1904-1928, 1905-1929, 1906-1930, 1907-1931, 1908-1932, 1909-1933, 1910-1934, 1911-1935, 1912-1936, 1913-1937, 1914-1938, 1915-1939, 1916-1940, 1917-1941, 1918-1942, 1919-1943, 1920-1944, 1921-1945, 1922-1946, 1923-1947, 1924-1948, 1925-1949, 1926-1950, 1927-1951, 1928-1952, 1929-1953, 1930-1954, 1931-1955, 1932-1956, 1933-1957, 1934-1958, 1935-1959, 1936-1960, 1937-1961, 1938-1962, 1939-1963, 1940-1964, 1941-1965, 1942-1966, 1943-1967, 1944-1968, 1945-1969, 1946-1970, 1947-1971, 1948-1972, 1949-1973, 1950-1974, 1951-1975, 1952-1976, 1953-1977, 1954-1978, 1955-1979, 1956-1980, 1957-1981, 1958-1982, 1959-1983, 1960-1984, 1961-1985, 1962-1986, 1963-1987, 1964-1988, 1965-1989, 1966-1990, 1967-1991, 1968-1992, 1969-1993, 1970-1994, 1971-1995, 1972-1996, 1973-1997, 1974-1998, 1975-1999, 1976-2000, 1977-2001, 1978-2002, 1979-2003, 1980-2004, 1981-2005, 1982-2006, 1983-2007, 1984-2008, 1985-2009, 1986-2010, 1987-2011, 1988-2012, 1989-2013, 1990-2014, 1991-2015, 1992-2016, 1993-2017, 1994-2018, 1995-2019, 1996-2020, 1997-2021, 1998-2022, 1999-2023, 2000-2024, 2001-2025, 2002-2026, 2003-2027, 2004-2028, 2005-2029, 2006-2030, 2007-2031, 2008-2032, 2009-2033, 2010-2034, 2011-2035, 2012-2036, 2013-2037, 2014-2038, 2015-2039, 2016-2040, 2017-2041, 2018-2042, 2019-2043, 2020-2044, 2021-2045, 2022-2046, 2023-2047, 2024-2048, 2025-2049, 2026-2050, 2027-2051, 2028-2052, 2029-2053, 2030-2054, 2031-2055, 2032-2056, 2033-2057, 2034-2058, 2035-2059, 2036-2060, 2037-2061, 2038-2062, 2039-2063, 2040-2064, 2041-2065, 2042-2066, 2043-2067, 2044-2068, 2045-2069, 2046-2070, 2047-2071, 2048-2072, 2049-2073, 2050-2074, 2051-2075, 2052-2076, 2053-2077, 2054-2078, 2055-2079, 2056-2080, 2057-2081, 2058-2082, 2059-2083, 2060-2084, 2061-2085, 2062-2086, 2063-2087, 2064-2088, 2065-2089, 2066-2090, 2067-2091, 2068-2092, 2069-2093, 2070-2094, 2071-2095, 2072-2096, 2073-2097, 2074-2098, 2075-2099, 2076-2100, 2077-2101, 2078-2102, 2079-2103, 2080-2104, 2081-2105, 2082-2106, 2083-2107, 2084-2108, 2085-2109, 2086-2110, 2087-2111, 2088-2112, 2089-2113, 2090-2114, 2091-2115, 2092-2116, 2093-2117, 2094-2118, 2095-2119, 2096-2120, 2097-2121, 2098-2122, 2099-2123, 2100-2124, 2101-2125, 2102-2126, 2103-2127, 2104-2128, 2105-2129, 2106-2130, 2107-2131, 2108-2132, 2109-2133, 2110-2134, 2111-2135, 2112-2136, 2113-2137, 2114-2138, 2115-2139, 2116-2140, 2117-2141, 2118-2142, 2119-2143, 2120-2144, 2121-2145, 2122-2146, 2123-2147, 2124-2148, 2125-2149, 2126-2150, 2127-2151, 2128-2152, 2129-2153, 2130-2154, 2131-2155, 2132-2156, 2133-2157, 2134-2158, 2135-2159, 2136-2160, 2137-2161, 2138-2162, 2139-2163, 2140-2164, 2141-2165, 2142-2166, 2143-2167, 2144-2168, 2145-2169, 2146-2170, 2147-2171, 2148-2172, 2149-2173, 2150-2174, 2151-2175, 2152-2176, 2153-2177, 2154-2178, 2155-2179, 2156-2180, 2157-2181, 2158-2182, 2159-2183, 2160-2184, 2161-2185, 2162-2186, 2163-2187, 2164-2188, 2165-2189, 2166-2190, 2167-2191, 2168-2192, 2169-2193, 2170-2194, 2171-2195, 2172-2196, 2173-2197, 2174-2198, 2175-2199, 2176-2200, 2177-2201, 2178-2202, 2179-2203, 2180-2204, 2181-2205, 2182-2206, 2183-2207, 2184-2208, 2185-2209, 2186-2210, 2187-2211, 2188-2212, 2189-2213, 2190-2214, 2191-2215, 2192-2216, 2193-2217, 2194-2218, 2195-2219, 2196-2220, 2197-2221, 2198-2222, 2199-2223, 2200-2224, 2201-2225, 2202-2226, 2203-2227, 2204-2228, 2205-2229, 2206-2230, 2207-2231, 2208-2232, 2209-2233, 2210-2234, 2211-2235, 2212-2236, 2213-2237, 2214-2238, 2215-2239, 2216-2240, 2217-2241, 2218-2242, 2219-2243, 2220-2244, 2221-2245, 2222-2246, 2223-2247, 2224-2248, 2225-2249, 2226-2250, 2227-2251, 2228-2252, 2229-2253, 2230-2254, 2231-2255, 2232-2256, 2233-2257, 2234-2258, 2235-2259, 2236-2260, 2237-2261, 2238-2262, 2239-2263, 2240-2264, 2241-2265, 2242-2266, 2243-2267, 2244-2268, 2245-2269, 2246-2270, 2247-2271, 2248-2272, 2249-2273, 2250-2274, 2251-2275, 2252-2276, 2253-2277, 2254-2278, 2255-2279, 2256-2280, 2257-2281, 2258-2282, 2259-2283, 2260-2284, 2261-2285, 2262-2286, 2263-2287, 2264-2288, 2265-2289, 2266-2290, 2267-2291, 2268-2292, 2269-2293, 2270-2294, 2271-2295, 2272-2296, 2273-2297, 2274-2298, 2275-2299, 2276-2300, 2277-2301, 2278-2302, 2279-2303, 2280-2304, 2281-2305, 2282-2306, 2283-2307, 2284-2308, 2285-2309, 2286-2310, 2287-2311, 2288-2312, 2289-2313, 2290-2314, 2291-2315, 2292-2316, 2293-2317, 2294-2318, 2295-2319, 2296-2320, 2297-2321, 2298-2322, 2299-2323, 2300-2324, 2301-2325, 2302-2326, 2303-2327, 2304-2328, 2305-2329, 2306-2330, 2307-2331, 2308-2332, 2309-2333, 2310-2334, 2311-2335, 2312-2336, 2313-2337, 2314-2338, 2315-2339, 2316-2340, 2317-2341, 2318-2342, 2319-2343, 2320-2344, 2321-2345, 2322-2346, 2323-2347, 2324-2348, 2325-2349, 2326-2350, 2327-2351, 2328-2352, 2329-2353, 2330-2354, 2331-2355, 2332-2356, 2333-2357, 2334-2358, 2335-2359, 2336-2360, 2337-2361, 2338-2362, 2339-2363, 2340-2364, 2341-2365, 2342-2366, 2343-2367, 2344-2368, 2345-2369, 2346-2370, 2347-2371, 2348-2372, 2349-2373, 2350-2374, 2351-2375, 2352-2376, 2353-2377, 2354-2378, 2355-2379, 2356-2380, 2357-2381, 2358-2382, 2359-2383, 2360-2384 and 2361-2385.

Degenerate polynucleotide sequences which encode amino acid sequences of the PAR-1 protein and variants, as well as homologous nucleotide sequences which are at least 65%, 75%, 85%, 90%, 95%, 98%, or 99% identical to the nucleotide sequence shown in SEQ ID NO:1, 2, 4, 5, 7, 8, 10, 11, 19 or 20, are also polynucleotide molecules of the invention. Percent sequence identity is determined by any method known in the art, for example, using computer programs which employ the Smith-Waterman algorithm, such as the MPSRCH program (Oxford Molecular), using an affine gap search with the following parameters: a gap open penalty of 12 and a gap extension penalty of 1.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides from SEQ ID NO:1, 2, 4, 5, 7, 8, 10, 11, 19 or 20. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence shown in SEQ ID NO:1, 2, 4, 5, 7, 8, 10, 11, 19 or 20 for use in identifying or amplifying the genes from human genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art. Polynucleotide Constructs Polynucleotide molecules comprising the coding sequences disclosed herein can be used in a polynucleotide construct, such as a DNA or RNA construct. Polynucleotide molecules of the invention can be used, for example, in an expression construct to express all or a portion of a secreted protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Host Cells

An expression construct can be introduced into a host cell. The host cell comprising the expression construct can be any suitable prokaryotic or eukaryotic cell. Expression systems in bacteria include those described in Chang et al., Nature (1978) 275: 615; Goeddel et al., Nature (1979) 281: 544; Goeddel et al, Nucleic Acids Res. (1980) 8: 4057; EP 36,776; U.S. Pat. No. 4,551,433; deBoer et al., Proc. Natl. Acad. Sci. USA (1983) 80: 21-25; and Siebenlist et al., Cell (1980) 20: 269.

Expression systems in yeast include those described in Hinnen et al., Proc. Natl. Acad. Sci. USA (1978) 75: 1929; Ito et al., J. Bacteriol. (1983) 153: 163; Kurtz et al., Mol Cell. Biol. (1986) 6: 142; Kunze et al., J Basic Microbiol. (1985) 25: 141; Gleeson et al, J. Gen. Microbiol. (1986) 132: 3459, Roggenkamp et al., Mol. Gen. Genet. (1986) 202 :302); Das et al., J Bacteriol. (1984) 158: 1165; De Louvencourt et al., J. Bacteriol. (1983) 154: 737, Van den Berg et al., Bio/Technology (1990) 8: 135; Kunze et al, J. Basic Microbiol. (1985) 25: 141; Cregg et al., Mol Cell Biol. (1985) 5: 3376; U.S. Pat. Nos. 4,837,148; 4,929,555; Beach and Nurse, Nature (1981) 300: 706; Davidow et al Curr. Genet. (1985) 1p: 380; Gaillardin et al., Curr. Genet. (1985) 10: 49; Ballance et al., Biochem. Biophys. Res. Commun. (1983) 112: 284-289; Tilburn et al, Gene (1983) 26: 205-22;, Yelton et al., Proc. Natl. Acad. Sci. USA (1984) 81: 1470-1474; Kelly and Hynes, EMBO J. (1985) 4: 475479; EP 244,234; and WO 91/00357.

Expression of heterologous genes in insects can be accomplished as described in U.S. Pat No. 4,745,051; Friesen et al (1986) “The Regulation of Baculovirus Gene Expression” in: THE MOLECULAR BIOLOGY OF BACULOVIRUSES (W. Doerfler, ed.); EP 127,839; EP 155,476; Vlak et al, J. Gen. Virol. (1988) 69: 765-776; Miller et al., Ann. Rev. Microbiol. (1988) 42: 177; Carbonell et al., Gene (1988) 73: 409, Maeda et al., Nature (1985) 315: 592-594; Lebacq-Verheyden et al., Mol. Cell Biol. (1988) 8: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6: 47-55, Miller et al, in GENERIC ENGINEERING (Setlow, J. K. et al. eds.), Vol. 8 (Plenum Publishing, 1986), pp. 277-279; and Maeda et al., Nature, (1985) 315: 592-594.

Mammalian expression can be accomplished as described in Dijkema et al., EMBO J (1985) 4: 761; Gormanetal., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth Enz. (1979) 58: 44; Barnes and Sato, Anal. Biochem. (1980) 102: 255; U.S. Pat. Nos. 4,767,704; 4,657,866; 4,927,762; 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides from the nucleotide sequence shown in SEQ ID NO:1, 2, 4, 5, 7, 8, 10, 11, 19 or 20. The transcription unit is located upstream to a coding sequence of the endogenous gene. The exogenous regulatory sequence directs transcription of the coding sequence of the endogenous gene.

PAR-1 can also include hybrid and modified forms of PAR-1 including fusion proteins, PAR-1 fragments and hybrid and modified forms in which certain amino acids have been deleted or replaced, modifications such as where one or more amino acids have been changed to a modified amino acid or unusual amino acid, and modifications such as glycosylations so long as the hybrid or modified form retains the biological activity of PAR-1. By retaining the biological activity of PAR-1, it is meant that not necessarily at the same level of potency as that of the PAR-1 isolated as described herein or that of the recombinantly produced mNkd.

Also included within the meaning of substantially homologous is any PAR-1 which may be isolated by virtue of cross-reactivity with antibodies to the PAR-1 described herein or whose encoding nucleotide sequences including genomic DNA, mRNA or cDNA may be isolated through hybridization with the complementary sequence of genomic or subgenomic nucleotide sequences or cDNA of the PAR-1 herein or fragments thereof. It will also be appreciated by one skilled in the art that degenerate DNA sequences can encode PAR-1 and these are also intended to be included within the present invention as are allelic variants of PAR-1.

Preferred PAR-1s of the present invention have been identified and isolated in purified forms as described. Also preferred is PAR-1 prepared by recombinant DNA technology. By “pure form” or “purified form” or “substantially purified form” it is meant that a PAR-1 composition is substantially free of other proteins which are not PAR-1.

The present invention also encompasses vectors comprising expression regulatory elements operably linked to any of the nucleic acid sequences included within the scope of the invention. This invention also includes host cells of any variety that have been transformed with vectors comprising expression regulatory elements operably linked to any of the nucleic acid sequences included within the scope of the present invention.

The present invention also includes therapeutic or pharmaceutical compositions comprising DAP 1A or mNkd in an effective amount for treating patients with disease, and a method comprising administering a therapeutically effective amount of PAR-1. These compositions and methods are useful for treating a number of diseases including cancer. One skilled in the art can readily use a variety of assays known in the art to determine whether PAR-1would be useful in promoting survival or functioning in a particular cell type.

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

PAR-1 can also be linked or conjugated with agents that provide desirable pharmaceutical or pharmacodynamic properties. For example, PAR-1 can be coupled to any substance known in the art to promote penetration or transport across the blood-brain barrier such as an antibody to the transferrin receptor, and administered by intravenous injection (see, for example, Friden et al., Science 259:373-377, 1993 which is incorporated by reference). Furthermore, PAR-1 can be stably linked to a polymer such as polyethylene glycol to obtain desirable properties of solubility, stability, half-life and other pharmaceutically advantageous properties. (See, for example, Davis et al., Enzyme Eng. 4:169-73, 1978; Burnham, Am. J. Hosp. Pharm. 51:210-218, 1994 which are incorporated by reference.)

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. PAR-1 can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing PAR-1 are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginales, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, PAR-1 may be therapeutically administered by implanting into patients vectors or cells capable of producing a biologically-active form of PAR-1 or a precursor of PAR-1, i.e., a molecule that can be readily converted to a biological-active form of PAR-1 by the body. In one approach cells that secrete PAR-1 may be encapsulated into semipermeable membranes for implantation into a patient. The cells can be cells that normally express PAR-1 or a precursor thereof or the cells can be transformed to express PAR-1 or a precursor thereof. It is preferred that the cell be of human origin and that the PAR-1 be human PAR-1 when the patient is human. However, the formulations and methods herein can be used for veterinary as well as human applications and the term “patient” as used herein is intended to include human and veterinary patients.

In a number of circumstances it would be desirable to determine the levels of PAR-1 in a patient. The identification of PAR-1 along with the present report showing expression of PAR-1 provides the basis for the conclusion that the presence of PAR-1 serves a normal physiological function related to cell growth and survival. Endogenously produced PAR-1 may also play a role in certain disease conditions.

The term “detection” as used herein in the context of detecting the presence of PAR-1 in a patient is intended to include the determining of the amount of PAR-1 or the ability to express an amount of PAR-1 in a patient, the estimation of prognosis in terms of probable outcome of a disease and prospect for recovery, the monitoring of the PAR-1 levels over a period of time as a measure of status of the condition, and the monitoring of PAR-1 levels for determining a preferred therapeutic regimen for the patient.

To detect the presence of PAR-1 in a patient, a sample is obtained from the patient. The sample can be a tissue biopsy sample or a sample of blood, plasma, serum, CSF or the like. PAR-1 tissue expression is disclosed discussed in Examples 5 and 6. Samples for detecting PAR-1 can be taken from these tissue. When assessing peripheral levels of PAR-1, it is preferred that the sample be a sample of blood, plasma or serum. When assessing the levels of PAR-1 in the central nervous system a preferred sample is a sample obtained from cerebrospinal fluid or neural tissue.

In some instances it is desirable to determine whether the PAR-1 gene is intact in the patient or in a tissue or cell line within the patient. By an intact PAR-1 gene it is meant that there are no alterations in the gene such as point mutations, deletions, insertions, chromosomal breakage, chromosomal rearrangements and the like wherein such alteration might alter production of PAR-1 or alter its biological activity, stability or the like to lead to disease processes. Thus, in one embodiment of the present invention a method is provided for detecting and characterizing any alterations in the PAR-1 gene. The method comprises providing an oligonucleotide that contains the PAR-1 cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize to the PAR-1 gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact PAR-1 gene or an PAR-1 gene abnormality.

Hybridization to a PAR-1 gene would involve denaturing the chromosomal DNA to obtain a single-stranded DNA; contacting the single-stranded DNA with a gene probe associated with the DAP 1A or mNkd gene sequence; and identifying the hybridized DNA-probe to detect chromosomal DNA containing at least a portion of a human PAR-1 gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The PAR-1 gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32° -40° C. and more preferably at 370-380 C. The time required for hybridization is from about 0.25 to about 96 hours, more preferably from about one to about 72 hours, and most preferably from about 4 to about 24 hours.

PAR-1 gene abnormalities can also be detected by using the PCR method and primers that flank or lie within the PAR-1 gene. The PCR method is well known in the art. Briefly, this method is performed using two oligonucleotide primers which are capable of hybridizing to the nucleic acid sequences flanking a target sequence that lies within an PAR-1gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, the method of physical denaturation is used involving heating the nucleic acid, typically to temperatures from about 80° C. to 105° C. for times ranging from about 2 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising PAR-1 or a fragment thereof is then directly sequenced and analyzed by comparison of the sequence with the sequences disclosed herein to identify alterations which might change activity or expression levels or the like.

In another embodiment, a method for detecting PAR-1 is provided based upon an analysis of tissue expressing the PAR-1 gene. The method comprises hybridizing a polynucleotide to mRNA from a sample of tissue that normally expresses the PAR-1 gene. The sample is obtained from a patient suspected of having an abnormality in the PAR-1 gene or in the PAR-1 gene of particular cells.

To detect the presence of mRNA encoding PAR-1 protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding PAR-1 protein or a derivative of the cDNA as a probe, high stringency conditions can be used in order to prevent false positives, that is the hybridization and apparent detection of PAR-1 nucleotide sequences when in fact an intact and functioning PAR-1 gene is not present. When using sequences derived from the PAR-1 cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. (Sambrook et al., 1989, supra).

In order to increase the sensitivity of the detection in a sample of mRNA encoding the PAR-1 protein, the technique of reverse transcription/polymerization chain reaction (RT/PCR) can be used to amplify cDNA transcribed from mRNA encoding the PAR-1 protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and PAR-1 specific primers. (Belyavsky et al., Nucl. Acid Res. 17:2919-2932, 1989; Krug and Berger, Methods in Enzymology, 152:316-325, Academic Press, NY, 1987 which are incorporated by reference).

The polymerase chain reaction method is performed as described above using two oligonucleotide primers that are substantially complementary to the two flanking regions of the DNA segment to be amplified.

Following amplification, the PCR product is then electrophoresed and detected by ethidium bromide staining or by phosphoimaging.

The present invention further provides for methods to detect the presence of the PAR-1 protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. (Basic and Clinical Immunology, 217-262, Sites and Terr, eds., Appleton & Lange, Norwalk, Conn., 1991, which is incorporated by reference). Preferred are binder-ligand immunoassay methods including reacting antibodies with an epitope or epitopes of the PAR-1 protein and competitively displacing a labeled PAR-1 protein or derivative thereof.

As used herein, a derivative of the PAR-1 protein is intended to include a polypeptide in which certain amino acids have been deleted or replaced or changed to modified or unusual amino acids wherein the PAR-1 derivative is biologically equivalent to PAR-1 and wherein the polypeptide derivative cross-reacts with antibodies raised against the PAR-1 protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the PAR-1 protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, usually a rabbit or a mouse.

Oligopeptides can be selected as candidates for the production of an antibody to the PAR-1 protein based upon the oligopeptides lying in hydrophilic regions, which are thus likely to be exposed in the mature protein.

Additional oligopeptides can be determined using, for example, the Antigenicity Index, Welling, G. W. et al., FEBS Lett. 188:215-218 (1985), incorporated herein by reference.

Methods for preparation of the PAR-1 protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J. Org. Chem. 37:3404, 1972 which is incorporated by reference).

Inhibitors of PAR-1 are Effective in Reducing PAR-1 Gene Expression

Inventive PAR-1 inhibitors include antisense molecules and ribozymes, proteins or polypeptides, antibodies or fragments thereof as well as small molecules. These PAR-1 inhibitors share the common feature that they reduce the expression and/or biological activity of PAR-1 and, as a consequence, modulate, inhibit, or prevent the growth of cancer cells. In addition to the exemplary PAR-1 inhibitors disclosed herein, alternative inhibitors may be obtained through routine experimentation utilizing methodology either specifically disclosed herein or as otherwise readily available to and within the expertise of the skilled artisan.

Antisense Molecules and Ribozymes

PAR-1 inhibitors of the present invention include antisense molecules that, when administered to mammalian cells, are effective in reducing, for example, intracellular levels of PAR-1 mRNA. Antisense molecules bind in a sequence-specific manner to nucleic acids, such as mRNA or DNA. When bound to mRNA that has complementary sequences, antisense molecules prevent translation of the mRNA (U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. Nucl. Acids Res. 21:3405-3411 (1993), which describes dumbbell antisense oligonucleotides).

Antisense technology can be used to control gene expression through triple-helix formation, which promotes the ability of the double helix to open sufficiently for the binding of polymerases, transcription factors or regulatory molecules. Gee et al., In Huber and Carr, “Molecular and Immunologic Approaches,” Futura Publishing Co. (Mt. Kisco, N.Y.; 1994). Alternatively, an antisense molecule may be designed to hybridize with a control region of the PAR-1 gene, e.g., promoter, enhancer or transcription initiation site, and block transcription of the gene; or block translation by inhibiting binding of a transcript to ribosomes. Hirashima et al. in Molecular Biology of RNA: New Perspectives (M. Inouye and B. S. Dudock, eds., 1987 Academic Press, San Diego, p. 401); Oligonucleotides: Antisense Inhibitors of Gene Expression (J. S. Cohen, ed., 1989 MacMillan Press, London); Stein and Cheng, Science 261:1004-1012 (1993); WO 95/10607; U.S. Pat. No. 5,359,051; WO 92/06693; and EP-A2-612844, each of which is incorporated herein by reference.

Briefly, such molecules are constructed such that they are complementary to, and able to form Watson-Crick base pairs with, a region of transcribed PAR-1 mRNA sequence. The resultant double-stranded nucleic acid interferes with subsequent processing of the mRNA, thereby preventing protein synthesis.

In general, a portion of a sequence complementary to the PAR-1 coding region may be used to modulate gene expression. The sequence of PAR-1 cDNA is presented herein as SEQ ID NOs:1, 2, 4, 5, 7, 8, 10, 11, 19 or 20. Alternatively, cDNA constructs that can be transcribed into antisense RNA may be introduced into cells or tissues to facilitate the production of antisense RNA. Thus, as used herein, the phrase “antisense molecules” broadly encompasses antisense oligonucleotides whether synthesized as DNA or RNA molecules as well as all plasmid constructs that, when introduced into a mammalian cell, promote the production of antisense RNA molecules. An antisense molecule may be used, as described herein, to inhibit expression of mRNA or protein, as well as any other gene that requires PAR-1 for its expression.

The present invention relates to antisense oligonucleotides designed to interfere with the normal function of PAR-1 polynucleotides. Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases as disclosed in U.S. Pat. No. 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation may be required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in Examples 6 and 8. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.) According to the present invention, cultures of HT1080 and SW620 cells were transfected with different antisense oligonucleotides designed to target PAR-1. These oligonucleotides are shown in SEQ ID NOs:13, 15 and 17. The effects of antisense treatment are described in Examples 6 and 8.

Antisense molecules for use as described herein can be synthesized by any method known to those of skill in this art including chemical synthesis by, for example, solid phase phosphoramidite chemical synthesis. WO 93/01286; U.S. Pat. Nos. 6,043,090; 5,218,088; 5,175,269; and 5,109,124, each of which is incorporated herein by reference. Alternatively, RNA molecules may be generated by in vitro or in vivo transcription of DNA sequences encoding the PAR-1 cDNA, or a portion thereof, provided that the DNA is incorporated into a vector downstream of a suitable RNA polymerase promoter (such as, e.g., T3, T7 or SP6). Large amounts of antisense RNA may be produced by incubating labeled nucleotides with a linearized PAR-1 cDNA fragment downstream of such a promoter in the presence of the appropriate RNA polymerase. Such antisense molecules are preferably at least 10, 15 or 20 nucleotides in length. More preferably, antisense molecules are at least 25 nucleotides in length. Within certain embodiments, an antisense molecule of the present invention will comprise a sequence that is unique to the PAR-1 CDNA sequence of SEQ ID NOs: or that can hybridize to the cDNA of SEQ ID NOs:1, 2, 4, 5, 7, 8, 10, 11, 19 or 20 under conditions of high stringency. Within the context of the present invention, high stringency means standard hybridization conditions such as, e.g., 5XSSPE, 0.5% SDS at 65° C. or the equivalent thereof. See Sambrook et al., supra and Molecular Biotechnology: Principles and Applications of Recombinant DNA, supra incorporated herein by reference.

Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. 17(12):4863-4871 (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein, in: Oligodeoxynucleotides. Antisense Inhibitors of Gene Expression, Cohen, Ed, Macmillan Press, London, pp. 97-117 (1989); Jager et al., Biochemistry 27:7237-7246 (1988)). Possible additional or alternative modifications include, but are not limited to, the addition of flanking sequences at the 5′ and/or 3′ ends and/or the inclusion of nontraditional bases such as inosine, queosine and wybutosine, as well as acetyl- methyl-, thio- and other modified forms of adenine, cytidine, guanine, thymine and uridine.

Within alternate embodiments of the present invention, PAR-1 inhibitors may be ribozymes. A ribozyme is an RNA molecule that specifically cleaves RNA substrates, such as mRNA, resulting in specific inhibition or interference with cellular gene expression. As used herein, the term “ribozymes” includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell 48:211-220 (1987); Haseloff and Gerlach, Nature 328:596-600 (1988); Walbot and Bruening, Nature 334:196 (1988); Haseloff and Gerlach, Nature 334:585 (1988)); the hairpin ribozyme (for example, as described by Haseloff et al., U.S. Pat. No. 5,254,678, issued Oct. 19, 1993 and Hempel et al., European Patent Publication No. 0 360 257, published March 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (U.S. Pat. Nos. 5,272,262; 5,144,019; and 5,168,053, 5,180,818, 5,116,742 and 5,093,246 to Cech et al.). According to certain embodiments of the invention, any such PAR-1 mRNA-specific ribozyme, or a nucleic acid encoding such a ribozyme, may be delivered to a host cell to effect inhibition of PAR-1 gene expression. Ribozymes and the like may therefore be delivered to the host cells by DNA encoding the ribozyme linked to a eukaryotic promoter, such as a eukaryotic viral promoter, such that upon introduction into the nucleus, the ribozyme will be directly transcribed. Proteins and Polypeptides In addition to the antisense molecules and ribozymes disclosed herein, PAR-1 modulators of the present invention also include proteins or polypeptides that are effective in either reducing PAR-1 gene expression or in decreasing one or more of PAR-i's biological activities. A variety of methods are readily available in the art by which the skilled artisan may, through routine experimentation, rapidly identify such PAR-1 inhibitors. The present invention is not limited by the following exemplary methodologies.

Inhibitors of PAR-1's biological activities encompass those proteins and/or polypeptides that interfere with cell proliferation, particularly tumor cell proliferation, especially colon cell proliferation. Such interference may occur indirectly through non- or un-competitive inhibition such as via binding to an allosteric site, or by binding to a region that normally binds to another protein. Accordingly, available methods for identifying proteins and/or polypeptides that bind to PAR-1 may be employed to identify lead compounds that may, through the methodology disclosed herein, be characterized for their PAR-1 inhibitory activity.

A vast body of literature is available to the skilled artisan that describes methods for detecting and analyzing protein-protein interactions. Phizicky, E. M. et al., Microbiological Reviews 59:94-123 (1995) incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive PAR-1 inhibitors may be identified through biological screening assays that rely on the direct interaction between the PAR-1 protein and a panel or library of potential inhibitor proteins. Biological screening methodologies, including the various “n-hybrid technologies,” are described in, for example, Vidal, M. et al., Nucl. Acids Res. 27(4):919-929 (1999); Frederickson, R. M., Curr. Opin. Biotechnol. 9(1):90-6 (1998); Brachmann, R. K. et al., Curr. Opin. Biotechnol. 8(5):561-568 (1997); and White, M. A., Proc. Natl. Acad. Sci. U.S.A. 93:10001-10003 (1996) each of which is incorporated herein by reference.

The two-hybrid screening methodology may be employed to search new or existing target cDNA libraries for PAR-1 binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien, C. -T. et al., Proc. Natl. Acad. Sci. U.S.A. 88:9578-9582 (1991); Dalton, S. et al., Cell 68:597-612 (1992); Durfee, T. K. et al., Genes Dev. 7:555-569 (1993); Vojtek, A. B. et al., Cell 74:205-214 (1993); and Zervos, A. S. et al., Cell 72:223-232 (1993). Commonly used reporter genes include the E. coli lacZ gene as well as selectable yeast genes such as HIS3 and LEU2. Fields, S. et al., Nature (London) 340:245-246 (1989); Durfee, T. K., supra; and Zervos, A. S., supra. A wide variety of activation domain libraries are readily available in the art such that the screening for interacting proteins may be performed through routine experimentation.

Suitable bait proteins for the identification of PAR-1 interacting proteins may be designed based on the PAR-1 cDNA sequence presented herein as SEQ ID NOs:1, 2, 4, 5, 7, 8, 10, 11, 19 or 20. Such bait proteins include either the full-length PAR-1 protein or fragments thereof.

Plasmid vectors, such as, e.g., pBTM116 and pAS2-1, for preparing PAR-1 bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

PAR-1 modulators of the present invention may alternatively be identified through one of the physical or biochemical methods available in the art for detecting protein-protein interactions.

PAR-1 is believed to interact with the other cell surface proteins. Through the protein affinity chromatography methodology, lead compounds to be tested as potential PAR-1 inhibitors may be identified by virtue of their specific retention to PAR-1 when either covalently or non-covalently coupled to a solid matrix such as, e.g., Sepharose beads. The preparation of protein affinity columns is described in, for example, Beeckmans, S. et al., Eur. J. Biochem. 117:527-535 (1981) and Formosa, T. et al., Methods Enzymol. 208:24-45 (1991). Cell lysates containing the full complement of cellular proteins may be passed through the PAR-1 affinity column. Proteins having a high affinity for PAR-1 will be specifically retained under low-salt conditions while the majority of cellular proteins will pass through the column. Such high affinity proteins may be eluted from the immobilized PAR-1 under conditions of high-salt, with chaotropic solvents or with sodium dodecyl sulfate (SDS). In some embodiments, it may be preferred to radiolabel the cells prior to preparing the lysate as an aid in identifying the PAR-1 specific binding proteins. Methods for radiolabeling manunalian cells are well known in the art and are provided, e.g., in Sopta, M. et al., J. Biol. Chem. 260:10353-10360 (1985).

Suitable PAR-1 proteins for affinity chromatography may be fused to a protein or polypeptide to permit rapid purification on an appropriate affinity resin. For example, the PAR-1cDNA may be fused to the coding region for glutathione S-transferase (GST) which facilitates the adsorption of fusion proteins to glutathione-agarose columns. Smith et al., Gene 67:31-40 (1988). Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of PAR-1 fusion proteins that is presented herein is the epitope for the influenza virus hemaglutinin (HA) against which monoclonal antibodies are readily available and from which antibodies an affinity column may be prepared.

Proteins that are specifically retained on a PAR-1 affinity column may be identified after subjecting to SDS polyacrylamide gel electrophoresis (SDS-PAGE). Thus, where cells are radiolabeled prior to the preparation of cell lysates and passage through the PAR-1 affinity column, proteins having high affinity for PAR-1 may be detected by autoradiography. The identity of PAR-1 specific binding proteins may be determined by protein sequencing techniques that are readily available to the skilled artisan, such as Mathews, C. K. et al., Biochemistry, The Benjamin/Cummings Publishing Company, Inc. pp.166-170 (1990).

Antibodies or Antibody Fragments

PAR-1 modulators (antagonists and agonists) of the present invention include antibodies and/or antibody fragments that are effective in modulating PAR-1 gene expression and/or biological activity. Suitable antibodies may be monoclonal, polyclonal or humanized monoclonal antibodies. Antibodies may be derived by conventional hybridoma based methodology, from antisera isolated from PAR-1 inoculated animals or through recombinant DNA technology. Alternatively, inventive antibodies or antibody fragments may be identified in vitro by use of one or more of the readily available phage display libraries. Exemplary methods are disclosed herein.

Polyclonal antibodies can be prepared by immunizing rabbits or other animals by injecting antigen followed by subsequent boosts at appropriate intervals. The animals are bled and sera assayed against purified PAR-1 protein usually by ELISA or by bioassay based upon the ability to block the action of PAR-1. In a non-limiting example, an antibody to PAR-1 can block the binding of PAR-1 to Dishevelled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the PAR-1 protein by treatment of a patient with specific antibodies to the PAR-1 protein.

Specific antibodies, either polyclonal or monoclonal, to the PAR-1 protein can be produced by any suitable method known in the art as discussed above. For example, murine or human monoclonal antibodies can be produced by hybridoma technology or, alternatively, the PAR-1 protein, or an immunologically active fragment thereof, or an anti-idiotypic antibody, or fragment thereof can be administered to an animal to elicit the production of antibodies capable of recognizing and binding to the PAR-1 protein. Such antibodies can be from any class of antibodies including, but not limited to IgG, IgA, IgM, IgD, and IgE or in the case of avian species, IgY and from any subclass of antibodies.

In one embodiment of the present invention, PAR-1 modulators are monoclonal antibodies that may be produced as follows. PAR-1 protein may be produced, for example, by expression of PAR-1 cDNA in a baculovirus based system. By this method, PAR-1 cDNA or a fragment thereof is ligated into a suitable plasmid vector that is subsequently used to transfect Sf9 cells to facilitate protein production. In addition, it may be advantageous to incorporate an epitope tag or other moiety to facilitate affinity purification of the PAR-1 protein. Clones of Sf9 cells expressing PAR-1 are identified, e.g., by enzyme linked immunosorbant assay (ELISA), lysates are prepared and the PAR-1 protein purified by affinity chromatography and the purified protein is injected, intraperitoneally, into BALB/c mice to induce antibody production. It may be advantageous to add an adjuvant, such as Freund's adjuvant, to increase the resulting immune response.

Serum is tested for the production of specific antibodies and spleen cells from animals having a positive specific antibody titer are used for cell fusions with myeloma cells to generate hybridoma clones. Supernatants derived from hybridoma clones are tested for the presence of monoclonal antibodies having specificity against PAR-1. For a general description of monoclonal antibody methodology, see, e.g., Harlow and Lane, Antibodies: A Laboratory Manual, Cold Spring Harbor Laboratory (1988).

In addition to the baculovirus expression system, other suitable bacterial or yeast expression systems may be employed for the expression of PAR-1 protein or polypeptides thereof. As with the baculovirus system, it may be advantageous to utilize one of the commercially available affinity tags to facilitate purification prior to inoculation of the animals. Thus, the PAR-1 cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998).

In other embodiments of the present invention, PAR-1 modulators are humanized anti-PAR-1 monoclonal antibodies. The phrase “humanized antibody” refers to an antibody derived from a non-human antibody—typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (U. S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci., USA., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFα:, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, PAR-1 polypeptides of the invention and variants thereof are used to immunize a transgenic animal as described above. Monoclonal antibodies are made using methods known in the art, and the specificity of the antibodies is tested using isolated PAR-1 polypeptides.

It will be appreciated that alternative PAR-1 inhibitor antibodies may be readily obtained by other methods commonly known in the art. One exemplary methodology for identifying antibodies having a high specificity for PAR-1 is the phage display technology.

Phage display libraries for the production of high-affinity antibodies are described in, for example, Hoogenboom, H. R. et al., Immunotechnology 4(1):1-20 (1998); Hoogenboom, H. R., Trends Biotechnol. 15:62-70 (1997) and McGuinness, B. et al., Nature Bio. Technol. 14:1149-1154 (1996) each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature 348:552-554 (1990) which is incorporated herein by reference. In short, the coding sequence of the antibody variable region is fused to the amino terminus of a phage minor coat protein (pIII). Expression of the antibody variable region-pIII fusion construct results in the antibody's “display” on the phage surface with the corresponding genetic material encompassed within the phage particle.

PAR-1 protein suitable for screening a phage library may be obtained by, for example, expression in baculovirus Sf9 cells as described, supra. Alternatively, the PAR-1 coding region may be PCR amplified using primers specific to the desired region of the PAR-1 protein. As discussed above, the PAR-1 protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-PAR-1 binding properties may subsequently be isolated by successive panning, in the case of a solid matrix, or by affinity adsorption to a PAR-1 antigen column. Phage having the desired PAR-1 inhibitory activities may be reintroduced into bacteria by infection and propagated by standard methods known to those skilled in the art. See Hoogenboom, H. R., Trends Biotechnol, supra for a review of methods for screening for positive antibody-pIII phage.

Small Molecules

The present invention also provides small molecule PAR-1 modulators (antagonists and agonists) that may be readily identified through routine application of high-throughput screening (HTS) methodologies. Persidis, A., Nature Biotechnology 16:488-489 (1998). HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. Gonzalez, J. E. et al., Curr. Opin. Biotechnol. 9(6):624-631 (1998) incorporated herein by reference.

HTS methodology may be employed, e.g., to screen for lead compounds that block one of PAR-1's biological activities, particularly its binding to Dsh/Dvl. By this method, PAR-1 protein may be immunoprecipitated from cells expressing the protein and applied to wells on an assay plate suitable for robotic screening. Individual test compounds may then be contacted with the immunoprecipitated protein and the effect of each test compound on PAR-1activity assessed.

Methods for Assessing the Efficacy of PAR-1 Modulators

Lead molecules or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to inhibit PAR-1 gene expression or biological activity. As discussed in further detail in the Examples, PAR-1 inhibitors of the present invention are effective in reducing PAR-1 expression levels and inhibiting cancer cell proliferation. Thus, the present invention further discloses methods that permit the skilled artisan to assess the effect of candidate inhibitors on each of these parameters.

As noted above and as presented in the Examples, candidate PAR-1 inhibitors may be tested by administration to cells that either express endogenous PAR-1 or that are made to express PAR-1 by transfection of a mammalian cell, such as SW620, with a recombinant PAR-1 plasmid construct.

Effective PAR-1 inhibitory molecules will reduce the levels of PAR-1 mRNA as determined, e.g., by Northern blot or RT-PCR analysis. Example 1; Sambrook et al., Molecular Cloning: A Laboratory Manual Cold Spring Harbor Press (1989) and Molecular Biotechnology: Principles and Applications of Recombinant DNA, ASM Press (ed. Glick, B. R. and Pasternak, J. J. 1998) incorporated herein by reference, or may reduce the levels of PAR-1 protein in the cell. The effectiveness of a given candidate antisense molecule may be assessed by comparison with a control “antisense” molecule known to have no substantial effect on PAR-1 expression when administered to a mammalian cell. Exemplary control molecules include the RC oligonucleotides disclosed in Example 2.

PAR-1 inhibitors effective in reducing PAR-1 gene expression and/or cell proliferation by one or more of the methods discussed herein may be further characterized in vivo for efficacy in one of the readily available animal model systems. The various animal model systems for study of cancer and genetic instability associated genes are discussed in, for example, Donehower, L. A. Cancer Surveys 29:329-352 (1997) incorporated herein by reference.

Administration of PAR-1 Inhibitors and Compositions Thereof

The present invention provides PAR-1 inhibitors and compositions comprising one or more PAR-1 inhibitor as well as methods that employ these inventive inhibitors in in vivo, ex vivo, and in vitro applications where it is advantageous to reduce or eliminate the expression or activity of PAR-1 or a functionally downstream molecule. PAR-1 inhibitors may find use as drugs for supplementing cancer therapeutics and other agents. PAR-1 inhibitors may also find use in other diseases of hyperproliferation.

Compositions may be administered parenterally, topically, orally or locally for therapeutic treatment. Preferably, the compositions are administered orally or parenterally, ie., intravenously, intraperitoneally, intradermally or intramuscularly.

Inventive compositions will include one or more PAR-1 inhibitor and may further comprise a pharmaceutically acceptable carrier or excipient. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

PAR-1 inhibitors useful in the treatment of disease in mammals will often be prepared substantially free of other naturally occurring immunoglobulins or other biological molecules. Preferred PAR-1 inhibitors will also exhibit minimal toxicity when administered to a mammal.

The compositions of the invention may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride and stabilizers (e.g., 1-20% maltose, etc.).

The selection of the appropriate method for administering PAR-1 inhibitors of the present invention will depend on the nature of the application envisioned as well as the nature of the PAR-1 inhibitor. Thus, for example, the precise methodology for administering a PAR-1 inhibitor will depend upon whether it is an antisense molecule, a protein and/or peptide, an antibody or antibody fragment or a small molecule. Other considerations include, for example, whether the PAR-1 inhibitor will be used to inhibit tumor cell growth, invasion, or metastasis, or as an adjunct to other cancer therapeutics.

A variety of methods are available in the art for the administration of antisense molecules. Exemplary methods include gene delivery techniques, including both viral and non-viral based methods as well as liposome mediated delivery methods.

Gene delivery methodologies will be effective to, for example, reduce tumor cell proliferation, or supplement radiation and/or chemotherapeutic treatment of tumors. Wheldon, T. E. et al., Radiother Oncol 48(1):5-13 (1998) (gene delivery methodologies for enhancement of fractionated radiotherapy). By these methodologies, substantial therapeutic benefit may be achieved despite transfection efficiencies significantly less than 100%, transient retention of the transfected inhibitor and/or existence of a subpopulation of target cells refractory to therapy.

Alternatively, gene delivery methodology may be used to directly knock out endogenous PAR-1 within tumor cells. For example, the PAR-1 gene may be targeted by transfection of a gene delivery vector carrying a PAR-1 inhibitor. Preferential transfection into or expression within tumor cells may be achieved through use of a tissue-specific or cell cycle-specific promoter, such as, e.g., promoters for prostate-specific antigen or for immunoglobulin genes (Vile, R. G. et al., Cancer Res. 53:962-967 (1993) and Vile, R. G., Semin. Cancer Biol. 5:437-443 (1994)) or through the use of trophic viruses that are confined to particular organs or structures, such as, e.g., a replication selective and neurotrophic virus that can only infect proliferating cells in the central nervous system.

Thus, to achieve therapeutic benefit, PAR-1 within the tumor cells should be preferentially inhibited. This can be accomplished by transfecting a gene expressing a PAR-1 inhibitor, a PAR-1 antisense molecule, a PAR-1 gene specific repressor, or an inhibitor of the protein product of the PAR-1 gene.

As used herein, the phrase “gene delivery vector” refers generally to a nucleic acid construct that carries and, within certain embodiments, is capable of directing the expression of an antisense molecule of interest, as described in, for example, Molecular Biotechnology: Principles and Applications of Recombinant DNA, Ch. 21, pp. 555-590 (ed. B. P. Glick and J. J. Pasternak, 2^{nd} ed. 1998); Jolly, Cancer Gene Ther. 1:51-64 (1994); Kimura, Human Gene Ther. 5:845-852 (1994); Connelly, Human Gene Ther. 6:185-193 (1995); and Kaplitt, Nat. Gen. 6:148-153 (1994).

A number of virus and non-virus based gene delivery vector systems have been described that are suitable for the administration of PAR-1 inhibitors. Virus based gene delivery systems include, but are not limited to retrovirus, such as Moloney murine leukemia virus, spumaviruses and lentiviruses; adenovirus; adeno-associated virus; and herpes-simplex virus vector systems. Viruses of each type are readily available from depositories or collections such as the American Type Culture Collection (ATCC; 10801 University Boulevard, Manassas, Virginia 20110-2209) or may be isolated from known sources using commonly available materials and techniques.

The gene delivery vector systems of the present invention will find applications both in in vivo as well as ex vivo therapeutic regimens. Each of these applications is described in further detail below.

1. Retroviral Gene Delivery Vector Systems

Within one aspect of the present invention, retroviral gene delivery vectors are provided that are constructed to carry or express a PAR-1 inhibitory antisense molecule. As used herein, the term “PAR-1 inhibitory antisense molecule” refers generally to a nucleic acid sequence having PAR-1 inhibitory activity. More specifically, such antisense molecules will reduce PAR-1 gene expression. Retroviral gene delivery vectors of the present invention may be readily constructed from a wide variety of retroviruses, including for example, B, C, and D type retroviruses as well as spumaviruses and lentiviruses. See RNA Tumor Viruses, Cold Spring Harbor Laboratory (2^{nd} ed. 1985).

Any of the above retroviruses may be readily utilized in order to assemble or construct retroviral gene delivery vectors given the disclosure provided herein, and standard recombinant DNA techniques. See, e.g., Sambrook et al, Molecular Cloning: A Laboratory Manual, Cold Spring Harbor Laboratory Press (2d ed. 1989) and Kunkle, Proc. Natl. Acad. Sci. USA. 82:488 (1985). In addition, within certain embodiments of the invention, portions of the retroviral gene delivery vectors may be derived from different retroviruses.

A retroviral vector, suitable for the expression of a PAR-1 inhibitory antisense molecule, must include at least one transcriptional promoter/enhancer or locus defining element(s), or other elements that control gene expression by other means such as alternate splicing, nuclear RNA export, post-translational modification of messenger, or post-transcriptional modification of protein. Such vector constructs must also include a packaging signal, long terminal repeats (LTRs) or portion thereof, and positive and negative strand primer binding sites appropriate to the retrovirus used (if these are not already present in the retroviral vector). Optionally, the retroviral vector may also include a signal that directs polyadenylation, selectable markers such as Neomycin resistance, TK, hygromycin resistance, phleomycin resistance histidinol resistance, or DHFR, as well as one or more restriction sites and a translation termination sequence. Within one aspect of the present invention, retroviral gene delivery vector constructs are provided comprising a 5′ LTR, a tRNA binding site, a packaging signal, one or more heterologous sequences, an origin of second strand DNA synthesis and a 3′ LTR, wherein the vector construct lacks gag/pol or env coding sequences.

Other retroviral gene delivery vectors may likewise be utilized within the context of the present invention, including, for example, those disclosed in the following each of which is incorporated herein by reference: EP 0,415,731; WO 90/07936; WO 94/03622; WO 93/25698; WO 93/25234; U.S. Pat. No. 5,219,740; WO 93/11230; WO 93/10218; Vile et al., Cancer Res. 53:3860-3864 (1993); Vile et al., Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127, GB 2,200,651, EP 0,345,242 and WO 91/02805.

Packaging cell lines suitable for use with the above-described retroviral gene delivery vector constructs may be readily prepared. See, e.g., U.S. Patent Nos. 5,716,832 and 5,591,624. These packaging cell lines may be utilized to create producer cell lines (also termed vector cell lines or “VCLs”) for the production of recombinant vector particles. It may be preferred to use packaging cell lines made from human (e.g., HT1080 cells) or mink parent cell lines, thereby allowing production of recombinant retroviruses that avoid inactivation in human serum.

2. Adeno-Associated Viral Gene Delivery Vector Systems

Adeno-associated viruses (AAV) possess a number of qualities that make them particularly suitable for the development of gene delivery vectors generally and for the delivery of polynucleotides encoding PAR-1 inhibitory antisense molecules in particular. For a general review of AAV expression systems, see Rabinowitz et al., Current Opin. Biotech. 9(5):470-475 (1998). AAV is a non-pathogenic, defective human parvovirus that is non-infective without an adeno or herpes helper virus. Thus, in the absence of a helper virus, AAV becomes integrated latently into the host genome. In addition, AAV has the advantage over the retroviruses, discussed above, in being able to transduce a wide range of both dividing and quiescent cell types.

A variety of AAV gene delivery vectors may be utilized to direct the expression of one or more PAR-1 inhibitor antisense molecule. Representative examples of such vectors include the AAV vectors disclosed by Srivastava in WO 93/09239; Samulski, et al. J. Virol.

63:3822-3828 (1989); Mendelson, et al. Virol. 166:154-165 (1988); and Flotte, et al. Proc. Natl. Acad. Sci. U.S.A. 90(22):10613-10617 (1993) incorporated herein by reference.

Briefly, an AAV gene delivery vector of the present invention may include, in order, a 5′ adeno-associated virus inverted terminal repeat; a polynucleotide encoding the PAR-1inhibitory antisense molecule; a sequence operably linked to the PAR-1 inhibitory antisense molecule that regulates its expression in a target tissue, organ or cell; and a 3′ adeno-associated virus inverted terminal repeat. A suitable regulatory sequence for the expression of PAR-1 inhibitory antisense molecule is, e.g., the enhancer/promoter sequence of cytomegalovirus (CMV). In addition, the AAV vector may preferably have a polyadenylation sequence such as the bovine growth hormone (BGH) polyadenylation sequence.

Generally, AAV vectors should have one copy of the AAV ITR at each end of the PAR-1 inhibitory antisense molecule, to allow replication, packaging, efficient integration into the host cell genome and rescue from the chromosome. The 5′ ITR sequence consists of nucleotides 1 to 145 at the 5′ end of the AAV DNA genome, and the 3′ ITR includes nucleotides 4681 to 4536 of the AAV genome. Preferably, the AAV vector may also include at least 10 nucleotides following the end of the ITR (i.e., a portion of the so-called “D region”).

Optimal packaging of an adeno-associated virus gene delivery vector requires that the 5′ and 3′ ITRs be separated by approximately 2-5 kb. It will be apparent, however, that the ideal spacing between ITR sequences may vary depending on the particular packaging system utilized. This spacing may be achieved by incorporating a “stuffer” or “filler” polynucleotide fragment to bring the total size of the nucleic acid sequence between the two ITRs to between 2 and 5 kb. Thus, where the PAR-1 inhibitory antisense molecule is smaller than 2-5 kb, a non- coding stuffer polynucleotide may be incorporated, for example, 3′ to the 5′ ITR sequence and 5′ of the PAR-1 inhibitory antisense molecule. The precise nucleotide sequence of the stuffer fragment is not an essential element of the final construct.

Depending upon the precise application contemplated, rather than incorporating a stuffer fragment, multiple copies of the PAR-1 inhibitory antisense molecule may be inserted, inter alia, to achieve the optimal ITR sequence spacing. It may be preferred to organize the polynucleotides as two or more separate transcription units each with its own promoter and polyadenylation signal.

Recombinant AAV vectors of the present invention may be generated from a variety of adeno-associated viruses, including for example, serotypes 1 through 6. For example, ITRs from any AAV serotype are expected to have similar structures and functions with regard to replication, integration, excision and transcriptional mechanisms.

Within certain embodiments of the invention, expression of the PAR-1 inhibitory antisense molecule may be accomplished by a separate promoter (e.g., a viral promoter). Representative examples of suitable promoters in this regard include a CMV promoter, an RSV promoter, an SV40 promoter, or a MoMLV promoter. Other promoters that may similarly be utilized within the context of the present invention include cell or tissue specific promoters or inducible promoters. Representative inducible promoters include tetracycline-response promoters (e.g., the “Tet” promoter) as described in Gossen et al., Proc. Natl. Acad. Sci. U.S.A. 89:5547-5551 (1992); Gossen et al., Science 268:1766-1769 (1995); Baron et al., Nucl. Acids Res. 25:2723-2729 (1997); Blau et al., Proc. Natl. Acad. Sci. U.S.A. 96:797-799 (1999); Bohl et al., Blood 92:1512-1517 (1998); and Haberman et al., Gene Therapy 5:1604-1611 (1998); the ecdysone promoter system as described in No et al., Proc. Natl. Acad. Sci. U.S.A. 93:3346-3351 (1996); and other regulated promoters or promoter systems as described in Rivera et al., Nat. Med. 2:1028-1032 (1996).

The AAV gene delivery vector may also contain additional sequences, for example from an adenovirus, which assist in effecting a desired function for the vector. Such sequences include, for example, those which assist in packaging the AAV gene delivery vector in adenovirus particles.

Packaging cell lines suitable for producing adeno-associated viral vectors may be routinely prepared given readily available techniques. See, e.g., U.S. Pat. No. 5,872,005, incorporated herein by reference. At a minimum, suitable packaging systems for AAV gene delivery systems of the present invention will include the AAV replication and capsid genes.

Preferred packaging cell lines may contain both an AAV helper virus as well as an AAV gene delivery vector containing the PAR-1 inhibitory antisense molecule. For detailed descriptions of representative packaging cell line systems, see, e.g. Holscher, C. et al., J. Virol. 68:7169-7177 (1994); Clark, K. R et al., Hum. Gene Ther. 6:1329-1341 (1995); and Tamayosa, K. et al., Hum. Gen. Ther. 7:507-513 (1996) which are incorporated herein by reference.

Alternatively, packaging of AAV may be achieved in vitro in a cell free system to obviate transfection protocols or packaging cell lines. Such in vitro systems incorporate an AAV gene delivery vector bearing the PAR-1 inhibitory antisense molecule and a source of Rep-protein, capsid-protein and Adenovirus proteins that supply helper-viral functions. The latter proteins are typically supplied in the form of a cell extract. Representative in vitro systems are further described in Ding, L. et al., Gen. Ther. 4:1167-1172 (1997) and Zhou, Z. et al., J. Virol. 72:3241-3247 (1998) which are incorporated herein by reference.

3. Other Viral Gene Delivery Vector Systems

In addition to retroviral vectors and adeno-associated virus-based vectors, numerous other viral gene delivery vector systems may also be utilized for the expression of PAR-1 inhibitory antisense molecules. For example, within one embodiment of the invention adenoviral vectors may be employed. Representative examples of such vectors include those described by, for example, Berkner, Biotechniques 6:616-627 (1988); Rosenfeld et al., Science 252:431-434 (1991); WO 93/9191; Kolls et al., Proc. Natl. Acad. Sci. U.S.A. 91(1):215-219 (1994); Kass-Eisler et al., Proc. Natl. Acad. Sci. U.S.A. 90(24):11498-502 (1993); Guzman et al., Circulation 88(6):2838-48 (1993); Guzman et al., Cir. Res. 73(6):1202-1207 (1993); Zabner et al., Cell 75(2):207-216 (1993); Li et al., Hum. Gene Ther. 4(4):403-409 (1993); Caillaud et al., Eur. J. Neurosci. 5(10):1287-1291 (1993); Vincent et al., Nat. Genet. 5(2):130-134 (1993); Jaffe et al., Nat. Genet. 1(5):372-378 (1992); and Levrero et al., Gene 10(2):195-202 (1991); and WO 93/07283; WO 93/06223; and WO 93/07282.

Gene delivery vectors of the present invention also include herpes vectors. Representative examples of such vectors include those disclosed by Kit in Adv. Exp. Med. Biol. 215:219-236 (1989); and those disclosed in U.S. Pat. No. 5,288,641 and EP 0176170 (Roizman). Additional exemplary herpes simplex virus vectors include HFEM/ICP6-LacZ disclosed in WO 95/04139 (Wistar Institute), pHSVlac described in Geller, Science 241:1667-1669 (1988), and in WO 90/09441 and WO 92/07945; HSV Us3::pgC-lacZ described in Fink, Human Gene Therapy 3:11-19 (1992); and HSV 7134, 2 RH 105 and GAL4 described in EP 0453242 (Breakefield), and those deposited with the ATCC as accession numbers ATCC VR-977 and ATCC VR-260.

Gene delivery vectors may also be generated from a wide variety of other viruses including, for example, poliovirus (Evans et al., Nature 339:385-388 (1989); and Sabin, J. Biol. Standardization 1:115-118 (1973)); rhinovirus; pox viruses, such as canary pox virus or vaccinia virus (Fisher-Hoch et al., Proc. Natl. Acad. Sci. U.S.A. 86:317-321 (1989); Flexner et al., Ann. N.Y Acad. Sci. 569:86-103 (1989); Flexner et al., Vaccine 8:17-21 (1990); U.S. Pat. Nos. 4,603,112, 4,769,330 and 5,017,487; WO 89/01973); SV40 (Mulligan et al., Nature 277:108-114 (1979); influenza virus (Luytjes et al., Cell 59:1107-1113 (1989); McMicheal et al., N. Eng. J. Med. 309:13-17 (1983); and Yap et al., Nature 273:238-239 (1978)); HIV (Poznansky, J. Virol. 65:532-536 (1991)); measles (EP 0 440,219); astrovirus (Munroe et al., J. Vir. 67:3611-3614 (1993)); and coronavirus, as well as other viral systems (e.g., EP 0,440,219; WO 92/06693; U.S. Pat. No. 5,166,057).

4. Non-viral Gene Delivery Vectors

Other gene delivery vectors and methods that may be employed for the expression of PAR-1 inhibitory antisense molecules such as, for example, nucleic acid expression vectors; polycationic condensed DNA linked or unlinked to killed adenovirus alone, for example, see Curiel, Hum Gene Ther 3:147-154 (1992); ligand linked DNA, for example, see Wu, J. Biol Chem 264:16985-16987 (1989); eucaryotic cell delivery vectors; deposition of photopolymerized hydrogel materials; hand-held gene delivery particle gun, as described in US Pat. No. 5,149,655; ionizing radiation as described in U.S. Pat. No. 5,206,152 and in WO 92/11033; nucleic charge neutralization or fusion with cell membranes. Additional approaches are described in Philip, Mol Cell Biol 14:2411-2418 (1994), and in Woffendin, Proc. Natl. Acad. Sci. 91:1581-1585 (1994).

Particle mediated gene delivery may be employed. Briefly, the PAR-1 inhibitory antisense molecule of interest can be inserted into conventional vectors that contain conventional control sequences for high level expression, and then be incubated with synthetic gene delivery molecules such as polymeric DNA-binding cations like polylysine, protamine, and albumin, linked to cell targeting ligands such as asialoorosomucoid, as described in Wu, et al., J. Biol. Chem. 262:4429-4432 (1987), insulin as described in Hucked, Biochem Pharmacol 40:253-263 (1990), galactose as described in Plank, Bioconjugate Chem 3:533-539 (1992), lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, PCT Patent Publication Nos. WO 95/13796, WO 94/23697, and WO 91/144445, and European Patent Publication No. 524,968. Nucleic acid sequences can be inserted into conventional vectors that contain conventional control sequences for high level expression, and then be incubated with synthetic gene delivery molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al., Proc. Natl. Acad. Sci. U.S.A. 91(24):11581-11585 (1994). Moreover, the coding sequence and the product of expression of such can be delivered through deposition of photopolymerized hydrogel materials.

Exemplary liposome and polycationic gene delivery vehicles are those described in U.S. Pat. Nos. 5,422,120 and 4,762,915, in PCT Patent Publication Nos. WO 95/13796, WO 94/23697, and WO 91/14445, in European Patent Publication No. 524,968 and in Starrier, Biochemistry, pp. 236-240 (1975) W.H. Freeman, San Francisco; Shokai, Biochem. Biophys. Acta. 600:1 (1980); Bayer, Biochem. Biophys. Acta. 550:464 (1979); Rivet, Methods Enzymol. 149:119 (1987); Wang, Proc. Natl. Acad. Sci. U.S.A. 84:7851 (1987); Plant, Anal. Biochem. 176:420 (1989).

The polynucleotides of the invention can be formulated as a diagnostic kit for detecting, for example, the expression of PAR-1 messenger RNA in a tumor cell. A diagnostic kit may contain at least one oligonucleotide capable of hybridizing to SEQ ID NOs:1, 4, 7 and 10 under stringent conditions. Preferably the polynucleotide will be at least 10 base pairs in length. In a preferred embodiment, the kit will comprise at least one oligonucleotide selected from the group consisting of SEQ ID NOs:1, 4, 7 and 10, and at least one control oligonucleotide that does not hybridize with a polynucleotide of SEQ ID NOs:1, 4, 7 and 10 under stringent conditions.

PAR-1 may also be used in screens to identify drugs for treatment of cancers which involve over-activity of the encoded protein, or new targets which would be useful in the identification of new drugs.

For all of the preceding embodiments, the clinician will determine, based on the specific condition, whether PAR-1 polypeptides or polynucleotides, antibodies to PAR-1, or small molecules such as peptide analogues or antagonists, will be the most suitable form of treatment. These forms are all within the scope of the invention.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### EXAMPLES

### Example 1

### Isolation Of PAR-1 from Drosophila

Dsh is known to be progressively phosphorylated in Drosophila during the stages of embryonic development known to require Wnt. The stoichiometry of this phosphorylation is high and supports the importance of this reaction. To identify the kinase that phosphorylates Dsh, various regions of Dsh were tested for their ability to physically interact with this kinase. Equal amounts of lysates prepared from staged Drosophila embryos were blotted with anti-Dsh antibody. Only the GST-fusion protein containing the middle (DM), but not the N-terminal or C-terminal domain of Dsh, interacted with this kinase activity from Drosophila embryos. The Dsh- associated kinase activity, which was precipitated with GST-DM, increased as Dsh became progressively phosphoylated during development.

To clone the Dsh-associated kinase, Dsh was immunoprecipitated from Drosophila embryos with preimmune and affinity-purified immune serum, and subjected to an in vitro kinase assay or blotted with anti-Dsh serum. Dsh immunoprecipitates were washed with 700 mM NaCl to elute the associated kinase. The eluted kinase was recovered after dilution by precipitation with GST-fusion proteins. The kinase was purified 60,000-fold from Drosophila embryos and several peptide sequences were then used to clone the cDNA of this kinase. After screening a Drosophila embryo cDNA library with oligo probes designed based on peptide sequences, the cDNA that encodes the kinase was cloned. The cDNA clones encode a protein kinase that is homologous to the C. elegans protein PAR-1 (85% identity in the kinase domain and 42% identity overall). This indicates that the Dsh-associated kinase is a PAR-1 homolog and was designated dPAR-1. Importantly, the kinase activity that was precipitated using GST-DM increased as Dsh became progressively phosphorylated during development. The kinase activity was also present in Dsh immunoprecipitates.

To demonstrate an association of Dsh with this kinase in vivo, endogenous Dsh was immunoprecipitated from either embryos or cells and analyzed by an in-gel in vitro kinase assay. Immunoprecipitated Dsh was phosphorylated in this in-gel kinase assay, indicating the presence of an associated kinase. The binding properties were the same for the kinase activity eluted from immunoprecipitated endogenous Dsh and that precipitated directly from crude lysate by GST-fusion proteins, indicating again that these two assays measured the same kinase. The in-gel kinase experiment showed that the kinase activity was associated with two major bands and a minor band on a polyacrylamide gel as these bands phosphorylated the Dsh substrate impregnated in the gel. These bands of 110 kDa, 64 kDa and 130 kDa (minor band) were ultimately shown to be the kinase and its major fragment. The region of Dsh that interacted with the kinase was mapped more precisely to a 36 amino acid segment, DM5, that is N-terminal to the PDZ domain, and is provided below:

QRLQVRKKPQRRKKRAPSMSRTSSYSSITDSTMSLN (SEQ ID NO:22). This region in Dsh is well conserved among Drosophila, C. elegans, Xenopus and mammals.

To perform the kinase assay, the embryos or cells were lysed in lysis buffer (50 mM HEPES [pH 7.6], 100 mM NaCl, 0.5% Nonidet P-40, 10 mM NaF, 5 mM NaPPi, 1.5 mM MgCl₂, 1 mM EGTA, 10% glycerol, and protease inhibitors). Cleared cell lysates were incubated with GST fusion proteins immobilized on glutathione agarose beads for two to four hours at 4° C. The glutathione beads were washed three times with lysis buffer and once with kinase buffer (50 mM Tris [pH7.6], 10 mM MgCl₂). The kinase reactions were carried out at room temperature for 30 minutes in a reaction volume of 20 μl containing 5 μl GST beads, 1 μl [γ-³²P]ATP (10 μCi at 5000 Ci/mmol, Amersham) and 14 μl kinase buffer. 20 μl 1×SDS sample buffer was added to stop reactions and samples were heated for 5 minutes at 100° C. The samples were subjected to 10% SDS-PAGE and transferred to nitrocellulose membranes. The membranes were exposed to X-ray films.

To demonstrate that dPAR-1 and Dsh form a complex in vivo, endogenous Dsh was immunoprecipitated from the Drosophila wing imaginal disc cell line Clone-8 cells with an affinity purified Dsh antibody and dPAR-1 was detected in the immunocomplex by Western blot using a PAR-1 antibody. The cells were lysed as described above. This confirms the observation that PAR-1 activity was physically associated with Dsh in Drosophila embryos and cells.

### Example 2

### Isolation of Human PAR-1

In searching the databases for the human homolog(s) of dPAR-1, two uncharacterized putative kinases, p78 and EMK, were found with significant homology to dPAR-1. A human expressed sequence tag sequence with homology to dPAR-1 was also identified and the entire coding region of this gene was cloned. The three human dPAR-1 homologs were designated hPAR-1A (p78), hPAR-1Bα,1-1Bβ (EMK) and hPAR-1C. The hPAR-1Bα and hPAR-1Bβ are isoforms that differ from each other in that hPAR-1Bβ does not have the N-terminal region. Each was PCR amplified from cDNAs of human fetal and adult brain. They were cloned into pcDNA3.1 (Invitrogen) with a Myc tag added at C-terminus. All three hPAR-1 were found to be widely expressed in various tissues, including brain, fetal brain, colon, prostate, breast, ovary, and testis.

The sequences for the human (h) and Drosophila (d) PAR-1 forms are provided in the Sequence Listing as follows:

| | |
|---|---|
| SEQ ID NO:1 and 2: | hPAR-1A DNA sequence |
| SEQ ID NO:3: | hPAR-1A amino acid sequence |
| SEQ ID NO:4 and 5: | hPAR-1Bα DNA sequence |
| SEQ ID NO:6: | hPAR-1Bα amino acid sequence |
| SEQ ID NO:7 and 8: | hPAR-1Bβ DNA sequence |
| SEQ ID NO:9 | hPAR-1Bβ amino acid sequence |
| SEQ ID NO:10 and 11: | hPAR-1C DNA sequence |
| SEQ ID NO:12 | hPAR-1C amino acid sequence |
| SEQ ID NO:19 and 20: | dPAR-1 DNA sequence |
| SEQ ID NO:21 | dPAR-1 amino acid sequence. |

It was found that hPAR-1A, hPAR-1Bα, −1Bβ and hPAR-1C have domain regions identified in dPAR-1. There is a kinase domain, a UBA domain, and an ELKL box. The percent homology and similarity for the various regions, compared to dPAR-1, is shown in Table 1, with % homology/% similarity in each column.

TABLE 1

| Protein | Kinase Domain | UBA | ELKL Box | Overall |
|---|---|---|---|---|
| PAR-1A | 88%/93% | 56%/87% | 75%/89% | 47%/53% |
| PAR-1Bα, 1Bβ | 87%/93% | 58%/76% | 67%/83% | 52%/57% |
| PAR-1C | 87%/92% | 53%/82% | 77%/88% | 52%/57% |

### Example 3

### phosphorylation of DVL in Cells Stimulated with WNT

The mammalian homolog of Dsh is Dvl. To determine if Dvl is phosphorylated in cells stimulated by Wnt, Chinese hamster ovary (CHO) cells were transfected with Wnt1 cDNA. The kinase assay was conducted as described in Example 1. CHO cells left unstimulated by Wnt1 served as the negative control. Twenty hours following transfection, cells were lysed as described in Example 1. Cell lysates were separated on a 10% SD S-PAGE gel and transferred to nitrocellulose membranes. The membranes were blotted with monoclonal antibodies to Dvl-1,-2, and −3 (Santa Cruz Biotech) or with an antibody to tubulin (Sigma). The membranes were exposed to autoradiography. CHO cells transfected with Wnt led to the phosphorylation of Dvl as shown by retarded mobility of Dvl proteins on the SD S-PAGE gel and increased mobility of Dvl proteins when treated with phosphatase. These results indicate that Dvl is phosphorylated in cells stimulated with Wnt1.

### Example 4

### Modulation of WNT Signaling by PAR-1

To investigate if PAR-1 is involved in Wnt signaling, PAR-1 activity was examined in the Drosophila wing imaginal disc cell line Clone-8 cells after stimulation with conditioned medium containing Wingless (Wg), a Drosophila homolog of Wnt. Stimulation of the cells confirmed that Dsh became phosphorylated, and Armadillo (Arm), a Drosophila homolog of β-catenin, was stabilized. The kinase activity of PAR-1 was also measured under the same conditions. Multiple experiments showed that although dPAR-1 specific activity increased in cells treated with soluble Wg, there was no change in the amount of PAR-1 protein that interacted with GST-DM5. Thus, treatment of cells with Wg increased the specific activity of PAR-1. The increased PAR-1 activity correlated with enhanced Dsh phosphorylation and increased Arm levels in Clone-8 cells. These experiments also indicate that the kinase activity detected is specific to PAR-1, since an anti-dPAR-1 antibody treatment of lysates before the assay depleted the kinase activity of Clone-8 cell lysates.

Regulation of the Wnt pathway was also investigated using a β-catenin regulated transcription assay (CRT/LEF1 assay). To perform this assay, Chinese hamster ovary (CHO) cells were seeded into 12-well plates the day before transfection. CHO cells were used because of good expression from transfected DNA and these cells have a well-characterized response to Wnt. Duplicate transfections with luciferase reporters and PAR-1 cDNAs (test CDNA) were carried out using Superfect (Qiagen). 24-26 hours after transfection, the cells were lysed and luciferase activities were measured using a dual luciferase assay system (Promega). The LEFI reporter's luciferase activities were normalized by Renilla luciferase activities for transfection efficiency. Fold stimulation was obtained by comparing with vector alone.

All three PAR-1 strongly potentiated Wnt1 or mouse Dvl-3 mediated CRT activation in mammalian (CHO) cells. Mouse Dvl-3 (Tsang 96) was PCR amplified and cloned into pcDNA3.1 with a Flag tag added at C-terminus. Expression of an unrelated protein, such as GST, had no effect on Wnt1-induced CRT in mammalian cells. PAR-1 did not activate the CRT on its own, but instead required coexpression of Wnt or Dvl to activate CRT. This indicates that interactions with components of Wnt signaling are required for PAR-1 function. It was found that PAR-1 did not affect CRT induced by overexpression of β-catenin in cells.

As disclosed earlier, Dsh also participates in the planar polarity pathway to activate JNK. The above-potentiated activation was suppressed by Axin, and was dependent on LEF1. Axin functions to negatively regulate the Wnt signaling pathway and to positively regulate the JNK MAPK pathway. LEF1 is a downstream transcription factor required for CRT activation in the Wnt pathway. In addition, PAR-1 also diminished the Dvl-3-mediated JNK activation.

### Example 5

### Mutant PAR-1 Blocks Phosphorylation of DSH/DVL in Cells Stimulated with WNT

To determine whether PAR-1 is required for the Wnt pathway in mammalian cells, kinase-negative PAR-1 (PAR-1 KN) was expressed to suppress endogenous PAR-1 activity in mammalian (CHO) cells to examine if it could block Wnt signaling. The kinase mutants were generated by converting the conserved lysine to alanine at the ATP binding site in the kinase domain of PAR-1A, -1 B or -1C, because this mutant form of a kinase is often dominant negative.

Chinese hamster ovary (CHO) cells were transfected with cDNAs for Wnt1 and for kinase-negative (KN) form of PAR-1A, -1B or -1C or GST. The three hallmarks of Wnt activity, Dsh/Dvl phosphorylation, β-catenin stabilization and transcriptional activation, were measured. Twenty hours following transfection, cells were lysed as described in Example 1. Expression of the kinase-negative (KN) form of PAR-1A, -1B or -1C strongly suppressed Wnt signaling (up to 95%) in a dose-dependent manner, whereas the same amount of GST had no effect. These results indicate that the kinase mutants are dominant negative forms of the kinase.

CHO cells transfected with Wnt and GST lead to the phosphorylation of Dvl as shown by retarded mobility of Dvl proteins on the SDS-PAGE gel and increased mobility of Dvl proteins when treated with phosphatase. However, CHO cells transfected with dominant negative (KN) form of PAR-1A, -1B or -1C and Wnt1 suppressed phosphorylation of Dvl by Wnt1 as shown by the reduced amount of a retarded Dvl band. This result is consistent with the data showing that PAR-1 phosphorylates Dsh in vitro and in cells.

Further it was determined that both human and Drosophila PAR-1 KN strongly suppressed Wnt-induced β-catenin stabilization. However, neither was able to inhibit the gene response mediated by overexpression of β-catenin. In addition, overexpression of a peptide from Dvl-3 consisting of the PAR-1-binding region of Dsh in CHO cells inhibited the ability of Wnt1 to activate CRT, whereas it had no effect on β-catenin-induced CRT activation. These results indicate that PAR-1 regulates Wnt signaling in a step upstream of P-catenin consistent with the finding that PAR-1 interacts with and phosphorylates Dsh/Dvl.

To test the specificity of the effects of PAR-1 KN, the HPAR KN (PAR-1Bα KN) was coexpressed with wild type hPAR (PAR-1Bα) in the CHO cells. The coexpression completely blocked the inhibitory effects of hPAR KN. These results support the conclusion that the kinase negative PAR-1 affects Wnt signaling by specifically interfering with endogenous PAR-1 activity in the cells.

As disclosed in Example 4, over-expression of PAR-1 diminished the Dvl-3-mediated JNK activation. It was further determined that this inhibitory effect was dependent on the kinase activity of PAR-1 since co-expression of a dominant negative HPAR-1 KN lead to the loss of inhibitory effect on JNK activation. These data indicate that PAR-1 promotes Dsh/Dvl function in the Wnt/β-catenin pathway but suppresses Dsh function in the JNK pathway, thereby acting as a switch.

### Example 6

### PAR-1 Antisense Oligonucleotides Suppress Wnt Responses in Human Cells

Antisense oligonucleotides were used to reduce endogenous PAR-1 protein levels in human cell line HT1080, which expresses all three PAR-1 forms. HT1080 cells were used because antisense oligonucleotides can be delivered to these cells with relative ease and also HT1080 cells have a very robust transcriptional response to Wnt. The antisense or control oligonucleotides (final concentration of 100-200 nm) were transfected into HT1080 cells using cationic peptoid reagents as described in Murphy et al, P.N.A.S. USA 95,1517 (1998). The cells were lysed 44 hours later and blotted with anti-PAR-12 antibody. The oligonucleotides used were as follows:

| | | | |
|---|---|---|---|
| PAR-1A: | antisense | (5′-CGTATGGAGGACTGCCACAAAACGT-3′) | (SEQ ID NO:13) and |
| | control | (5′-TGCAAAACACCGTCAGGAGGTATGC-3′ | (SEQ ID NO:14); |
| PAR-1B: | antisense | (5′-TGAGGTCTGAGCGTCTCCACTCGG-3′) | (SEQ ID NO:15) and |
| | control | (5′-GGCTCACCTCTGCGAGTCTGGAGT-3′) | (SEQ ID NO:16); and |
| PAR-1C: | antisense | (5′-GAGAATGACGCCCAGACTCCACACA-3′) | (SEQ ID NO:17) and |
| | control | (5′-ACACACCTCAGACCCGCAGTAAGAG-3′) | (SEQ ID NO:18). |

The antisense oligonucleotides specifically reduced these target messenger by 75-90% but control oligonucleotides had no effect. The antisense oligonucleotides also significantly reduced endogenous PAR-1 protein levels but the control oligonucleotides had no effect on them. A cellular protein unrelated to PAR-1, tubulin, was not affected by the antisense oligonucleotides.

HT1080 cells were also transfected with an individual PAR-1 antisense oligonucleotide as indicated above and cells were lysed about 30 hours later. PAR-1 activity was measured by precipitation with GST fusion protein containing the 36 amino acid residues PAR-1binding fragment from Dv13 and followed by an in vitro kinase assay. Each PAR-1 antisense oligonucleotide reduced PAR-1 kinase activity in cells, supporting the observation that they all interact and phosphorylate Dvl. Knocking out individual PAR-1 by single antisense oligonucleotide resulted in a 25-40% reduction of Wnt signaling in the cells but the control oligonucleotides had minimal effects on Wnt signaling.

Cells in duplicate were first transfected with oligonucleotides, 24 hours later cells were transfected with Wnt1 and LEF 1 reporter. The reporter activities were measured 24 hours later after that. The CRT activation was obtained as described in Example 4. Simultaneously knocking out two PAR-1 with two antisense oligos resulted in further reduction, up to 60%, in Wnt response, indicating the existence of synergy among three endogenous PAR-1 in the Wnt pathway. The partial inhibition of Wnt response after antisense treatment also indicated that these three PAR-1 play a redundant role in Wnt signaling in the cells.

### Example 7

### Suppression of PAR-1 Inhibits Wnt Signaling in Xenopus

The role of PAR-1 in Wnt signaling in vertebrates was examined by injecting PAR-1 mRNA into Xenopus embryos. For Xenopus RNA injection, PAR-1Bα WT, PAR-1Bα KN, PAR-1A KN and green fluorescent protein (GFP) were each cloned into the pCS2 vector. mRNAs were synthesized by using a mMESSAGE mMACHINE kit (Ambion).

As shown in Table 2, the RNAs as indicated were injected into the ventral side of four-cell stage blastomeres. The injected embryos were scored for axis duplication at 72 hours. To evaluate suppression effect of dominant negative PAR-1, injected embryos were scored as no duplication (0), partial duplication (1), or second axis with a head and cement gland (2) as described in Peters et al., Nature, 401, 345 (1999). Ventral blastomere injection into 4-cell embryos of XWnt8 RNA (1 pg or 1.2 pg) resulted in significant axis duplication. Co-injection of human RNA of the dominant-negative PAR-1Bα (PAR-1Bα KN) or Drosophila RNA of the dominant-negative dPAR-1 KN (0.2ng or 1.0 ng) significantly inhibited XWnt8-induced axis duplication in injected embryos, but co-expression of a similar dose of green fluorescent protein (GFP)-RNA had no effect. This inhibition was partially rescued by co-expression of wild-type PAR-1 Bα or dPAR-1, respectively. At the same dose, PAR-1Bα KN alone had no effect on development when injected into the ventral side of embryos.

TABLE 2

| | | Mean Axis |
|---|---|---|
| Sample | N | Duplication |
| H₂O | 65 | 0.0 |
| Xwnt8 (1.2 pg) | 62 | 1.5 |
| Xwnt8 + PAR-1Bα KN (0.2 ng) | 61 | 0.9 |
| Xwnt8 + PAR-1Bα KN + PAR-1Bα WT (0.2 ng) | 64 | 1.2 |
| PAR-1Bα KN | 62 | 0.0 |
| PAR-1Bα WT | 65 | 0.0 |
| H₂O | 80 | 0.0 |
| Xwnt8 (1.0 pg) | 82 | 1.6 |
| Xwnt8 + PAR-1A KN (0.2 ng) | 85 | 0.7 |
| Xwnt8 + PAR-1A KN (1.0 ng) | 89 | 0.6 |
| PAR-1A KN (1.0 ng) | 82 | 0.0 |

### Example 8

### PAR-1 Antisense Suppresses Cancer Cell Foci Formation

To characterize hPAR-1's role in tumorigenicity, hPAR-1s were tested for their function in a soft agar (anchorage-independent) assay. The cells used in this assay were human colon cancer SW620 cells, which carry elevated levels of β-catenin protein due to a mutation in the tumor suppressor APC gene. SW620 cells were treated with the hPAR-1A or hPAR1-C antisense oligonucleotides or reverse control oligonucleotides of Example 6 and seeded in growth medium containing 0.3% agar in dishes. Colonies formed in one to two weeks. Fields of colonies were counted by eye. It was found that treatment of the cells with the HPAR-1A or HPAR-1-C antisense oligonucleotides significantly reduced the number of colonies as compared to treatment with the reverse control oligonucleotides. These results indicate that PAR-1 is involved in maintaining a cancer phenotype.

From the foregoing it will be appreciated that, although specific embodiments of the invention have been described herein for purposes of illustration, various modifications may be made without departing from the spirit and scope of the invention. Accordingly, the invention is not limited except as by the appended claims.

What is claimed is:

1. An isolated nucleic acid molecule comprising a polynucleotide having a sequence selected from the group consisting of:
(a) a sequence encoding amino acids from about 1 to about 744 of SEQ ID NO:3;
(b) a sequence encoding amino acids from about 2 to about 744 of SEQ ID NO:3;
(c) a sequence encoding amino acids from about 1 to about 691 of SEQ ID NO:6;
(d) a sequence encoding amino acids from about 2 to about 691 of SEQ ID NO:6;
(e) a sequence encoding amino acids from about 1 to about 724 of SEQ ID NO:9;
(f) a sequence encoding amino acids from about 2 to about 724 of SEQ ID NO:9;
(g) a sequence encoding amino acids from about 1 to about 795 of SEQ ID NO:12;
(h) a sequence encoding amino acids from about 2 to about 795 of SEQ ID NO:12;
(i) complements of the sequences of (a)-(h);
(j) a sequence having 50-2232 contiguous nucleotides from the coding region of SEQ ID NO:1;
(k) a sequence having 50-2073 contiguous nucleotides from the coding region of SEQ ID NO:4;
(l) a sequence having 50-2172 contiguous nucleotides from the coding region of SEQ ID NO:7;
(m) a sequence having 50-2385 contiguous nucleotides from the coding region of SEQ ID NO:10;
(n) sequences having at least 90% identity to the sequences of (a)-(m);
(o) sequences having 100-1500 contiguous nucleotides from the coding region of SEQ ID NO:1, SEQ ID NO:4, SEQ ID NO:7 or SEQ ID NO:10;
(p) sequences having 500-1000 contiguous nucleotides from the coding region of SEQ ID NO:1, SEQ ID NO:4, SEQ ID NO:7 or SEQ ID NO:10;
(r) sequences of (a)-(h), except for at least one amino acid substitution in the encoded amino acid sequence; and
(s) sequences of (a)-(h), expect for a conversion of a conserved lysine to an alanine at an ATP binding site of the encoded amino acid sequence.

2. A method of making a vector comprising inserting a nucleic acid molecule of claim 1 into said vector in operable linkage to a promoter.

3. A vector produced by the method of claim 2.

4. A method of making a host cell comprising transforming or transfecting a vector of claim 3 into a cell.

5. A host cell produced by the method of claim 4.

6. A method of making a polypeptide, comprising culturing the host cell of claim 5 under conditions such that said polypeptide is expressed and recovering said polypeptide.

7. An isolated polypeptide comprising an amino acid sequence selected from the group consisting of:
(a) sequences having at least 95%
identity to an amino acid sequence of:

| | |
|---|---|
| (i) | amino acids from about 1 to about 744 of SEQ ID NO:3, |
| (ii) | amino acids from about 2 to about 744 of SEQ ID NO:3, |
| (iii) | amino acids from about 1 to about 691 of SEQ ID NO:6, |
| (iv) | amino acids from about 2 to about 691 of SEQ ID NO:6, |
| (v) | amino acids from about 1 to about 724 of SEQ ID NO:9, |
| (vi) | amino acids from about 2 to about 724 of SEQ ID NO:9, |
| (vii) | amino acids from about 1 to about 795 of SEQ ID NO:12, or |
| (viii) | amino acids from about 2 to about 795 of SEQ ID NO:12; |

(b) sequences having, expect for at least one amino acid substitution, an amino acid sequence of: (i)-(viii);
(c) sequences having, expect for at least one amino acid substitution, an amino acid sequence of: (i)-(viii); and
(d) sequences having, expect for a conversion of a conserved lysine to an alanine at the ATP binding site of said polypeptide, an amino acid sequence of: (i)-(viii).

8. An epitope-bearing portion of a polypeptide selected from the group consisting of SEQ ID NO:3, SEQ ID NO:6, SEQ ID NO:9 and SEQ ID NO:12.

9. The epitope-bearing portion of claim 8, which comprises about 5 to about 50 contiguous amino acids.

10. An isolated antibody that binds to the polypeptide of claim 7.

11. A complex comprising a polypeptide of claim 7 and a Dishevelled protein.

12. A complex comprising a fragment of a polypeptide of claim 7 and a Dishevelled protein.

13. A method of identifying an inhibitor or enhancer of PAR-1phosphorylation activity, comprising:
(a) contacting a cell transfected with at least an expression vector encoding Wnt with a candidate inhibitor or enhancer; and
(b) detecting an increase or decrease in Dsh phosphorylation,
wherein a decrease in Dsh phosphorylation indicates the presence of an inhibitor and an increase in Dsh phosphorylation indicates the presence of an enhancer.

14. An isolated PAR-1 modulator selected from the group consisting of an antisense oligonucleotide, a ribozyme, a protein, a polypeptide, and a small molecule.

15. The isolated PAR-1 modulator of claim 14, wherein said PAR-1modulator is an antisense molecule or the complement thereof.

16. The isolated PAR-1 modulator of claim 15, wherein said antisense molecule or the complement thereof has at least 15 consecutive nucleic acids of the sequence of SEQ ID NO:3, SEQ ID NO:6, SEQ ID NO:9 or SEQ ID NO:12 or which hybridizes under high stringency conditions to said at least 15 consecutive nucleic acids of the sequence of SEQ ID NO:3, SEQ ID NO:6, SEQ ID NO:9 or SEQ ID NO:12.

17. The isolated PAR-1 modulator of claim 15, wherein said antisense molecule is selected from the group consisting of SEQ ID NO:13, SEQ ID NO:15 and SEQ ID NO:17.

18. The isolated PAR-1 modulator of claim 14, wherein said PAR-1 modulator is selected from the group consisting of an antibody and an antibody fragment.

19. The isolated PAR-1 modulator of claim 14, wherein said polypeptide has an amino sequence with at least 95% identity to the amino acid sequence provided in SEQ ID NO:22.

20. A composition, comprising a therapeutically effective amount of a PAR-1modulator of claim 14 in a pharmaceutically acceptable carrier.

21. A method of treating a mammal with a disease or disorder associated with a PAR-1 polypeptide, comprising administering to the mammal a composition including a therapeutically effective amount of a PAR-1 modulator of claim 14.

22. The method of claim 23, wherein said PAR-1 modulator is an antisense molecule is selected from the group consisting of SEQ ID NO:13, SEQ ID NO:15 and SEQ ID NO:17.

23. The method of claim 21, wherein said PAR-1 modulator is a polypeptide that has an amino sequence with at least 95% identity to the amino acid sequence provided in SEQ ID NO:22.

24. The method of claim 21, wherein said PAR-1 modulator is selected from the group consisting of an antibody and an antibody fragment.

25. The method of claim 21, wherein said PAR-1 modulator is administered ex vivo to said mammalian cell.
